SEQUENCE LISTING (77 sequences)

SEQ ID NO: 1 - protein, 15 aa, synthetic construct
GGGGSGGGGSGGGGS

SEQ ID NO: 2 - protein, 19 aa, synthetic construct
GGGGSGGGGSGGGSGGGGS

SEQ ID NO: 3 - protein, 24 aa, synthetic construct
GGGGSGGGGSGGGGSGGGSGGGGS

SEQ ID NO: 4 - protein, 29 aa, synthetic construct
GGGGSGGGGSGGGGSGGGGSGGGSGGGGS

SEQ ID NO: 5 - protein, 5 aa, synthetic construct
GGGGS

SEQ ID NO: 6 - protein, 10 aa, synthetic construct
GGGGSGGGGS

SEQ ID NO: 7 - protein, 140 aa, Homo sapiens
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 8 - DNA, 420 bp, Homo sapiens
  source 1..420: mol_type = genomic DNA
atccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 9 - protein, 143 aa, Homo sapiens
IPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 10 - DNA, 429 bp, Homo sapiens
  source 1..429: mol_type = genomic DNA
attcccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 11 - DNA, 4627 bp, Homo sapiens
  source 1..4627: mol_type = genomic DNA
atatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacagtattctaaggacgccagaaagctgttgatcggcttcaagcaggggagggacacctaatttgcttttcttttttttttttttttttttttttttttttgagatggagttttgctcttgttgcccaggctggagtgcaatggtgcatcttggctcactgcaacctccgcctcccaggttcaagtgattctcctgcctcagcctcccgagtagctgagattacaggcacccgccaccatgcctggctaattttttgtatttttagtagagacagggtttcactatgttggccaggctggtctcgaactcctgacctcaggtgatccacccgcttcagcctcccaaagtgctgggattacaggcgtgagccaccacacccggcctgcttttcttaaagatcaatctgagtgctgtacggagagtgggttgtaagccaagagtagaagcagaaagggagcagttgcagcagagagatgatggaggcctgggcagggtggtggcagggaggtaaccaacaccattcaggtttcaaaggtagaaccatgcagggatgagaaagcaaagaggggatcaaggaaggcagctggattttggcctgagcagctgagtcaatgatagtgccgtttactaagaagaaaccaaggaaaaaatttggggtgcagggatcaaaactttttggaacatatgaaagtacgtgtttatactctttatggcccttgtcactatgtatgcctcgctgcctccattggactctagaatgaagccaggcaagagcagggtctatgtgtgatggcacatgtggccagggtcatgcaacatgtactttgtacaaacagtgtatattgagtaaatagaaatggtgtccaggagccgaggtatcggtcctgccagggccaggggctctccctagcaggtgctcatatgctgtaagttccctccagatctctccacaaggaggcatggaaaggctgtagttgttcacctgcccaagaactaggaggtctggggtgggagagtcagcctgctctggatgctgaaagaatgtctgtttttccttttagaaagttcctgtgatgtcaagctggtcgagaaaagctttgaaacaggtaagacaggggtctagcctgggtttgcacaggattgcggaagtgatgaacccgcaataaccctgcctggatgagggagtgggaagaaattagtagatgtgggaatgaatgatgaggaatggaaacagcggttcaagacctgcccagagctgggtggggtctctcctgaatccctctcaccatctctgactttccattctaagcactttgaggatgagtttctagcttcaatagaccaaggactctctcctaggcctctgtattcctttcaacagctccactgtcaagagagccagagagagcttctgggtggcccagctgtgaaatttctgagtcccttagggatagccctaaacgaaccagatcatcctgaggacagccaagaggttttgccttctttcaagacaagcaacagtactcacataggctgtgggcaatggtcctgtctctcaagaatcccctgccactcctcacacccaccctgggcccatattcatttccatttgagttgttcttattgagtcatccttcctgtggtagcggaactcactaaggggcccatctggacccgaggtattgtgatgataaattctgagcacctaccccatccccagaagggctcagaaataaaataagagccaagtctagtcggtgtttcctgtcttgaaacacaatactgttggccctggaagaatgcacagaatctgtttgtaaggggatatgcacagaagctgcaagggacaggaggtgcaggagctgcaggcctcccccacccagcctgctctgccttggggaaaaccgtgggtgtgtcctgcaggccatgcaggcctgggacatgcaagcccataaccgctgtggcctcttggttttacagatacgaacctaaactttcaaaacctgtcagtgattgggttccgaatcctcctcctgaaagtggccgggtttaatctgctcatgacgctgcggctgtggtccagctgaggtgaggggccttgaagctgggagtggggtttagggacgcgggtctctgggtgcatcctaagctctgagagcaaacctccctgcagggtcttgcttttaagtccaaagcctgagcccaccaaactctcctacttcttcctgttacaaattcctcttgtgcaataataatggcctgaaacgctgtaaaatatcctcatttcagccgcctcagttgcacttctcccctatgaggtaggaagaacagttgtttagaaacgaagaaactgaggccccacagctaatgagtggaggaagagagacacttgtgtacaccacatgccttgtgttgtacttctctcaccgtgtaacctcctcatgtcctctctccccagtacggctctcttagctcagtagaaagaagacattacactcatattacaccccaatcctggctagagtctccgcaccctcctcccccagggtccccagtcgtcttgctgacaactgcatcctgttccatcaccatcaaaaaaaaactccaggctgggtgcgggggctcacacctgtaatcccagcactttgggaggcagaggcaggaggagcacaggagctggagaccagcctgggcaacacagggagaccccgcctctacaaaaagtgaaaaaattaaccaggtgtggtgctgcacacctgtagtcccagctacttaagaggctgagatgggaggatcgcttgagccctggaatgttgaggctacaatgagctgtgattgcgtcactgcactccagcctggaagacaaagcaagatcctgtctcaaataataaaaaaaataagaactccagggtacatttgctcctagaactctaccacatagccccaaacagagccatcaccatcacatccctaacagtcctgggtcttcctcagtgtccagcctgacttctgttcttcctcattccagatctgcaagattgtaagacagcctgtgctccctcgctccttcctctgcattgcccctcttctccctctccaaacagagggaactctcctacccccaaggaggtgaaagctgctaccacctctgtgcccccccggcaatgccaccaactggatcctacccgaatttatgattaagattgctgaagagctgccaaacactgctgccaccccctctgttcccttattgctgcttgtcactgcctgacattcacggcagaggcaaggctgctgcagcctcccctggctgtgcacattccctcctgctccccagagactgcctccgccatcccacagatgatggatcttcagtgggttctcttgggctctaggtcctgcagaatgttgtgaggggtttatttttttttaatagtgttcataaagaaatacatagtattcttcttctcaagacgtggggggaaattatctcattatcgaggccctgctatgctgtgtatctgggcgtgttgtatgtcctgctgccgatgccttc

SEQ ID NO: 12 - protein, 178 aa, Homo sapiens
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 13 - DNA, 534 bp, Homo sapiens
  source 1..534: mol_type = genomic DNA
gatctgaaaaacgtgttccctcctgaagtggctgtctttgaaccatccgaggccgagatttcccatacccagaaagcaactctggtctgtctggccactggattctaccccgatcacgtggaactgtcttggtgggtgaacggcaaggaagtccattccggagtctctaccgaccctcagcccctcaaggagcagcctgctctcaacgattctcggtactgcctgtcatctcgactgagagtgtctgccaccttctggcagaaccctagaaaccactttcggtgtcaggtccagttttacggcctgagcgagaacgatgagtggacacaggatagagccaaacctgtgacacagattgtgagcgccgaggcttggggacgagccgattgtggcttcacatccgagtcttaccagcagggagtgctgtctgctacaatcctctacgaaattctcctggggaaggccaccctgtacgctgtcctcgtgtctgctctggtgctcatggctatggtcaaacgaaaggactctagaggc

SEQ ID NO: 14 - protein, 180 aa, Homo sapiens
LEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 15 - DNA, 540 bp, Homo sapiens
  source 1..540: mol_type = genomic DNA
ctggaggatctgaaaaacgtgttccctcctgaagtggctgtctttgaaccatccgaggccgagatttcccatacccagaaagcaactctggtctgtctggccactggattctaccccgatcacgtggaactgtcttggtgggtgaacggcaaggaagtccattccggagtctctaccgaccctcagcccctcaaggagcagcctgctctcaacgattctcggtactgcctgtcatctcgactgagagtgtctgccaccttctggcagaaccctagaaaccactttcggtgtcaggtccagttttacggcctgagcgagaacgatgagtggacacaggatagagccaaacctgtgacacagattgtgagcgccgaggcttggggacgagccgattgtggcttcacatccgagtcttaccagcagggagtgctgtctgctacaatcctctacgaaattctcctggggaaggccaccctgtacgctgtcctcgtgtctgctctggtgctcatggctatggtcaaacgaaaggactctagaggc

SEQ ID NO: 16 - protein, 175 aa, Homo sapiens
LNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 17 - protein, 176 aa, Homo sapiens
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 18 - DNA, 528 bp, Homo sapiens
  source 1..528: mol_type = genomic DNA
gacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 19 - DNA, 1448 bp, Homo sapiens
  source 1..1448: mol_type = genomic DNA
aggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcaggtgagtggggcctggggagatgcctggaggagattaggtgagaccagctaccagggaaaatggaaagatccaggtagcagacaagactagatccaaaaagaaaggaaccagcgcacaccatgaaggagaattgggcacctgtggttcattcttctcccagattctcagcccaacagagccaagcagctgggtcccctttctatgtggcctgtgtaactctcatctgggtggtgccccccatccccctcagtgctgccacatgccatggattgcaaggacaatgtggctgacatctgcatggcagaagaaaggaggtgctgggctgtcagaggaagctggtctgggcctgggagtctgtgccaactgcaaatctgactttacttttaattgcctatgaaaataaggtctctcatttattttcctctccctgctttctttcagactgtggctttacctcgggtaagtaagcccttccttttcctctccctctctcatggttcttgacctagaaccaaggcatgaagaactcacagacactggagggtggagggtgggagagaccagagctacctgtgcacaggtacccacctgtccttcctccgtgccaacagtgtcctaccagcaaggggtcctgtctgccaccatcctctatgagatcctgctagggaaggccaccctgtatgctgtgctggtcagcgcccttgtgttgatggccatggtaagcaggagggcaggatggggccagcaggctggaggtgacacactgacaccaagcacccagaagtatagagtccctgccaggattggagctgggcagtagggagggaagagatttcattcaggtgcctcagaagataacttgcacctctgtaggatcacagtggaagggtcatgctgggaaggagaagctggagtcaccagaaaacccaatggatgttgtgatgagccttactatttgtgtggtcaatgggccctactactttctctcaatcctcacaactcctggctcttaataacccccaaaactttctcttctgcaggtcaagagaaaggatttctga

SEQ ID NO: 20 - DNA, 1489 bp, Homo sapiens
  source 1..1489: mol_type = genomic DNA
aggacctgaaaaacgtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcaggtgagtggggcctggggagatgcctggaggagattaggtgagaccagctaccagggaaaatggaaagatccaggtagcggacaagactagatccagaagaaagccagagtggacaaggtgggatgatcaaggttcacagggtcagcaaagcacggtgtgcacttcccccaccaagaagcatagaggctgaatggagcacctcaagctcattcttccttcagatcctgacaccttagagctaagctttcaagtctccctgaggaccagccatacagctcagcatctgagtggtgtgcatcccattctcttctggggtcctggtttcctaagatcatagtgaccacttcgctggcactggagcagcatgagggagacagaaccagggctatcaaaggaggctgactttgtactatctgatatgcatgtgtttgtggcctgtgagtctgtgatgtaaggctcaatgtccttacaaagcagcattctctcatccatttttcttcccctgttttctttcagactgtggcttcacctccggtaagtgagtctctcctttttctctctatctttcgccgtctctgctctcgaaccagggcatggagaatccacggacacaggggcgtgagggaggccagagccacctgtgcacaggtgcctacatgctctgttcttgtcaacagagtcttaccagcaaggggtcctgtctgccaccatcctctatgagatcttgctagggaaggccaccttgtatgccgtgctggtcagtgccctcgtgctgatggccatggtaaggaggagggtgggatagggcagatgatgggggcaggggatggaacatcacacatgggcataaaggaatctcagagccagagcacagcctaatatatcctatcacctcaatgaaaccataatgaagccagactggggagaaaatgcagggaatatcacagaatgcatcatgggaggatggagacaaccagcgagccctactcaaattaggcctcagagcccgcctcccctgccctactcctgctgtgccatagcccctgaaaccctgaaaatgttctctcttccacaggtcaagagaaaggattccagaggctag

SEQ ID NO: 21 - protein, 173 aa, Homo sapiens
DKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 22 - protein, 189 aa, Homo sapiens
DKQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTVDPKYNYSKDANDVITMDPKDNWSKDANDTLLLQLTNTSAYYTYLLLLLKSVVYFAIITCCLLRRTAFCCNGEKS

SEQ ID NO: 23 - DNA, 5591 bp, Homo sapiens
  source 1..5591: mol_type = genomic DNA
ataaacaacttgatgcagatgtttcccccaagcccactatttttcttccttcaattgctgaaacaaagctccagaaggctggaacatacctttgtcttcttgagaaatttttccctgatgttattaagatacattggcaagaaaagaagagcaacacgattctgggatcccaggaggggaacaccatgaagactaacgacacatacatgaaatttagctggttaacggtgccagaaaagtcactggacaaagaacacagatgtatcgtcagacatgagaataataaaaacggagttgatcaagaaattatctttcctccaataaagacaggtatgtgtttacgcatatcatctgtcagaacacttctttgaaagtgaatgctgcattttttcctttcagtattaatgaaaaacaaacataaatctttcttaaatattgttacatttaatggtagcataaatgccctgctacttttctatagaattaaaatggtataggttttggagaaaacaaaattgaaaaagttactgaaggtttgtcagcctcagctccattatccaaaataagaaagtcacgtgctggtttttagggttgttagatggattaaagaaacaacatacacagaagcatctagcaacgtgacacgtggtaaacgctcaaaaagtgttctcccttcttttgatgactttacttgatcaggaaataacatatatatgtctttcaggaatgttctgcccaagcaggagagtcactcacctcaatcttgctacccacaaagtttaacctaaaaacaacgggttcattgttgacaaaatgatgtttatctgttgttgacagaatgatgtttatctaaaaacagttccaattttctatttcctttgctgagacacaaaggggaggcaaatgtgcaaagcttgagggtagtcttaccactgtgcttaagtgttctgatttttctagtgatcagggcaaaataaaaagtatagtaagttccaaggcagtgaatattatacaggagagaagttacagttttataatgtgttttcctttacactaaattctaaaagtaaaaagtctttttttttttttgacagagtttcactcttgttgcccaagcaggtgtgctatggtatgatctcagctcactgcaacctccacctcccgggttcaagtgattctcttacttcagcctcccgacaggctgggattgcaggcgcctgccaccacacctggctaatttttgtgtttttagtagagatggggtttcaccatgttggccaggctggtctcaaattcctgacctcaagtgatccatccacctcggcctccaagtgctgggattatgggcgtcagccactgtgcccagcctaaaagtaaaatgtctttcatgagcttcccaaggcagctacgttaaggaggacacttctcttaatgtcattctacagtagatttctaatgctctttcttggaagtttgtttttctgagaaaagctaaaaatataacatggaagtgatcatattatataatcaatgaagtgcttttcaaggagataaaactaatctggtccacacttgcaaccaaccttgattgagagagagagagaactcaggatacacttgaagattttattatggggaacagttactttattctttttacctcaatcaatgcatggaaataagtgatagtcattttcatttatcttttaataaatgaagtcaccatgaggaaaataaaaagacattgaaaacccattaaagtcagcccttaaagatatttggacatgcagacttgataactaacgtttgcattcttgagacttacccaaaacccatacctcaagtccaagtttttagaattcatgaaataaagatctcagtgagtgcataaaattgcgcaccagaatcatatccgtatagacaagaacacatctactagaaaaataataaaccaacacaccaatgcaactgtgttttcttctgttttaaagtatgttgtctttgtatgcatgtttgcttcttcctttttttttttaacatcacagataaattcaactctcacctcaggttttattgagagaactgtcaatgtgacttggcctctgtctttctagtcccagaaagaattgcactgaaatctgagctcctgtaataaaaacaaccatttgctgagagtaattaacatactgaaagagattttcttagagtacacaatggtgacattatattgcctctttataaataactttctatctatttctgtggattattcctacaaagtacttttcatatgtccaatttcttttcttcccctacaactactgtctgaatactggctctgctatttgctgatatgattctcggcaagttgcctgcactttttaaactttatttcctcattcagaacatggggccatacataatacaactcacttcagtgttattggggaattaaacaaaaaatgcatgggaagcatttaacatagtgcctgacacaataatgagtactcagtagatgttagcttttattaatattgttgttgttatgtccagaaacactatacctccagaaaatcatgggtacttgctggggacattggggatatgcatgatttggaaaagaatgactgctttttttgcttagatgagaaatttttctaagccagactccttcaaatatgtaagattctgttgtggattcaaggactgaaagaattcttggccgagtgtggtggcttatccctgtaatcccagcattttgtgaggacaaggcaggaagattgcttgagtccaggagtttgaaaccagcctgcgcaacatggcgaaaccctgtctctacaaaaaatacaaacattagctcggagtgagtgctgacatgtgcctgtactcccagctactcagaaggctgagatgggaggatctcatgagcctggggagtttgaggcttcagtgagccgtgatgacaccgtactatactccactccagcctgggtgacagtgagaccctgcctcaaaaaacaaacaaacaaacaaacaaaacaaaattaatctttttgctgatgtcatgtcagcagtgtgtgttgaaggctgtaaagcagccatttgttcagtttatttttccattgaacaagtatttatcaaaaacatactttgtggcagtcactatgctaggagctatgaatacagaaggaaaagtaaatgctcttggatactacactccagttgtgataaaaaagaaaaaatgtattcttcaccaacttcaacatcttgatgtgcaaaaacataatacatgaattagatctacctaattacacagaattagaccaattgtttctggaattgtgggctcatatttttaataactgtcctcctgcctctctgtcgacaggttttataaatattcatttaattacacacacacacacgaacaattgactagtacttgctctcattcttctagatgtcatcacaatggatcccaaagacaattgttcaaaagatgcaaatggtaagcttttgtgtttttcccttcctcctgatcattttgttttgaacttctctggcttgaaaaatcagggaatggattttgctaggttggatgctgcagaatggacctagtgatattttaaattagtccctcattttctaggagttgtattaacaaacctaactactgctttggggtatgagatgactgtaaattagagagggtacagtggtatagtgatatgcttttaattatttcaaaaaaaagattttattcattcatgtgtcttttttctttttcttttcttttttttttttttttggacagagtcttgctctgtcacccaggctggagtgcggtggcagtatctcagctcaccacaacctccgcctcccggcttcaagtgattctcctgcctcagcttctcgagtagctgggactacaggcgcgtgccaccatgcccggctaatttttgtatttttagtagagttggggtttcaccatgttggccaggatggcctcgaatttgtgacctcgtgatctgccccctcgccctcccgaactgttgggattacaggcgtgagtcactgtgcccggcctcctgtcctgtcttttgtttaatgactgggaaaaacatgataccatgttgcttctcgagttgttttgttttagtctttggtctttgctagtagctaataacacgaactagtgtttatcaagtgctttttacacagaagggcttgggctgtgttctgcattttcttgtttaaccctcttaaaactcctataaaatggtacatatttttctcccaatttacagtccctttaaagcaaataattataaaaatccctatacatgtcacacagctagatctgggatttcaaatcaggccatcaaacaaagagtttatgtacttagtaagttttctgttctttttctacaatagagtcagatagcaagaaattaccaagccaggaacctgaaacaaaacggacatcatgtggggctgggtgggtgcatgggctttgcagactggactttcactccagctcttttaatgattaggtgtaagtgacctacattttgtgagcaacagttttctcatcagccaacaaagaataattacaccagattcacagttattgaagagataaaggcatgaatgtgagatgtctggcatagggcatctcatttagcagacacagaatgagtacttgtttctggctttttctctctacatatgcacaaagaatgcgactagaagcatgggctctagccctgctcaactttcctctatttccaataccaaggggctctgacttaggctgccacaccaggcaaggagggcagtaccacctcacttgaccaagggcagggagtcacggacacatcacttcttgagatccttttccacaccaaggactgatgtttctggaattctcactttatgaagacaaaacatataaatggaaattttctcaggtagagactcactcttgtagctcattgagtaggcactagtggtccacccccactgtctttacttattccttgacatcacatatctcttgcaaaacctcaaataatattaaatgcaatcacccaataatagcatagccataattagaggcatttaggaaagacaggtgagtgtgccacaactacctaacacatcagcaaatctggattaaccactttctttgattttccacaatgcaaccttactttttaatagttgggaatgttctaagtgaatttagcagaggttgttaatcaacttgaaagctgaattctgacttgtctgactcttggtggtgctggtagcagtagatgtttacttttaggttttggtggtggtggaatatcacttcaacgtaaatcatcagaaataagtatttgtgaacccctctcgcattaatgtatcttattctgtaaaaagaacatgtgcaatttctcttagatacactactgctgcagctcacaaacacctctgcatattacatgtacctcctcctgctcctcaagagtgtggtctattttgccatcatcacctgctgtctgcttagaagaacggctttctgctgcaatggagagaaatcataa

SEQ ID NO: 24 - DNA, 9549 bp, Homo sapiens
  source 1..9549: mol_type = genomic DNA
ataaacaacttgatgcagatgtttcccccaagcccactatttttcttccttcgattgctgaaacaaaactccagaaggctggaacatacctttgtcttcttgagaaatttttcccagatattattaagatacattggcaagaaaagaagagcaacacgattctgggatcccaggaggggaacaccatgaagactaacgacacatacatgaaatttagctggttaacggtgccagaagagtcactggacaaagaacacagatgtatcgtcagacatgagaataataaaaacggaattgatcaagaaattatctttcctccaataaagacaggtatgtgtttacacatatcatctgtcagaacacttctttgaaagtgaatgctgcattttttcctttcagtattaatgaaaaacataaatctttcttaaaaattgttacatttaatggtagcgtaaatgccctgctacttttctatagaattaaaatggtataggttttggagaaaacaaaattgaaaaagttgctgaaggtttgtcagcctcagctccattatccaaaataagaaagtcacgtgctggtttttagggttgttagatggattaaagaaacaacatacacagaagcatctagcaacgtgacacgtggtaaacgctcaaaaagtgttctcccttcttttgatgactttacttgatcaggaaataacatatatatgtctttcaggaatgttctgcccaagcaggagagtcactcacctcaatcttgctacccacaaagtttaacctaaaaacaacgggttcattgttgacaaaataatgtttatctgaagataactgtagatcatatttatctgtagataatgtttatctgtggagtgtggctctacaaaacatagaatagtcttggtcactgcagttttatagaggccttgggtttttcagagtttcattttatatatcaccataaagtaacatttcataattacaggttggtaaggcttacatgtacaaacattcttccattttccataataaatgcatttcctgccattggtgaatgcagctcaataaacatttattgtacaattatgacacgccaggcttagtggaaatgtggatgaacagacaaggatgagttactgtcctaaggatgatgcatgacagtgcagagaatatactctcttcctgatcactcagggtcactcatgattcatgcgcgaggtcccaaaacagtgcctttgatgcagattctgtacatctctagacgattggtccaagggctgaatgtgctctggcccagtggtccagtctgtcactatatgtcaacatcctgaatatgaacataacagtccaacatctcaagagtgggcatgaaaaggactcattttgtgctttttcctgtggttaacaagtcctttttagcctgggggaacaagcattaacaaaatgtttgaagatctttgccacgtaccattccaaatttctagggtaagtctttagcttttcagatcctgagtttctgcaatgatcaaatgtgatttggacagttgcgttgactttctcctggggctataatggagtgcaaaggaaacaatggcagggaaaatgcttgctttcaaaatggtagcatggatgtgttcattcgtgtagttactgtattaggtatagcctttcctgaaactaactgaagtggggttataaaaacagtcccaattttctatttcctttgctgagacacaaagaggagacaaaagagcaaagcttgagggtagttttaccactgtgcttaagtgttctgatttttccagtgatcagggtgaaataaaaagcatagtaagttccagggcagtgaataccatacaggagacaagttacagttttataatgtgttttactttacactaaattctaaaagtaaaatgtctttttttttttccgagacagagtttcactcttgtagcccaggcaggagtgctatggtgtgatctcggctcacagcaacctccacctcccagtttcaagcgattcttctgcctcagcctcccgagaagttgaaattacaggtgcctggcaccatatctcgctaattattctatttttagtagagatcgggttttaccatgttggccaggctggtctcgaactcctgacttcaagtgatccacccgcctcagcctcccaaagtgctgggattacaggtgtgagtcactgtgccggacctaacagtaaaatgtctttcatgtgcttctcaaggcaactacattaaggaggacacatctcttaatgtcattctacagtagatttctaatgctctttcttggaagtttgtttttctgagaagagctaaaaatataataacatggaagtgatcatattatataatcaatgaagtgctttcaaaggagataaaactaacctggtctgcatttgcaaccagccttgattgagagagagagaactcaggatacacttagagattttattatggggaatagttactttattcattttacctcaatcaatgcatggaaataagtgacagtcattttcatttatcttttaataaataaagtcaccatgaggaaaatgaaaacccattaaagtcagtccttaaagatatttggacatgcagacatgataactaacatttccattcgtgagacttacccaaaacctatacctcaagtccatttcttagaatacatgaaataaagatctcagtgagtgtataaaactgcacaccagaatcatatccgtatagacaagaatacatctactagaaaaatataaaccaaaacaccaaggtgactctgtttttttctgttttaaaatatgttgtctttgtatgcatgtttgcttcttcctttttttttttaaacatcgcagataaattcaactctcacctcagttgagagagaactgtcaatgtgacttggcctctctctttctagtcccagaaagaattgcactgaaatgctgagctcctgtaataaaaatgaccatttgctgagagtaattaacatactgaaagagattttcttagaatagtgcacaatggcccaatggtgacattatattgtctctttataaattattttctatctatttctgtggattatttctacaaagcacttttcatatgtccaattccttttattcccctacaagtactgactgactactggctctgctgttcactgatatgactttcggcaagttgcctgcactttttaaacgttatttcctcattcagaacatggggccatacaaaatacaactcacttcagtgttattggggaattaaacaaataaatgcatgggaagcatttaacatagtgcctgacacaataatgagcactcagtagatgttagcttttattaatattgttgttgctatgtccagaaacactatacctccagaaaatcatgggtacttgctggggacgttggggatatgcatgattttgaaaggagtgactgctctttactgctcagatgagaaatttttctaagccagactccttcaaacatgtaagattctgttgtggattctaggactgaaagaattcttggccgagtgtggtggcttatcctggtaatctcatcatttgggaggacaaggcaggaagattgcttgagcccaggagttggaaacaagcctggacaacatggcgaaaccctgtctctacaaaaaatacaaacattagctggtcatgggagtgagtgcctgtactcccagctactcaggaggctaagataggaggatcacctgagcctgggcagtttgaggtttcagtgagccgtgatgacaccatactatactccactccagcctgggtgacagtgacatcctgcctcaaaaaaacccccaaaattattctttttgctgatttcatgtcagcagtgtgtgctgaaggctgtaaagtagccacttgttctgtttatttttccattgaacaagtatttatcaaaaacgtactttgtggaaggcactgtgctaggaactatgcatacagaaggaaaaccaaatgttcttggatactacactccagttgtgataaaaaagaaaaaagtattcttcacaaacttcaacattttgatgtgcaaaaacataatatatgaattagatctacctaactacacagaattagaccaattatttctgggattatgggctcatatttttaataactgtcctcctacctctctgttgacaggttttataaatattcatttaattacacacagtcacagacacactcagacacacacacatacacacacacacacaccttgacaaataatgggcatgaacaattgactggtacttgctctcattcttctagatgtcaccacagtggatcccaaatacaattattcaaaggatgcaaatggtaagtttttgtgttttttatttcctcctgatcattttaagttttgaacttctctggcttgaaaaatcagggaatggattttgctaggttggatgctgcagaatggacctaatcatattttaaattagtccctctttttctaggagttgtattaacaaacctaactactgcttcatgtaagagatgactgtaaattgaagggtacagtgatatgctttcagttatttcaaaaaacagactttactcatccatgtgtcttttttcttttcttttttttcttttttgagacggagtctcgctctgttgaacaggctggattgcagtgacgcgatctcacctcactacaacctccgcctctggagttcaagcgattctccagcctcagcttctcaagtagctgggactacaggcacatgccaccatgtccgggtcatctttgtatttttagcagagaccgggtttcactatgttggccaggctggtctagaattcctgacttcgtgatctgccccctcagccctccgaagtgctgggattacagacgtgagtcactgtgcccggcctaacagtaaaatgtctttcatgcgcttctcaaggcaactacgttaaggaggacacttctcttaatgtcattctacagtagatttctaatgctctttcttggaagtttgtttttctgagaaaagctaaaaatataacatggaagtgatcatattgtataatcaatgaagtgcttttcaaggagataaaactaatctggtccacgtttgcaaccaaccttgattgagagagagagagaactcaggatacacttggagattttattatggggaatagttactttattcttttttcctcaatcaattcatggaaataagtgatagtcatattcatttatcttttaataaatgaagtcaccatgaggaaaataaaaagacattgaaaacccattaaagttagcccttaaagatatttggacatgcagacttgataactaacgtttgcattcttgagacttacccaaaacccatacctcaagtccatgtttttagaattcatgaaataaagatctcagtgagtgcataaaattgcgcaccagaatcatatccgtatagacaagaacacatctactagaaaaataataaaccaacacaccaatgcaactgtgttttcttctgttttaaaatatgttgtctttgtatgcatgtttgcttcttccttttttttttttaacatcacagataaattcaactctcacctcaggttttattgagagaactgtcaatgtgacttggcctctgtctttctagtcccagaaagaatcgcactgaaatgctgagctcctgtaataaaaatgaccatttgctgagagtaattaacatactgaaagagattttcttagagtacacaatggtgacattatattgtctctttataaataactttctatctatttctgtggattattcctacaaagtacttttcatatgtccagtttcttttcttcccctacaactaccgtctgaatactggctctgctatttgctgatatgattctcggcaagttgcctgcactttttaaactttatttcctcattcagaacatggggccatgtaatactcatgtacgtgagtattacgtaataatgctcacttaagtgttactggggaattaaacaaaaaaatgcatggcaagcatttaacatagtgcctgacacaataatgagcactcagtagatgttagattttattaatattgttgttgttatgtccggaaacactatacctccagaaaatcatgggtacttgcttgggatgttggggatatgcatgatttggaaaggtatgactgcttttttctgcttagatgagaaatttttctaagccagactccttcaaatatgtaagattctgttgtggattctaggacggaaagaattcttggtcaggtgtggtttcttatccctgtaatcccagaattttgggaggacaaggcaggaagattgcttgagcccaggagtttgaaaccagcctgggcaacaagacgaaaccctgtctctacaaaagtacataaattagcttggcttggtggtgtgtgcctgtattaccagctattcgggagactgagatgggaggatctcctgaacctgtgaagtttgaggcttcagtgagccgtgatgacaccatactatactcgactccagcctgtgcgacagtgagactctgcgtcaaaaaaaaaaccccaaaattattgtttttgctgatttcaggtcagcagtgtgtgctgaagggtgtaaagtagccacttgatcagtttatttttccactgaacaagtatttatcaaaaacatactttgtggtctgtttttgataaataaaaaggcactgtgctaggagccatgaatacagaaggaaaaccaaatgttcttggatactacactccagttgtgataaaaaagaaaaatgtattcttcacgaacttcaacattttgatatgcaaaaacatagtatataaattagatctacctgattacgtagaatcagaccaattatttctggaattgagggctcatatttttaataactgtcctcctgcctctctgttgacaggttttataaatattcatttaattacacacacacacacacacaccttgacaaataatggacatgaacaattgactagtacttgctctcattcttctagatgtcatcacaatggatcccaaagacaattggtcaaaagatgcaaatggtaagcttttgtgtttttcctttcctcctgatcattttaagttttgaacttctctggcttgaaaaatcagggaatgggccgggtgcggtggctcacgcctgtaatcccagcactttgggaggccgaggcgggcggatcacgaggtcaggagatcgagaccatcccggctaaaacggtgaaaccccgtctctactaaaaatacaaaaaattagccgggcttagtggcgggcgcctgtagtcccagctacttgggaggctgaggcaggagaatggcgtgaacccgggaggcggagcttgcagtgagccgagattgcgccactgcactccactccagcctgggcgacagagcgagactccgtctcaaaaaaaaaaaaaaaaaaaaaaaaagaaaaatcagggaatggattttgctaggttggatgctgcagaatggacctagtgatattttaaattagtccctctttttctaggagttgtattaacaaacctaactactgcttcgggtatgagatgactgtaaattagagggtacagtgatatgctttcagttatttcaaaaaacagactttattcatccgtctgtcttttttttttttttttttttttttttttgagacggaggagtctcactctatcacccaggctggagtgcagtggcgcgatctcggctcaccataacctccgccttactggttcaagcgattctccagcctcagcttctcaagtagctgggactacaggtgcacaccaccatacctggctaatttttgtatttttaatagagatggggtttcaccacgctggccaggatggtcttgaattcttgacctcgtgatctgccccctcgggctcccaaacttctgggattataggcgtgagccactgtgcccggccttctgtcttttgttataatgactggggaaaacatgataccatgttgcttcttgagttgttttgttttagtctttggtctttgctagtagctaataacacgaactagtgtttatcaagtgctttttacacagaagggcttgttctgcattttctagtttaatcatcttaatactcctataaagtagtacaatatattttctcccattttacagtccctttaaagtaaataactataaaaatcccttatacatgtcacacagctaggtctggcatttcaaatcaggacatcaaacaaagaattcgtgcagttactaagtcctctattttttctacaatagaaaaaatagcaagaattacagatagcaagacattacaaggcaggaatctgaaacgaaagggacataatgtggggctgggtgggtgcatgagctttgcagactagactttcattccagctcttttaatgattaggtgtaagtgacctacattttgtgagtaacagttttctcatcagccaactaagaataattacaccagattcacagttattgaagagataagggcatgaatgtgagatgtctggcgtagggtatctcatttagcagacacagaatgaatacttgtttctggctttttctctctacatatgcacaaagaatgtgactagaagcattggctctagccctgctcaactttcctctatttccaataccaaggggctctgacttaggctgccacaccaggcaaggaggggcagtaccacctcacttgaccaagggcagggagtcacggacacatcacttcctgagatccttttccacaccaaggactgatgtttctggaattctcactttatgaagacaaaacatataaatggaaatttctgcaggaagagactcactcttgtagctcattgagtaggcactagtggtccacccccactgtctttacttattccttgacatcacatatctcttgtaaaacctcaaataatgttaaatgcaatcacccaataatagcatagccataattagaggcatttaggaaagacaggtgagtgtgccacaactacctaacacatcagcaaatctggattaaccactttctttgattttccacaatgcaaccttactttttaatagttgggaatgttctaagtgaatttagcagaggttgttaatcaacttgaaagctgaattctgacttgtctgactcttggtggtgctggtagcagtagatgtttacttttaggttttggtggtggtggaatatcacttcaacgtaaatcatcagaaataagtatttgtgaacccctctcgcattaatatatcttattctgtaaaaagaacatgtgcaatttctcttagatacactactgctgcagctcacaaacacctctgcatattacacgtacctcctcctgctcctcaagagtgtggtctattttgccatcatcacctgctgtctgcttagaagaacggctttctgctgcaatggagagaaatcataa

SEQ ID NO: 25 - protein, 153 aa, Homo sapiens
SQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 26 - protein, 10 aa, synthetic construct
IPNIQNPDPA

SEQ ID NO: 27 - DNA, 30 bp, synthetic construct
  source 1..30: mol_type = other DNA
attcccaatatccagaaccctgaccctgcc

SEQ ID NO: 28 - protein, 11 aa, synthetic construct
LEDLKNVFPPE

SEQ ID NO: 29 - DNA, 33 bp, synthetic construct
  source 1..33: mol_type = other DNA
ctggaggatctgaaaaacgtgttccctcctgaa

SEQ ID NO: 30 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 31 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 32 - protein, 127 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRTADTQALLRNDQVYQPLRDRDDAQYSHLGGNWARNK

SEQ ID NO: 33 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 34 - protein, 5 aa, synthetic construct
SYIMH

SEQ ID NO: 35 - protein, 17 aa, synthetic construct
VISYDGRNKYYADSVKG

SEQ ID NO: 36 - protein, 9 aa, synthetic construct
DTDGYDFDY

SEQ ID NO: 37 - protein, 11 aa, synthetic construct
RASQSVSSYLA

SEQ ID NO: 38 - protein, 7 aa, synthetic construct
DASNRAT

SEQ ID NO: 39 - protein, 9 aa, synthetic construct
QQRTNWPLT

SEQ ID NO: 40 - protein, 135 aa, synthetic construct
MELGLSWIFLLAILKGVQCQVQLVESGGGVVQPGRSLRLSCAASGFTFSSYIMHWVRQAPGKGLEWVAVISYDGRNKYYADSVKGRFTISRDNSKNTLYLQMNSLRAEDTAVYYCARDTDGYDFDYWGQGTLVTV

SEQ ID NO: 41 - DNA, 405 bp, synthetic construct
  source 1..405: mol_type = other DNA
atggaactcggtctgtcctggatttttctgcttgcgatcttgaaaggagtgcagtgccaggtgcagctggtggagtctgggggaggcgtggtccagcctgggaggtccctgagactctcctgtgcagcctctggatttaccttcagtagctatattatgcactgggtccgccaggctccaggcaaggggctggagtgggtggcagttatatcatatgatggaagaaacaaatactacgcagactccgtgaagggccgattcaccatctccagagacaattccaagaacacgctgtatctgcaaatgaacagcctgagagctgaggacacggctgtgtattactgtgcgagagatacggatggctacgattttgactactggggccagggaaccctggtcaccgtc

SEQ ID NO: 42 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRTNWPLTFGGGTKVEIK

SEQ ID NO: 43 - DNA, 378 bp, synthetic construct
  source 1..378: mol_type = other DNA
atggaagccccagctcagcttctcttcctcctgctactctggctcccagataccaccggagaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtaccaactggccgctcactttcggcggagggaccaaggtggagatc

SEQ ID NO: 44 - protein, 20 aa, Homo sapiens
MYRMQLLSCIALSLALVTNS

SEQ ID NO: 45 - protein, 20 aa, Mus sp.
MYSMQLASCVTLTLVLLVNS

SEQ ID NO: 46 - protein, 20 aa, Homo sapiens
METPAQLLFLLLLWLPDTTG

SEQ ID NO: 47 - protein, 20 aa, Mus sp.
METDTLLLWVLLLWVPGSTG

SEQ ID NO: 48 - protein, 21 aa, Homo sapiens
MALPVTALLLPLALLLHAARP

SEQ ID NO: 49 - protein, 18 aa, Homo sapiens
MALPVTALLLPLALLLHA

SEQ ID NO: 50 - protein, 16 aa, Homo sapiens
MKWVTFISLLFSSAYS

SEQ ID NO: 51 - protein, 30 aa, Homo sapiens
MDSKGSSQKGSRLLLLLVVSNLLLCQGVVS

SEQ ID NO: 52 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 53 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 54 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 55 - protein, 22 aa, synthetic construct
QNQLYNELNLGRREEYDVLDKR

SEQ ID NO: 56 - DNA, 66 bp, synthetic construct
  source 1..66: mol_type = other DNA
cagaaccagctctataacgagctcaatctaggacgaagagaggagtacgatgttttggacaagaga

SEQ ID NO: 57 - protein, 22 aa, synthetic construct
QNQLFNELNLGRREEFDVLDKR

SEQ ID NO: 58 - DNA, 66 bp, synthetic construct
  source 1..66: mol_type = other DNA
cagaaccagctctttaacgagctcaatctaggacgaagagaggagttcgatgttttggacaagaga

SEQ ID NO: 59 - protein, 23 aa, synthetic construct
QEGLYNELQKDKMAEAYSEIGMK

SEQ ID NO: 60 - DNA, 69 bp, synthetic construct
  source 1..69: mol_type = other DNA
caggaaggcctgtacaatgaactgcagaaagataagatggcggaggcctacagtgagattgggatgaaa

SEQ ID NO: 61 - protein, 23 aa, synthetic construct
QEGLFNELQKDKMAEAFSEIGMK

SEQ ID NO: 62 - DNA, 69 bp, synthetic construct
  source 1..69: mol_type = other DNA
caggaaggcctgttcaatgaactgcagaaagataagatggcggaggccttcagtgagattgggatgaaa

SEQ ID NO: 63 - protein, 22 aa, synthetic construct
HDGLYQGLSTATKDTYDALHMQ

SEQ ID NO: 64 - DNA, 66 bp, synthetic construct
  source 1..66: mol_type = other DNA
cacgatggcctttaccagggtctcagtacagccaccaaggacacctacgacgcccttcacatgcag

SEQ ID NO: 65 - protein, 22 aa, synthetic construct
HDGLFQGLSTATKDTFDALHMQ

SEQ ID NO: 66 - DNA, 66 bp, synthetic construct
  source 1..66: mol_type = other DNA
cacgatggccttttccaggggctcagtacagccaccaaggacaccttcgacgcccttcacatgcag

SEQ ID NO: 67 - protein, 112 aa, synthetic construct
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 68 - DNA, 336 bp, synthetic construct
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccaggggctcagtacagccaccaaggacaccttcgacgcccttcacatgcaggccctgccccctcgc

SEQ ID NO: 69 - DNA, 123 bp, Homo sapiens
  source 1..123: mol_type = genomic DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 70 - protein, 218 aa, Mus musculus
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 71 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 72 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 73 - DNA, 762 bp, Homo sapiens
  source 1..762: mol_type = genomic DNA
atggaatacgcctctgacgcttcactggaccccgaagccccgtggcctcccgcgccccgcgctcgcgcctgccgcgtactgccttgggccctggtcgcggggctgctgctgctgctgctgctcgctgccgcctgcgccgtcttcctcgcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaa

SEQ ID NO: 74 - protein, 288 aa, Homo sapiens
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 75 - DNA, 864 bp, Homo sapiens
  source 1..864: mol_type = genomic DNA
atgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgta

SEQ ID NO: 76 - protein, 309 aa, synthetic construct
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFKRGRKKLLYIFKQPFMRPVQTTQEEDGCSCRFPEEEEGGCEL

SEQ ID NO: 77 - protein, 349 aa, synthetic construct
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFRSKRSRLLHSDYMNMTPRRPGPTRKHYQPYAPPRDFAAYRKRGRKKLLYIFKQPFMRPVQTTQEEDGCSCRFPEEEEGGCEL

(19) United States
(12) Patent Application Publication
(10) Pub. No.: US 2025/0249040 A1
(43) Pub. Date: Aug. 7, 2025

(54) CELLS EXPRESSING ANTI-CD70 CHIMERIC RECEPTOR AND USES THEREOF

(71) Applicants: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(72) Inventors: Michel Sadelain, New York, NY (US); Sophie Alexandra Hanina, New York, NY (US)

(73) Assignees: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(21) Appl. No.: 19/184,470

(22) Filed: Apr. 21, 2025

Related U.S. Application Data
(63) Continuation of application No. PCT/US2023/077498, filed on Oct. 23, 2023.
(60) Provisional application No. 63/380,482, filed on Oct. 21, 2022.

(51) Int. Cl. A61K 35/17; A61K 40/11; A61K 40/31; A61K 40/42; C07K 14/705; C07K 14/725; C07K 16/28; C12N 5/0783

(52) U.S. Cl. CPC A61K 35/17; A61K 40/11; A61K 40/31; A61K 40/4224; C07K 14/7051; C07K 14/70578; C07K 16/2875; C12N 5/0636; A61K 2239/13; A61K 2239/21; C07K 2317/31; C07K 2317/565; C07K 2317/92; C07K 2319/02; C07K 2319/03

(57) ABSTRACT

The presently disclosed subject matter provides cells, compositions and methods for enhancing immune responses toward tumor antigens. It relates to cells comprising a first antigen-recognizing receptor that targets CD70. These cells have improved activity and/or efficiency.

### CROSS-REFERENCE TO RELATED APPLICATIONS

This application is a continuation of International Patent Application No. PCT/US23/77498, filed Oct. 23, 2023, which claims priority to U.S. Provisional Application No. 63/380,482, filed Oct. 21, 2022, the contents of which are incorporated by reference in their entireties, and to which priority is claimed.

### SEQUENCE LISTING

A Sequence Listing conforming to the rules of WIPO Standard ST.26 is hereby incorporated by reference. Said Sequence Listing has been filed as an electronic document via Patent Center encoded as XML in UTF-8 text. The electronic document, created on Oct. 18, 2023, is entitled “072734_1498_SL”, and is 102,400 bytes in size.

### INTRODUCTION

The presently disclosed subject matter provides cells, compositions and methods for enhancing immune responses toward tumor antigens. It relates to cells comprising an antigen-recognizing receptor (e.g., a TCR like fusion molecule) targeting CD70. The presently disclosed cells have improved the activity against solid tumors (e.g., renal cell carcinoma).

### BACKGROUND OF THE INVENTION

Cell-based immunotherapy is a therapy with curative potential for the treatment of cancer. T cells and other immune cells may be modified to target tumor antigens through the introduction of genetic material coding for an antigen recognizing receptor, e.g., a TCR like fusion molecule. Patient-engineered CAR T cells have demonstrated remarkable efficacy against a range of liquid and solid malignancies. However, treatment failure and relapses occur in a large fraction of patients. Therefore, there remain needs of improved immunotherapy.

### SUMMARY OF THE INVENTION

The presently disclosed subject matter provides methods of reducing tumor burden, and/or preventing and/or treating a tumor in a subject having a renal cell carcinoma, a pancreatic cancer, or an ovarian cancer. Additionally or alternatively, the presently disclosed subject matter provides methods of reducing tumor burden, and/or preventing and/or treating a tumor in a subject, wherein the tumor is a renal cell carcinoma, a pancreatic cancer, or an ovarian cancer.

In certain embodiments, the methods comprise administering to the subject an effective amount of a cell comprising a TCR-like fusion molecule that targets CD70. In certain embodiments, the methods reduce the number of tumor cells, reduces tumor size, and/or eradicates the tumor in the subject.

The presently disclosed subject matter further provides methods of preventing and/or treating a tumor in a subject in need thereof comprising obtaining a tumor sample that has undetectable CD70 polypeptide levels from the subject, detecting a CD70 polynucleotide by FISH, and administering to the subject an effective amount of a cell comprising a TCR-like fusion molecule that targets CD70 if the CD70 polynucleotide is detected. Moreover, the presently disclosed subject matter further provides methods of preventing and/or treating a tumor in a subject in need thereof comprising obtaining a tumor sample that has undetectable CD70 polypeptide levels from the subject, b) contacting the sample with a Ezh2 inhibitor, and administering to the subject an effective amount of a cell comprising a TCR-like fusion molecule that targets CD70 if the CD70 polypeptide is detected in the sample.

In certain embodiments, the TCR-like fusion molecule comprises i) a first antigen-binding chain comprising an antigen-binding fragment of a heavy chain variable region (VH) of an antibody; and ii) a second antigen-binding chain comprising an antigen-binding fragment of a light chain variable region (VL) of the antibody; wherein the first and second antigen-binding chains a) each comprise the TRAC polypeptide or the TRBC polypeptide, and b) bind to the second antigen, wherein the TCR-like fusion molecule binds to the second antigen in an HLA-independent manner.

In certain embodiments, wherein at least one of the TRAC polypeptide and the TRBC polypeptide is endogenous. In certain embodiments, the first and the second antigen-binding chains bind to the second antigen with a dissociation constant (KD) of about 1×10⁻⁸ M or less. In certain embodiments, the first and the second antigen-binding chains bind to the second antigen with a dissociation constant (KD) of about 5×10⁻⁹ M or less.

In certain embodiments, the first antigen-binding chain comprises an antigen-binding fragment of a VH of an antibody and a TRBC polypeptide, and the second antigen-binding chain comprises an antigen-binding fragment of a VL of the antibody and a TRAC polypeptide. In certain embodiments, the first antigen-binding chain comprises an antigen-binding fragment of a VH of an antibody and a TRAC polypeptide, and the second antigen-binding chain comprises an antigen-binding fragment of a VL of the antibody and a TRBC polypeptide.

In certain embodiments, i) the first antigen-binding chain comprises a CDR1 comprising the amino acid sequence set forth in SEQ ID NO: 34, a CDR2 comprising the amino acid sequence set forth in SEQ ID NO: 35, and a CDR3 comprising the amino acid sequence set forth in SEQ ID NO: 36, and the second antigen-binding chain comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 39. In certain embodiments, the first antigen-binding chain comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 39, and the second antigen-binding chain comprises a CDR1 comprising the amino acid sequence set forth in SEQ ID NO: 34, a CDR2 comprising the amino acid sequence set forth in SEQ ID NO: 35, and a CDR3 comprising the amino acid sequence set forth in SEQ ID NO: 36.

In certain embodiments, the first antigen-binding chain comprises a CDR1, a CDR2, and a CDR3 of the heavy chain variable region set forth in SEQ ID NO: 40, and the second antigen-binding chain comprises a CDR1, a CDR2, and a CDR3 of the light chain variable region set forth in SEQ ID NO: 42. In certain embodiments, the first antigen-binding chain comprises a CDR 1, a CDR2, and a CDR3 of the light chain variable region set forth in SEQ ID NO: 42, and the second antigen-binding chain comprises a CDR 1, a CDR2, and a CDR3 of the heavy chain variable region set forth in SEQ ID NO: 40. In certain embodiments, the first antigen-binding chain comprises the heavy chain variable region set forth in SEQ ID NO: 40, and the second antigen-binding chain comprises a CDR1, a CDR2, and a CDR3 of the light chain variable region set forth in SEQ ID NO: 42. In certain embodiments, the first antigen-binding chain light chain variable region set forth in SEQ ID NO: 42; and the second antigen-binding chain comprises the heavy chain variable region set forth in SEQ ID NO: 40.

In certain embodiments, the first and second antigen binding chains are capable of associating with a CD3ζ polypeptide. In certain embodiments, the first and second antigen binding chains, upon binding to the second antigen, are capable of activating the CD3ζ polypeptide. In certain embodiments, the activation of the CD3ζ polypeptide is capable of activating the cell.

In certain embodiments, the cell further comprises a gene disruption of a TRAC locus. In certain embodiments, the cell further comprises a gene disruption of a CD70 locus. In certain embodiments, the cell further comprises a gene disruption of a TRAC locus and a CD70.

In certain embodiments, the tumor comprises tumor cells having a CD70 low antigen density. In certain embodiments, the tumor CD70+ tumor cells having low tumor cell frequency. In certain embodiments, the tumor comprises a CD70 polypeptide that is not detectable by immunodiffusion, immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, or a combination thereof. In certain embodiments, the tumor and/or neoplasm comprises a CD70 polypeptide that is not detectable by immunohistochemistry (IHC). In certain embodiments, the tumor comprises a CD70 polypeptide that is not detectable by immunohistochemistry (IHC).

In certain embodiments, the cell is a cell of the lymphoid lineage or a cell of the myeloid lineage. In certain embodiments, the cell of the lymphoid lineage is selected from the group consisting of a T cell, a B cell, a Natural Killer (NK) cell, and a dendritic cell. In certain embodiments, the cell is a T cell. In certain embodiments, the T cell is derived from an induced pluripotent stem cell. In certain embodiments, the T cell is a CD8⁺ T cell. In certain embodiments, the CD8⁺ T cell is CD4 independent. In certain embodiments, the T cell is selected from the group consisting of a cytotoxic T lymphocyte (CTL), a γδ T cell, a tumor-infiltrating lymphocyte (TIL), a regulatory T cell, and a Natural Killer T (NKT) cell.

In certain embodiments, the cell further comprises a chimeric antigen receptor (CAR) that targets a second antigen. In certain embodiments, the CAR comprises an extracellular antigen-binding domain that binds to the first antigen, and an intracellular signaling domain that is capable of delivering an activation signal to the cell. In certain embodiments, the intracellular signaling domain of the CAR comprises a CD3ζ polypeptide. In certain embodiments, the CD3ζ polypeptide is a native CD3ζ polypeptide or a modified CD3ζ polypeptide. In certain embodiments, the modified CD3ζ polypeptide comprises a native ITAM1, an ITAM2 variant consisting of two loss-of-function mutations, and an ITAM3 variant consisting of two loss-of-function mutations. In certain embodiments, the intracellular signaling domain of the CAR further comprises at least one costimulatory signaling region. In certain embodiments, the at least one costimulatory signaling region comprises at least an intracellular domain of a co-stimulatory molecule or a portion thereof. In certain embodiments, the costimulatory molecule is selected from the group consisting of CD28, 4-1BB, OX40, CD27, CD40, CD154, CD97, CD11a/CD18, ICOS, DAP-10, CD2, CD150, CD226, and NKG2D. In certain embodiments, the CAR comprises a transmembrane domain.

In certain embodiments, the cell further comprises a chimeric co-stimulating receptor (CCR). In certain embodiments, the CCR comprises an extracellular antigen-binding domain that binds to the third antigen and an intracellular domain that is capable of delivering a costimulatory signal to the cell but does not alone deliver an activation signal to the cell. In certain embodiments, the intracellular domain of the CCR comprises at least an intracellular domain of a co-stimulatory molecule or a portion thereof. In certain embodiments, the costimulatory molecule is selected from the group consisting of CD28, 4-1BB, OX40, CD27, CD40, CD154, CD97, CD11a/CD18, ICOS, DAP-10, CD2, CD150, CD226, and NKG2D.

In certain embodiments, the second antigen is a tumor antigen or a pathogen antigen. In certain embodiments, the tumor antigen is selected from the group consisting of CD19, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell (e.g. a cell surface antigen), ANO9, AQP2, ASIC3, ASPRV1, ATP6VOA4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD30, CD300LF, CD312, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-α, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, LICAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PR1), PSD2, PTPRJ, RDH16, receptor tyrosine-protein kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TASIR3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, and ZDHHC11.

In certain embodiments, the cell further comprises at least one exogenous costimulatory ligand. In certain embodiments, the at least one exogenous co-stimulatory ligand is selected from the group consisting of a tumor necrosis factor (TNF) family member, an immunoglobulin (Ig) superfamily member, and combinations thereof. In certain embodiments, the TNF family member is selected from the group consisting of 4-1BBL, OX40L, CD70, FasL, GITRL, TNF-related apoptosis-inducing ligand (TRAIL), CD30L, LIGHT (TNFSF14), CD40L.

In certain embodiments, the Ig superfamily member is selected from the group consisting of CD80, CD86, ICOSLG, and combinations thereof. In certain embodiments, the at least one exogenous costimulatory ligand comprises CD80. In certain embodiments, the at least one exogenous a costimulatory ligand comprises 4-1BBL. In certain embodiments, the cell comprises two exogenous costimulatory ligands. In certain embodiments, the at least two exogenous costimulatory ligands comprise CD80 and 4-1BBL.

In certain embodiments, the cell further comprises a fusion polypeptide comprising: a) an extracellular domain and a transmembrane domain of a co-stimulatory ligand, and b) an intracellular domain of a first co-stimulatory molecule. In certain embodiments, the co-stimulatory ligand is selected from the group consisting of a tumor necrosis factor (TNF) family member, an immunoglobulin (Ig) superfamily member, and combinations thereof. In certain embodiments, the TNF family member is selected from the group consisting of 4-1BBL, OX40L, CD70, GITRL, CD40L, and combinations thereof. In certain embodiments, the Ig superfamily member is selected from the group consisting of CD80, CD86, ICOSLG, and combinations thereof. In certain embodiments, the co-stimulatory ligand is CD80. In certain embodiments, the first co-stimulatory molecule is selected from the group consisting of CD28, 4-1BB, OX40, ICOS, DAP-10, CD27, CD40, NKG2D, CD2, and combinations thereof. In certain embodiments, the first co-stimulatory molecule is 4-1BB. In certain embodiments, the co-stimulatory ligand is CD80 and the first co-stimulatory molecule is 4-1BB. In certain embodiments, the fusion polypeptide further comprises an intracellular domain of a second co-stimulatory molecule. In certain embodiments, the second co-stimulatory molecule is selected from the group consisting of CD28, 4-1BB, OX40, ICOS, DAP-10, CD27, CD40, NKG2D, CD2, and combinations thereof. In certain embodiments, the second co-stimulatory molecule is CD28. In certain embodiments, the co-stimulatory ligand is CD80, the first co-stimulatory molecule is 4-1BB, and the second co-stimulatory molecule is CD28.

In certain embodiments, the cell is autologous. In certain embodiments, the cell is allogeneic.

### BRIEF DESCRIPTION OF THE FIGURES

The following Detailed Description, given by way of example, but not intended to limit the presently disclosed subject matter to specific embodiments described, may be understood in conjunction with the accompanying drawings.

FIG. 1 depicts a cartoon illustrating challenges facing CARs in solid tumors.

FIG. 2A shows characteristics of two RCC PDX models, ‘K5’ and ‘K7’ (histology, previous treatment and genetic mutations). FIG. 2B shows CD70 expression in in vitro K5 and K7 cells. FIG. 2C shows a schematic of the CD70-2821XX CAR with CD28 co-stimulatory domain and an attenuated zeta chain signaling domain, termed ‘1XX’.

FIGS. 3A-3E depict set up of renal cell carcinoma PDX models. FIG. 3A shows CD70 CAR-mediated killing in vitro. FIG. 3B shows K7 RCC PDX lung metastatic model obtained by tail-vein administration. FIG. 3C shows K5 RCC PDX lung metastatic model obtained by tail-vein administration. FIG. 3D shows K5 and K7 RCC PDX primary site models obtained by orthotopic kidney administration.

FIGS. 4A and 4B depict effects of anti-CD70 CAR T cells in vivo. FIG. 4A shows effect of anti-CD70 CAR T cells in K7 RCC PDX lung metastatic model obtained by tail-vein administration. FIG. 4B shows effect of anti-CD70 CAR T cells in K5 RCC PDX model obtained by orthotopic kidney administration. FIG. 4C shows effect of anti-CD70 CAR T cells in K5 RCC PDX lung metastatic model obtained by tail-vein administration. FIG. 4D shows effect of anti-CD70 CAR T cells in K7 RCC PDX primary site model obtained by orthotopic kidney administration.

FIG. 5 shows distribution of RCC tumors (labeled with firefly luciferase) and CAR T cells (labeled with Gaussia luciferase) in lung metastatic model, orthotopic model and in mice that have tumor at both lung and kidney tumor site, indicating that while tumor cleared from lungs but not kidney, CAR T cells trafficked to both sites.

FIG. 6 depicts FACS analysis of CD70 expressed in lung and kidney of untreated mice. CD70 is homogeneous and high in lungs hence complete tumor eradication in lung metastatic models by CD70 CAR T cells. CD70 expression is heterogeneous at kidney orthotopic site (MFI negative in CD70 negative population) explaining incomplete tumor eradication by CD70 CAR T cells.

FIG. 7 depicts sort and bulk RNA sequencing of lung, heterogeneous CD70lo or CD70⁺ kidney tumor from untreated mice. Bulk RNA sequencing shows very low level CD70 expression by transcript in CD70lo kidney tumor population. Other genes not expressed in RCC tumor are listed as comparison.

FIGS. 8A and 8B depict FACS analysis of in vitro cell culture restoring expression of CD70. FIG. 8A shows culture of untreated K5 kidney tumor with low density CD70 (CD70 MFI negative population) in vivo kidney populations restored CD70 expression in vitro. FIG. 8B shows culture of untreated K7 with low density CD70 (CD70 MFI negative population) in vivo kidney populations restored CD70 expression in vitro.

FIGS. 9A-9D depict epigenetic analysis. FIG. 9A shows epigenetic analysis of CD70 locus in the indicated cell types. FIG. 9B shows epigenetic analysis of CAIX locus in the indicated cell types. FIG. 9C shows interaction of certain methylation regulators (Ezh2, H3K4mc3, H3K27mc3) and CD70 locus. FIG. 9D shows the effects of tazemetostat (Ezh2 inhibitor) on ex vivo culture of sorted heterogeneous kidney tumor and treatment with Ezh2 inhibitor for 8 days.

FIGS. 10A-10E depict effect of CD70 overexpression on tumor clearance. FIG. 10A shows constructs including mCerulean CD70 and its expression. FIG. 10B shows effect of overexpression of mCerulean CD70 fusion protein on NALM6. FIG. 10C shows that overexpression of mCerulean CD70 Fusion Protein on NALM6 did not impair in vitro killing in 18 hr CTL. FIGS. 10D and 10E show that overexpression of CD70 on K5 and K7 PDX orthotopic models resulted in tumor clearance in vivo.

FIGS. 11A-11C depict expression levels of CAIX. FIG. 11A shows FACS analysis of CAIX expression in vitro. FIG. 11B shows CD70/CAIX expression in K5 untreated mice. FIG. 11C shows CD70/CAIX expression in K7 untreated mice.

FIGS. 12A-12I depict dual targeting approach using CD70 and CAIX. FIG. 12A shows CAIX2821XX CAR T in vitro killing. FIG. 12B shows that dual targeting of CD70 and CAIX improved tumor control for K5. FIG. 12C shows residual CD70 positive population post CAIX 28z1XX CAR treatment. FIGS. 12D and 12E show that CD70 and CAIX dual targeting did not result in tumor clearance of residual low density antigen populations. FIG. 12F shows FACS analysis illustrating comparison of tumor profiles. FIGS. 12G-12I show effects of anti-CD70 CAR, anti-CAIX CAR, and double transduced T cells on tumor models.

FIGS. 13A-13I depict analysis of tumor cells expressing low density antigen of CD70 and CAIX. FIG. 13A shows CD70/CAIX expression in untreated kidney tumor (4 quadrants sorted: CD70−CAIX−/CD70+CAIX−/CD70−CAIX+/CD70+CAIX+), and in untreated tumor at lung site (CD70+CAIX− population sorted). FIG. 13B shows bulk RNA sequencing of CD70/CAIX sorted tumor populations from kidney and lung site of K5 RCC PDX. FIG. 13C show bulk RNA sequencing of CD70/CAIX sorted tumor populations from kidney and lung site of K7 RCC PDX. FIG. 13D shows mass spectrometry analysis of CD70/CAIX sorted tumor populations. FIG. 13E shows a time-course of CD70/CAIX expression in untreated kidney tumors. FIG. 13F shows CD70 antigen quantification in untreated kidney tumors: #mol/cell (CD70−CAIX− population is negative for CD70 by MFI and quantification). FIG. 13G shows CAIX antigen quantification in untreated kidney tumors: #mol/cell. FIG. 13H shows CD70 antigen quantification at day 2 ex vivo CD70−CAIX− sorted kidney tumor with very low level restoration of CD70 expression after 48 hrs of culture. FIG. 13I shows CAIX antigen quantification at day 2 ex vivo sorted kidney tumor (48 hrs in culture).

FIGS. 14A-14E depict effects of the low antigen targeting strategy. FIGS. 14A-14D show that 70-HIT expressing a costimulatory ligand (80/41BBL) cleared K5 orthotopic kidney tumor. FIG. 14E shows that 70-HIT expressing a costimulatory ligand (80/41BBL) cleared K7 orthotopic kidney tumors.

FIG. 15 depicts characterization of T cell phenotype at kidney tumor site for both K5 and K7 at d7 and d14 post CD70 HIT (expressing a CD80 and 4-1BBL polypeptide) or CD7028z1XX CAR T cell treatment. Number of CAR+ or HIT+ T cells (left column), number of tumor cells (middle column), and expression profile of triple inhibitory receptors PD-1, TIM-3 and LAG-3 (right column) are shown.

FIGS. 16A-16C show in vivo efficacy of T cells expressing the CD70 HIT, a CD80 polypeptide, and a 4-1BBL polypeptide cannot be accounted for by bystander killing of CD70 negative tumor cells. FIG. 16A depicts in vitro cytotoxicity assays of HIT CD70 against K5 and K7 PDX lines with a CD70 knockout. FIG. 16B and FIG. 16C show that CD70 HIT T cells expressing a CD80 and 4-1BBL polypeptide cannot eradicate RCC tumor with a CD70 knockout in vivo. Furthermore, mixing 75-80% wild-type RCC PDX tumor with 20-25% CD70 knockout RCC PDX tumor did not lead to bystander killing of knockout tumor in vivo.

FIGS. 17A-17E depicts flow cytometric characterization of a CD70 heterogeneous pancreatic ductal adenocarcinoma (PDAC2) PDX line, the mechanism of CD70 regulation and cytotoxic effects of T cells expressing the CD70 HIT, a CD80 polypeptide, and a 4-1BBL polypeptide on PDAC2-derived orthotopic pancreatic cancer. FIG. 17A shows characterization of PDAC2 (Pancreatic Ductal Adenocarcinoma) PDX Line. FIG. 17B show cytotoxic effects on PDAC2 cancer in vitro. FIG. 17C depict effect of Ezh2 inhibitors on PDAC2 cells. FIG. 17D shows immunoblotting of PDAC2 in vitro tumors treated with vehicle, 3 μM Ezh2 inhibitor, or 10 μM Ezh2 inhibitor. FIG. 17E shows in vivo efficacy of HIT CD70 T cells expressing co-stimulation (CD80/4-1BBL) on PDAC2 orthotopic pancreatic PDX model.

FIGS. 18A and 18B depict characterization of PANC-1 cells as a model of CD70⁺ tumor cells for pancreatic cancer and in vivo efficacy of HIT CD70 T cells on pancreatic cancer.

FIG. 19A shows expression profile of CD70 in SK-OV3 ovarian cancer cell line. FIG. 19B shows tumor engraftment of SK-OV3 cell lines. FIGS. 19C-19E show cytotoxic effects on SK-OV3-derived orthotopic and intra-peritoneal ovarian cancer.

FIGS. 20A and 20B depict immunohistochemistry (IHC) and FISH analysis of CD70 in samples. FIG. 20A shows IHC and FISH analysis of CD70 in RCC kidney tumor patient and K7 PDX samples. FIG. 20B shows FISH analysis of CD70 in PDAC2 cell samples.

### DETAILED DESCRIPTION OF THE INVENTION

The presently disclosed subject matter provides compositions, e.g., modified immune cells, useful for immunotherapy (e.g., T cell immunotherapy). The presently disclosed compositions, e.g., modified immune cells, comprise: a TCR-like fusion molecule that targets CD70. The presently disclosed subject matter also provides methods for producing such compositions, and methods of using such compositions for treating and/or preventing tumors (e.g., cancer, e.g., a solid tumor, e.g., renal cell carcinoma (RCC)). The presently disclosed subject matter is based, at least in part, on the discovery that 70-HIT T cells can eradicate solid tumors (e.g., renal cell carcinoma, ovarian cancer, pancreatic cancer).

Non-limiting embodiments of the presently disclosed subject matter are described by the present specification and Examples.

For purposes of clarity of disclosure and not by way of limitation, the detailed description is divided into the following subsections:
1. Definitions;
2. Cells;
3. Nucleic Acid Compositions and Vectors;
4. Formulations and Administration;
5. Methods of Treatment;
6. Kits; and
7. Exemplary Embodiments.

### 1. DEFINITIONS

Unless defined otherwise, all technical and scientific terms used herein have the meaning commonly understood by a person skilled in the art. The following references provide one of skill with a general definition of many of the terms used in the presently disclosed subject matter: Singleton et al., Dictionary of Microbiology and Molecular Biology (2^{nd} ed. 1994); The Cambridge Dictionary of Science and Technology (Walker ed., 1988); The Glossary of Genetics, 5^{th} Ed., R. Rieger et al. (eds.), Springer Verlag (1991); and Hale & Marham, The Harper Collins Dictionary of Biology (1991).

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold or within 2-fold, of a value.

As used herein, a “co-stimulatory molecule” refer to a cell surface molecule other than an antigen receptor or its ligand that can provide an efficient response of lymphocytes to an antigen. In certain embodiments, a co-stimulatory molecule can provide optimal lymphocyte activation.

As used herein, a “co-stimulatory ligand” refers to a molecule that upon binding to its receptor (e.g., a co-stimulatory molecule) produces a co-stimulatory response, e.g., an intracellular response that effects the stimulation provided when an antigen-recognizing receptor (e.g., a chimeric antigen receptor (CAR)) binds to its target antigen.

By “immunoresponsive cell” is meant a cell that functions in an immune response or a progenitor, or progeny thereof. In certain embodiments, the immunoresponsive cell is a cell of lymphoid lineage. Non-limiting examples of cells of lymphoid lineage include T cells, Natural Killer (NK) cells, B cells, and stem cells from which lymphoid cells may be differentiated. In certain embodiments, the immunoresponsive cell is a cell of myeloid lineage.

By “activates an immunoresponsive cell” is meant induction of signal transduction or changes in protein expression in the cell resulting in initiation of an immune response. For example, when CD3 Chains cluster in response to ligand binding and immunoreceptor tyrosine-based inhibition motifs (ITAMs) a signal transduction cascade is produced. In certain embodiments, when an endogenous TCR or an exogenous CAR binds to an antigen, a formation of an immunological synapse occurs that includes clustering of many molecules near the bound receptor (e.g. CD4 or CD8, CD3γ/δ/ε/ζ, etc.). This clustering of membrane bound signaling molecules allows for ITAM motifs contained within the CD3 chains to become phosphorylated. This phosphorylation in turn initiates a T cell activation pathway ultimately activating transcription factors, such as NF-κB and AP-1. These transcription factors induce global gene expression of the T cell to increase IL-2 production for proliferation and expression of master regulator T cell proteins in order to initiate a T cell mediated immune response.

By “stimulates an immunoresponsive cell” is meant a signal that results in a robust and sustained immune response. In various embodiments, this occurs after immune cell (e.g., T-cell) activation or concomitantly mediated through receptors including, but not limited to, CD28, CD137 (4-1BB), OX40, CD40, ICOS, DAP-10, CD27, NKG2D, CD2, CD150, CD226. Receiving multiple stimulatory signals can be important to mount a robust and long-term T cell mediated immune response. T cells can quickly become inhibited and unresponsive to antigen. While the effects of these co-stimulatory signals may vary, they generally result in increased gene expression in order to generate long lived, proliferative, and anti-apoptotic T cells that robustly respond to antigen for complete and sustained eradication.

As used herein, the term “antigen heterogeneity” refers to the differential expression of a number of antigens (e.g., tumor antigens, e.g., CD70, CD312) which results in variation in the tumor cell phenotype and distribution of tumor antigen-positive cells.

As used herein, the term “low antigen density” refers to a target molecule (e.g., an antigen) having a cell surface density of less than about 5,000 molecules per cell. In certain embodiments, the low antigen density is a cell surface density is less than about 4,000 molecules per cell, less than about 3,000 molecules per cell, less than about 2,000 molecules per cell, less than about 1,500 molecules per cell, less than about 1,000 molecules per cell, less than about 500 molecules per cell, less than about 200 molecules per cell, or less than about 100 molecules per cell. In certain embodiments, the low antigen density is a cell surface density is less than about 2,000 molecules per cell. In certain embodiments, the low antigen density is a cell surface density is less than about 1,500 molecules per cell. In certain embodiments, the low antigen density is a cell surface density is less than about 1,000 molecules per cell. In certain embodiments, the low antigen density is a cell surface density is between about 4,000 molecules per cell and about 2,000 molecules per cell, between about 2,000 molecules per cell and about 1,000 molecules per cell, between about 1,500 molecules per cell and about 1,000 molecules per cell, between about 2,000 molecules per cell and about 500 molecules per cell, between about 1,000 molecules per cell and about 200 molecules per cell, or between about 1,000 molecules per cell and about 100 molecules per cell.

As used herein, the term “low tumor cell frequency” refers to a target cell having a target cell frequency of less than about 50% per tumor. In certain embodiments, the low tumor cell frequency is less than about 40% per tumor, less than about 30% per tumor, less than about 20% per tumor, less than about 15% per tumor, less than about 10% per tumor, less than about 5% per tumor, less than about 2% per tumor, or less than about 1% per tumor. In certain embodiments, the low tumor cell frequency is less than about 2% per tumor. In certain embodiments, the low tumor cell frequency is less than about 1.5% per tumor. In certain embodiments, the low tumor cell frequency is less than about 1% per tumor. In certain embodiments, the low tumor cell frequency is between about 40% per tumor and about 20% per tumor, between about 20% per tumor and about 10% per tumor, between about 15% per tumor and about 10% per tumor, between about 20% per tumor and about 5% per tumor, between about 10% per tumor and about 2% per tumor, or between about 10% per tumor and about 1% per tumor.

The term “antigen-recognizing receptor” as used herein refers to a receptor that is capable of activating an immune or immunoresponsive cell (e.g., a T-cell) in response to its binding to an antigen.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fe fragment of intact antibody, clear more rapidly from the circulation, and may have less non-specific tissue binding of an intact antibody (Wahl et al., J. Nucl. Med. 24:316-325 (1983). As used herein, antibodies include whole native antibodies, bispecific antibodies; chimeric antibodies; Fab, Fab′, single chain variable fragment (scFv), fusion polypeptides, and unconventional antibodies. In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (C_{H}) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further sub-divided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1 q) of the classical complement system.

As used herein, “CDRs” are defined as the complementarity determining region amino acid sequences of an antibody which are the hypervariable regions of immunoglobulin heavy and light chains. See, e.g., Kabat et al., Sequences of Proteins of Immunological Interest, 4^{th} U. S. Department of Health and Human Services, National Institutes of Health (1987). Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated using the Kabat system (Kabat, E. A., et al. (1991) Sequences of Proteins of Immunological Interest, Fifth Edition, U.S. Department of Health and Human Services, NIH Publication No. 91-3242). In certain embodiments, the CDRs regions are delineated using the PyIgClassify system (Adolf-Bryfogle et al., Nucleic acids research 43.D1 (2015): D432-D438).

As used herein, the term “Linker” shall mean a functional group (e.g., chemical or polypeptide) that covalently attaches two or more polypeptides or nucleic acids so that they are connected to one another. As used herein, a “peptide linker” refers to one or more amino acids used to couple two proteins together (e.g., to couple VH and VL domains). In certain embodiments, the linker is a G4S linker. In certain embodiments, the linker comprises or consists of the amino acid sequence set forth in SEQ ID NO: 1, which is provided below:

[SEQ ID NO: 1]
SGGGGSGGGGS

In certain embodiments, the linker comprises or consists of the amino acid sequence set forth in SEQ ID NO: 2, which is provided below:

[SEQ ID NO: 2]
GGGGSGGGGSGGGSGGGGS

In certain embodiments, the linker comprises or consists of the amino acid sequence set forth in SEQ ID NO: 3, which is provided below:

[SEQ ID NO: 3]
GGGGSGGGGSGGGGSGGGSGGGGS

In certain embodiments, the linker comprises or consists of the amino acid sequence set forth in SEQ ID NO: 4, which is provided below:

[SEQ ID NO: 4]
GGGGSGGGGSGGGGSGGGGSGGGSGGGGS

In certain embodiments, the linker comprises or consists of the amino acid sequence set forth in SEQ ID NO: 5, which is provided below:

[SEQ ID NO: 5]
GGGGS

In certain embodiments, the linker comprises or consists of the amino acid sequence set forth in SEQ ID NO: 6, which is provided below:

[SEQ ID NO: 6]
GGGGSGGGGS

As used herein, the term “single-chain variable fragment” or “scFv” is a fusion protein of the variable regions of the heavy (V_{H}) and light chains (V_{L}) of an immunoglobulin covalently linked to form a V_{H}::V_{L} heterodimer. The V_{H} and V_{L} are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. Despite removal of the constant regions and the introduction of a linker, scFv proteins retain the specificity of the original immunoglobulin. Single chain Fv polypeptide antibodies can be expressed from a nucleic acid including V_{H}- and V_{L}-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008 27(6):455-51; Peter et al., J Cachexia Sarcopenia Muscle 2012 Aug. 12; Shieh et al., J Imunol2009 183(4):2277-85; Giomarelli et al., Thromb Haemost 2007 97(6):955-63; Fife eta., J Clin Invst 2006 116(8):2252-61; Brocks et al., Immunotechnology 1997 3(3):173-84; Moosmayer et al., Ther Immunol 1995 2(10:31-40). Agonistic scFvs having stimulatory activity have been described (see, e.g., Peter et al., J Bioi Chern 2003 25278(38):36740-7; Xie et al., Nat Biotech 1997 15(8):768-71; Ledbetter et al., Crit Rev Immunol1997 17(5-6):427-55; Ho et al., BioChim Biophys Acta 2003 1638(3):257-66).

As used herein, the term “affinity” is meant a measure of binding strength. Affinity can depend on the closeness of stercochemical fit between antibody combining sites and antigen determinants, on the size of the area of contact between them, and/or on the distribution of charged and hydrophobic groups. As used herein, the term “affinity” also includes “avidity”, which refers to the strength of the antigen-antibody bond after formation of reversible complexes. Methods for calculating the affinity of an antibody for an antigen are known in the art, including, but not limited to, various antigen-binding experiments, e.g., functional assays (e.g., flow cytometry assay).

The term “chimeric antigen receptor” or “CAR” as used herein refers to a molecule comprising an extracellular antigen-binding domain that is fused to an intracellular signaling domain that is capable of activating or stimulating an immune or immunoresponsive cell, and a transmembrane domain. In certain embodiments, the extracellular antigen-binding domain of a CAR comprises an scFv. The scFv can be derived from fusing the variable heavy and light regions of an antibody. Alternatively or additionally, the scFv may be derived from Fab's (instead of from an antibody, e.g., obtained from Fab libraries). In certain embodiments, the scFv is fused to the transmembrane domain and then to the intracellular signaling domain. In certain embodiments, the CAR is selected to have high binding affinity or avidity for the antigen.

As used herein, the term “substantially identical” or “substantially homologous” refers to a polypeptide or a nucleic acid molecule exhibiting at least about 50% identical or homologous to a reference amino acid sequence (for example, any of the amino acid sequences described herein) or a reference nucleic acid sequence (for example, any of the nucleic acid sequences described herein). In certain embodiments, such a sequence is at least about 60%, at least about 65%, at least about 70%, at least about 75%, at least about 80%, at least about 85%, at least about 90%, at least about 95%, at least about 99%, or at least about 100% identical or homologous to the amino acid sequence or the nucleic acid sequence used for comparison.

Sequence identity can be measured by using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences (e.g., heavy and light chain variable region sequences) disclosed herein. To obtain gapped alignments for comparison purposes, Gapped BLAST can be utilized as described in Altschul et al., (1997) Nucleic Acids Res. 25 (17): 3389-3402. When utilizing BLAST and Gapped BLAST programs, the default parameters of the respective programs (e.g., XBLAST and NBLAST) can be used.

As used herein, the term “a conservative sequence modification” refers to an amino acid modification that does not significantly affect or alter the binding characteristics of the presently disclosed antigen recognizing receptors (e.g., the extracellular antigen-binding domain of the CAR) comprising the amino acid sequence. Conservative modifications can include amino acid substitutions, additions and deletions. Modifications can be introduced into the extracellular antigen-binding domain of the presently disclosed CAR by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (l) above) using the functional assays described herein. In certain embodiments, no more than one, no more than two, no more than three, no more than four, no more than five residues within a specified sequence or a CDR region are altered.

By “disease” is meant any condition, disease or disorder that damages or interferes with the normal function of a cell, tissue, or organ, e.g., neoplasm, and pathogen infection of cell.

By “effective amount” is meant an amount sufficient to have a therapeutic effect. In certain embodiments, an “effective amount” is an amount sufficient to arrest, ameliorate, or inhibit the continued proliferation, growth, or metastasis (e.g., invasion, or migration) of a neoplasm.

By “endogenous” is meant a nucleic acid molecule or polypeptide that is normally expressed in a cell or tissue.

By “exogenous” is meant a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

By “increase” is meant to alter positively by at least about 5%. An alteration may be by about 5%, about 10%, about 25%, about 30%, about 50%, about 75%, about 100% or more.

By “reduce” is meant to alter negatively by at least about 5%. An alteration may be by about 5%, about 10%, about 25%, about 30%, about 50%, about 75%, or even by about 100%.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated cell” is meant a cell that is separated from the molecular and/or cellular components that naturally accompany the cell.

The term “antigen-binding domain” as used herein refers to a domain capable of specifically binding a particular antigenic determinant or set of antigenic determinants present on a cell.

By “neoplasm” or “malignancy” is meant a disease characterized by the pathological proliferation of a cell or tissue and its subsequent migration to or invasion of other tissues or organs. Neoplasm growth is typically uncontrolled and progressive, and occurs under conditions that would not elicit, or would cause cessation of, multiplication of normal cells. Neoplasm can affect a variety of cell types, tissues, or organs, including but not limited to an organ selected from bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasms include cancers, such as sarcomas, carcinomas, or plasmacytomas (malignant tumor of the plasma cells). In certain embodiments, the neoplasm is cancer.

By “specifically binds” is meant a polypeptide or a fragment thereof that recognizes and binds to a biological molecule of interest (e.g., a polypeptide), but which does not substantially recognize and bind other molecules in a sample, for example, a biological sample, which naturally includes a presently disclosed polypeptide.

The term “tumor antigen” as used herein refers to an antigen (e.g., a polypeptide) that is uniquely or differentially expressed on a tumor cell compared to a normal or non-neoplastic cell. In certain embodiments, a tumor antigen includes any polypeptide expressed by a tumor that is capable of activating or inducing an immune response via an antigen recognizing receptor or capable of suppressing an immune response via receptor-ligand binding.

The terms “comprises”, “comprising”, and are intended to have the broad meaning ascribed to them in U.S. Patent Law and can mean “includes”, “including” and the like.

As used herein, “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys. The term “immunocompromised” as used herein refers to a subject who has an immunodeficiency. The subject is very vulnerable to opportunistic infections, infections caused by organisms that usually do not cause disease in a person with a healthy immune system, but can affect people with a poorly functioning or suppressed immune system.

As used herein, “a functional fragment” of a molecule or polypeptide includes a fragment of the molecule or polypeptide that retains at least about 80%, at least about 85%, at least about 90%, at least about 95%, or at least about 100% of the primary function of the molecule or polypeptide.

Other aspects of the presently disclosed subject matter are described in the following disclosure and are within the ambit of the presently disclosed subject matter.

### 2. CELLS

The presently disclosed subject matter provides cells comprising an antigen-recognizing receptor that targets CD70. In certain embodiments, the antigen-recognizing receptor is a TCR-like fusion molecule.

In certain embodiments, the cell is selected from the group consisting of cells of lymphoid lineage and cells of myeloid lineage. In certain embodiments, the cell is an immunoresponsive cell. In certain embodiments, the immunoresponsive cell is a cell of lymphoid lineage.

In certain embodiments, the cell is a cell of the lymphoid lineage. Cells of the lymphoid lineage can provide production of antibodies, regulation of cellular immune system, detection of foreign agents in the blood, detection of cells foreign to the host, and the like. Non-limiting examples of cells of the lymphoid lineage include T cells, Natural Killer (NK) cells, B cells, dendritic cells, stem cells from which lymphoid cells may be differentiated. In certain embodiments, the stem cell is a pluripotent stem cell (e.g., embryonic stem cell). In certain embodiments, the cell is a T cell. T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., TEM cells and TEMRA cells, Regulatory T cells (also known as suppressor T cells), tumor-infiltrating lymphocyte (TIL), Natural Killer T cells, Mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of an antigen-recognizing receptor, e.g., a CAR or a TCR. The T cell can be a CD4⁺ T cell or a CD8⁺ T cell. In certain embodiments, the T cell is a CD4⁺ T cell. In certain embodiments, the T cell is a CD8⁺ T cell. In certain embodiments, the CD8⁺ T cell is CD4 independent. In certain embodiments, the T cell is derived from an induced pluripotent stem cell (iPSC). In certain embodiments, the T cell is a CD8⁺ T cell that is CD4 independent, and the CD8⁺ T cell is derived from an iPSC.

In certain embodiments, the cell is a NK cell. Natural Killer (NK) cells can be lymphocytes that are part of cell-mediated immunity and act during the innate immune response. NK cells do not require prior activation in order to perform their cytotoxic effect on target cells.

Types of human lymphocytes of the presently disclosed subject matter include, without limitation, peripheral donor lymphocytes, e.g., those disclosed in Sadelain, M., et al. 2003 Nat Rev Cancer 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the α and β heterodimer), in Panelli, M. C., et al. 2000 J Immunol 164:495-504; Panelli, M. C., et al. 2000 J Immunol 164:4382-4392 (disclosing lymphocyte cultures derived from tumor infiltrating lymphocytes (TILs) in tumor biopsies), and in Dupont, J., et al. 2005 Cancer Res 65:5417-5427; Papanicolaou, G. A., et al. 2003 Blood 102:2498-2505 (disclosing selectively in vitro-expanded antigen-specific peripheral blood leukocytes employing artificial antigen-presenting cells (AAPCs) or pulsed dendritic cells).

In certain embodiments, the cell (e.g., T cell) is autologous. In certain embodiments, the cell (e.g., T cell) is non-autologous. In certain embodiments, the cell (e.g., T cell) is allogeneic. In certain embodiments, the cell (e.g., T cell) is derived in vitro from an engineered progenitor or stem cell.

In certain embodiments, the cell is a cell of the myeloid lineage. Non-limiting examples of cells of the myeloid lineage include monocytes, macrophages, neutrophils, basophils, cosinophils, erythrocytes, megakaryocytes, and stem cells from which myeloid cells may be differentiated.

In certain embodiments, the stem cell is a pluripotent stem cell (e.g., an embryonic stem cell or an induced pluripotent stem cell).

### 2.1. Antigen-Recognizing Receptor

The antigen-recognizing receptor (e.g., a first antigen-recognizing receptor) targets an antigen (e.g., a first antigen). The antigen (e.g., a first antigen) can be a tumor antigen or a pathogen antigen. In certain embodiments, the antigen-recognizing receptor (e.g., a first antigen-recognizing receptor) is a chimeric receptor. In certain embodiments, the chimeric receptor is a TCR-like fusion molecule.

### 2.1.1. Antigen

In certain embodiments, the antigen is a tumor antigen. In certain embodiments, the tumor antigen is an antigen with low antigen density. In certain embodiments, the tumor antigen is expressed on a cell with low tumor cell frequency.

Any tumor antigen (antigenic peptide) can be used in the tumor-related embodiments described herein. Sources of antigen include, but are not limited to, cancer proteins. The antigen (e.g., a first antigen) can be expressed as a peptide or as an intact protein or a portion thereof. The intact protein or portion thereof can be native or mutagenized. Non-limiting examples of tumor antigens include CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell (e.g. a cell surface antigen), ANO9, AQP2, ASIC3, ASPRV1, ATP6VOA4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD276, CD30, CD300LF, CD312, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-a, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, LICAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1,GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PR1), PSD2, PTPRJ, RDH16, receptor tyrosine-protein kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TASIR3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, and ZDHHC11. In certain embodiments, the antigen is CD70.

In certain embodiments, the antigen (e.g., a first antigen) is a pathogen antigen. Non-limiting examples of viruses include, Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Naira viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

Non-limiting examples of bacteria include Pasteurella, Staphylococci, Streptococcus, Escherichia coli, Pseudomonas species, and Salmonella species. Specific examples of infectious bacteria include but are not limited to, Helicobacter pyloris, Borelia burgdorferi, Legionella, Legionella pneumophilia, Mycobacteria sps (e.g. M. tuberculosis, M. avium, M. intracellulare, M. kansaii, M. gordonae, M. leprae), Staphylococcus aureus, Staphylococcus epidermidis, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Campylobacter jejuni, Enterococcus sp., Haemophilus influenzae, Bacillus antracis, Corynebacterium diphtheriae, Corynebacterium sp., Erysipelothrix rhusiopathiae, Clostridium spp., Clostridium perfringers, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Rickettsia, and Actinomyces israelli. Mycoplasma, Pseudomonas aeruginosa, Pseudomonas fluorescens, Corynobacteria diphtheriae, Bartonella henselae, Bartonella quintana, Coxiella burnetii, chlamydia, shigella, Yersinia enterocolitica, Yersinia pseudotuberculosis, Listeria monocytogenes, Mycoplasma spp., Vibrio cholerae, Borrelia, Francisella, Brucella melitensis, Proteus mirabilis, and Proteus.

In certain embodiments, the pathogen antigen is a viral antigen present in Cytomegalovirus (CMV), a viral antigen present in Epstein Barr Virus (EBV), a viral antigen present in Human Immunodeficiency Virus (HIV), or a viral antigen present in influenza virus.

### 2.1.2. TCR-Like Fusion Molecules

In certain embodiments, the antigen-recognizing receptor is a TCR-like fusion molecule. Non-limiting examples of TCR fusion molecules include HLA-Independent TCR-based Chimeric Antigen Receptor (also known as “HIT”, e.g., those disclosed in International Patent Application No. PCT/US19/017525, which is incorporated by reference in its entirety), and T cell receptor fusion constructs (TRuCs) (e.g., those disclosed in Baeuerle et al., “Synthetic TRUC receptors engaging the complete T cell receptor for potent anti-tumor response,” Nature Communications volume 10, Article number: 2087 (2019), which is incorporated by reference in its entirety).

In certain embodiments, the TCR-like fusion molecule is a recombinant T cell receptor (TCR). In certain embodiments, the recombinant TCR comprises at least one antigen-binding chain. In certain embodiments, the antigen-binding domain of the recombinant TCR comprises a ligand for a cell-surface receptor, a receptor for a cell surface ligand, an antigen binding portion of an antibody or a fragment thereof, or an antigen binding portion of a TCR. In certain embodiments, the recombinant TCR comprises two antigen binding chains, i.e., a first antigen binding chain and a second antigen binding chain. In certain embodiments, the first and second antigen-binding chains each comprises a constant domain. In certain embodiments, the recombinant TCR binds to an antigen (e.g., a first antigen or a second antigen) in an HLA-independent manner. Thus, in certain embodiments, the recombinant TCR is an HLA-independent (or non-HLA restricted) TCR (referred to as “HIT”).

In certain embodiments, the first antigen-binding chain comprises an antigen-binding fragment of a heavy chain variable region (V_{H}) of an antibody. In certain embodiments, the second antigen-binding chain comprises an antigen-binding fragment of a light chain variable region (V_{L}) of an antibody. In certain embodiments, the first antigen-binding chain comprises an antigen-binding fragment of a V_{H} of an antibody, and the second antigen-binding chain comprises an antigen-binding fragment of a V_{L} of the antibody.

In certain embodiments, the constant domain comprises a TCR constant region selected from the group consisting of a native or modified TRAC polypeptide, a native or modified TRBC polypeptide, a native or modified TRDC polypeptide, a native or modified TRGC polypeptide and any variants or functional fragments thereof. In certain embodiments, the constant domain comprises a native or modified TRAC polypeptide. In certain embodiments, the constant domain comprises a native or modified TRBC polypeptide. In certain embodiments, the first antigen-binding chain comprises a TRAC polypeptide, and the second antigen-binding chain comprises a TRBC polypeptide. In certain embodiments, the first antigen-binding chain comprises a TRBC polypeptide, and the second antigen-binding chain comprises a TRAC polypeptide.

In certain embodiments, the first antigen-binding chain comprises a V_{H} of an antibody and a TRAC polypeptide, and the second antigen-binding chain comprises a V_{L} of an antibody and a TRBC polypeptide.

In certain embodiments, the first antigen-binding chain comprises a V_{H} of an antibody and a TRBC polypeptide, and the second antigen-binding chain comprises a V_{L} of an antibody and a TRAC polypeptide.

In certain embodiments, at least one of the TRAC polypeptide and the TRBC polypeptide is endogenous. In certain embodiments, the TRAC polypeptide is endogenous. In certain embodiments, the TRBC polypeptide is endogenous. In certain embodiments, both the TRAC polypeptide and the TRBC polypeptide are endogenous.

In certain embodiments, the antigen binding chain is capable of associating with a CD3ζ polypeptide. In certain embodiments, the antigen binding chain, upon binding to an antigen, is capable of activating the CD3ζ polypeptide associated to the antigen binding chain. In certain embodiments, the activation of the CD3ζ polypeptide is capable of activating an immunoresponsive cell. In certain embodiments, the TCR-like fusion molecule is capable of integrating with a CD3 complex and providing HLA-independent antigen recognition. In certain embodiments, the TCR-like fusion molecule replaces an endogenous TCR in a CD3/TCR complex.

In certain embodiments, the first and second antigen binding chains bind to an antigen with a dissociation constant (K_{D}) of about 2×10⁻⁷ M or less. In certain embodiments, the first and second antigen binding chains bind to an antigen with a high binding affinity. In certain embodiments, the K_{D} is about 2×10⁻⁷ M or less, about 1×10⁻⁷ M or less, about 9×10⁻⁸ M or less, about 1×10⁻⁸ M or less, about 9×10⁻⁹ M or less, about 5×10⁻⁹ M or less, about 4×10⁻⁹ M or less, about 3×10⁻⁹ or less, about 2×10⁻⁹ M or less, or about 1×10⁻⁹ M or less. In certain embodiments, the K_{D} is about 1×10⁻⁸ M or less. In certain embodiments, the K_{D} is about 3×10⁻⁹ M or less. In certain embodiments, the K_{D} is about 5×10⁻⁹M or less. In certain embodiments, the K_{D} is from about 1×10⁻⁹ M to about 1×10⁻⁸ M. In certain embodiments, the K_{D} is from about 1.5×10⁻⁹ M to about 1×10⁻⁸ M. In certain embodiments, the K_{D} is from about 5×10⁻⁹ M to about 1×10⁻⁸ M.

In certain embodiments, the constant domain comprises a TCR constant region, e.g., T cell receptor alpha constant region (TRAC), T cell receptor beta constant region (TRBC, e.g., TRBC1 or TRBC2), T cell receptor gamma constant region (TRGC, e.g., TRGC1 or TRGC2), T cell receptor delta constant region (TRDC) or any variants or functional fragments thereof.

In certain embodiments, the first antigen binding chain or the second antigen binding chain comprises a constant domain that comprises a native or modified TRAC polypeptide. In certain embodiments, the TRAC polypeptide comprises an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 7 or a fragment thereof, and/or may optionally comprise up to one or up to two or up to three conservative amino acid substitutions. In certain embodiments, the TRAC polypeptide comprises or consists of the amino acid sequence set forth in SEQ ID NO: 7. SEQ ID NO: 7 is provided below.

[SEQ ID NO: 7]
IQNPDPAVYQLRDSKSSDKSVCLFTDFDSQTNVSQSKDSDVYITD
KTVLDMRSMDFKSNSAVAWSNKSDEACANAENNSIIPEDTFFPSP
ESSCDVKLVEKSFETDTNLNFQNLSVIGFRILLLKVAGENLLMTL
RLWSS

An exemplary nucleotide sequence encoding the amino acid sequence of SEQ ID NO: 7 is set forth in SEQ ID NO: 8, which is provided below.

[SEQ ID NO: 8]
ATCCAGAACCCTGACCCTGCCGTGTACCAGCTGAGAGACTCTAAA
TCCAGTGACAAGTCTGTCTGCCTATTCACCGATTTTGATTCTCAA
ACAAATGTGTCACAAAGTAAGGATTCTGATGTGTATATCACAGAC
AAAACTGTGCTAGACATGAGGTCTATGGACTTCAAGAGCAACAGT
GCTGTGGCCTGGAGCAACAAATCTGACTTTGCATGTGCAAACGCC
TTCAACAACAGCATTATTCCAGAAGACACCTTCTTCCCCAGCCCA
GAAAGTTCCTGTGATGTCAAGCTGGTCGAGAAAAGCTTTGAAACA
GATACGAACCTAAACTTTCAAAACCTGTCAGTGATTGGGTTCCGA
ATCCTCCTCCTGAAAGTGGCCGGGTTTAATCTGCTCATGACGCTG
CGGCTGTGGTCCAGC

In certain embodiments, the TRAC polypeptide comprises an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 9 or a fragment thereof, and/or may optionally comprise up to one or up to two or up to three conservative amino acid substitutions. In certain embodiments, the TRAC polypeptide comprises or consists of the amino acid sequence set forth in SEQ ID NO: 9. SEQ ID NO: 9 is provided below.

[SEQ ID NO: 9]
IPNIQNPDPAVYQLRDSKSSDKSVCLFTDFDSQTNVSQSKDSDVY
ITDKTVLDMRSMDFKSNSAVAWSNKSDFACANAFNNSIIPEDTFF
PSPESSCDVKLVEKSFETDTNLNFQNLSVIGFRILLLKVAGENLL
MTLRLWSS

An exemplary nucleotide sequence encoding the amino acid sequence of SEQ ID NO: 9 is set forth in SEQ ID NO: 10, which is provided below.

[SEQ ID NO: 10]
ATTCCCAATATCCAGAACCCTGACCCTGCCGTGTACCAGCTGAGA
GACTCTAAATCCAGTGACAAGTCTGTCTGCCTATTCACCGATTTT
GATTCTCAAACAAATGTGTCACAAAGTAAGGATTCTGATGTGTAT
ATCACAGACAAAACTGTGCTAGACATGAGGTCTATGGACTTCAAG
AGCAACAGTGCTGTGGCCTGGAGCAACAAATCTGACTTTGCATGT
GCAAACGCCTTCAACAACAGCATTATTCCAGAAGACACCTTCTTC
CCCAGCCCAGAAAGTTCCTGTGATGTCAAGCTGGTCGAGAAAAGC
TTTGAAACAGATACGAACCTAAACTTTCAAAACCTGTCAGTGATT
GGGTTCCGAATCCTCCTCCTGAAAGTGGCCGGGTTTAATCTGCTC
ATGACGCTGCGGCTGTGGTCCAGC

In certain embodiments, the TRAC polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence encoded by a transcript expressed by the gene of NCBI Genbank ID: 28755, NG_001332.3, range 925603 to 930229 (SEQ ID NO: 11) or a fragment thereof, and/or may optionally comprise up to one or up to two or up to three conservative amino acid substitutions. In certain embodiments, the TRAC polypeptide comprises or consists of the amino acid sequence encoded by the nucleotide sequence of SEQ ID NO: 11. SEQ ID NO: 11 is provided below.

[SEQ ID NO: 11]
ATATCCAGAACCCTGACCCTGCCGTGTACCAGCTGAGAGACTCTAA
ATCCAGTGACAAGTCTGTCTGCCTATTCACCGATTTTGATTCTCA
AACAAATGTGTCACAAAGTAAGGATTCTGATGTGTATATCACAGA
CAAAACTGTGCTAGACATGAGGTCTATGGACTTCAAGAGCAACAG
TGCTGTGGCCTGGAGCAACAAATCTGACTTTGCATGTGCAAACGC
CTTCAACAACAGCATTATTCCAGAAGACACCTTCTTCCCCAGCCC
AGGTAAGGGCAGCTTTGGTGCCTTCGCAGGCTGTTTCCTTGCTTC
AGGAATGGCCAGGTTCTGCCCAGAGCTCTGGTCAATGATGTCTAA
AACTCCTCTGATTGGTGGTCTCGGCCTTATCCATTGCCACCAAAA
CCCTCTTTTTACTAAGAAACAGTGAGCCTTGTTCTGGCAGTCCAG
AGAATGACACGGGAAAAAAGCAGATGAAGAGAAGGTGGCAGGAGA
GGGCACGTGGCCCAGCCTCAGTCTCTCCAACTGAGTTCCTGCCTG
CCTGCCTTTGCTCAGACTGTTTGCCCCTTACTGCTCTTCTAGGCC
TCATTCTAAGCCCCTTCTCCAAGTTGCCTCTCCTTATTTCTCCCT
GTCTGCCAAAAAATCTTTCCCAGCTCACTAAGTCAGTCTCACGCA
GTCACTCATTAACCCACCAATCACTGATTGTGCCGGCACATGAAT
GCACCAGGTGTTGAAGTGGAGGAATTAAAAAGTCAGATGAGGGGT
GTGCCCAGAGGAAGCACCATTCTAGTTGGGGGAGCCCATCTGTCA
GCTGGGAAAAGTCCAAATAACTTCAGATTGGAATGTGTTTTAACT
CAGGGTTGAGAAAACAGCTACCTTCAGGACAAAAGTCAGGGAAGG
GCTCTCTGAAGAAATGCTACTTGAAGATACCAGCCCTACCAAGGG
CAGGGAGAGGACCCTATAGAGGCCTGGGACAGGAGCTCAATGAGA
AAGGAGAAGAGCAGCAGGCATGAGTTGAATGAAGGAGGCAGGGCC
GGGTCACAGGGCCTTCTAGGCCATGAGAGGGTAGACAGTATTCTA
AGGACGCCAGAAAGCTGTTGATCGGCTTCAAGCAGGGGAGGGACA
CCTAATTTGCTTTTCTTTTTTTTTTTTTTTTTTTTTTTTTTTTTT
GAGATGGAGTTTTGCTCTTGTTGCCCAGGCTGGAGTGCAATGGTG
CATCTTGGCTCACTGCAACCTCCGCCTCCCAGGTTCAAGTGATTC
TCCTGCCTCAGCCTCCCGAGTAGCTGAGATTACAGGCACCCGCCA
CCATGCCTGGCTAATTTTTTGTATTTTTAGTAGAGACAGGGTTTC
ACTATGTTGGCCAGGCTGGTCTCGAACTCCTGACCTCAGGTGATC
CACCCGCTTCAGCCTCCCAAAGTGCTGGGATTACAGGCGTGAGCC
ACCACACCCGGCCTGCTTTTCTTAAAGATCAATCTGAGTGCTGTA
CGGAGAGTGGGTTGTAAGCCAAGAGTAGAAGCAGAAAGGGAGCAG
TTGCAGCAGAGAGATGATGGAGGCCTGGGCAGGGTGGTGGCAGGG
AGGTAACCAACACCATTCAGGTTTCAAAGGTAGAACCATGCAGGG
ATGAGAAAGCAAAGAGGGGATCAAGGAAGGCAGCTGGATTTTGGC
CTGAGCAGCTGAGTCAATGATAGTGCCGTTTACTAAGAAGAAACC
AAGGAAAAAATTTGGGGTGCAGGGATCAAAACTTTTTGGAACATA
TGAAAGTACGTGTTTATACTCTTTATGGCCCTTGTCACTATGTAT
GCCTCGCTGCCTCCATTGGACTCTAGAATGAAGCCAGGCAAGAGC
AGGGTCTATGTGTGATGGCACATGTGGCCAGGGTCATGCAACATG
TACTTTGTACAAACAGTGTATATTGAGTAAATAGAAATGGTGTCC
AGGAGCCGAGGTATCGGTCCTGCCAGGGCCAGGGGCTCTCCCTAG
CAGGTGCTCATATGCTGTAAGTTCCCTCCAGATCTCTCCACAAGG
AGGCATGGAAAGGCTGTAGTTGTTCACCTGCCCAAGAACTAGGAG
GTCTGGGGTGGGAGAGTCAGCCTGCTCTGGATGCTGAAAGAATGT
CTGTTTTTCCTTTTAGAAAGTTCCTGTGATGTCAAGCTGGTCGAG
AAAAGCTTTGAAACAGGTAAGACAGGGGTCTAGCCTGGGTTTGCA
CAGGATTGCGGAAGTGATGAACCCGCAATAACCCTGCCTGGATGA
GGGAGTGGGAAGAAATTAGTAGATGTGGGAATGAATGATGAGGAA
TGGAAACAGCGGTTCAAGACCTGCCCAGAGCTGGGTGGGGTCTCT
CCTGAATCCCTCTCACCATCTCTGACTTTCCATTCTAAGCACTTT
GAGGATGAGTTTCTAGCTTCAATAGACCAAGGACTCTCTCCTAGG
CCTCTGTATTCCTTTCAACAGCTCCACTGTCAAGAGAGCCAGAGA
GAGCTTCTGGGTGGCCCAGCTGTGAAATTTCTGAGTCCCTTAGGG
ATAGCCCTAAACGAACCAGATCATCCTGAGGACAGCCAAGAGGTT
TTGCCTTCTTTCAAGACAAGCAACAGTACTCACATAGGCTGTGGG
CAATGGTCCTGTCTCTCAAGAATCCCCTGCCACTCCTCACACCCA
CCCTGGGCCCATATTCATTTCCATTTGAGTTGTTCTTATTGAGTC
ATCCTTCCTGTGGTAGCGGAACTCACTAAGGGGCCCATCTGGACC
CGAGGTATTGTGATGATAAATTCTGAGCACCTACCCCATCCCCAG
AAGGGCTCAGAAATAAAATAAGAGCCAAGTCTAGTCGGTGTTTCC
TGTCTTGAAACACAATACTGTTGGCCCTGGAAGAATGCACAGAAT
CTGTTTGTAAGGGGATATGCACAGAAGCTGCAAGGGACAGGAGGT
GCAGGAGCTGCAGGCCTCCCCCACCCAGCCTGCTCTGCCTTGGGG
AAAACCGTGGGTGTGTCCTGCAGGCCATGCAGGCCTGGGACATGC
AAGCCCATAACCGCTGTGGCCTCTTGGTTTTACAGATACGAACCT
AAACTTTCAAAACCTGTCAGTGATTGGGTTCCGAATCCTCCTCCT
GAAAGTGGCCGGGTTTAATCTGCTCATGACGCTGCGGCTGTGGTC
CAGCTGAGGTGAGGGGCCTTGAAGCTGGGAGTGGGGTTTAGGGAC
GCGGGTCTCTGGGTGCATCCTAAGCTCTGAGAGCAAACCTCCCTG
CAGGGTCTTGCTTTTAAGTCCAAAGCCTGAGCCCACCAAACTCTC
CTACTTCTTCCTGTTACAAATTCCTCTTGTGCAATAATAATGGCC
TGAAACGCTGTAAAATATCCTCATTTCAGCCGCCTCAGTTGCACT
TCTCCCCTATGAGGTAGGAAGAACAGTTGTTTAGAAACGAAGAAA
CTGAGGCCCCACAGCTAATGAGTGGAGGAAGAGAGACACTTGTGT
ACACCACATGCCTTGTGTTGTACTTCTCTCACCGTGTAACCTCCT
CATGTCCTCTCTCCCCAGTACGGCTCTCTTAGCTCAGTAGAAAGA
AGACATTACACTCATATTACACCCCAATCCTGGCTAGAGTCTCCG
CACCCTCCTCCCCCAGGGTCCCCAGTCGTCTTGCTGACAACTGCA
TCCTGTTCCATCACCATCAAAAAAAAACTCCAGGCTGGGTGCGGG
GGCTCACACCTGTAATCCCAGCACTTTGGGAGGCAGAGGCAGGAG
GAGCACAGGAGCTGGAGACCAGCCTGGGCAACACAGGGAGACCCC
GCCTCTACAAAAAGTGAAAAAATTAACCAGGTGTGGTGCTGCACA
CCTGTAGTCCCAGCTACTTAAGAGGCTGAGATGGGAGGATCGCTT
GAGCCCTGGAATGTTGAGGCTACAATGAGCTGTGATTGCGTCACT
GCACTCCAGCCTGGAAGACAAAGCAAGATCCTGTCTCAAATAATA
AAAAAAATAAGAACTCCAGGGTACATTTGCTCCTAGAACTCTACC
ACATAGCCCCAAACAGAGCCATCACCATCACATCCCTAACAGTCC
TGGGTCTTCCTCAGTGTCCAGCCTGACTTCTGTTCTTCCTCATTC
CAGATCTGCAAGATTGTAAGACAGCCTGTGCTCCCTCGCTCCTTC
CTCTGCATTGCCCCTCTTCTCCCTCTCCAAACAGAGGGAACTCTC
CTACCCCCAAGGAGGTGAAAGCTGCTACCACCTCTGTGCCCCCCC
GGCAATGCCACCAACTGGATCCTACCCGAATTTATGATTAAGATT
GCTGAAGAGCTGCCAAACACTGCTGCCACCCCCTCTGTTCCCTTA
TTGCTGCTTGTCACTGCCTGACATTCACGGCAGAGGCAAGGCTGC
TGCAGCCTCCCCTGGCTGTGCACATTCCCTCCTGCTCCCCAGAGA
CTGCCTCCGCCATCCCACAGATGATGGATCTTCAGTGGGTTCTCT
TGGGCTCTAGGTCCTGCAGAATGTTGTGAGGGGTTTATTTTTTTT
TAATAGTGTTCATAAAGAAATACATAGTATTCTTCTTCTCAAGAC
GTGGGGGGAAATTATCTCATTATCGAGGCCCTGCTATGCTGTGTA
TCTGGGCGTGTTGTATGTCCTGCTGCCGATGCCTTC

In certain embodiments, the first antigen binding chain or the second antigen binding chain comprises a constant domain comprising a native or modified TRBC polypeptide. In certain embodiments, the TRBC polypeptide is a TRBC2 polypeptide. In certain embodiments, the TRBC2 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 12 or a fragment thereof, and/or may optionally comprise up to one or up to two or up to three conservative amino acid substitutions. In certain embodiments, the TRBC2 polypeptide comprises or consists of the amino acid sequence set forth in SEQ ID NO: 12. SEQ ID NO: 12 is provided below.

[SEQ ID NO: 12]
DLKNVFPPEVAVFEPSEAEISHTQKATLVCLATGFYPDHVELSWW
VNGKEVHSGVSTDPQPLKEQPALNDSRYCLSSRLRVSATFWQNPR
NHFRCQVQFYGLSENDEWTQDRAKPVTQIVSAEAWGRADCGFTSE
SYQQGVLSATILYEILLGKATLYAVLVSALVLMAMVKRKDSRG

An exemplary nucleotide sequence encoding the amino acid sequence of SEQ ID NO: 12 is set forth in SEQ ID NO: 13, which is provided below.

[SEQ ID NO: 13]
GATCTGAAAAACGTGTTCCCTCCTGAAGTGGCTGTCTTTGAACCA
TCCGAGGCCGAGATTTCCCATACCCAGAAAGCAACTCTGGTCTGT
CTGGCCACTGGATTCTACCCCGATCACGTGGAACTGTCTTGGTGG
GTGAACGGCAAGGAAGTCCATTCCGGAGTCTCTACCGACCCTCAG
CCCCTCAAGGAGCAGCCTGCTCTCAACGATTCTCGGTACTGCCTG
TCATCTCGACTGAGAGTGTCTGCCACCTTCTGGCAGAACCCTAGA
AACCACTTTCGGTGTCAGGTCCAGTTTTACGGCCTGAGCGAGAAC
GATGAGTGGACACAGGATAGAGCCAAACCTGTGACACAGATTGTG
AGCGCCGAGGCTTGGGGACGAGCCGATTGTGGCTTCACATCCGAG
TCTTACCAGCAGGGAGTGCTGTCTGCTACAATCCTCTACGAAATT
CTCCTGGGGAAGGCCACCCTGTACGCTGTCCTCGTGTCTGCTCTG
GTGCTCATGGCTATGGTCAAACGAAAGGACTCTAGAGGC

In certain embodiments, the TRBC polypeptide is a TRBC2 polypeptide. In certain embodiments, the TRBC2 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 14 or a fragment thereof, and/or may optionally comprise up to one or up to two or up to three conservative amino acid substitutions. In certain embodiments, the TRBC2 polypeptide comprises or consists of the amino acid sequence set forth in SEQ ID NO: 14. SEQ ID NO: 14 is provided below.

[SEQ ID NO: 14]
LEDLKNVEPPEVAVFEPSEAEISHTQKATLVCLATGFYPDHVELS
WWVNGKEVHSGVSTDPQPLKEQPALNDSRYCLSSRLRVSATFWQN
PRNHFRCQVQFYGLSENDEWTQDRAKPVTQIVSAEAWGRADCGFT
SESYQQGVLSATILYEILLGKATLYAVLVSALVLMAMVKRKDSRG

An exemplary nucleotide sequence encoding the amino acid sequence of SEQ ID NO: 14 is set forth in SEQ ID NO: 15, which is provided below.

[SEQ ID NO: 15]
CTGGAGGATCTGAAAAACGTGTTCCCTCCTGAAGTGGCTGTCTTT
GAACCATCCGAGGCCGAGATTTCCCATACCCAGAAAGCAACTCTG
GTCTGTCTGGCCACTGGATTCTACCCCGATCACGTGGAACTGTCT
TGGTGGGTGAACGGCAAGGAAGTCCATTCCGGAGTCTCTACCGAC
CCTCAGCCCCTCAAGGAGCAGCCTGCTCTCAACGATTCTCGGTAC
TGCCTGTCATCTCGACTGAGAGTGTCTGCCACCTTCTGGCAGAAC
CCTAGAAACCACTTTCGGTGTCAGGTCCAGTTTTACGGCCTGAGC
GAGAACGATGAGTGGACACAGGATAGAGCCAAACCTGTGACACAG
ATTGTGAGCGCCGAGGCTTGGGGACGAGCCGATTGTGGCTTCACA
TCCGAGTCTTACCAGCAGGGAGTGCTGTCTGCTACAATCCTCTAC
GAAATTCTCCTGGGGAAGGCCACCCTGTACGCTGTCCTCGTGTCT
GCTCTGGTGCTCATGGCTATGGTCAAACGAAAGGACTCTAGAGGC

In certain embodiments, the TRBC polypeptide is a TRBC1 polypeptide. In certain embodiments, the TRBC1 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 16 or a fragment thereof, and/or may optionally comprise up to one or up to two or up to three conservative amino acid substitutions. In certain embodiments, the TRBC1 polypeptide comprises or consists of the amino acid sequence set forth in SEQ ID NO: 16. SEQ ID NO: 16 is provided below.

[SEQ ID NO: 16]
LNKVFPPEVAVFEPSEAEISHTQKATLVCLATGFFPDHVELSWWV
NGKEVHSGVSTDPQPLKEQPALNDSRYCLSSRLRVSATFWQNPRN
HFRCQVQFYGLSENDEWTQDRAKPVTQIVSAEAWGRADCGFTSVS
YQQGVLSATILYEILLGKATLYAVLVSALVLMAMVKRKDF

In certain embodiments, the TRBC1 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 17 or a fragment thereof, and/or may optionally comprise up to one or up to two or up to three conservative amino acid substitutions. In certain embodiments, the TRBC1 polypeptide comprises or consists of the amino acid sequence set forth in SEQ ID NO: 17. SEQ ID NO: 17 is provided below.

[SEQ ID NO: 17]
DLNKVFPPEVAVFEPSEAFISHTQKATLVCLATGFFPDHVELSWW
VNGKEVHSGVSTDPQPLKEQPALNDSRYCLSSRLRVSATFWQNPR
NHERCQVQFYGLSENDEWTQDRAKPVTQIVSAEAWGRADCGFTSV
SYQQGVLSATILYEILLGKATLYAVLVSALVLMAMVKRKDF

An exemplary nucleotide sequence encoding the amino acid sequence of SEQ ID NO: 17 is set forth in SEQ ID NO: 18, which is provided below.

[SEQ ID NO: 18]
GACCTGAACAAGGTGTTCCCACCCGAGGTCGCTGTGTTTGAGCCA
TCAGAAGCAGAGATCTCCCACACCCAAAAGGCCACACTGGTGTGC
CTGGCCACAGGCTTCTTCCCCGACCACGTGGAGCTGAGCTGGTGG
GTGAATGGGAAGGAGGTGCACAGTGGGGTCAGCACAGACCCGCAG
CCCCTCAAGGAGCAGCCCGCCCTCAATGACTCCAGATACTGCCTG
AGCAGCCGCCTGAGGGTCTCGGCCACCTTCTGGCAGAACCCCCGC
AACCACTTCCGCTGTCAAGTCCAGTTCTACGGGCTCTCGGAGAAT
GACGAGTGGACCCAGGATAGGGCCAAACCCGTCACCCAGATCGTC
AGCGCCGAGGCCTGGGGTAGAGCAGACTGTGGCTTTACCTCGGTG
TCCTACCAGCAAGGGGTCCTGTCTGCCACCATCCTCTATGAGATC
CTGCTAGGGAAGGCCACCCTGTATGCTGTGCTGGTCAGCGCCCTT
GTGTTGATGGCCATGGTCAAGAGAAAGGATTTC

In certain embodiments, the TRBC polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence encoded by a transcript expressed by a gene of NCBI Genbank ID: 28639, NG_001333.2, range 645749 to 647196 (TRBC1, SEQ ID NO: 19), NCBI Genbank ID: 28638, NG_001333.2 range 655095 to 656583 (TRBC2, SEQ ID NO: 20) or a fragment thereof, and/or may optionally comprise up to one or up to two or up to three conservative amino acid substitutions. In certain embodiments, the TRBC polypeptide comprises or consists of the amino acid sequence encoded by the nucleotide sequence of SEQ ID NO: 19. In certain embodiments, the TRBC polypeptide comprises or consists of the amino acid sequence encoded by the nucleotide sequence of SEQ ID NO: 20. SEQ ID NO: 19 and 20 are provided below.

[SEQ ID NO: 19]
AGGACCTGAACAAGGTGTTCCCACCCGAGGTCGCTGTGTTTGAGC
CATCAGAAGCAGAGATCTCCCACACCCAAAAGGCCACACTGGTGT
GCCTGGCCACAGGCTTCTTCCCCGACCACGTGGAGCTGAGCTGGT
GGGTGAATGGGAAGGAGGTGCACAGTGGGGTCAGCACAGACCCGC
AGCCCCTCAAGGAGCAGCCCGCCCTCAATGACTCCAGATACTGCC
TGAGCAGCCGCCTGAGGGTCTCGGCCACCTTCTGGCAGAACCCCC
GCAACCACTTCCGCTGTCAAGTCCAGTTCTACGGGCTCTCGGAGA
ATGACGAGTGGACCCAGGATAGGGCCAAACCCGTCACCCAGATCG
TCAGCGCCGAGGCCTGGGGTAGAGCAGGTGAGTGGGGCCTGGGGA
GATGCCTGGAGGAGATTAGGTGAGACCAGCTACCAGGGAAAATGG
AAAGATCCAGGTAGCAGACAAGACTAGATCCAAAAAGAAAGGAAC
CAGCGCACACCATGAAGGAGAATTGGGCACCTGTGGTTCATTCTT
CTCCCAGATTCTCAGCCCAACAGAGCCAAGCAGCTGGGTCCCCTT
TCTATGTGGCCTGTGTAACTCTCATCTGGGTGGTGCCCCCCATCC
CCCTCAGTGCTGCCACATGCCATGGATTGCAAGGACAATGTGGCT
GACATCTGCATGGCAGAAGAAAGGAGGTGCTGGGCTGTCAGAGGA
AGCTGGTCTGGGCCTGGGAGTCTGTGCCAACTGCAAATCTGACTT
TACTTTTAATTGCCTATGAAAATAAGGTCTCTCATTTATTTTCCT
CTCCCTGCTTTCTTTCAGACTGTGGCTTTACCTCGGGTAAGTAAG
CCCTTCCTTTTCCTCTCCCTCTCTCATGGTTCTTGACCTAGAACC
AAGGCATGAAGAACTCACAGACACTGGAGGGTGGAGGGTGGGAGA
GACCAGAGCTACCTGTGCACAGGTACCCACCTGTCCTTCCTCCGT
GCCAACAGTGTCCTACCAGCAAGGGGTCCTGTCTGCCACCATCCT
CTATGAGATCCTGCTAGGGAAGGCCACCCTGTATGCTGTGCTGGT
CAGCGCCCTTGTGTTGATGGCCATGGTAAGCAGGAGGGCAGGATG
GGGCCAGCAGGCTGGAGGTGACACACTGACACCAAGCACCCAGAA
GTATAGAGTCCCTGCCAGGATTGGAGCTGGGCAGTAGGGAGGGAA
GAGATTTCATTCAGGTGCCTCAGAAGATAACTTGCACCTCTGTAG
GATCACAGTGGAAGGGTCATGCTGGGAAGGAGAAGCTGGAGTCAC
CAGAAAACCCAATGGATGTTGTGATGAGCCTTACTATTTGTGTGG
TCAATGGGCCCTACTACTTTCTCTCAATCCTCACAACTCCTGGCT
CTTAATAACCCCCAAAACTTTCTCTTCTGCAGGTCAAGAGAAAGG
ATTTCTGA
[SEQ ID NO: 20]
AGGACCTGAAAAACGTGTTCCCACCCGAGGTCGCTGTGTTTGAGC
CATCAGAAGCAGAGATCTCCCACACCCAAAAGGCCACACTGGTAT
GCCTGGCCACAGGCTTCTACCCCGACCACGTGGAGCTGAGCTGGT
GGGTGAATGGGAAGGAGGTGCACAGTGGGGTCAGCACAGACCCGC
AGCCCCTCAAGGAGCAGCCCGCCCTCAATGACTCCAGATACTGCC
TGAGCAGCCGCCTGAGGGTCTCGGCCACCTTCTGGCAGAACCCCC
GCAACCACTTCCGCTGTCAAGTCCAGTTCTACGGGCTCTCGGAGA
ATGACGAGTGGACCCAGGATAGGGCCAAACCCGTCACCCAGATCG
TCAGCGCCGAGGCCTGGGGTAGAGCAGGTGAGTGGGGCCTGGGGA
GATGCCTGGAGGAGATTAGGTGAGACCAGCTACCAGGGAAAATGG
AAAGATCCAGGTAGCGGACAAGACTAGATCCAGAAGAAAGCCAGA
GTGGACAAGGTGGGATGATCAAGGTTCACAGGGTCAGCAAAGCAC
GGTGTGCACTTCCCCCACCAAGAAGCATAGAGGCTGAATGGAGCA
CCTCAAGCTCATTCTTCCTTCAGATCCTGACACCTTAGAGCTAAG
CTTTCAAGTCTCCCTGAGGACCAGCCATACAGCTCAGCATCTGAG
TGGTGTGCATCCCATTCTCTTCTGGGGTCCTGGTTTCCTAAGATC
ATAGTGACCACTTCGCTGGCACTGGAGCAGCATGAGGGAGACAGA
ACCAGGGCTATCAAAGGAGGCTGACTTTGTACTATCTGATATGCA
TGTGTTTGTGGCCTGTGAGTCTGTGATGTAAGGCTCAATGTCCTT
ACAAAGCAGCATTCTCTCATCCATTTTTCTTCCCCTGTTTTCTTT
CAGACTGTGGCTTCACCTCCGGTAAGTGAGTCTCTCCTTTTTCTC
TCTATCTTTCGCCGTCTCTGCTCTCGAACCAGGGCATGGAGAATC
CACGGACACAGGGGCGTGAGGGAGGCCAGAGCCACCTGTGCACAG
GTGCCTACATGCTCTGTTCTTGTCAACAGAGTCTTACCAGCAAGG
GGTCCTGTCTGCCACCATCCTCTATGAGATCTTGCTAGGGAAGGC
CACCTTGTATGCCGTGCTGGTCAGTGCCCTCGTGCTGATGGCCAT
GGTAAGGAGGAGGGTGGGATAGGGCAGATGATGGGGGCAGGGGAT
GGAACATCACACATGGGCATAAAGGAATCTCAGAGCCAGAGCACA
GCCTAATATATCCTATCACCTCAATGAAACCATAATGAAGCCAGA
CTGGGGAGAAAATGCAGGGAATATCACAGAATGCATCATGGGAGG
ATGGAGACAACCAGCGAGCCCTACTCAAATTAGGCCTCAGAGCCC
GCCTCCCCTGCCCTACTCCTGCTGTGCCATAGCCCCTGAAACCCT
GAAAATGTTCTCTCTTCCACAGGTCAAGAGAAAGGATTCCAGAGG
CTAG

In certain embodiments, the first antigen binding chain or the second antigen binding chain comprises a constant domain comprising a native or modified TRGC polypeptide. In certain embodiments, the TRGC polypeptide is a native or modified TRGC1 polypeptide. In certain embodiments, the TRGC1 polypeptide comprises or consists of an amino acid sequence that is at least about 85%, about 90%, about 95%, about 96%, about 97%, about 98%, about 99% or 100% homologous or identical to the amino acid sequence set forth in SEQ ID NO: 21, which is provided below. In certain embodiments, the TRGC1 polypeptide comprises or consists of the amino acid sequence set forth in SEQ ID NO: 21.

[SEQ ID NO: 21]
DKQLDADVSPKPTIFLPSIAETKLQKAGTYLCLLEKFFPDVIKIH
WQEKKSNTILGSQEGNTMKINDTYMKFSWLTVPEKSLDKEHRCIV
RHENNKNGVDQEIIFPPIKTDVITMDPKDNCSKDANDTLLLQLTN
TSAYYMYLLLLLKSVVYFAIITCCLLRRTAFCCNGEKS

In certain embodiments, the TRGC polypeptide is a native or modified TRGC2 polypeptide. In certain embodiments, the TRGC2 polypeptide comprises or consists of an amino acid sequence that is at least about 85%, about 90%, about 95%, about 96%, about 97%, about 98%, about 99% or 100% homologous or identical to the amino acid sequence set forth in SEQ ID NO: 22, which is provided below. In certain embodiments, the TRGC2 polypeptide comprises or consists of the amino acid sequence set forth in SEQ ID NO: 22.

[SEQ ID NO: 22]
DKQLDADVSPKPTIFLPSIAETKLQKAGTYLCLLEKFFPDIIKIH
WQEKKSNTILGSQEGNTMKINDTYMKFSWLTVPEESLDKEHRCIV
RHENNKNGIDQEIIFPPIKTDVTTVDPKYNYSKDANDVITMDPKD
NWSKDANDTLLLQLTNTSAYYTYLLLLLKSVVYFAIITCCLLRRT
AFCCNGEKS

In certain embodiments, the TRGC polypeptide comprises or consists of an amino acid sequence that is at least about 85%, about 90%, about 95%, about 96%, about 97%, about 98%, about 99% or 100% homologous or identical to the amino acid sequence encoded by a transcript expressed by a gene of NCBI Genbank ID: 6966, NG_001336.2, range 108270 to 113860 (TRGC1, SEQ ID NO: 23), NCBI Genbank ID: 6967, NG_001336.2, range 124376 to 133924 (TRGC2, SEQ ID NO: 24) or a fragment thereof, and/or may optionally comprise up to one or up to two or up to three conservative amino acid substitutions. In certain embodiments, the TRGC polypeptide comprises or consists of the amino acid sequence encoded by the nucleotide sequence of SEQ ID NO: 5236. In certain embodiments, the TRGC polypeptide comprises or consists of the amino acid sequence encoded by the nucleotide sequence of SEQ ID NO: 24. SEQ ID NO: 23 and 24 are provided below.

[SEQ ID NO: 23]
ATAAACAACTTGATGCAGATGTTTCCCCCAAGCCCACTATTTTTCTTCCTTCAATTGCTGAAACAAAGCT
CCAGAAGGCTGGAACATACCTTTGTCTTCTTGAGAAATTTTTCCCTGATGTTATTAAGATACATTGGCAA
GAAAAGAAGAGCAACACGATTCTGGGATCCCAGGAGGGGAACACCATGAAGACTAACGACACATACATGA
AATTTAGCTGGTTAACGGTGCCAGAAAAGTCACTGGACAAAGAACACAGATGTATCGTCAGACATGAGAA
TAATAAAAACGGAGTTGATCAAGAAATTATCTTTCCTCCAATAAAGACAGGTATGTGTTTACGCATATCA
TCTGTCAGAACACTTCTTTGAAAGTGAATGCTGCATTTTTTCCTTTCAGTATTAATGAAAAACAAACATA
AATCTTTCTTAAATATTGTTACATTTAATGGTAGCATAAATGCCCTGCTACTTTTCTATAGAATTAAAAT
GGTATAGGTTTTGGAGAAAACAAAATTGAAAAAGTTACTGAAGGTTTGTCAGCCTCAGCTCCATTATCCA
AAATAAGAAAGTCACGTGCTGGTTTTTAGGGTTGTTAGATGGATTAAAGAAACAACATACACAGAAGCAT
CTAGCAACGTGACACGTGGTAAACGCTCAAAAAGTGTTCTCCCTTCTTTTGATGACTTTACTTGATCAGG
AAATAACATATATATGTCTTTCAGGAATGTTCTGCCCAAGCAGGAGAGTCACTCACCTCAATCTTGCTAC
CCACAAAGTTTAACCTAAAAACAACGGGTTCATTGTTGACAAAATGATGTTTATCTGTTGTTGACAGAAT
GATGTTTATCTAAAAACAGTTCCAATTTTCTATTTCCTTTGCTGAGACACAAAGGGGAGGCAAATGTGCA
AAGCTTGAGGGTAGTCTTACCACTGTGCTTAAGTGTTCTGATTTTTCTAGTGATCAGGGCAAAATAAAAA
GTATAGTAAGTTCCAAGGCAGTGAATATTATACAGGAGAGAAGTTACAGTTTTATAATGTGTTTTCCTTT
ACACTAAATTCTAAAAGTAAAAAGTCTTTTTTTTTTTTTGACAGAGTTTCACTCTTGTTGCCCAAGCAGG
TGTGCTATGGTATGATCTCAGCTCACTGCAACCTCCACCTCCCGGGTTCAAGTGATTCTCTTACTTCAGC
CTCCCGACAGGCTGGGATTGCAGGCGCCTGCCACCACACCTGGCTAATTTTTGTGTTTTTAGTAGAGATG
GGGTTTCACCATGTTGGCCAGGCTGGTCTCAAATTCCTGACCTCAAGTGATCCATCCACCTCGGCCTCCA
AGTGCTGGGATTATGGGCGTCAGCCACTGTGCCCAGCCTAAAAGTAAAATGTCTTTCATGAGCTTCCCAA
GGCAGCTACGTTAAGGAGGACACTTCTCTTAATGTCATTCTACAGTAGATTTCTAATGCTCTTTCTTGGA
AGTTTGTTTTTCTGAGAAAAGCTAAAAATATAACATGGAAGTGATCATATTATATAATCAATGAAGTGCT
TTTCAAGGAGATAAAACTAATCTGGTCCACACTTGCAACCAACCTTGATTGAGAGAGAGAGAGAACTCAG
GATACACTTGAAGATTTTATTATGGGGAACAGTTACTTTATTCTTTTTACCTCAATCAATGCATGGAAAT
AAGTGATAGTCATTTTCATTTATCTTTTAATAAATGAAGTCACCATGAGGAAAATAAAAAGACATTGAAA
ACCCATTAAAGTCAGCCCTTAAAGATATTTGGACATGCAGACTTGATAACTAACGTTTGCATTCTTGAGA
CTTACCCAAAACCCATACCTCAAGTCCAAGTTTTTAGAATTCATGAAATAAAGATCTCAGTGAGTGCATA
AAATTGCGCACCAGAATCATATCCGTATAGACAAGAACACATCTACTAGAAAAATAATAAACCAACACAC
CAATGCAACTGTGTTTTCTTCTGTTTTAAAGTATGTTGTCTTTGTATGCATGTTTGCTTCTTCCTTTTTT
TTTTTAACATCACAGATAAATTCAACTCTCACCTCAGGTTTTATTGAGAGAACTGTCAATGTGACTTGGC
CTCTGTCTTTCTAGTCCCAGAAAGAATTGCACTGAAATCTGAGCTCCTGTAATAAAAACAACCATTTGCT
GAGAGTAATTAACATACTGAAAGAGATTTTCTTAGAGTACACAATGGTGACATTATATTGCCTCTTTATA
AATAACTTTCTATCTATTTCTGTGGATTATTCCTACAAAGTACTTTTCATATGTCCAATTTCTTTTCTTC
CCCTACAACTACTGTCTGAATACTGGCTCTGCTATTTGCTGATATGATTCTCGGCAAGTTGCCTGCACTT
TTTAAACTTTATTTCCTCATTCAGAACATGGGGCCATACATAATACAACTCACTTCAGTGTTATTGGGGA
ATTAAACAAAAAATGCATGGGAAGCATTTAACATAGTGCCTGACACAATAATGAGTACTCAGTAGATGTT
AGCTTTTATTAATATTGTTGTTGTTATGTCCAGAAACACTATACCTCCAGAAAATCATGGGTACTTGCTG
GGGACATTGGGGATATGCATGATTTGGAAAAGAATGACTGCTTTTTTTGCTTAGATGAGAAATTTTTCTA
AGCCAGACTCCTTCAAATATGTAAGATTCTGTTGTGGATTCAAGGACTGAAAGAATTCTTGGCCGAGTGT
GGTGGCTTATCCCTGTAATCCCAGCATTTTGTGAGGACAAGGCAGGAAGATTGCTTGAGTCCAGGAGTTT
GAAACCAGCCTGCGCAACATGGCGAAACCCTGTCTCTACAAAAAATACAAACATTAGCTCGGAGTGAGTG
CTGACATGTGCCTGTACTCCCAGCTACTCAGAAGGCTGAGATGGGAGGATCTCATGAGCCTGGGGAGTTT
GAGGCTTCAGTGAGCCGTGATGACACCGTACTATACTCCACTCCAGCCTGGGTGACAGTGAGACCCTGCC
TCAAAAAACAAACAAACAAACAAACAAAACAAAATTAATCTTTTTGCTGATGTCATGTCAGCAGTGTGTG
TTGAAGGCTGTAAAGCAGCCATTTGTTCAGTTTATTTTTCCATTGAACAAGTATTTATCAAAAACATACT
TTGTGGCAGTCACTATGCTAGGAGCTATGAATACAGAAGGAAAAGTAAATGCTCTTGGATACTACACTCC
AGTTGTGATAAAAAAGAAAAAATGTATTCTTCACCAACTTCAACATCTTGATGTGCAAAAACATAATACA
TGAATTAGATCTACCTAATTACACAGAATTAGACCAATTGTTTCTGGAATTGTGGGCTCATATTTTTAAT
AACTGTCCTCCTGCCTCTCTGTCGACAGGTTTTATAAATATTCATTTAATTACACACACACACACGAACA
ATTGACTAGTACTTGCTCTCATTCTTCTAGATGTCATCACAATGGATCCCAAAGACAATTGTTCAAAAGA
TGCAAATGGTAAGCTTTTGTGTTTTTCCCTTCCTCCTGATCATTTTGTTTTGAACTTCTCTGGCTTGAAA
AATCAGGGAATGGATTTTGCTAGGTTGGATGCTGCAGAATGGACCTAGTGATATTTTAAATTAGTCCCTC
ATTTTCTAGGAGTTGTATTAACAAACCTAACTACTGCTTTGGGGTATGAGATGACTGTAAATTAGAGAGG
GTACAGTGGTATAGTGATATGCTTTTAATTATTTCAAAAAAAAGATTTTATTCATTCATGTGTCTTTTTT
CTTTTTCTTTTCTTTTTTTTTTTTTTTTGGACAGAGTCTTGCTCTGTCACCCAGGCTGGAGTGCGGTGGC
AGTATCTCAGCTCACCACAACCTCCGCCTCCCGGCTTCAAGTGATTCTCCTGCCTCAGCTTCTCGAGTAG
CTGGGACTACAGGCGCGTGCCACCATGCCCGGCTAATTTTTGTATTTTTAGTAGAGTTGGGGTTTCACCA
TGTTGGCCAGGATGGCCTCGAATTTGTGACCTCGTGATCTGCCCCCTCGCCCTCCCGAACTGTTGGGATT
ACAGGCGTGAGTCACTGTGCCCGGCCTCCTGTCCTGTCTTTTGTTTAATGACTGGGAAAAACATGATACC
ATGTTGCTTCTCGAGTTGTTTTGTTTTAGTCTTTGGTCTTTGCTAGTAGCTAATAACACGAACTAGTGTT
TATCAAGTGCTTTTTACACAGAAGGGCTTGGGCTGTGTTCTGCATTTTCTTGTTTAACCCTCTTAAAACT
CCTATAAAATGGTACATATTTTTCTCCCAATTTACAGTCCCTTTAAAGCAAATAATTATAAAAATCCCTA
TACATGTCACACAGCTAGATCTGGGATTTCAAATCAGGCCATCAAACAAAGAGTTTATGTACTTAGTAAG
TTTTCTGTTCTTTTTCTACAATAGAGTCAGATAGCAAGAAATTACCAAGCCAGGAACCTGAAACAAAACG
GACATCATGTGGGGCTGGGTGGGTGCATGGGCTTTGCAGACTGGACTTTCACTCCAGCTCTTTTAATGAT
TAGGTGTAAGTGACCTACATTTTGTGAGCAACAGTTTTCTCATCAGCCAACAAAGAATAATTACACCAGA
TTCACAGTTATTGAAGAGATAAAGGCATGAATGTGAGATGTCTGGCATAGGGCATCTCATTTAGCAGACA
CAGAATGAGTACTTGTTTCTGGCTTTTTCTCTCTACATATGCACAAAGAATGCGACTAGAAGCATGGGCT
CTAGCCCTGCTCAACTTTCCTCTATTTCCAATACCAAGGGGCTCTGACTTAGGCTGCCACACCAGGCAAG
GAGGGCAGTACCACCTCACTTGACCAAGGGCAGGGAGTCACGGACACATCACTTCTTGAGATCCTTTTCC
ACACCAAGGACTGATGTTTCTGGAATTCTCACTTTATGAAGACAAAACATATAAATGGAAATTTTCTCAG
GTAGAGACTCACTCTTGTAGCTCATTGAGTAGGCACTAGTGGTCCACCCCCACTGTCTTTACTTATTCCT
TGACATCACATATCTCTTGCAAAACCTCAAATAATATTAAATGCAATCACCCAATAATAGCATAGCCATA
ATTAGAGGCATTTAGGAAAGACAGGTGAGTGTGCCACAACTACCTAACACATCAGCAAATCTGGATTAAC
CACTTTCTTTGATTTTCCACAATGCAACCTTACTTTTTAATAGTTGGGAATGTTCTAAGTGAATTTAGCA
GAGGTTGTTAATCAACTTGAAAGCTGAATTCTGACTTGTCTGACTCTTGGTGGTGCTGGTAGCAGTAGAT
GTTTACTTTTAGGTTTTGGTGGTGGTGGAATATCACTTCAACGTAAATCATCAGAAATAAGTATTTGTGA
ACCCCTCTCGCATTAATGTATCTTATTCTGTAAAAAGAACATGTGCAATTTCTCTTAGATACACTACTGC
TGCAGCTCACAAACACCTCTGCATATTACATGTACCTCCTCCTGCTCCTCAAGAGTGTGGTCTATTTTGC
CATCATCACCTGCTGTCTGCTTAGAAGAACGGCTTTCTGCTGCAATGGAGAGAAATCATAA
[SEQ ID NO: 24]
ATAAACAACTTGATGCAGATGTTTCCCCCAAGCCCACTATTTTTCTTCCTTCGATTGCTGAAACAAAACT
CCAGAAGGCTGGAACATACCTTTGTCTTCTTGAGAAATTTTTCCCAGATATTATTAAGATACATTGGCAA
GAAAAGAAGAGCAACACGATTCTGGGATCCCAGGAGGGGAACACCATGAAGACTAACGACACATACATGA
AATTTAGCTGGTTAACGGTGCCAGAAGAGTCACTGGACAAAGAACACAGATGTATCGTCAGACATGAGAA
TAATAAAAACGGAATTGATCAAGAAATTATCTTTCCTCCAATAAAGACAGGTATGTGTTTACACATATCA
TCTGTCAGAACACTTCTTTGAAAGTGAATGCTGCATTTTTTCCTTTCAGTATTAATGAAAAACATAAATC
TTTCTTAAAAATTGTTACATTTAATGGTAGCGTAAATGCCCTGCTACTTTTCTATAGAATTAAAATGGTA
TAGGTTTTGGAGAAAACAAAATTGAAAAAGTTGCTGAAGGTTTGTCAGCCTCAGCTCCATTATCCAAAAT
AAGAAAGTCACGTGCTGGTTTTTAGGGTTGTTAGATGGATTAAAGAAACAACATACACAGAAGCATCTAG
CAACGTGACACGTGGTAAACGCTCAAAAAGTGTTCTCCCTTCTTTTGATGACTTTACTTGATCAGGAAAT
AACATATATATGTCTTTCAGGAATGTTCTGCCCAAGCAGGAGAGTCACTCACCTCAATCTTGCTACCCAC
AAAGTTTAACCTAAAAACAACGGGTTCATTGTTGACAAAATAATGTTTATCTGAAGATAACTGTAGATCA
TATTTATCTGTAGATAATGTTTATCTGTGGAGTGTGGCTCTACAAAACATAGAATAGTCTTGGTCACTGC
AGTTTTATAGAGGCCTTGGGTTTTTCAGAGTTTCATTTTATATATCACCATAAAGTAACATTTCATAATT
ACAGGTTGGTAAGGCTTACATGTACAAACATTCTTCCATTTTCCATAATAAATGCATTTCCTGCCATTGG
TGAATGCAGCTCAATAAACATTTATTGTACAATTATGACACGCCAGGCTTAGTGGAAATGTGGATGAACA
GACAAGGATGAGTTACTGTCCTAAGGATGATGCATGACAGTGCAGAGAATATACTCTCTTCCTGATCACT
CAGGGTCACTCATGATTCATGCGCGAGGTCCCAAAACAGTGCCTTTGATGCAGATTCTGTACATCTCTAG
ACGATTGGTCCAAGGGCTGAATGTGCTCTGGCCCAGTGGTCCAGTCTGTCACTATATGTCAACATCCTGA
ATATGAACATAACAGTCCAACATCTCAAGAGTGGGCATGAAAAGGACTCATTTTGTGCTTTTTCCTGTGG
TTAACAAGTCCTTTTTAGCCTGGGGGAACAAGCATTAACAAAATGTTTGAAGATCTTTGCCACGTACCAT
TCCAAATTTCTAGGGTAAGTCTTTAGCTTTTCAGATCCTGAGTTTCTGCAATGATCAAATGTGATTTGGA
CAGTTGCGTTGACTTTCTCCTGGGGCTATAATGGAGTGCAAAGGAAACAATGGCAGGGAAAATGCTTGCT
TTCAAAATGGTAGCATGGATGTGTTCATTCGTGTAGTTACTGTATTAGGTATAGCCTTTCCTGAAACTAA
CTGAAGTGGGGTTATAAAAACAGTCCCAATTTTCTATTTCCTTTGCTGAGACACAAAGAGGAGACAAAAG
AGCAAAGCTTGAGGGTAGTTTTACCACTGTGCTTAAGTGTTCTGATTTTTCCAGTGATCAGGGTGAAATA
AAAAGCATAGTAAGTTCCAGGGCAGTGAATACCATACAGGAGACAAGTTACAGTTTTATAATGTGTTTTA
CTTTACACTAAATTCTAAAAGTAAAATGTCTTTTTTTTTTTCCGAGACAGAGTTTCACTCTTGTAGCCCA
GGCAGGAGTGCTATGGTGTGATCTCGGCTCACAGCAACCTCCACCTCCCAGTTTCAAGCGATTCTTCTGC
CTCAGCCTCCCGAGAAGTTGAAATTACAGGTGCCTGGCACCATATCTCGCTAATTATTCTATTTTTAGTA
GAGATCGGGTTTTACCATGTTGGCCAGGCTGGTCTCGAACTCCTGACTTCAAGTGATCCACCCGCCTCAG
CCTCCCAAAGTGCTGGGATTACAGGTGTGAGTCACTGTGCCGGACCTAACAGTAAAATGTCTTTCATGTG
CTTCTCAAGGCAACTACATTAAGGAGGACACATCTCTTAATGTCATTCTACAGTAGATTTCTAATGCTCT
TTCTTGGAAGTTTGTTTTTCTGAGAAGAGCTAAAAATATAATAACATGGAAGTGATCATATTATATAATC
AATGAAGTGCTTTCAAAGGAGATAAAACTAACCTGGTCTGCATTTGCAACCAGCCTTGATTGAGAGAGAG
AGAACTCAGGATACACTTAGAGATTTTATTATGGGGAATAGTTACTTTATTCATTTTACCTCAATCAATG
CATGGAAATAAGTGACAGTCATTTTCATTTATCTTTTAATAAATAAAGTCACCATGAGGAAAATGAAAAC
CCATTAAAGTCAGTCCTTAAAGATATTTGGACATGCAGACATGATAACTAACATTTCCATTCGTGAGACT
TACCCAAAACCTATACCTCAAGTCCATTTCTTAGAATACATGAAATAAAGATCTCAGTGAGTGTATAAAA
CTGCACACCAGAATCATATCCGTATAGACAAGAATACATCTACTAGAAAAATATAAACCAAAACACCAAG
GTGACTCTGTTTTTTTCTGTTTTAAAATATGTTGTCTTTGTATGCATGTTTGCTTCTTCCTTTTTTTTTT
TAAACATCGCAGATAAATTCAACTCTCACCTCAGTTGAGAGAGAACTGTCAATGTGACTTGGCCTCTCTC
TTTCTAGTCCCAGAAAGAATTGCACTGAAATGCTGAGCTCCTGTAATAAAAATGACCATTTGCTGAGAGT
AATTAACATACTGAAAGAGATTTTCTTAGAATAGTGCACAATGGCCCAATGGTGACATTATATTGTCTCT
TTATAAATTATTTTCTATCTATTTCTGTGGATTATTTCTACAAAGCACTTTTCATATGTCCAATTCCTTT
TATTCCCCTACAAGTACTGACTGACTACTGGCTCTGCTGTTCACTGATATGACTTTCGGCAAGTTGCCTG
CACTTTTTAAACGTTATTTCCTCATTCAGAACATGGGGCCATACAAAATACAACTCACTTCAGTGTTATT
GGGGAATTAAACAAATAAATGCATGGGAAGCATTTAACATAGTGCCTGACACAATAATGAGCACTCAGTA
GATGTTAGCTTTTATTAATATTGTTGTTGCTATGTCCAGAAACACTATACCTCCAGAAAATCATGGGTAC
TTGCTGGGGACGTTGGGGATATGCATGATTTTGAAAGGAGTGACTGCTCTTTACTGCTCAGATGAGAAAT
TTTTCTAAGCCAGACTCCTTCAAACATGTAAGATTCTGTTGTGGATTCTAGGACTGAAAGAATTCTTGGC
CGAGTGTGGTGGCTTATCCTGGTAATCTCATCATTTGGGAGGACAAGGCAGGAAGATTGCTTGAGCCCAG
GAGTTGGAAACAAGCCTGGACAACATGGCGAAACCCTGTCTCTACAAAAAATACAAACATTAGCTGGTCA
TGGGAGTGAGTGCCTGTACTCCCAGCTACTCAGGAGGCTAAGATAGGAGGATCACCTGAGCCTGGGCAGT
TTGAGGTTTCAGTGAGCCGTGATGACACCATACTATACTCCACTCCAGCCTGGGTGACAGTGACATCCTG
CCTCAAAAAAACCCCCAAAATTATTCTTTTTGCTGATTTCATGTCAGCAGTGTGTGCTGAAGGCTGTAAA
GTAGCCACTTGTTCTGTTTATTTTTCCATTGAACAAGTATTTATCAAAAACGTACTTTGTGGAAGGCACT
GTGCTAGGAACTATGCATACAGAAGGAAAACCAAATGTTCTTGGATACTACACTCCAGTTGTGATAAAAA
AGAAAAAAGTATTCTTCACAAACTTCAACATTTTGATGTGCAAAAACATAATATATGAATTAGATCTACC
TAACTACACAGAATTAGACCAATTATTTCTGGGATTATGGGCTCATATTTTTAATAACTGTCCTCCTACC
TCTCTGTTGACAGGTTTTATAAATATTCATTTAATTACACACAGTCACAGACACACTCAGACACACACAC
ATACACACACACACACACCTTGACAAATAATGGGCATGAACAATTGACTGGTACTTGCTCTCATTCTTCT
AGATGTCACCACAGTGGATCCCAAATACAATTATTCAAAGGATGCAAATGGTAAGTTTTTGTGTTTTTTA
TTTCCTCCTGATCATTTTAAGTTTTGAACTTCTCTGGCTTGAAAAATCAGGGAATGGATTTTGCTAGGTT
GGATGCTGCAGAATGGACCTAATCATATTTTAAATTAGTCCCTCTTTTTCTAGGAGTTGTATTAACAAAC
CTAACTACTGCTTCATGTAAGAGATGACTGTAAATTGAAGGGTACAGTGATATGCTTTCAGTTATTTCAA
AAAACAGACTTTACTCATCCATGTGTCTTTTTTCTTTTCTTTTTTTTCTTTTTTGAGACGGAGTCTCGCT
CTGTTGAACAGGCTGGATTGCAGTGACGCGATCTCACCTCACTACAACCTCCGCCTCTGGAGTTCAAGCG
ATTCTCCAGCCTCAGCTTCTCAAGTAGCTGGGACTACAGGCACATGCCACCATGTCCGGGTCATCTTTGT
ATTTTTAGCAGAGACCGGGTTTCACTATGTTGGCCAGGCTGGTCTAGAATTCCTGACTTCGTGATCTGCC
CCCTCAGCCCTCCGAAGTGCTGGGATTACAGACGTGAGTCACTGTGCCCGGCCTAACAGTAAAATGTCTT
TCATGCGCTTCTCAAGGCAACTACGTTAAGGAGGACACTTCTCTTAATGTCATTCTACAGTAGATTTCTA
ATGCTCTTTCTTGGAAGTTTGTTTTTCTGAGAAAAGCTAAAAATATAACATGGAAGTGATCATATTGTAT
AATCAATGAAGTGCTTTTCAAGGAGATAAAACTAATCTGGTCCACGTTTGCAACCAACCTTGATTGAGAG
AGAGAGAGAACTCAGGATACACTTGGAGATTTTATTATGGGGAATAGTTACTTTATTCTTTTTTCCTCAA
TCAATTCATGGAAATAAGTGATAGTCATATTCATTTATCTTTTAATAAATGAAGTCACCATGAGGAAAAT
AAAAAGACATTGAAAACCCATTAAAGTTAGCCCTTAAAGATATTTGGACATGCAGACTTGATAACTAACG
TTTGCATTCTTGAGACTTACCCAAAACCCATACCTCAAGTCCATGTTTTTAGAATTCATGAAATAAAGAT
CTCAGTGAGTGCATAAAATTGCGCACCAGAATCATATCCGTATAGACAAGAACACATCTACTAGAAAAAT
AATAAACCAACACACCAATGCAACTGTGTTTTCTTCTGTTTTAAAATATGTTGTCTTTGTATGCATGTTT
GCTTCTTCCTTTTTTTTTTTTAACATCACAGATAAATTCAACTCTCACCTCAGGTTTTATTGAGAGAACT
GTCAATGTGACTTGGCCTCTGTCTTTCTAGTCCCAGAAAGAATCGCACTGAAATGCTGAGCTCCTGTAAT
AAAAATGACCATTTGCTGAGAGTAATTAACATACTGAAAGAGATTTTCTTAGAGTACACAATGGTGACAT
TATATTGTCTCTTTATAAATAACTTTCTATCTATTTCTGTGGATTATTCCTACAAAGTACTTTTCATATG
TCCAGTTTCTTTTCTTCCCCTACAACTACCGTCTGAATACTGGCTCTGCTATTTGCTGATATGATTCTCG
GCAAGTTGCCTGCACTTTTTAAACTTTATTTCCTCATTCAGAACATGGGGCCATGTAATACTCATGTACG
TGAGTATTACGTAATAATGCTCACTTAAGTGTTACTGGGGAATTAAACAAAAAAATGCATGGCAAGCATT
TAACATAGTGCCTGACACAATAATGAGCACTCAGTAGATGTTAGATTTTATTAATATTGTTGTTGTTATG
TCCGGAAACACTATACCTCCAGAAAATCATGGGTACTTGCTTGGGATGTTGGGGATATGCATGATTTGGA
AAGGTATGACTGCTTTTTTCTGCTTAGATGAGAAATTTTTCTAAGCCAGACTCCTTCAAATATGTAAGAT
TCTGTTGTGGATTCTAGGACGGAAAGAATTCTTGGTCAGGTGTGGTTTCTTATCCCTGTAATCCCAGAAT
TTTGGGAGGACAAGGCAGGAAGATTGCTTGAGCCCAGGAGTTTGAAACCAGCCTGGGCAACAAGACGAAA
CCCTGTCTCTACAAAAGTACATAAATTAGCTTGGCTTGGTGGTGTGTGCCTGTATTACCAGCTATTCGGG
AGACTGAGATGGGAGGATCTCCTGAACCTGTGAAGTTTGAGGCTTCAGTGAGCCGTGATGACACCATACT
ATACTCGACTCCAGCCTGTGCGACAGTGAGACTCTGCGTCAAAAAAAAAACCCCAAAATTATTGTTTTTG
CTGATTTCAGGTCAGCAGTGTGTGCTGAAGGGTGTAAAGTAGCCACTTGATCAGTTTATTTTTCCACTGA
ACAAGTATTTATCAAAAACATACTTTGTGGTCTGTTTTTGATAAATAAAAAGGCACTGTGCTAGGAGCCA
TGAATACAGAAGGAAAACCAAATGTTCTTGGATACTACACTCCAGTTGTGATAAAAAAGAAAAATGTATT
CTTCACGAACTTCAACATTTTGATATGCAAAAACATAGTATATAAATTAGATCTACCTGATTACGTAGAA
TCAGACCAATTATTTCTGGAATTGAGGGCTCATATTTTTAATAACTGTCCTCCTGCCTCTCTGTTGACAG
GTTTTATAAATATTCATTTAATTACACACACACACACACACACCTTGACAAATAATGGACATGAACAATT
GACTAGTACTTGCTCTCATTCTTCTAGATGTCATCACAATGGATCCCAAAGACAATTGGTCAAAAGATGC
AAATGGTAAGCTTTTGTGTTTTTCCTTTCCTCCTGATCATTTTAAGTTTTGAACTTCTCTGGCTTGAAAA
ATCAGGGAATGGGCCGGGTGCGGTGGCTCACGCCTGTAATCCCAGCACTTTGGGAGGCCGAGGCGGGCGG
ATCACGAGGTCAGGAGATCGAGACCATCCCGGCTAAAACGGTGAAACCCCGTCTCTACTAAAAATACAAA
AAATTAGCCGGGCTTAGTGGCGGGCGCCTGTAGTCCCAGCTACTTGGGAGGCTGAGGCAGGAGAATGGCG
TGAACCCGGGAGGCGGAGCTTGCAGTGAGCCGAGATTGCGCCACTGCACTCCACTCCAGCCTGGGCGACA
GAGCGAGACTCCGTCTCAAAAAAAAAAAAAAAAAAAAAAAAAGAAAAATCAGGGAATGGATTTTGCTAGG
TTGGATGCTGCAGAATGGACCTAGTGATATTTTAAATTAGTCCCTCTTTTTCTAGGAGTTGTATTAACAA
ACCTAACTACTGCTTCGGGTATGAGATGACTGTAAATTAGAGGGTACAGTGATATGCTTTCAGTTATTTC
AAAAAACAGACTTTATTCATCCGTCTGTCTTTTTTTTTTTTTTTTTTTTTTTTTTTTGAGACGGAGGAGT
CTCACTCTATCACCCAGGCTGGAGTGCAGTGGCGCGATCTCGGCTCACCATAACCTCCGCCTTACTGGTT
CAAGCGATTCTCCAGCCTCAGCTTCTCAAGTAGCTGGGACTACAGGTGCACACCACCATACCTGGCTAAT
TTTTGTATTTTTAATAGAGATGGGGTTTCACCACGCTGGCCAGGATGGTCTTGAATTCTTGACCTCGTGA
TCTGCCCCCTCGGGCTCCCAAACTTCTGGGATTATAGGCGTGAGCCACTGTGCCCGGCCTTCTGTCTTTT
GTTATAATGACTGGGGAAAACATGATACCATGTTGCTTCTTGAGTTGTTTTGTTTTAGTCTTTGGTCTTT
GCTAGTAGCTAATAACACGAACTAGTGTTTATCAAGTGCTTTTTACACAGAAGGGCTTGTTCTGCATTTT
CTAGTTTAATCATCTTAATACTCCTATAAAGTAGTACAATATATTTTCTCCCATTTTACAGTCCCTTTAA
AGTAAATAACTATAAAAATCCCTTATACATGTCACACAGCTAGGTCTGGCATTTCAAATCAGGACATCAA
ACAAAGAATTCGTGCAGTTACTAAGTCCTCTATTTTTTCTACAATAGAAAAAATAGCAAGAATTACAGAT
AGCAAGACATTACAAGGCAGGAATCTGAAACGAAAGGGACATAATGTGGGGCTGGGTGGGTGCATGAGCT
TTGCAGACTAGACTTTCATTCCAGCTCTTTTAATGATTAGGTGTAAGTGACCTACATTTTGTGAGTAACA
GTTTTCTCATCAGCCAACTAAGAATAATTACACCAGATTCACAGTTATTGAAGAGATAAGGGCATGAATG
TGAGATGTCTGGCGTAGGGTATCTCATTTAGCAGACACAGAATGAATACTTGTTTCTGGCTTTTTCTCTC
TACATATGCACAAAGAATGTGACTAGAAGCATTGGCTCTAGCCCTGCTCAACTTTCCTCTATTTCCAATA
CCAAGGGGCTCTGACTTAGGCTGCCACACCAGGCAAGGAGGGGCAGTACCACCTCACTTGACCAAGGGCA
GGGAGTCACGGACACATCACTTCCTGAGATCCTTTTCCACACCAAGGACTGATGTTTCTGGAATTCTCAC
TTTATGAAGACAAAACATATAAATGGAAATTTCTGCAGGAAGAGACTCACTCTTGTAGCTCATTGAGTAG
GCACTAGTGGTCCACCCCCACTGTCTTTACTTATTCCTTGACATCACATATCTCTTGTAAAACCTCAAAT
AATGTTAAATGCAATCACCCAATAATAGCATAGCCATAATTAGAGGCATTTAGGAAAGACAGGTGAGTGT
GCCACAACTACCTAACACATCAGCAAATCTGGATTAACCACTTTCTTTGATTTTCCACAATGCAACCTTA
CTTTTTAATAGTTGGGAATGTTCTAAGTGAATTTAGCAGAGGTTGTTAATCAACTTGAAAGCTGAATTCT
GACTTGTCTGACTCTTGGTGGTGCTGGTAGCAGTAGATGTTTACTTTTAGGTTTTGGTGGTGGTGGAATA
TCACTTCAACGTAAATCATCAGAAATAAGTATTTGTGAACCCCTCTCGCATTAATATATCTTATTCTGTA
AAAAGAACATGTGCAATTTCTCTTAGATACACTACTGCTGCAGCTCACAAACACCTCTGCATATTACACG
TACCTCCTCCTGCTCCTCAAGAGTGTGGTCTATTTTGCCATCATCACCTGCTGTCTGCTTAGAAGAACGG
CTTTCTGCTGCAATGGAGAGAAATCATAA

In certain embodiments, the first antigen binding chain or the second antigen binding chain comprises a constant domain comprising a native or modified TRDC polypeptide. In certain embodiments, the TRDC polypeptide comprises or consists of an amino acid sequence that is at least about 85%, about 90%, about 95%, about 96%, about 97%, about 98%, about 99% or 100% homologous or identical to the amino acid sequence set forth in SEQ ID NO: 25, which is provided below. In certain embodiments, the TRDC polypeptide comprises or consists of the amino acid sequence set forth in SEQ ID NO: 25.

[SEQ ID NO: 25]
SQPHTKPSVFVMKNGTNVACLVKEFYPKDIRINLVSSKKITEFDP
AIVISPSGKYNAVKLGKYEDSNSVTCSVQHDNKTVHSTDFEVKTD
STDHVKPKETENTKQPSKSCHKPKAIVHTEKVNMMSLTVLGLRML
FAKTVAVNELLTAKLEFL

In certain embodiments, the TCR-like fusion molecule comprises a hinge/spacer region that links the first antigen binding chain to the constant domain. In certain embodiments, the TCR-like fusion molecule comprises a hinge/spacer region that links the second antigen binding chain to the constant domain. The hinge/spacer region can be flexible enough to allow the antigen binding chain to orient in different directions to facilitate antigen recognition. In certain embodiments, the hinge/spacer region can be the hinge region from IgG1, the CH2CH3 region of immunoglobulin and portions of CD3, a portion of a TCRα polypeptide, a portion of a TCRβ polypeptide, a portion of a CD28 polypeptide, a portion of a CD8 polypeptide, or a synthetic spacer sequence. In certain embodiments, the hinge/spacer region comprises a portion of a TCRα polypeptide. In certain embodiments, the hinge/spacer region comprises a portion of the variable region (TRAV), a portion of the diversity region (TRAD), a portion of the joining region (TRAJ), a portion of the constant region (TRAC), or a combination thereof. In certain embodiments, the hinge/spacer region comprises a portion of the TRAJ region and a portion of the TRAC region of the TCRα polypeptide. In certain embodiments, the hinge/spacer region comprises or consists of the amino acid sequence set forth in SEQ ID NO: 26. In certain embodiments, the hinge/spacer region comprises or consists of amino acids 1 to 3 of the sequence set forth in SEQ ID NO: 26. An exemplary nucleic acid sequence encoding the amino acid sequence of SEQ ID NO: 26 is set forth in SEQ ID NO: 27. SEQ ID NO: 26 and 27 are provided below.

[SEQ ID NO: 26]
IPNIQNPDPA
[SEQ ID NO: 27]
ATTCCCAATATCCAGAACCCTGACCCTGCC

In certain embodiments, the hinge/spacer region comprises a portion of a TCRβ polypeptide. In certain embodiments, the hinge/spacer region comprises a portion of the variable region (TRBV), a portion of the diversity region (TRBD), a portion of the joining region (TRBJ), a portion of the constant region (TRBC), or a combination thereof. In certain embodiments, the hinge/spacer region comprises a portion of the TRBJ region and a portion of the TRAC region (C) of the TCRβ polypeptide. In certain embodiments, the hinge/spacer region comprises or consists of the amino acid sequence set forth in SEQ ID NO: 28. In certain embodiments, the hinge/spacer region comprises or consists of amino acid 1 to 2 of the sequence set forth in SEQ ID NO: 28. An exemplary nucleic acid sequence encoding the amino acid sequence of SEQ ID NO: 28 is set forth in SEQ ID NO: 29. SEQ ID NO: 28 and 29 are provided below.

[SEQ ID NO: 28]
LEDLKNVEPPE
[SEQ ID NO: 29]
CTGGAGGATCTGAAAAACGTGTTCCCTCCTGAA

In certain embodiments, the antigen binding chain does not comprise an intracellular domain. In certain embodiments, the antigen binding chain is capable of associating with a CD3ζ polypeptide. In certain embodiments, the antigen binding chain associating with the CD3ζ polypeptide via the constant domain. In certain embodiments, the CD3ζ polypeptide is endogenous. In certain embodiments, the CD3ζ polypeptide is exogenous. In certain embodiments, binding of the antigen binding chain to a target antigen is capable of activating the CD3ζ polypeptide associated to the antigen binding chain. In certain embodiments, the exogenous CD3ζ polypeptide is fused to or integrated with a costimulatory molecule disclosed herein.

In certain embodiments, the TCR-like fusion molecule comprises an antigen binding chain that comprises an intracellular domain. In certain embodiments, the intracellular domain comprises a CD3ζ polypeptide. In certain embodiments, binding of the antigen binding chain to an antigen is capable of activating the CD32 polypeptide of the antigen binding chain.

In certain embodiments, the CD3ζ polypeptide comprises or consists of an amino acid sequence that is at least about 85%, about 90%, about 95%, about 96%, about 97%, about 98%, about 99% or about 100% homologous to the amino acid sequence set forth in SEQ ID NO: 53 or a fragment thereof, and/or may optionally comprise up to one or up to two or up to three conservative amino acid substitutions. In certain embodiments, the CD3ζ polypeptide comprises or consists of an amino acid sequence that is a consecutive portion of SEQ ID NO: 53, which is at least about 20, or at least about 30, or at least about 40, or at least about 50, and up to about 164 amino acids in length. In certain embodiments, the CD3ζ comprises or consists of the amino acid sequence of amino acids 1 to 164, 1 to 50, 50 to 100, 52 to 164, 100 to 150, or 150 to 164 of SEQ ID NO: 53. In certain embodiments, the CD3ζ polypeptide comprises or consists of amino acids 52 to 164 of SEQ ID NO: 53.

In certain embodiments, the CD3ζ polypeptide comprises or consists of an amino acid sequence that is at least about 85%, about 90%, about 95%, about 96%, about 97%, about 98%, about 99% or about 100% homologous or identical to SEQ ID NO: 54 or a fragment thereof, and/or may optionally comprise up to one or up to two or up to three conservative amino acid substitutions. In certain embodiments, the CD3ζ polypeptide comprises or consists of the amino acid sequence set forth in SEQ ID NO: 54.

In certain embodiments, the TCR-like fusion molecule comprises an antigen binding chain that comprises an intracellular domain, wherein the intracellular domain comprises a co-stimulatory signaling region. In certain embodiments, the intracellular domain comprises a co-stimulatory signaling region and a CD3ζ polypeptide. In certain embodiments, the intracellular domain comprises a co-stimulatory signaling region and does not comprise a CD3ζ polypeptide. In certain embodiments, the co-stimulatory signaling region comprises at least an intracellular domain of a co-stimulatory molecule disclosed herein.

In certain embodiments, the TCR-like fusion molecule is capable of associating with a CD3 complex (also known as “T-cell co-receptor”). In certain embodiments, the TCR-like fusion molecule and the CD3 complex form an antigen recognizing receptor complex similar to a native TCR/CD3 complex. In certain embodiments, the CD3 complex is endogenous. In certain embodiments, the CD3 complex is exogenous. In certain embodiments, the TCR-like fusion molecule replaces a native and/or an endogenous TCR in the CD3/TCR complex. In certain embodiments, the CD3 complex comprises a CD3γ chain, a CD3δ chain, and two CD3ε chains.

In certain embodiments, the CD3γ chain comprises or consists of an amino acid sequence that is at least about 85%, about 90%, about 95%, about 96%, about 97%, about 98%, about 99% or about 100% homologous or identical to the amino acid sequence having a NCBI reference number: NP_000064.1 (SEQ ID NO: 30) or a fragment thereof, and/or may optionally comprise up to one or up to two or up to three conservative amino acid substitutions. SEQ ID NO: 30 is provided below.

[SEQ ID NO: 30]
MEQGKGLAVLILAIILLQGTLAQSIKGNHLVKVYDYQEDGSVLLT
CDAEAKNITWEKDGKMIGELTEDKKKWNLGSNAKDPRGMYQCKGS
QNKSKPLQVYYRMCQNCIELNAATISGELFAEIVSIFVLAVGVYF
IAGQDGVRQSRASDKQTLLPNDQLYQPLKDREDDQYSHLQGNQLR
RN

In certain embodiments, the CD38 chain comprises or consists of an amino acid sequence that is at least about 85%, about 90%, about 95%, about 96%, about 97%, about 98%, about 99% or about 100% homologous or identical to the amino acid sequence having a NCBI reference numbers: NP_000723.1 (SEQ ID NO: 31) or a fragment thereof, or the amino acid sequence having a NCBI reference numbers: NP_001035741.1 (SEQ ID NO: 32) or a fragment thereof, and/or may optionally comprise up to one or up to two or up to three conservative amino acid substitutions. SEQ ID NO: 31 and 32 are provided below.

[SEQ ID NO: 31]
MEHSTFLSGLVLATLLSQVSPEKIPIEELEDRVFVNCNTSITWVEGTVGT
LLSDITRLDLGKRILDPRGIYRCNGTDIYKDKESTVQVHYRMCQSCVELD
PATVAGIIVTDVIATLLLALGVFCFAGHETGRLSGAADTQALLRNDQVYQ
PLRDRDDAQYSHLGGNWARNK
[SEQ ID NO: 32]
MEHSTFLSGLVLATLLSQVSPFKIPIEELEDRVFVNCNTSITWVEGTVGT
LLSDITRLDLGKRILDPRGIYRCNGTDIYKDKESTVQVHYRTADTQALLR
NDQVYQPLRDRDDAQYSHLGGNWARNK

In certain embodiments, the CD3& chain comprises or consists of an amino acid sequence that is at least about 85%, about 90%, about 95%, about 96%, about 97%, about 98%, about 99% or about 100% homologous or identical to the amino acid sequence having a NCBI reference number: NP_000724.1 (SEQ ID NO: 33) or a fragment thereof, and/or may optionally comprise up to one or up to two or up to three conservative amino acid substitutions. SEQ ID NO: 33 is provided below.

[SEQ ID NO: 33]
MQSGTHWRVLGLCLLSVGVWGQDGNEEMGGITQTPYKVSISGTTVILTCP
QYPGSEILWQHNDKNIGGDEDDKNIGSDEDHLSLKEFSELEQSGYYVCYP
RGSKPEDANFYLYLRARVCENCMEMDVMSVATIVIVDICITGGLLLLVYY
WSKNRKAKAKPVTRGAGAGGRQRGQNKERPPPVPNPDYEPIRKGQRDLYS
GLNQRRI

In certain embodiments, the TCR-like fusion molecule exhibits a greater antigen sensitivity than a CAR targeting the same antigen. In certain embodiments, the TCR-like fusion molecule is capable of inducing an immune response when binding to an antigen that has a low antigen density on the surface of a tumor cell. In certain embodiments, cells comprising the TCR-like fusion molecule can be used to treat a subject having tumor cells with a low expression level of a surface antigen, e.g., from a relapse of a disease, wherein the subject received treatment which leads to residual tumor cells. In certain embodiments, the tumor cells have a low antigen density of a target molecule on the surface of the tumor cells. In certain embodiments, a target molecule having a low antigen density on the cell surface has a density of less than about 5,000 molecules per cell, less than about 4,000 molecules per cell, less than about 3,000 molecules per cell, less than about 2,000 molecules per cell, less than about 1,500 molecules per cell, less than about 1,000 molecules per cell, less than about 500 molecules per cell, less than about 200 molecules per cell, or less than about 100 molecules per cell. In certain embodiments, a target molecule having a low antigen density on the cell surface has a density of less than about 2,000 molecules per cell. In certain embodiments, a target molecule having a low antigen density on the cell surface has a density of less than about 1,500 molecules per cell. In certain embodiments, a target molecule having a low antigen density on the cell surface has a density of less than about 1,000 molecules per cell. In certain embodiments, a target molecule having a low antigen density on the cell surface has a density of between about 4,000 molecules per cell and about 2,000 molecules per cell, between about 2,000 molecules per cell and about 1,000 molecules per cell, between about 1,500 molecules per cell and about 1,000 molecules per cell, between about 2,000 molecules per cell and about 500 molecules per cell, between about 1,000 molecules per cell and about 200 molecules per cell, or between about 1,000 molecules per cell and about 100 molecules per cell.

In certain embodiments, the antigen-recognizing receptor is a TCR-like fusion molecule that comprises a first antigen binding chain comprising a V_{H} of an antibody and a constant domain comprising a TRBC polypeptide; and a second antigen binding chain comprising a V_{L} of an antibody and a constant domain comprising a TRAC polypeptide. In certain embodiments, the first antigen binding chain is designated as “V_{H}-TRBC chain”. In certain embodiments, the second antigen binding chain is designated as “V_{L}-TRAC chain”. In certain embodiments, the first antigen binding chain comprises a hinge region between the V_{H} and the TRBC polypeptide. In certain embodiments, the hinge region comprises or consists of the amino acid sequence set forth in SEQ ID NO: 26 or SEQ ID NO: 28. In certain embodiments, the second antigen binding chain comprises a hinge region between the V_{L} and the TRAC polypeptide. In certain embodiments, the hinge region comprises or consists of the amino acid sequence set forth in SEQ ID NO: 26 or SEQ ID NO: 28.

In certain embodiments, the antigen-recognizing receptor is a TCR-like fusion molecule that comprises a first antigen binding chain comprising a V_{H} of an antibody and a constant domain comprising a TRAC polypeptide; and a second antigen binding chain comprising a V_{L} of an antibody and a constant domain comprising a TRBC polypeptide. In certain embodiments, the first antigen binding chain is designated as “V_{H}-TRAC chain”. In certain embodiments, the second antigen binding chain is designated as “V_{L}-TRBC chain”. In certain embodiments, the first antigen binding chain comprises a hinge region between the V_{H} and the TRAC polypeptide. In certain embodiments, the second antigen binding chain comprises a hinge region between the V_{L} and the TRBC polypeptide. In certain embodiments, the first antigen binding chain and the second antigen binding chain bind to an antigen (e.g., human CD70).

In certain embodiments, the antigen-recognizing receptor is a TCR-like fusion molecule that comprises a first antigen binding chain comprising a V_{H} of an antibody and a constant domain comprising a TRBC polypeptide; and a second antigen binding chain comprising a V_{L} of an antibody and a constant domain comprising a TRAC polypeptide. In certain embodiments, the first antigen binding chain is designated as “V_{H}-TRBC chain”. In certain embodiments, the second antigen binding chain is designated as “V_{L}-TRAC chain”. In certain embodiments, the first antigen binding chain comprises a hinge region between the V_{H} and the TRAC polypeptide. In certain embodiments, the second antigen binding chain comprises a hinge region between the V_{L} and the TRBC polypeptide. In certain embodiments, the first antigen binding chain and the second antigen binding chain bind to an antigen (e.g., human CD70).

In certain embodiments, the antigen-recognizing receptor is a TCR-like fusion molecule that binds to CD70 (e.g., human CD70) and comprises two antigen binding chains, e.g., a first antigen binding chain that comprises a V_{H} and a TRBC polypeptide (“V_{H}-TRBC chain”) and a second antigen binding chain that comprises a V_{L} and a TRBC polypeptide (“V_{L}-TRAC chain”), which are capable of dimerizing and binding to CD70. In certain embodiments, the V_{H} comprises a CDR1 comprising the amino acid sequence set forth in SEQ ID NO: 34, a CDR2 comprising the amino acid sequence set forth in SEQ ID NO: 35, and a CDR3 comprising the amino acid sequence set forth in SEQ ID NO: 36. In certain embodiments, the V_{H} comprises the amino acid sequence set forth in SEQ ID NO: 40. In certain embodiments, the V_{L} comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 39. In certain embodiments, the V_{L} comprises the amino acid sequence set forth in SEQ ID NO: 42. In certain embodiments, the TRAC polypeptide comprises the amino acid sequence set forth in SEQ ID NO: 7 or SEQ ID NO: 9. In certain embodiments, the TRBC polypeptide is a TRBC2 polypeptide. In certain embodiments, the TRBC2 polypeptide comprises the amino acid sequence set forth in SEQ ID NO: 12 or SEQ ID NO: 14. In certain embodiments, the TCR-like fusion molecule is designated as “70-HIT” or “70H”. SEQ ID NO: 34-43 are provided in Table 1 below.

In certain embodiments, the CDRs regions/sequences disclosed herein are delineated using the Kabat system (Swindells et al., J Mol Biol. 2017 Feb. 3; 429(3):356-364).

TABLE 1

| | CDRS | | |
|---|---|---|---|
| | 1 | 2 | 3 |
| V_{H} | SYIMH [SEQ ID NO: | VISYDGRNKYYADSVKG | DTDGYDEDY [SEQ |
| | 34] | [SEQ ID NO: 35] | ID NO: 36] |
| V_{L} | RASQSVSSYLA [SEQ | DASNRAT [SEQ ID | QQRTNWPLT [SEQ |
| | ID NO: 37] | NO: 38] | ID NO: 39] |

| | |
|---|---|
| Full V_{H} | MELGLSWIFLLAILKGVQCQVQLVESGGGVVQPGRSLRLSCAASGFTESSYIMHWVRQ |
| | APGKGLEWVAVISYDGRNKYYADSVKGRFTISRDNSKNTLYLQMNSLRAEDTAVYYCA |
| | RDTDGYDFDYWGQGTLVTV [SEQ ID NO: 40] |
| Full V_{H} - | ATGGAACTCGGTCTGTCCTGGATTTTTCTGCTTGCGATCTTGAAAGGAGTGCAGTGCC |
| DNA | AGGTGCAGCTGGTGGAGTCTGGGGGAGGCGTGGTCCAGCCTGGGAGGTCCCTGAGACT |
| | CTCCTGTGCAGCCTCTGGATTTACCTTCAGTAGCTATATTATGCACTGGGTCCGCCAG |
| | GCTCCAGGCAAGGGGCTGGAGTGGGTGGCAGTTATATCATATGATGGAAGAAACAAAT |
| | ACTACGCAGACTCCGTGAAGGGCCGATTCACCATCTCCAGAGACAATTCCAAGAACAC |
| | GCTGTATCTGCAAATGAACAGCCTGAGAGCTGAGGACACGGCTGTGTATTACTGTGCG |
| | AGAGATACGGATGGCTACGATTTTGACTACTGGGGCCAGGGAACCCTGGTCACCGTC |
| | [SEQ ID NO: 41] |
| Full V_{L} | EIVLTQSPATLSLSPGERATLSCRASQSVSSYLAWYQQKPGQAPRLLIYDASNRATGI |
| | PARFSGSGSGTDFTLTISSLEPEDFAVYYCQQRTNWPLTFGGGTKVEIK [SEQ ID |
| | NO: 42] |
| Full V_{L} - | ATGGAAGCCCCAGCTCAGCTTCTCTTCCTCCTGCTACTCTGGCTCCCAGATACCACCG |
| DNA | GAGAAATTGTGTTGACACAGTCTCCAGCCACCCTGTCTTTGTCTCCAGGGGAAAGAGC |
| | CACCCTCTCCTGCAGGGCCAGTCAGAGTGTTAGCAGCTACTTAGCCTGGTACCAACAG |
| | AAACCTGGCCAGGCTCCCAGGCTCCTCATCTATGATGCATCCAACAGGGCCACTGGCA |
| | TCCCAGCCAGGTTCAGTGGCAGTGGGTCTGGGACAGACTTCACTCTCACCATCAGCAG |
| | CCTAGAGCCTGAAGATTTTGCAGTTTATTACTGTCAGCAGCGTACCAACTGGCCGCTC |
| | ACTTTCGGCGGAGGGACCAAGGTGGAGATC [SEQ ID NO: 43] |

In certain embodiments, the antigen-recognizing receptor is a TCR-like fusion molecule that binds to CD70 (e.g., human CD70) and comprises two antigen binding chains, e.g., a first antigen binding chain that comprises a V_{H} and a TRBC polypeptide (“V_{H}-TRBC chain”) and a second antigen binding chain that comprises a V_{L} and a TRBC polypeptide (“V_{L}-TRAC chain”), which are capable of dimerizing and binding to CD70. In certain embodiments, the V_{H} comprises a CDR1, a CDR2, and a CDR3 of a V_{H} sequence of an anti-CD70 antibody disclosed in International Patent Publication No. WO 2007/038637, which is incorporated by reference in its entirety. In certain embodiments, the V_{H} comprises a CDR1, a CDR2, and a CDR3 of a V_{H} sequence of an anti-CD70 antibody 2H5 disclosed in International Patent Publication No. WO 2007/038637. In certain embodiments, the V_{L} comprises a CDR1, a CDR2, and a CDR3 of a V_{H} sequence of an anti-CD70 antibody disclosed in International Patent Publication No. WO 2007/038637. In certain embodiments, the V_{L} comprises a CDR1, a CDR2, and a CDR3 of a V_{H} sequence of an anti-CD70 antibody 2H5 disclosed in International Patent Publication No. WO 2007/038637.

Various TCR-like fusion molecules are disclosed in International Patent Application Publication No. WO2019/133969, which is incorporated by reference hereby in its entirety.

### 2.1.3. Delivery of the Antigen-Recognizing Receptor

In certain embodiments, the antigen-recognizing receptor is delivered to the cell by a viral method. In certain embodiments, the viral method comprises a viral vector. In certain embodiments, the viral vector is a retroviral vector (e.g., a gamma-retroviral vector or a lentiviral vector). Other viral vectors include adenoviral vectors, adeno-associated viral vectors, vaccinia viruses, bovine papilloma viruses, and herpes viruses (e.g., such as Epstein-Barr Virus).

In certain embodiments, the antigen-recognizing receptor is delivered to the cell by a non-viral method. Any targeted genome editing methods can also be used to deliver the first antigen-recognizing receptor to the cell. In certain embodiments, the antigen-recognizing receptor is delivered to the cell by a method comprising homologous recombination, a Zinc finger nuclease, a meganuclease, a Transcription activator-like effector nuclease (TALEN), a Clustered regularly-interspaced short palindromic repeats (CRISPR) system, or a combination thereof. In certain embodiments, a CRISPR system is used to deliver the first antigen-recognizing receptor to the cell.

In certain embodiments, the cell is a T cell, and the first antigen-recognizing receptor is integrated at a locus within the genome of the T cell. Non-limiting examples of loci include a TRAC locus, a TRBC locus, a TRDC locus, and a TRGC locus. In certain embodiments, the locus is a TRAC locus or a TRBC locus. In certain embodiments, the cell is a T cell, and the first antigen-recognizing receptor is integrated at a TRAC locus. Methods of targeting a CAR to a site within the genome of T cell are disclosed in WO2017180989 and Eyquem et al., Nature. (2017 Mar. 2); 543 (7643): 113-117, both of which are incorporated by reference in their entireties. In certain embodiments, the cell is a T cell, the first antigen-recognizing receptor is a CAR, and the first antigen-recognizing receptor is integrated at a TRAC locus. In certain embodiments, the cell further comprises a gene disruption of a TRBC locus. In certain embodiments, the gene disruption of a TRBC locus results in knockout of TRBC locus.

### 2.1.4. Second Antigen Recognizing Receptors

In certain embodiments, the presently disclosed cells comprising the antigen-recognizing receptor (e.g., a first antigen-recognizing receptor) further comprise a second antigen recognizing receptor that targets a second antigen. In certain embodiments, the second antigen-recognizing receptor is a chimeric receptor. In certain embodiments, the chimeric receptor is a chimeric antigen receptor (CAR). In certain embodiments, the chimeric receptor is a chimeric ligand receptor. In certain embodiments, the chimeric receptor is a CCR. In certain embodiments, the chimeric receptor is a T cell receptor (TCR).

### 2.1.4.1. Second Antigen

In certain embodiments, the second antigen is a tumor antigen, e.g., one disclosed in Section 2.1.1. In certain embodiments, the tumor antigen is an antigen with low antigen density. In certain embodiments, the tumor antigen is expressed on a cell with low tumor cell frequency.

In certain embodiments, the second antigen is selected from the group consisting of CD19, CD70, IL1RAP, ABcG2, AChR, ACKR6, ADAMTS13, ADGRE2, ADGRE2 (EMR2), ADORA3, ADRA1D, AGER, ALS2, an antigen of a cytomegalovirus (CMV) infected cell (e.g. a cell surface antigen), ANO9, AQP2, ASIC3, ASPRV1, ATP6VOA4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD276, CD30, CD300LF, CD312, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-a, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, LICAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1,GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PR1), PSD2, PTPRJ, RDH16, receptor tyrosine-protein kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TASIR3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, and ZDHHC11.

In certain embodiments, the second antigen is a pathogen antigen, e.g., one disclosed in Section 2.1.1.

### 2.1.4.2. Chimeric Antigen Receptors (CARs)

CARs are engineered receptors, which graft or confer a specificity of interest onto an immune effector cell. CARs can be used to graft the specificity of a monoclonal antibody onto a T cell; with transfer of their coding sequence facilitated by retroviral vectors.

There are three generations of CARS. “First generation” CARs are typically composed of an extracellular antigen-binding domain (e.g., an scFv) that binds to a target antigen, and an intracellular signaling domain. In certain embodiments, the CAR further comprises a transmembrane domain. “First generation” CARs can provide de novo antigen recognition and cause activation of both CD4⁺ and CD8⁺ T cells through their CD3ζ chain signaling domain in a single fusion molecule, independent of HLA-mediated antigen presentation. “Second generation” CARs include a signaling domain of a co-stimulatory molecule (e.g., CD28, 4-1BB, ICOS, OX40, CD27, CD40,NKG2D, DAP-10, CD2, CD150, CD226) to the intracellular signaling domain of the CAR to provide co-stimulation signals to the cell (e.g., T cell or NK cell). “Second generation” CARs comprise those that provide both co-stimulation (e.g., CD28 or 4-1BB) and activation (CD3ζ). “Third generation” CARs comprise those that provide multiple co-stimulation (e.g., CD28 and 4-1BB) and activation (CD3ζ).

In certain embodiments, the second antigen-recognizing receptor is a CAR comprising an extracellular antigen-binding domain that binds to a second antigen, and an intracellular signaling domain. In certain embodiments, the CAR further comprises a transmembrane domain. In certain embodiments, the CAR further comprises a hinger/spacer region.

In certain embodiments, the extracellular antigen-binding domain of the CAR (for example, an scFv) binds to the first antigen with a dissociation constant (K_{D}) of about 5×10⁻⁷ M or less, about 1×10⁻⁷ M or less, about 5×10⁻⁸ M or less, about 1×10⁻⁸ M or less, about 5×10⁻⁹ M or less, or about 1×10⁻⁹ M or less, or about 1×10⁻¹⁰ M or less. In certain embodiments, the extracellular antigen-binding domain of the CAR (for example, an scFv) binds to the first antigen with a K_{D} of about 1×10⁻⁸ M or less.

Binding of the extracellular antigen-binding domain (for example, in an scFv) can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or an scFv) specific for the complex of interest. For example, the scFv can be radioactively labeled and used in a radioimmunoassay (RIA) (scc, for example, Weintraub, B., Principles of Radioimmunoassays, Seventh Training Course on Radioligand Assay Techniques, The Endocrine Society, March 1986, which is incorporated by reference herein). The radioactive isotope can be detected by such means as the use of a γ counter or a scintillation counter or by autoradiography. In certain embodiments, the extracellular antigen-binding domain of the CAR is labeled with a fluorescent marker. Non-limiting examples of fluorescent markers include green fluorescent protein (GFP), blue fluorescent protein (e.g., EBFP, EBFP2, Azurite, and mKalama1), cyan fluorescent protein (e.g., ECFP, Cerulean, and CyPet), and yellow fluorescent protein (e.g., YFP, Citrine, Venus, and YPet).

The extracellular antigen-binding domain can comprise or be an scFv, a Fab (which is optionally crosslinked), or a F(ab)₂. In certain embodiments, any of the foregoing molecules may be comprised in a fusion protein with a heterologous sequence to form the extracellular antigen-binding domain. In certain embodiments, the extracellular antigen-binding domain comprises or is an scFv. In certain embodiments, the scFv is a human scFv. In certain embodiments, the scFv is a humanized scFv. In certain embodiments, the scFv is a murine scFv.

In addition, the extracellular antigen-binding domain of the CAR can comprise a leader or a signal peptide that directs the nascent protein into the endoplasmic reticulum. Signal peptide or leader can be essential if the CAR is to be glycosylated and anchored in the cell membrane. The signal sequence or leader can be a peptide sequence (about 5, about 10, about 15, about 20, about 25, or about 30 amino acids long) present at the N-terminus of newly synthesized proteins that directs their entry to the secretory pathway. In certain embodiments, the signal peptide is covalently joined to the 5′ terminus (N-terminus) of the extracellular antigen-binding domain of the CAR. Exemplary leader sequences include, but is not limited to, a human IL-2 signal sequence (e.g., a human IL-2 signal sequence comprising or consisting of the amino acid sequence set forth in SEQ ID NO: 44), a mouse IL-2 signal sequence (e.g., a mouse IL-2 signal sequence comprising or consisting of the amino acid sequence set forth in SEQ ID NO: 45); a human kappa leader sequence (e.g., a human kappa leader sequence comprising or consisting of the amino acid sequence set forth in SEQ ID NO: 46), a mouse kappa leader sequence (e.g., a mouse kappa leader sequence comprising or consisting of the amino acid sequence set forth in SEQ ID NO: 47); a human CD8 leader sequence (e.g., a human CD8 leader sequence comprising or consisting of the amino acid sequence set forth in SEQ ID NO: 48); a truncated human CD8 signal peptide (e.g., a truncated human CD8 signal peptide comprising or consisting of the amino acid sequence set forth in SEQ ID NO: 49); a human albumin signal sequence (e.g., a human albumin signal sequence comprising or consisting of the amino acid sequence set forth in SEQ ID NO: 50); and a human prolactin signal sequence (e.g., a human prolactin signal sequence comprising or consisting of the amino acid sequence set forth in SEQ ID NO: 51). SEQ ID NO: 44-51 are provided below.

| [SEQ ID NO: 44] | |
|---|---|
| | MYRMQLLSCIALSLALVINS |
| [SEQ ID NO: 45] | |
| | MYSMQLASCVTLTLVLLVNS |
| [SEQ ID NO: 46] | |
| | METPAQLLFLLLLWLPDTTG |
| [SEQ ID NO: 47] | |
| | METDTLLLWVLLLWVPGSTG |
| [SEQ ID NO: 48] | |
| | MALPVTALLLPLALLLHAARP |
| [SEQ ID NO: 49] | |
| | MALPVTALLLPLALLLHA |
| [SEQ ID NO: 50] | |
| | MKWVTFISLLESSAYS |
| [SEQ ID NO: 51] | |
| | MDSKGSSQKGSRLLLLLVVSNLLLCQGVVS |

In certain embodiments, the signal peptide comprises a CD8 polypeptide, e.g., the CAR comprises a truncated CD8 signal peptide. In certain embodiments, the signal peptide comprises or consists of the amino acid sequence set forth in SEQ ID NO: 49.

In certain embodiments, the second antigen-recognizing receptor is a CAR that comprises a transmembrane domain. Different transmembrane domains result in different receptor stability. After antigen recognition, receptors cluster and a signal are transmitted to the cell. In accordance with the presently disclosed subject matter, the transmembrane domain of the first antigen-recognizing receptor can comprise a native or modified transmembrane domain of a CD8 polypeptide, a CD28 polypeptide, a CD3ζ polypeptide, a CD40 polypeptide, a 4-1 BB polypeptide, an OX40 polypeptide, a CD84 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, a NKG2D polypeptide, a synthetic polypeptide (not based on a protein associated with the immune response), or a combination thereof.

In certain embodiments, the transmembrane domain of the CAR comprises a CD28 polypeptide (e.g., the transmembrane domain of CD28 or a portion thereof). In certain embodiments, the transmembrane domain of the CAR comprises a transmembrane domain of human CD28 or a portion thereof. In certain embodiments, the CD28 polypeptide comprises or consists of an amino acid sequence that is a consecutive portion of the amino acid sequence having a NCBI Reference No: NP_006130 (SEQ ID NO: 52), which is at least about 20, or at least about 25, or at least about 30, and/or up to about 220 amino acids in length. In certain embodiments, the CD28 polypeptide comprises or consists of an amino acid sequence of amino acids 1 to 220, 1 to 50, 50 to 100, 100 to 150, 114 to 220, 150 to 200, 153 to 179, or 200 to 220 of SEQ ID NO: 52. In certain embodiments, the transmembrane domain of the CAR comprises a CD28 polypeptide that comprises or consists of amino acids 153 to 179 of SEQ ID NO: 52. SEQ ID NO: 52 is provided below.

[SEQ ID NO: 52]
MLRLLLALNLFPSIQVTGNKILVKQSPMLVAYDNAVNLSCKYSYNLESRE
FRASLHKGLDSAVEVCVVYGNYSQQLQVYSKTGENCDGKLGNESVTFYLQ
NLYVNQTDIYFCKIEVMYPPPYLDNEKSNGTIIHVKGKHLCPSPLFPGPS
KPFWVLVVVGGVLACYSLLVTVAFIIFWVRSKRSRLLHSDYMNMTPRRPG
PTRKHYQPYAPPRDFAAYRS

In certain embodiments, the second antigen-recognizing receptor is a CAR that further comprises a hinge/spacer region that links the extracellular antigen-binding domain to the transmembrane domain. The hinge/spacer region can be flexible enough to allow the antigen binding domain to orient in different directions to facilitate antigen recognition. In certain embodiments, the hinge/spacer region of the CAR can comprise a native or modified hinge region of a CD8 polypeptide, a CD28 polypeptide, a CD3ζ polypeptide, a CD40 polypeptide, a 4-1BB polypeptide, an OX40 polypeptide, a CD84 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, a NKG2D polypeptide, a synthetic polypeptide (not based on a protein associated with the immune response), or a combination thereof. The hinge/spacer region can be the hinge region from IgG1, or the CH2CH3 region of immunoglobulin and portions of CD3, a portion of a CD28 polypeptide (e.g., a portion of SEQ ID NO: 52), a portion of a CD8 polypeptide, or a synthetic spacer sequence.

In certain embodiments, the second antigen-recognizing receptor is a CAR that further comprises a hinge/spacer region comprising a native or modified hinge region of a CD28 polypeptide. In certain embodiments, the hinge/spacer region of the first antigen-recognizing receptor (e.g., a CAR) comprises a CD28 polypeptide comprising or consisting of amino acids 114 to 152 of SEQ ID NO: 52.

In certain embodiments, the hinge/spacer region is positioned between the extracellular antigen-binding domain and the transmembrane domain. In certain embodiments, the hinge/spacer region comprises a CD8 polypeptide, a CD28 polypeptide, a CD3ζ polypeptide, a CD4 polypeptide, a 4-1BB polypeptide, an OX40 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, a NKG2D polypeptide, a synthetic polypeptide (not based on a protein associated with the immune response), or a combination thereof. In certain embodiments, the transmembrane domain comprises a CD8 polypeptide, a CD28 polypeptide, a CD3ζ polypeptide, a CD4 polypeptide, a 4-1BB polypeptide, an OX40 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40 polypeptide, a NKG2D polypeptide, a synthetic polypeptide (not based on a protein associated with the immune response), or a combination thereof.

In certain embodiments, the transmembrane domain and the hinge/spacer region are derived from the same molecule. In certain embodiments, the transmembrane domain and the hinge/spacer region are derived from different molecules. In certain embodiments, the hinge/spacer region comprises a CD28 polypeptide and the transmembrane domain comprises a CD28 polypeptide. In certain embodiments, the hinge/spacer region comprises a CD28 polypeptide and the transmembrane domain comprises a CD28 polypeptide. In certain embodiments, the hinge/spacer region comprises a CD84 polypeptide and the transmembrane domain comprises a CD84 polypeptide. In certain embodiments, the hinge/spacer region comprises a CD166 polypeptide and the transmembrane domain comprises a CD166 polypeptide. In certain embodiments, the hinge/spacer region comprises a CD8a polypeptide and the transmembrane domain comprises a CD8a polypeptide. In certain embodiments, the hinge/spacer region comprises a CD8b polypeptide and the transmembrane domain comprises a CD8b polypeptide. In certain embodiments, the hinge/spacer region comprises a CD28 polypeptide and the transmembrane domain comprises an ICOS polypeptide.

In certain embodiments, the second antigen-recognizing receptor is a CAR that comprises an intracellular signaling domain. In certain embodiments, the intracellular signaling domain of the CAR comprises a CD3ζ polypeptide. CD3ζ can activate or stimulate a cell (e.g., a cell of the lymphoid lineage, e.g., a T-cell). Wild type (“native”) CD3ζ comprises three functional immunoreceptor tyrosine-based activation motifs (ITAMs), three functional basic-rich stretch (BRS) regions (BRS1, BRS2 and BRS3). CD3 transmits an activation signal to the cell (e.g., a cell of the lymphoid lineage, e.g., a T-cell) after antigen is bound. The intracellular signaling domain of the CD3ζ-chain is the primary transmitter of signals from endogenous TCRs.

In certain embodiments, the intracellular signaling domain of the CAR comprises a native CD3ζ. In certain embodiments, the native CD3ζ comprises or consists of an amino acid sequence that is at least about 85%, about 90%, about 95%, about 96%, about 97%, about 98%, about 99% or about 100% identical or homologous to the amino acid sequence having a NCBI Reference No: NP_932170 (SEQ ID NO: 53) or a fragment thereof, and/or may optionally comprise up to one or up to two or up to three conservative amino acid substitutions. In certain embodiments, the CD3ζ polypeptide comprises or consists of an amino acid sequence that is a consecutive portion of SEQ ID NO: 53, which is at least about 20, or at least about 30, or at least about 40, or at least about 50, and up to about 164 amino acids in length. In certain embodiments, the native CD3 comprises or consists of the amino acid sequence of amino acids 1 to 164, 1 to 50, 50 to 100, 52 to 164, 100 to 150, or 150 to 164 of SEQ ID NO: 53. In certain embodiments, the intracellular signaling domain of the CAR comprises a native CD3ζ comprising or consisting of the amino acid sequence of amino acids 52 to 164 of SEQ ID NO: 53. SEQ ID NO: 53 is provided below:

[SEQ ID NO: 53]
MKWKALFTAAILQAQLPITEAQSFGLLDPKLCYLLDGILFIYGVILTALF
LRVKESRSADAPAYQQGQNQLYNELNLGRREEYDVLDKRRGRDPEMGGKP
QRRKNPQEGLYNELQKDKMAEAYSEIGMKGERRRGKGHDGLYQGLSTATK
DTYDALHMQALPPR

In certain embodiments, the native CD3ζ comprises or consists of an amino acid sequence that is at least about 85%, about 90%, about 95%, about 96%, about 97%, about 98%, about 99% or about 100% identical or homologous to the amino acid sequence set forth in SEQ ID NO: 54. SEQ ID NO: 54 is provided below:

[SEQ ID NO: 54]
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR

In certain embodiments, the intracellular signaling domain of the CAR comprises a modified CD3ζ polypeptide. In certain embodiments, the modified CD3ζ polypeptide comprises one, two or three ITAMs. In certain embodiments, the modified CD3ζ polypeptide comprises a native ITAM1. In certain embodiments, the native ITAM1 comprises or consists of the amino acid sequence set forth in SEQ ID NO: 55.

| [SEQ ID NO: 55] | |
|---|---|
| | QNQLYNELNLGRREEYDVLDKR |

An exemplary nucleic acid sequence encoding the amino acid sequence of SEQ ID NO: 55 is set forth in SEQ ID NO: 56, which is provided below.

[SEQ ID NO: 56]
CAGAACCAGCTCTATAACGAGCTCAATCTAGGACGAAGAGAGGAGTACGA
TGTTTTGGACAAGAGA

In certain embodiments, the modified CD3ζ polypeptide comprises an ITAM1 variant comprising one or more loss-of-function mutations. In certain embodiments, the ITAM1 variant comprises or consists of two loss-of-function mutations. In certain embodiments, each of the one or more (e.g., two) loss of function mutations comprises a mutation of a tyrosine residue in ITAM1. In certain embodiments, the ITAM1 variant consists of two loss-of-function mutations. In certain embodiments, the ITAM1 variant comprises or consists of the amino acid sequence set forth in SEQ ID NO: 57, which is provided below.

| [SEQ ID NO: 57] | |
|---|---|
| | QNQLFNELNLGRREEFDVLDKR |

An exemplary nucleic acid sequence encoding the amino acid sequence of SEQ ID NO: 57 is set forth in SEQ ID NO: 58, which is provided below.

[SEQ ID NO: 58]
CAGAACCAGCTCTTTAACGAGCTCAATCTAGGACGAAGAGAGGAGTTCGA
TGTTTTGGACAAGAGA

In certain embodiments, the modified CD3ζ polypeptide comprises a native ITAM2. In certain embodiments, the native ITAM2 comprises or consists of the amino acid sequence set forth in SEQ ID NO: 59, which is provided below.

[SEQ ID NO: 59]
QEGLYNELQKDKMAEAYSEIGMK

An exemplary nucleic acid sequence encoding the amino acid sequence of SEQ ID NO: 59 is set forth in SEQ ID NO: 60, which is provided below.

[SEQ ID NO: 60]
CAGGAAGGCCTGTACAATGAACTGCAGAAAGATAAGATGGCGGAGGCCTA
CAGTGAGATTGGGATGAAA

In certain embodiments, the modified CD3ζ polypeptide comprises an ITAM2 variant. In certain embodiments, the ITAM2 variant comprises or consists of one or more loss-of-function mutations. In certain embodiments, the ITAM2 variant comprises or consists of two loss-of-function mutations. In certain embodiments, each of the one or more (e.g., two) the loss of function mutations comprises a mutation of a tyrosine residue in ITAM2. In certain embodiments, the ITAM1 variant consists of two loss-of-function mutations. In certain embodiments, the ITAM2 variant comprises or consists of the amino acid sequence set forth in SEQ ID NO: 61, which is provided below.

| [SEQ ID NO: 61] | |
|---|---|
| | QEGLENELQKDKMAEAFSEIGMK |

An exemplary nucleic acid sequence encoding the amino acid sequence of SEQ ID NO: 61 is set forth in SEQ ID NO: 62, which is provided below.

[SEQ ID NO: 62]
CAGGAAGGCCTGTTCAATGAACTGCAGAAAGATAAGATGGCGGAGGCCTT
CAGTGAGATTGGGATGAAA

In certain embodiments, the modified CD3ζ polypeptide comprises a native ITAM3. In certain embodiments, the native ITAM3 comprises or consists of the amino acid sequence set forth in SEQ ID NO: 63, which is provided below.

| [SEQ ID 63] | |
|---|---|
| | HDGLYQGLSTATKDTYDALHMQ |

An exemplary nucleic acid sequence encoding the amino acid sequence of SEQ ID NO: 63 is set forth in SEQ ID NO: 64, which is provided below.

[SEQ ID NO: 64]
CACGATGGCCTTTACCAGGGTCTCAGTACAGCCACCAAGGACACCTACGA
CGCCCTTCACATGCAG

In certain embodiments, the modified CD32 polypeptide comprises an ITAM3 variant. In certain embodiments, the ITAM3 variant comprises or consists of two loss-of-function mutations. In certain embodiments, each of the one or more (e.g., two) the loss of function mutations comprises a mutation of a tyrosine residue in ITAM3. In certain embodiments, the ITAM3 variant comprises or consists of two loss-of-function mutations. In certain embodiments, the ITAM3 variant comprises or consists of the amino acid sequence set forth in SEQ ID NO: 65, which is provided below.

[SEQ ID NO: 65]
HDGLFQGLSTATKDTFDALHMQ

An exemplary nucleic acid sequence encoding the amino acid sequence of SEQ ID NO: 65 is set forth in SEQ ID NO: 66, which is provided below.

[SEQ ID NO: 66]
CACGATGGCCTTTTCCAGGGGCTCAGTACAGCCACCAAGGACACCTTCGA
CGCCCTTCACATGCAG

Various modified CD3ζ polypeptides and CARs comprising modified CD3ζ polypeptides are disclosed in International Patent Application Publication No. WO2019/133969, which is incorporated by reference hereby in its entirety.

In certain embodiments, the intracellular signaling domain of the CAR comprises a modified CD3ζ polypeptide comprising a native ITAM1, an ITAM2 variant comprising or consisting of one or more (e.g., two) loss-of-function mutations, and an ITAM3 variant comprising or consisting of one or more (e.g., two) loss-of-function mutations. In certain embodiments, the intracellular signaling domain of the CAR comprises a modified CD3ζ polypeptide comprising a native ITAM1, an ITAM2 variant consisting of two loss-of-function mutations, and an ITAM3 variant consisting of two loss-of-function mutations. In certain embodiments, the intracellular signaling domain of the CAR comprises a modified CD3ζ polypeptide comprising a native ITAM1 consisting of the amino acid sequence set forth in SEQ ID NO: 55, an ITAM2 variant consisting of the amino acid sequence set forth in SEQ ID NO: 59, and an ITAM3 variant consisting of the amino acid sequence set forth in SEQ ID NO: 63. In certain embodiments, the CAR is designated as “1XX”. In certain embodiments, the modified CD3ζ polypeptide comprises or consists of the amino acid sequence set forth in SEQ ID NO: 67. SEQ ID NO: 67 is provided below:

[SEQ ID NO: 67]
RVKFSRSADAPAYQQGQNQLYNELNLGRREEYDVLDKRRGRDPEMGGKPR
RKNPQEGLFNELQKDKMAEAFSEIGMKGERRRGKGHDGLFQGLSTATKDT
FDALHMQALPPR

In certain embodiments, the intracellular signaling domain of the CAR comprises a modified CD3ζ polypeptide comprising or consisting of an amino acid sequence that is at least about 80%, at least about 85%, at least about 90%, at least about 95%, at least about 96%, at least about 97%, at least about 98%, or at least about 99%, at least about 100% identical to SEQ ID NO: 67 or a fragment thereof, and/or may optionally comprise up to one or up to two or up to three conservative amino acid substitutions.

An exemplary nucleic acid sequence encoding the amino acid sequence of SEQ ID NO: 67 is set forth in SEQ ID NO: 68, which is provided below.

[SEQ ID NO: 68]
AGAGTGAAGTTCAGCAGGAGCGCAGACGCCCCCGCGTACCAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGAGA
AGGAAGAACCCTCAGGAAGGCCTGTTCAATGAACTGCAGAAAGATAAGAT
GGCGGAGGCCTTCAGTGAGATTGGGATGAAAGGCGAGCGCCGGAGGGGCA
AGGGGCACGATGGCCTTTTCCAGGGGCTCAGTACAGCCACCAAGGACACC
TTCGACGCCCTTCACATGCAGGCCCTGCCCCCTCGC

In certain embodiments, the intracellular signaling domain of the CAR further comprises at least one co-stimulatory signaling region. In certain embodiments, the at least one co-stimulatory region comprises a co-stimulatory molecule or a portion thereof. In certain embodiments, the at least one co-stimulatory region comprises at least an intracellular domain of at least one co-stimulatory molecule or a portion thereof. Non-limiting examples of costimulatory molecules include CD28, 4-1BB, OX40, CD27, CD40, CD154, CD97, CD11a/CD18, ICOS, DAP-10, CD2, CD150, CD226, and NKG2D.

In certain embodiments, the intracellular signaling domain of the CAR comprises a co-stimulatory signaling region that comprises a CD28 polypeptide, e.g., an intracellular domain of CD28 or a portion thereof. In certain embodiments, the intracellular signaling domain of the CAR comprises a co-stimulatory signaling region that comprises an intracellular domain of human CD28 or a portion thereof.

In certain embodiments, the CD28 polypeptide comprised in the co-stimulatory signaling region of the second antigen-recognizing receptor comprise or consists of an amino acid sequence that is at least about 80%, at least about 85%, at least about 90%, at least about 95%, at least about 96%, at least about 97%, at least about 98%, or at least about 99%, at least about 100% identical or homologous to the amino acid sequence set forth in SEQ ID NO: 52 or a fragment thereof, and/or may optionally comprise up to one or up to two or up to three conservative amino acid substitutions. In certain embodiments, the CD28 polypeptide comprised in the co-stimulatory signaling region of the CAR comprises or consist of an amino acid sequence that is a consecutive portion of SEQ ID NO: 52, which is at least about 20, or at least about 30, or at least about 40, or at least about 50, and up to about 220 amino acids in length. Alternatively or additionally, in certain embodiments, the CD28 polypeptide comprised in the co-stimulatory signaling region of the CAR comprises or consists of amino acids 1 to 220, 1 to 50, 50 to 100, 100 to 150, 114 to 220, 150 to 200, 180 to 220, or 200 to 220 of SEQ ID NO: 52. In certain embodiments, the intracellular signaling domain of the CAR comprises a co-stimulatory signaling region that comprises a CD28 polypeptide comprising or consisting of amino acids 180 to 220 of SEQ ID NO: 52.

An exemplary nucleic acid sequence encoding the amino acid sequence of amino acids 180 to 220 of SEQ ID NO: 52 is set forth in SEQ ID NO: 69, which is provided below.

[SEQ ID NO: 69]
AGGAGTAAGAGGAGCAGGCTCCTGCACAGTGACTACATGAACATGACTCC
CCGCCGCCCCGGGCCCACCCGCAAGCATTACCAGCCCTATGCCCCACCAC
GCGACTTCGCAGCCTATCGCTCC

In certain embodiments, the intracellular signaling domain of the second antigen-recognizing receptor comprises a co-stimulatory signaling region that comprises an intracellular domain of mouse CD28 or a portion thereof. In certain embodiments, the CD28 polypeptide comprised in the co-stimulatory signaling region comprises or consists of an amino acid sequence that is at least about 80%, at least about 85%, at least about 90%, at least about 95%, at least about 96%, at least about 97%, at least about 98%, or at least about 99%, at least about 100% identical or homologous to the amino acid sequence having a NCBI Reference No: NP_031668.3 (or SEQ ID NO: 70) or a fragment thereof, and/or may optionally comprise up to one or up to two or up to three conservative amino acid substitutions. In certain embodiments, the CD28 polypeptide comprised in the co-stimulatory signaling region of the CAR comprises or consists of an amino acid sequence that is a consecutive portion of SEQ ID NO: 70, which is at least about 20, or at least about 30, or at least about 40, or at least about 50, and up to 218 amino acids in length. In certain embodiments, the CD28 polypeptide comprised in the co-stimulatory signaling region of the CAR comprises or consists of the amino acid sequence of amino acids 1 to 218, 1 to 50, 50 to 100, 100 to 150, 150 to 218, 178 to 218, or 200 to 218 of SEQ ID NO: 70. In certain embodiments, the co-stimulatory signaling region of the CAR comprises a CD28 polypeptide that comprises or consists of amino acids 178 to 218 of SEQ ID NO: 70. SEQ ID NO: 70 is provided below.

[SEQ ID NO: 70]
MTLRLLFLAL NFFSVQVTEN KILVKQSPLL VVDSNEVSLS
CRYSYNLLAK EFRASLYKGV NSDVEVCVGN GNFTYQPQFR
SNAEFNCDGD FDNETVTFRL WNLHVNHTDI YFCKIEFMYP
PPYLDNERSN GTIIHIKEKH LCHTQSSPKL FWALVVVAGV
LFCYGLLVTV ALCVIWTNSR RNRLLQSDYM NMTPRRPGLT
RKPYQPYAPA RDFAAYRP

In certain embodiments, the intracellular signaling domain of the CAR comprises a co-stimulatory signaling region that comprises a 4-1BB polypeptide, e.g., an intracellular domain of 4-1BB or a portion thereof. In certain embodiments, the co-stimulatory signaling region comprises an intracellular domain of human 4-1BB or a portion thereof. In certain embodiments, the 4-1BB comprised in the co-stimulatory signaling region of the CAR comprises or consists of an amino acid sequence that is at least about 80%, at least about 85%, at least about 90%, at least about 95%, at least about 96%, at least about 97%, at least about 98%, or at least about 99%, at least about 100% identical or homologous to the sequence having a NCBI Ref. No.: NP_001552 (SEQ ID NO: 71) or a fragment thereof, and/or may optionally comprise up to one or up to two or up to three conservative amino acid substitutions. In certain embodiments, the 4-1BB comprised in the co-stimulatory signaling region of the CAR comprises or consists of an amino acid sequence that is a consecutive portion of SEQ ID NO: 71, which is at least about 20, or at least about 30, or at least about 40, or at least about 50, and/or up to about 50, up to about 60, up to about 70, up to about 80, up to about 90, up to about 100, up to about 200, or up to about 255 amino acids in length. In certain embodiments, the co-stimulatory signaling region of the CAR comprises a 4-1BB polypeptide that comprises or consists of the amino acid sequence of amino acids 1 to 255, 1 to 50, 50 to 100, 100 to 150, 150 to 200, or 200 to 255 of SEQ ID NO: 71. In certain embodiments, the co-stimulatory signaling region of the CAR comprises a 4-1BB polypeptide comprising or consisting of the amino acid sequence of amino acids 214 to 255 of SEQ ID NO: 71. SEQ ID NO: 71 is provided below.

[SEQ ID NO: 71]
MGNSCYNIVATLLLVLNFERTRSLQDPCSNCPAGTFCDNNRNQICSPCPP
NSFSSAGGQRTCDICRQCKGVFRTRKECSSTSNAECDCTPGFHCLGAGCS
MCEQDCKQGQELTKKGCKDCCFGTFNDQKRGICRPWTNCSLDGKSVLVNG
TKERDVVCGPSPADLSPGASSVTPPAPAREPGHSPQIISFFLALTSTALL
FLLFFLTLRFSVVKRGRKKLLYIFKQPFMRPVQTTQEEDGCSCRFPEEEE
GGCEL

In certain embodiments, the intracellular signaling domain of the CAR comprises two co-stimulatory signaling regions, wherein the first co-stimulatory signaling region comprises an intracellular domain of a first co-stimulatory molecule or a portion thereof, and the second co-stimulatory signaling region comprises an intracellular domain of a second co-stimulatory molecule or a portion thereof. The first and second co-stimulatory molecules are independently selected from the group consisting of CD28, 4-1BB, OX40, CD27, CD40, CD154, CD97, CD11a/CD18, ICOS, DAP-10, CD2, CD150, CD226, and NKG2D. In certain embodiments, the intracellular signaling domain of the CAR comprises two co-stimulatory signaling regions, wherein the first co-stimulatory signaling region comprises an intracellular domain of CD28 or a portion thereof and the second co-stimulatory signaling region comprises an intracellular domain of 4-1BB or a portion thereof.

### 2.1.4.3. Chimeric Ligand Receptors

In certain embodiments, the second antigen-recognizing receptor is a chimeric ligand receptor that comprises a ligand or a portion thereof that binds to the first antigen. In certain embodiments, the chimeric ligand receptor further comprises a transmembrane domain and an intracellular signaling domain.

In certain embodiments, the transmembrane domain is fused to the ligand or portion thereof. In certain embodiments, the transmembrane domain is fused to the intracellular signaling domain. In certain embodiments, the transmembrane domain is positioned between the ligand or portion thereof and the intracellular signaling domain. In certain embodiments the transmembrane domain of the chimeric ligand receptor is a transmembrane domain disclosed in Section 2.1.5.2. In certain embodiments, the intracellular signaling domain of the chimeric ligand receptor comprises a CD3ζ polypeptide (e.g., as disclosed in Section 2.1.5.2).

Additional information on the presently disclosed chimeric ligand receptor can be found in Sauer et al., Blood (2021) 138 (4): 318-330, the content of which is incorporated by reference in its entirety.

### 2.1.4.4. CCRs

In certain embodiments, a presently disclosed cell comprising a first antigen-recognizing receptor further comprises a CCR. The term “chimeric co-stimulating receptor” or “CCR” refers to a chimeric receptor that binds to an antigen and provides a co-stimulatory signal, but does not provide a T-cell activation signal to a cell comprising the CCR. Various CCRs are described in US20020018783 the contents of which are incorporated by reference in their entireties. CCRs mimic co-stimulatory signals, but unlike, CARs, do not provide a T-cell activation signal. In certain embodiments, the CCR lacks a CD3ζ polypeptide.

CCRs provide co-stimulation signal (e.g., a CD28-like signal or 4-1BB-like signal), in the absence of the natural co-stimulatory ligand on the antigen-presenting cell. A combinatorial antigen recognition, i.e., use of a CCR in combination with a CAR, can augment T-cell reactivity against the dual-antigen expressing T cells, thereby improving selective tumor targeting. Kloss et al., describe a strategy that integrates combinatorial antigen recognition, split signaling, and, critically, balanced strength of T-cell activation and co-stimulation to generate T cells that eliminate target cells that express a combination of antigens while sparing cells that express each antigen individually (Kloss et al., Nature Biotechnology (2013); 31(1):71-75, the content of which is incorporated by reference in its entirety). With this approach, T-cell activation requires CAR-mediated recognition of one antigen, whereas co-stimulation is independently mediated by a CCR specific for a second antigen. To achieve tumor selectivity, the combinatorial antigen recognition approach diminishes the efficiency of T-cell activation to a level where it is ineffective without rescue provided by simultaneous CCR recognition of the second antigen.

In certain embodiments, the CCR comprises an extracellular antigen-binding domain that binds to a third antigen and an intracellular domain that is capable of delivering a costimulatory signal to the cell but does not alone deliver an activation signal to the cell. In certain embodiments, the CCR further comprises a transmembrane domain. In certain embodiments, the intracellular domain of the CCR comprises at least an intracellular domain of a co-stimulatory molecule or a portion thereof. In certain embodiments, the co-stimulatory molecule is selected from the group consisting of CD28, 4-1BB, OX40, CD27, CD40, CD154, CD97, CD11a/CD18, ICOS, DAP-10, CD2, CD150, CD226, and NKG2D.

In certain embodiments, the CCR comprises an intracellular domain of CD28 or a portion thereof. In certain embodiments, the CCR comprises an intracellular domain of 4-1BB or a portion thereof. In certain embodiments, the CCR comprises an intracellular domain of CD28 or a portion thereof, and an intracellular domain of 4-1BB or a portion thereof.

In certain embodiments, the second antigen is selected so that expression of both of the first antigen and the second antigen is restricted to the targeted cells (e.g., cancerous tissue or cancerous cells). Similar to a CAR, the extracellular antigen-binding domain can be an scFv, a Fab, a F(ab)₂, or a fusion protein with a heterologous sequence to form the extracellular antigen-binding domain.

In certain embodiments, the cell comprising the first antigen-recognizing receptor and the CCR exhibits a greater degree of cytolytic activity against cells that are positive for both the first/second antigen and the third antigen as compared to against cells that are singly positive for the first/second antigen. In certain embodiments, the cell comprising the first antigen-recognizing receptor and the CCR exhibits substantially no or negligible cytolytic activity against cells that are singly positive for the first/second antigen.

In certain embodiments, the first antigen recognizing receptor (e.g., one disclosed in Section 2.1.2) binds to the antigen with a low binding affinity, e.g., a dissociation constant (K_{D})) of about 1×10⁻⁸ M or more, about 5×10⁻⁸ M or more, about 1×10⁻⁷ M or more, about 5×10⁻⁷ M or more, or about 1×10⁻⁶ M or more, or from about 1×10⁻⁸ M to about 1×10⁻⁶ M. In certain embodiments, the antigen recognizing receptor (e.g., a CAR, a TCR, or a TCR-like fusion molecule) binds to the antigen with a low binding avidity. In certain embodiments, the antigen recognizing receptor (e.g., a TCR-like fusion molecule) binds to the antigen at an epitope of low accessibility. In certain embodiments, the antigen recognizing receptor (e.g., a TCR-like fusion molecule) binds to the antigen with a binding affinity that is lower compared to the binding affinity with which the second antigen-recognizing receptor (e.g., a CCR) binds to the second antigen. In certain embodiments, the CCR binds to the second antigen with a binding affinity K_{D} of from about 1×10⁻⁹ M to about 1×10⁻⁷ M, e.g., about 1×10⁻⁷ M or less, about 1×10⁻⁸ M or less, or about 1×10⁻⁹ M or less.

### 2.1.5.5. T Cell Receptors (TCRs)

In certain embodiments, a presently disclosed cell comprising a first antigen-recognizing receptor further comprises a TCR. A TCR is a disulfide-linked heterodimeric protein consisting of two variable chains expressed as part of a complex with the invariant CD3 chain molecules. A TCR is found on the surface of T cells, and is responsible for recognizing antigens as peptides bound to major histocompatibility complex (MHC) molecules. In certain embodiments, a TCR comprises an alpha chain and a beta chain (encoded by TRA and TRB, respectively). In certain embodiments, a TCR comprises a gamma chain and a delta chain (encoded by TRG and TRD, respectively).

Each chain of a TCR is composed of two extracellular domains: Variable (V) region and a Constant (C) region. The Constant region is proximal to the cell membrane, followed by a transmembrane region and a short cytoplasmic tail. The variable region binds to the peptide/MHC complex. The variable domain of both chains each has three complementarity determining regions (CDRs).

In certain embodiments, a TCR can form a receptor complex with three dimeric signaling modules CD3δ/ε, CD3γ/ε and CD247 ζ/ζ or ζ/n. When a TCR complex engages with its antigen and MHC (peptide/MHC), the T cell expressing the TCR complex is activated.

In certain embodiments, the TCR is an endogenous TCR. In certain embodiments, the TCR is naturally occurring TCR.

In certain embodiments, the TCR is an exogenous TCR. In certain embodiments, the TCR is a recombinant TCR. In certain embodiments, the TCR is a non-naturally occurring TCR. In certain embodiments, the non-naturally occurring TCR differs from any naturally occurring TCR by at least one amino acid residue. In certain embodiments, the non-naturally occurring TCR differs from any naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues. In certain embodiments, the non-naturally occurring TCR is modified from a naturally occurring TCR by at least one amino acid residue. In certain embodiments, the non-naturally occurring TCR is modified from a naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues.

### 2.1.5.6. Delivery of the Second Antigen-Recognizing Receptor

In certain embodiments, the second antigen-recognizing receptor is delivered to the cell by a viral method. In certain embodiments, the viral method comprises a viral vector. In certain embodiments, the viral vector is a retroviral vector (e.g., a gamma-retroviral vector or a lentiviral vector). Other viral vectors include adenoviral vectors, adeno-associated viral vectors, vaccinia viruses, bovine papilloma viruses, and herpes viruses (e.g., such as Epstein-Barr Virus).

In certain embodiments, the second antigen-recognizing receptor is delivered to the cell by a non-viral method. Any targeted genome editing methods can also be used to deliver the second antigen-recognizing receptor to the cell. In certain embodiments, the second antigen-recognizing receptor is delivered to the cell by a method comprising homologous recombination, a Zinc finger nuclease, a meganuclease, a Transcription activator-like effector nuclease (TALEN), a Clustered regularly-interspaced short palindromic repeats (CRISPR) system, or a combination thereof. In certain embodiments, a CRISPR system is used to deliver the second antigen-recognizing receptor to the cell.

In certain embodiments, the cell is a T cell, and the second antigen-recognizing receptor is integrated at a locus within the genome of the T cell. Non-limiting examples of loci include a TRAC locus, a TRBC locus, a TRDC locus, and a TRGC locus. In certain embodiments, the locus is a TRAC locus or a TRBC locus. In certain embodiments, the cell is a T cell, and the second antigen-recognizing receptor is integrated at a TRAC locus.

### 2.2. Co-Stimulatory Ligands

In certain embodiments, a presently disclosed cell comprising an antigen-recognizing receptor (e.g., a first antigen-recognizing receptor, e.g., one disclosed in section 2.1.2) further comprises at least one recombinant or exogenous co-stimulatory ligand. For example, a presently disclosed cell can be further transduced with at least one co-stimulatory ligand, such that the cell expresses or is induced to express the first antigen-recognizing receptor, the second antigen-recognizing receptor, and the at least one co-stimulatory ligand. The at least one co-stimulatory ligand provides a co-stimulation signal to the cell.

Non-limiting examples of co-stimulatory ligands include, but are not limited to, members of the tumor necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share a number of common features. The majority of TNF superfamily members are synthesized as type II transmembrane proteins (extracellular C-terminus) containing a short cytoplasmic segment and a relatively long extracellular region. Non-limiting examples of TNF superfamily members include nerve growth factor (NGF), CD40L (also known as “CD154”), 4-1BBL, TNF-α, OX40L, CD70, Fas ligand (FasL), CD30L, tumor necrosis factor beta (TNFβ)/lymphotoxin-alpha (LTα), lymphotoxin-beta (LTβ), CD257/B cell-activating factor (BAFF)/Blys/THANK/Tall-1, glucocorticoid-induced TNF Receptor ligand (GITRL), TNF-related apoptosis-inducing ligand (TRAIL), and LIGHT (TNFSF14). The immunoglobulin (Ig) superfamily is a large group of cell surface and soluble proteins that are involved in the recognition, binding, or adhesion processes of cells. These proteins share structural features with immunoglobulins—they possess an immunoglobulin domain (fold). Non-limiting examples of immunoglobulin superfamily ligands include CD80, CD86, and ICOSLG. In certain embodiments, the at least one co-stimulatory ligand is selected from the group consisting of 4-1BBL, CD80, CD86, CD70, GITRL, CD40L, OX40L, CD30L, TNFRSF14, ICOSLG, TRAIL, and combinations thereof.

In certain embodiments, the cell further comprises one exogenous co-stimulatory ligand that is 4-1BBL. In certain embodiments, the co-stimulatory ligand is human 4-1BBL. In certain embodiments, the 4-1BBL comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence having a Uniprot Reference No: P41273-1 (SEQ ID NO: 72) or a fragment thereof, and/or may optionally comprise up to one or up to two or up to three conservative amino acid substitutions. In certain embodiments, the 4-1BBL comprises or consists of an amino acid sequence that is a consecutive portion of the amino acid sequence of SEQ ID NO: 72. SEQ ID NO: 72 is provided below.

[SEQ ID NO: 72]
MEYASDASLDPEAPWPPAPRARACRVLPWALVAGLLLLLLLAAACAVFLA
CPWAVSGARASPGSAASPRLREGPELSPDDPAGLLDLRQGMFAQLVAQNV
LLIDGPLSWYSDPGLAGVSLTGGLSYKEDTKELVVAKAGVYYVFFQLELR
RVVAGEGSGSVSLALHLQPLRSAAGAAALALTVDLPPASSEARNSAFGFQ
GRLLHLSAGQRLGVHLHTEARARHAWQLTQGATVLGLFRVTPEIPAGLPS
PRSE

An exemplary nucleotide sequence encoding the amino acid sequence of SEQ ID NO: 72 is set forth in SEQ ID NO: 73.

[SEQ ID NO: 73]
ATGGAATACGCCTCTGACGCTTCACTGGACCCCGAAGCCCCGTGGCCTCC
CGCGCCCCGCGCTCGCGCCTGCCGCGTACTGCCTTGGGCCCTGGTCGCGG
GGCTGCTGCTGCTGCTGCTGCTCGCTGCCGCCTGCGCCGTCTTCCTCGCC
TGCCCCTGGGCCGTGTCCGGGGCTCGCGCCTCGCCCGGCTCCGCGGCCAG
CCCGAGACTCCGCGAGGGTCCCGAGCTTTCGCCCGACGATCCCGCCGGCC
TCTTGGACCTGCGGCAGGGCATGTTTGCGCAGCTGGTGGCCCAAAATGTT
CTGCTGATCGATGGGCCCCTGAGCTGGTACAGTGACCCAGGCCTGGCAGG
CGTGTCCCTGACGGGGGGCCTGAGCTACAAAGAGGACACGAAGGAGCTGG
TGGTGGCCAAGGCTGGAGTCTACTATGTCTTCTTTCAACTAGAGCTGCGG
CGCGTGGTGGCCGGCGAGGGCTCAGGCTCCGTTTCACTTGCGCTGCACCT
GCAGCCACTGCGCTCTGCTGCTGGGGCCGCCGCCCTGGCTTTGACCGTGG
ACCTGCCACCCGCCTCCTCCGAGGCTCGGAACTCGGCCTTCGGTTTCCAG
GGCCGCTTGCTGCACCTGAGTGCCGGCCAGCGCCTGGGCGTCCATCTTCA
CACTGAGGCCAGGGCACGCCATGCCTGGCAGCTTACCCAGGGCGCCACAG
TCTTGGGACTCTTCCGGGTGACCCCCGAAATCCCAGCCGGACTCCCTTCA
CCGAGGTCGGAA

In certain embodiments, the cell further comprises one exogenous co-stimulatory ligand that is CD80. In certain embodiments, the co-stimulatory ligand is human CD80. In certain embodiments, the CD80 comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence having a NCBI Reference No: NP_005182 (SEQ ID NO: 74) or a fragment thereof, and/or may optionally comprise up to one or up to two or up to three conservative amino acid substitutions. In certain embodiments, the CD80 comprises or consists of an amino acid sequence that is a consecutive portion of the amino acid sequence of SEQ ID NO: 74. SEQ ID NO: 74 is provided below.

[SEQ ID NO: 74]
MGHTRRQGTSPSKCPYLNFFQLLVLAGLSHFCSGVIHVTKEVKEVATLSC
GHNVSVEELAQTRIYWQKEKKMVLTMMSGDMNIWPEYKNRTIFDITNNLS
IVILALRPSDEGTYECVVLKYEKDAFKREHLAEVTLSVKADFPTPSISDF
EIPTSNIRRIICSTSGGFPEPHLSWLENGEELNAINTTVSQDPETELYAV
SSKLDFNMTTNHSFMCLIKYGHLRVNQTFNWNTTKQEHFPDNLLPSWAIT
LISVNGIFVICCLTYCFAPRCRERRRNERLRRESVRPV

An exemplary nucleotide sequence encoding the amino acid sequence of SEQ ID NO: 74 is set forth in SEQ ID NO: 75. SEQ ID NO: 75 is provided below.

[SEQ ID NO: 75]
ATGGGCCACACACGGAGGCAGGGAACATCACCATCCAAGTGTCCATACCT
CAATTTCTTTCAGCTCTTGGTGCTGGCTGGTCTTTCTCACTTCTGTTCAG
GTGTTATCCACGTGACCAAGGAAGTGAAAGAAGTGGCAACGCTGTCCTGT
GGTCACAATGTTTCTGTTGAAGAGCTGGCACAAACTCGCATCTACTGGCA
AAAGGAGAAGAAAATGGTGCTGACTATGATGTCTGGGGACATGAATATAT
GGCCCGAGTACAAGAACCGGACCATCTTTGATATCACTAATAACCTCTCC
ATTGTGATCCTGGCTCTGCGCCCATCTGACGAGGGCACATACGAGTGTGT
TGTTCTGAAGTATGAAAAAGACGCTTTCAAGCGGGAACACCTGGCTGAAG
TGACGTTATCAGTCAAAGCTGACTTCCCTACACCTAGTATATCTGACTTT
GAAATTCCAACTTCTAATATTAGAAGGATAATTTGCTCAACCTCTGGAGG
TTTTCCAGAGCCTCACCTCTCCTGGTTGGAAAATGGAGAAGAATTAAATG
CCATCAACACAACAGTTTCCCAAGATCCTGAAACTGAGCTCTATGCTGTT
AGCAGCAAACTGGATTTCAATATGACAACCAACCACAGCTTCATGTGTCT
CATCAAGTATGGACATTTAAGAGTGAATCAGACCTTCAACTGGAATACAA
CCAAGCAAGAGCATTTTCCTGATAACCTGCTCCCATCCTGGGCCATTACC
TTAATCTCAGTAAATGGAATTTTTGTGATATGCTGCCTGACCTACTGCTT
TGCCCCAAGATGCAGAGAGAGAAGGAGGAATGAGAGATTGAGAAGGGAAA
GTGTACGCCCTGTA

In certain embodiments, the cell further comprises two exogenous co-stimulatory ligands that are 4-1BBL and CD80. In certain embodiments, the cell further comprises two exogenous co-stimulatory ligands that are 4-1BBL and CD80, wherein the 4-1BBL comprises or consists of the amino acid sequence set forth in SEQ ID NO: 72, and the CD80 comprises or consists of the amino acid sequence set forth in SEQ ID NO: 74.

Receptor-comprising cells comprising at least one exogenous co-stimulatory ligand are described in U.S. Pat. No. 8,389,282, which is incorporated by reference in its entirety.

### 2.3. Fusion Polypeptides

In certain embodiments, a presently disclosed cell comprising an antigen-recognizing receptor (e.g., a first antigen-recognizing receptor, e.g., one disclosed in section 2.1.2) further comprises a fusion polypeptide. For example, a presently disclosed cell can be further transduced with the fusion polypeptide, such that the cell expresses or is induced to express the first antigen-recognizing receptor, the second antigen-recognizing receptor, and the fusion polypeptide. The fusion polypeptide provides a co-stimulation signal to the cell. The fusion polypeptides are capable of enhancing the activity and/or efficacy of a cell comprising the first antigen-recognizing receptor (e.g., a CAR or a TCR-like fusion molecule). In certain embodiments, the fusion polypeptide comprises a) an extracellular domain and a transmembrane domain of a co-stimulatory ligand, and b) an intracellular domain of a first co-stimulatory molecule.

Non-limiting examples of the co-stimulatory ligand include tumor necrosis factor (TNF) family members, immunoglobulin (Ig) superfamily members, and combinations thereof. The TNF family member can be selected from the group consisting of 4-1BBL, OX40L, CD70, GITRL, CD40L, and combinations thereof. The Ig superfamily member can be selected from the group consisting of CD80, CD86, ICOS ligand (ICOSLG (also known as “CD275”), and combinations thereof. In certain embodiments, the co-stimulatory ligand is selected from the group consisting of 4-1BBL, OX40L, CD70, GITRL, CD40L, CD80, CD86, ICOSLG, and combinations thereof.

In certain embodiments, the fusion polypeptide comprises an extracellular domain and a transmembrane domain of a co-stimulatory ligand that is CD80. In certain embodiments, the co-stimulatory ligand is human CD80. In certain embodiments, the CD80 comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 69 or a fragment thereof, and/or may optionally comprise up to one or up to two or up to three conservative amino acid substitutions. In certain embodiments, the CD80 comprises or consists of an amino acid sequence that is a consecutive portion of the amino acid sequence of SEQ ID NO: 74.

In certain embodiments, the extracellular domain of CD80 comprises or consists of an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to amino acids 1-242 of SEQ ID NO: 74. In certain embodiments, the extracellular domain of CD80 comprises or consists of amino acids 1-242 of SEQ ID NO: 74 or a functional fragment thereof. A functional fragment can be a consecutive portion of amino acids 1-242 of SEQ ID NO: 74, which is at least about 50, at least about 75, at least about 100, at least about 125, at least about 150, at least about 175, or at least about 200, or at least about 220 amino acids in length. In certain embodiments, the functional fragment retains at least about 80%, at least about 85%, at least about 90%, at least about 95%, or at least about 100% of the primary function of the extracellular domain of CD80. Non-limiting examples of the primary functions of the extracellular domain of CD80 include binding to/interacting with CD28, binding to/interacting with CTLA-4, binding to/interacting with PD-L1, and contributing to CD80 homodimerization. In certain embodiments, an extracellular domain of CD80 comprises or consists of amino acids 1-242 of SEQ ID NO: 74.

In certain embodiments, the transmembrane domain of CD80 comprises or consists of an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to amino acids 243-263 of SEQ ID NO: 74. In certain embodiments, the transmembrane domain of CD80 comprises or consists of amino acids 243-263 of SEQ ID NO: 74 or a fragment thereof. Such fragment can be at least about 5, at least about 10, at least about 15, or at least about 20 amino acids in length. In certain embodiments, the transmembrane domain of CD80 comprises or consists of amino acids 243-263 of SEQ ID NO: 74.

Non-limiting examples of co-stimulatory molecules include CD28, 4-1BB, OX40, ICOS, DAP-10, CD27, CD40, NKG2D, CD2, and combinations thereof.

In certain embodiments, the fusion polypeptide comprises an extracellular domain and a transmembrane domain of a co-stimulatory molecule that is 4-1BB. In certain embodiments, the co-stimulatory molecule is human 4-1BB. In certain embodiments, the 4-1BB comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 71 or a fragment thereof, and/or may optionally comprise up to one or up to two or up to three conservative amino acid substitutions. In certain embodiments, the 4-1BB comprises or consists of an amino acid sequence that is a consecutive portion of the amino acid sequence of SEQ ID NO: 71. In certain embodiments, the intracellular domain of 4-1BB comprises or consists of an amino acid sequence that is at least about 80%, at least about 85%, at least about 90%, at least about 95%, at least about 96%, at least about 97%, at least about 98%, or at least about 99%, at least about 100% homologous or identical to amino acids 214-255 of SEQ ID NO: 71 or a fragment thereof, and/or may optionally comprise up to one or up to two or up to three conservative amino acid substitutions. In certain embodiments, the intracellular domain of 4-1BB comprises or consists of amino acids 214-255 of SEQ ID NO: 71 or a functional fragment thereof. Such functional fragment can be a consecutive portion of amino acids 214-255 of SEQ ID NO: 71, which is at least about 20, at least about 25, at least about 30, at least about 35, or at least about 40 amino acids in length. In certain embodiments, the functional fragment of amino acids 214-255 of SEQ ID NO: 71 retains at least about 80%, at least about 85%, at least about 90%, at least about 95%, or at least about 100% of the primary functions of the intracellular domain of 4-1BB. Non-limiting examples of the primary functions of the intracellular domain of 4-1BB include providing co-stimulatory signaling for the activation and proliferation of an immunoresponsive cell (e.g., a T cell), and interacting and activating downstream adaptors (e.g., TRAFs). In certain embodiments, the intracellular domain of 4-1BB comprises or consists of amino acids 214-255 of SEQ ID NO: 71.

In certain embodiments, the co-stimulatory molecule is CD28. In certain embodiments, the co-stimulatory molecule is human CD28. In certain embodiments, the CD28 comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 52 or a fragment thereof, and/or may optionally comprise up to one or up to two or up to three conservative amino acid substitutions. In certain embodiments, the CD28 comprises or consists of an amino acid sequence that is a consecutive portion of the amino acid sequence of SEQ ID NO: 52. In certain embodiments, the intracellular domain of CD28 comprises or consists of an amino acid sequence that is at least about 80%, at least about 85%, at least about 90%, at least about 95%, at least about 96%, at least about 97%, at least about 98%, or at least about 99%, at least about 100% homologous or identical to amino acids 180 to 219 of SEQ ID NO: 52 or a fragment thereof, and/or may optionally comprise up to one or up to two or up to three conservative amino acid substitutions. In certain embodiments, the intracellular domain of CD28 comprises or consists of amino acids 180 to 219 of SEQ ID NO: 52 or a functional fragment thereof. A functional fragment of amino acids 180 to 219 of SEQ ID NO: 52 can be a consecutive portion of amino acids 180 to 219 of SEQ ID NO: 52, which is at least about 20, at least about 25, at least about 30, or at least about 35 amino acids in length. In certain embodiments, such functional fragment retains at least about 80%, at least about 85%, at least about 90%, at least about 95%, or at least about 100% of the primary function of the intracellular domain of CD28. Non-limiting examples of the primary functions of the intracellular domain of CD28 include providing co-stimulatory signaling for the activation and proliferation of an immunoresponsive cell (e.g., a T cell), and interacting with protein adaptors (e.g., PI3K, GRB2, and LCK). In certain embodiments, the intracellular domain of CD28 comprises or consists of amino acids 180 to 219 of SEQ ID NO: 52.

In certain embodiments, the fusion polypeptide comprises an intracellular domain of a second co-stimulatory molecule. In certain embodiments, the fusion polypeptide comprises an intracellular domain of a third co-stimulatory molecule. In certain embodiments, the fusion polypeptide comprises an intracellular domain of a fourth co-stimulatory molecule. In certain embodiments, the fusion polypeptide comprises an intracellular domain of a fifth co-stimulatory molecule. In certain embodiments, the first, second, third, fourth, and fifth co-stimulatory molecule can be the same or different among each other.

In certain embodiments, the fusion polypeptide comprises an extracellular domain and a transmembrane domain of a co-stimulatory ligand that is CD80, and an intracellular domain of a co-stimulatory molecule that is 4-1BB. In certain embodiments, the fusion polypeptide comprises or consists of an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 76. In certain embodiments, the fusion polypeptide comprises or consists of the amino acid sequence set forth in SEQ ID NO: 76. SEQ ID NO: 76 is provided below.

[SEQ ID NO: 76]
MGHTRRQGTSPSKCPYLNFFQLLVLAGLSHFCSGVIHVTKEVKEVATLSC
GHNVSVEELAQTRIYWQKEKKMVLTMMSGDMNIWPEYKNRTIFDITNNLS
IVILALRPSDEGTYECVVLKYEKDAFKREHLAEVTLSVKADFPTPSISDF
EIPTSNIRRIICSTSGGFPEPHLSWLENGEELNAINTTVSQDPETELYAV
SSKLDFNMTTNHSFMCLIKYGHLRVNQTFNWNTTKQEHFPDNLLPSWAIT
LISVNGIFVICCLTYCFKRGRKKLLYIFKQPFMRPVQTTQEEDGCSCRFP
EEEEGGCEL

In certain embodiments, the fusion polypeptide comprises an extracellular domain and a transmembrane domain of a co-stimulatory ligand that is CD80, an intracellular domain of a first co-stimulatory molecule that is 4-1BB, and an intracellular domain of a second co-stimulatory molecule that is CD28.

In certain embodiments, the fusion polypeptide comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 77. In certain embodiments, the fusion polypeptide comprises or consists of the amino acid sequence set forth in SEQ ID NO: 77. SEQ ID NO: 77 is provided below.

[SEQ ID NO: 77]
MGHTRRQGTSPSKCPYLNFFQLLVLAGLSHFCSGVIHVTKEVKEVATLSC
GHNVSVEELAQTRIYWQKEKKMVLTMMSGDMNIWPEYKNRTIFDITNNLS
IVILALRPSDEGTYECVVLKYEKDAFKREHLAEVTLSVKADFPTPSISDF
EIPTSNIRRIICSTSGGFPEPHLSWLENGEELNAINTTVSQDPETELYAV
SSKLDFNMTTNHSFMCLIKYGHLRVNQTFNWNTTKQEHFPDNLLPSWAIT
LISVNGIFVICCLTYCFRSKRSRLLHSDYMNMTPRRPGPTRKHYQPYAPP
RDFAAYRKRGRKKLLYIFKQPFMRPVQTTQEEDGCSCRFPEEEEGGCEL

Various modified fusion polypeptides are disclosed in International Patent Application No. PCT/US20/42753, which is incorporated by reference hereby in its entirety.

### 2.4. Gene Disruptions and Gene Modifications

In certain embodiments, a presently disclosed cell comprising an antigen-recognizing receptor (e.g., a first antigen-recognizing receptor, e.g., one disclosed in section 2.1.2) further comprises a gene disruption of a CD70 locus. The gene disruption of the CD70 locus can result in a non-functional CD70 protein or a knockout of the CD70 gene expression. In certain embodiments, the gene disruption of the CD70 locus results in knockout of the CD70 gene expression.

Non-limiting examples of gene disruptions include substitutions, deletions, insertions, or combinations thereof. In certain embodiments, the mutation comprises a missense mutation, a nonsense mutation, or a combination thereof. In certain embodiments, the deletion comprises a non-frameshift deletion, a frameshift deletion, or a combination thereof. In certain embodiments, the insertion comprises a non-frameshift insertion, a frameshift insertion, or a combination thereof.

In certain embodiments, the CD70 locus is a human CD70 locus. The gene disruption of the CD70 locus can be generated by any suitable gene editing methods. In certain embodiments, the gene disruption of the CD70 locus (e.g., knockout of the CD70 locus) is generated using a viral method. In certain embodiments, the viral method comprises a viral vector. In certain embodiments, the viral vector is a retroviral vector (e.g., a gamma-retroviral vector or a lentiviral vector). Other viral vectors include adenoviral vectors, adena-associated viral vectors, vaccinia viruses, bovine papilloma viruses, and herpes viruses (e.g., such as Epstein-Barr Virus).

In certain embodiments, the gene disruption of the CD70 locus (e.g., knockout of the CD70 locus) is generated using a non-viral method. Non-viral approaches can also be employed for genetic modification of a cell. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g. Zinc finger nucleases, meganucleases, or TALE nucleases, CRISPR). Transient expression may be obtained by RNA electroporation.

Any targeted genome editing methods can also be used to generate the gene disruption of the CD70 locus. In certain embodiments, the gene disruption of the CD70 locus is generated by a method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

In certain embodiments, a CRISPR system is used to generate the gene disruption of the CD70 locus.

Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying CAR expression cassette need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells.

In certain embodiments, the CRISPR system comprises base editors. In certain embodiments, the CRISPR system comprises transposases/recombinases. In certain embodiments, the CRISPR system comprises prime editors. In certain embodiments, the CRISPR system comprises an epigenetic modulator. In certain embodiments, the CRISPR system comprises is a CRISPRoff system. Additional details on the CRISPR systems of the presently disclosed subject matter can be found in Anzalone et al., Nature biotechnology 38.7 (2020): 824-844 and in Nuñez et al., Cell 184.9 (2021): 2503-2519, the contents of each of which are incorporated by reference in their entireties.

In certain embodiments, the CD70 locus is disrupted using a gRNA molecule to knockout expression of CD70. The gRNA molecule can target a coding sequence of a CD70 gene (e.g., a human CD70 gene) or a non-coding sequence of a CD70 gene (e.g., a human CD70 gene). In certain embodiments, the gRNA molecule targets a coding sequence of a CD70 gene (e.g., a human CD70 gene). In certain embodiments, the gRNA molecule targets a target sequence within a human CD70 gene.

In certain embodiments, zinc-finger nucleases are used to generate the gene disruption of the CD70 locus. A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying CAR expression cassette, ZFNs can be used to insert the CAR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome.

In certain embodiments, a TALEN system is used to generate the gene disruption of the CD70 locus. Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome.cDNA expression for use in polynucleotide therapy methods can be directed from any suitable promoter (e.g., the human cytomegalovirus (CMV), simian virus 40 (SV40), or metallothioncin promoters), and regulated by any appropriate mammalian regulatory element or intron (e.g. the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

In certain embodiments, the gene disruption of the CD70 locus can be a disruption of the coding region of the CD70 locus and/or a disruption of the non-coding region of the CD70 locus. In certain embodiments, the gene disruption of the CD70 locus comprises a disruption of the coding region of the CD70 locus. In certain embodiments, the gene disruption of the CD70 locus comprises an insertion at the coding region of the CD70 locus. Human CD70 protein comprises three exons: exon 1, exon 2, and exon 3. In certain embodiments, the gene disruption of the CD70 locus comprises a disruption at one or more of exon 1, exon 2, and exon 3 of the CD70 locus. In certain embodiments, the gene disruption of the CD70 locus comprises a disruption at exon 1 of the CD70 locus. In certain embodiments, the gene disruption of the CD70 locus comprises an insertion at exon 1 of the CD70 locus.

In certain embodiments, a presently disclosed cell comprising an antigen recognizing-receptor (e.g., a first antigen-recognizing receptor, e.g., one disclosed in section 2.1.2) further comprises a gene disruption of a TRAC locus. In certain embodiments, the gene disruption of the TRAC locus results in a non-functional TCR. In certain embodiments, the gene disruption of the TRAC locus results in knockout of the TCR gene expression.

Any methods to generate the gene disruption of the CD70 locus as disclosed above can be used to generate the gene disruption of the TRAC locus. In certain embodiments, the gene disruption of the TRAC locus is generated by a method comprising a gene editing method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

In certain embodiments, a presently disclosed cell further comprises a gene modification of a CD70 gene. The gene modification of the CD70 gene can result in a non-functional CD70 protein or a knockdown of the CD70 gene expression. In certain embodiments, the gene modification of the CD70 gene results in knockout of the CD70 gene expression.

In certain embodiments, the modification of the CD70 gene comprises use of an RNAi agent, including, but not limited to, shRNA, siRNA, LNA, dsRNA, and miRNA. In certain embodiments, the RNAi agent comprises a shRNA. In certain embodiments, the RNAi agent (e.g., shRNA) targets one or more isoforms of the CD70 gene and thereby reduces or eliminates the expression of the CD70 gene or CD70 protein. In certain embodiments, the RNAi agent (e.g., shRNA) is expressed from the same construct that expresses the first antigen-recognizing receptor and/or the second antigen-recognizing receptor disclosed herein. In certain embodiments, the expressions of the RNAi agent (e.g., shRNA), the first antigen-recognizing receptor, and the second antigen-recognizing receptor are driven by identical promoters (e.g., a same promoter). In certain embodiments, the expressions of the shRNA, the first antigen-recognizing receptor, and the second antigen-recognizing receptor disclosed herein are driven by different promoters.

In certain embodiments, the RNAi agent (e.g., shRNA) comprises a nucleotide sequence that is at least about 75%, at least about 80%, at least about 85%, at least about 90%, at least about 95%, at least about 96%, at least about 97%, at least about 98%, at least about 99%, or about 100% homologous or identical to at least a portion of a CD70 nucleic acid sequence. In certain embodiments, the RNAi agent (e.g., shRNA) comprises a nucleotide sequence complementary to the CD70 gene that is at least about 10 nucleotides, at least about 15 nucleotides, at least about 20 nucleotides, at least about 25 nucleotides, at least about 30 nucleotides. In certain embodiments, the RNAi agent (e.g., shRNA) comprises a nucleotide sequence that is up to 15 nucleotides, up to 20 nucleotides, up to 25 nucleotides, up to 30 nucleotides, up to 35 nucleotides, up to 40 nucleotides, up to 55 nucleotides, up to 60 nucleotides, up to 65 nucleotides, up to 70 nucleotides, up to 75 nucleotides, up to 80 nucleotides, up to 85 nucleotides, up to 90 nucleotides, up to 95 nucleotides, or up to 100 nucleotides in length. In certain embodiments, the RNAi agent comprises DNA or atypical or non-naturally occurring residues, for example, but not limited to, phosphorothioate residues.

In certain embodiments, the RNAi agent reduces the expression (e.g., endogenous expression) of CD70 by about 5%, about 10%, about 20%, about 30%, about 40%, about 50%, about 60%, about 70%, about 80%, about 90%, about 95%, about 100% or any intermediate value or range thereof.

In certain embodiments, the gene disruption of the TRAC locus can be a disruption of the coding region of the TRAC locus and/or a disruption of the non-coding region of the TRAC locus. In certain embodiments, the gene disruption of the TRAC locus comprises a disruption of the coding region of the TRAC locus. In certain embodiments, the gene disruption of the TRAC locus comprises an insertion at the coding region of the TRAC locus. Human TRAC protein comprises 4 exons: exon 1, exon 2, exon 3, and exon 4. In certain embodiments, the coding region of the TRAC locus comprises exon 1, exon 2, exon 3, and exon 4. In certain embodiments, the gene disruption of the TRAC locus comprises a disruption at one or more of exon 1 through exon 4 of the TRAC locus. In certain embodiments, the gene disruption of the TRAC locus comprises a disruption at exon 1 of the TRAC locus. In certain embodiments, the gene disruption of the TRAC locus comprises an insertion at exon 1 of the TRAC locus.

The presently disclosed cells can be isolated and activated by using CD3/CD28 antibodies before generation of a gene disruption. In certain embodiments, the presently disclosed cell comprising an antigen-recognizing receptor (e.g., a first antigen-recognizing receptor, e.g., one disclosed in section 2.1.2) further comprises a gene disruption of a TRAC locus and of a CD70 locus.

In certain embodiments, the gene disruption of the TRAC locus and the gene disruption of the CD70 locus are generated after isolation and activation of the cells (e.g., T cells). In certain embodiments, the gene disruption of the TRAC locus and the gene disruption of the CD70 locus are generated before isolation and activation of the cells (e.g., T cells).

In certain embodiments, the gene disruption of the TRAC locus is generated before isolation and activation of the cells (e.g., T cells) and the gene disruption of the CD70 locus is generated after isolation and activation of the cells (e.g., T cells). In certain embodiments, the gene disruption of the CD70 locus is generated before isolation and activation of the cells (e.g., T cells) and the gene disruption of the TRAC locus is generated after isolation and activation of the cells (e.g., T cells).

### 3. NUCLEIC ACID COMPOSITIONS AND VECTORS

The presently disclosed subject matter provides nucleic acid compositions comprising a first polynucleotide encoding an antigen-recognizing receptor disclosed herein (e.g., disclosed in Section 2.1). Also provided are cells comprising such nucleic acid compositions. In certain embodiments, the nucleic acid composition further comprises a promoter that is operably linked to the antigen-recognizing receptor. In certain embodiments, the nucleic acid composition further comprises a second promoter that is operably linked to the second antigen-recognizing receptor (e.g., disclosed in Section 2.1).

In addition, the presently disclosed subject matter provides nucleic acid compositions comprising a polynucleotide encoding an antigen-recognizing receptor disclosed herein (e.g., disclosed in Section 2.1). Also provided are cells comprising such nucleic acid compositions. In certain embodiments, the nucleic acid composition further comprises a second promoter that is operably linked to the second antigen-recognizing receptor.

In certain embodiments, the presently disclosed subject matter provides nucleic acid compositions comprising a polynucleotide encoding an antigen-recognizing receptor disclosed herein (e.g., disclosed in Section 2.1) and co-stimulatory ligands (e.g., disclosed in Section 2.2). In certain embodiments, the nucleic acid composition further comprises a promoter that is operably linked to the antigen-recognizing receptor. In certain embodiments, the nucleic acid composition further comprises a second promoter that is operably linked to the co-stimulatory ligand (e.g., disclosed in Section 2.2). In certain embodiments, the nucleic acid composition further comprises a third promoter that is operably linked to a second co-stimulatory ligand (e.g., disclosed in Section 2.2).

Additionally or alternatively, the presently disclosed subject matter provides nucleic acid compositions comprising a polynucleotide encoding an antigen-recognizing receptor disclosed herein (e.g., disclosed in Section 2.1) and a fusion polypeptide (e.g., disclosed in Section 2.3). In certain embodiments, the nucleic acid composition further comprises a first promoter that is operably linked to the antigen-recognizing receptor. In certain embodiments, the nucleic acid composition further comprises a second promoter that is operably linked to the fusion polypeptide.

In certain embodiments, one or both of the first and second promoters are endogenous or exogenous.

In certain embodiments, the exogenous promoter is selected from an elongation factor (EF)-1 promoter, a CMV promoter, a SV40 promoter, a PGK promoter, and a metallothionein promoter. In certain embodiments, one or both of the first and second promoters are inducible promoters. In certain embodiment, the inducible promoter is selected from a NFAT transcriptional response element (TRE) promoter, a CD69 promoter, a CD25 promoter, and an IL-2 promoter.

In certain embodiments, the first and/or the second antigen-recognizing receptors are integrated at a locus within the genome of the T cell, e.g., a TRAC locus, a TRBC locus, a TRDC locus, or a TRGC locus. In certain embodiments, the locus is a TRAC locus. In certain embodiments, the expression of the first and/or second antigen-recognizing receptors are under the control of an endogenous promoter. Non-limiting examples of endogenous promoters include an endogenous TRAC promoter, an endogenous TRBC promoter, an endogenous TRDC promoter, and an endogenous TRGC promoter. In certain embodiments, the endogenous promoter is an endogenous TRAC promoter.

In certain embodiments, the nucleic acid composition is a vector. In certain embodiments, the vector is a retroviral vector (e.g., a gamma-retroviral vector or a lentiviral vector). In certain embodiments, the vector is viral vectors selected from the group consisting of adenoviral vectors, adena-associated viral vectors, vaccinia viruses, bovine papilloma viruses, and herpes viruses (e.g., such as Epstein-Barr Virus).

Additionally, the nucleic acid compositions can be administered to subjects or and/delivered into cells by art-known methods or as described herein. Genetic modification of a cell (e.g., a T cell or a NK cell) can be accomplished by transducing a substantially homogeneous cell composition with a recombinant DNA construct. In certain embodiments, a retroviral vector (either gamma-retroviral or lentiviral) is employed for the introduction of the nucleic acid compositions into the cell. For example, the first polynucleotide and the second polynucleotide can be cloned into a retroviral vector and expression can be driven from its endogenous promoter, from the retroviral long terminal repeat, or from a promoter specific for a target cell type of interest. Non-viral vectors may be used as well.

The first polynucleotide and the second polynucleotide can be constructed in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-κB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides). Combinations of retroviral vector and an appropriate packaging line are also suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114 or GALV envelope and any other known in the art.

Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Other transducing viral vectors can be used to modify a cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adena-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for genetic modification of a cell. For example, a nucleic acid molecule can be delivered into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Transient expression may be obtained by RNA electroporation.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

### 3.1. Methods of Delivering

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

In certain embodiments, the delivery methods include use of colloids. As used herein, the term “colloid” refers to systems in which there are two or more phases, with one phase (e.g., the dispersed phase) distributed in the other phase (e.g., the continuous phase). Moreover, at least one of the phases has small dimensions (in the range of about 10 9 to about 10 6 m). Non-limiting examples of colloids encompassed by the presently disclosed subject matter include macromolecule complexes, nanocapsules, microspheres, beads, and lipid-based systems (e.g., micelles, liposomes, and lipid nanoparticles).

In certain embodiments, the delivery methods include use of liposomes. The term “liposome,” as used herein, refers to single- or multi-layered spherical lipid bilayer structures produced from lipids dissolved in organic solvents and then dispersed in aqueous media. Experimentally and therapeutically used for delivering an active pharmaceutical ingredient (e.g., nucleic acid compositions disclosed herein) to cells, liposomes fuse with cell membranes so the contents are transferred into the cytoplasm.

In certain embodiments, the delivery methods include use of lipid nanoparticles. As used herein, the term “lipid nanoparticle” refers to a particle having at least one dimension in the order of nanometers (e.g., from about 1 nm to about 1,000 nm) and including at least one lipid. In certain embodiments, the lipid nanoparticles can include an active pharmaceutical ingredient (e.g., nucleic acid compositions disclosed herein) for delivering to cells. The morphology of the lipid nanoparticles can be different from liposomes. While liposomes are characterized by a lipid bilayer surrounding a hydrophilic core, lipid nanoparticles have an electron-dense core where cationic lipids and/or ionizable lipids are organized into inverted micelles around an active pharmaceutical ingredient (e.g., nucleic acid compositions disclosed herein). Additional information on the morphology and properties of lipid nanoparticles and liposomes can be found in Wilczewska, et al., Pharmacological reports 64, no. 5 (2012): 1020-1037; Eygeris et al., Accounts of Chemical Research 55, no. 1 (2021): 2-12; Zhang et al., Chemical Reviews 121, no. 20 (2021): 12181-12277; and Fan et al., Journal of pharmaceutical and biomedical analysis 192 (2021): 113642.

In certain embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm.

In certain embodiments, the lipid nanoparticles can include a cationic lipid or an ionizable lipid. The term “cationic lipid” refers to lipids including a head group with permanent positive charges. Non-limiting examples of cationic lipids encompassed by the presently disclosed subject matter include 1,2-di-O-octadecenyl-3-trimethylammonium-propane (DOTMA), 1,2-diolcoyl-3-trimethylammonium-propane (DOTAP), 2,3-diolcyloxy-N-[2-(sperminccarboxamido)ethyl]-N,N-dimethyl-1-propanaminium trifluoroacetate (DOSPA), and ethylphosphatidylcholine (cPC).

As used herein, the term “ionizable lipid” refers to lipids that are protonated at low pH and are neutral at physiological pH. The pH-sensitivity of ionizable lipids is particularly beneficial for delivery in vivo (e.g., delivery of nucleic acid compositions disclosed herein), because neutral lipids have less interactions with the anionic membranes of blood cells and, thus, improve the biocompatibility of the lipid nanoparticles. Once trapped in endosomes, ionizable lipids are protonated and promote membrane destabilization to allow the endosomal escape of the nanoparticles. Non-limiting example of ionizable lipids encompassed by the presently disclosed subject matter include tetrakis (8-methylnonyl) 3,3′,3″,3″-(((methylazancdiyl) bis(propane-3,1 diyl))bis (azanetriyl))tetrapropionate; decyl (2-(dioctylammonio)ethyl) phosphate; ((4-hydroxybutyl)azanediyl)bis(hexane-6,1-diyl)bis(2-hexyldecanoate); bis(2-(dodecyldisulfanyl)ethyl) 3,3′-((3-methyl-9-oxo-10-oxa-13,14-dithia-3,6-diazahexacosyl)azanediyl)dipropionate; 1,1′-((2-(4-(2-((2-(bis(2-hydroxydodecyl)amino)ethyl) (2-hydroxydodecyl)amino)ethyl) piperazin-1-yl)ethyl)azanediyl) bis(dodecan-2-ol); cKK-E12, 3,6-bis(4-(bis(2-hydroxydodecyl)amino)butyl)piperazine-2,5-dione; (6Z,9Z,28Z,31Z)-heptatriaconta-6,9,28,31-tetracn-19-yl 4-(dimethylamino) butanoate; hexa(octan-3-yl) 9,9′,9″,9′″,9″″,9′″″-((((benzene-1,3,5-tricarbonyl)yris(azanediyl)) tris (propane-3,1-diyl)) tris(azanctriyl))hexanonanoatc; heptadecan-9-yl 8-((2-hydroxycthyl) (6-oxo-6-(undecyloxy)hexyl)amino) octanoate; and (((3,6-dioxopiperazine-2,5-diyl) bis(butane-4, 1-diyl))bis(azanetriyl))tetrakis(ethane-2,1-diyl) (9Z,9′Z,9″Z,9′″Z,12Z,12′Z, 12″Z, 12′″Z)-tetrakis (octadeca-9,12-dienoate).

Additionally, in certain embodiments, the lipid nanoparticles can include other lipids. For example, but without any limitation, the lipid nanoparticles of the presently disclosed subject matter can include phospholipids, cholesterol, polyethylene glycol (PEG)-functionalized lipids (PEG-lipids). These lipids can improve certain properties of the lipid nanoparticles (e.g., stability, biodistribution, etc.). For example, cholesterol enhances the stability of the lipid nanoparticles by modulating the integrity and rigidity. Non-limiting examples of other lipids present in lipid nanoparticles include cholesterol, DC-cholesterol, β-sitosterol, BHEM-cholesterol, ALC-0159, distearoylphosphatidylcholine (DSPC), diolcoylphosphatidylcholine (DOPC), (DPPC), diolcoylphosphatidylglycerol (DOPG), dipalmitoylphosphatidylcholine dipalmitoylphosphatidylglycerol (DPPG), diolcoylphosphatidylethanolamine (DOPE), palmitoylolcoylphosphatidylcholine (POPC), palmitoyloleoyl-phosphatidylethanolamine (POPE) and diolcoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoylphosphatidylethanolamine (DSPE), 16-O-monomethyl PE, 16-O-dimethyl PE, 18-1-trans PE, 1-stearoyl-2-oleoyl-phosphatidyethanol amine (SOPE), and 1,2-dielaidoyl-sn-glycero-3-phophoethanolamine (transDOPE).

In certain embodiments, the lipid nanoparticles can include a targeting moiety that binds to a ligand. The use of the targeting moieties allows selective delivery of an active pharmaceutical ingredient (e.g., nucleic acid compositions disclosed herein) to target cells expressing the ligand (e.g., T cells). In certain embodiments, the targeting moiety can be an antibody or antigen-binding fragment thereof that binds to a cell surface receptor. For example, but without any limitation, the targeting domain is an antibody or antigen-binding fragment thereof that binds to a receptor expressed on the surface of a T cell (e.g., CD3, CD4, CD8, CD16, CD40L, CD95, FasL, CTLA-4, OX40, GITR, LAG3, ICOS, and PD-1).

In certain embodiments, the delivery methods are in vivo delivery methods. In certain embodiments, the delivery methods are ex vivo delivery methods.

### 4. FORMULATIONS AND ADMINISTRATION

The presently disclosed subject matter provides compositions comprising presently disclosed cells (e.g., disclosed in Section 2). In certain embodiments, the compositions are pharmaceutical compositions that further comprise a pharmaceutically acceptable excipient.

Compositions comprising the presently disclosed cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Compositions comprising the presently disclosed cells can be provided systemically or directly to a subject for inducing and/or enhancing an immune response to an antigen and/or treating and/or preventing a neoplasm. In certain embodiments, the presently disclosed cells or compositions comprising thereof are directly injected into an organ of interest (e.g., an organ affected by a neoplasm). Alternatively, the presently disclosed cells or compositions comprising thereof are provided indirectly to the organ of interest, for example, by administration into the circulatory system (e.g., the tumor vasculature). Expansion and differentiation agents can be provided prior to, during or after administration of the cells or compositions to increase production of cells in vitro or in vivo.

The quantity of cells to be administered can vary for the subject being treated. In certain embodiments, between about 10⁴ and about 10¹⁰, between about 10⁴ and about 10⁷, between about 10⁵ and about 10⁷, between about 10⁵ and about 10⁹, or between about 10⁶ and about 10⁸ of the presently disclosed cells are administered to a subject. In certain embodiments, between about 10⁵ and about 10⁷ of the presently disclosed cells are administered to a subject. More effective cells may be administered in even smaller numbers. Usually, at least about 1×10⁵ cells will be administered, eventually reaching about 1×10¹⁰ or more. In certain embodiments, at least about 1×10⁵, about 5×10⁵, about 1×10⁶, about 5×10⁶, about 1×10⁷, about 5×10⁷, about 1×10⁸, or about 5×10⁸ of the presently disclosed cells are administered to a subject. In certain embodiments, about 1×10⁵ of the presently disclosed cells are administered to a subject. In certain embodiments, about 5×10⁵ of the presently disclosed cells are administered to a subject. In certain embodiments, about 1×10⁶ of the presently disclosed cells are administered to a subject. The precise determination of what would be considered an effective dose can be based on factors individual to each subject, including their size, age, sex, weight, and condition of the particular subject. Dosages can be readily ascertained by those skilled in the art from this disclosure and the knowledge in the art.

The presently disclosed cells and compositions can be administered by any method known in the art including, but not limited to, intravenous administration, subcutaneous administration, intranodal administration, intratumoral administration, intrathecal administration, intrapleural administration, intraosseous administration, intraperitoneal administration, pleural administration, and direct administration to the subject. The presently disclosed cells can be administered in any physiologically acceptable vehicle, normally intravascularly, although they may also be introduced into bone or other convenient site where the cells may find an appropriate site for regeneration and differentiation (e.g., thymus). The cells can be introduced by injection, catheter, or the like.

Compositions comprising the presently disclosed cells can be provided systemically or directly to a subject for inducing and/or enhancing an immune response to an antigen and/or treating and/or preventing a neoplasm (e.g., cancer), pathogen infection, or infectious disease. In certain embodiments, the presently disclosed cells, compositions, or nucleic acid compositions are directly injected into an organ of interest (e.g., an organ affected by a neoplasm). Alternatively, the presently disclosed cells, compositions, or nucleic acid compositions are provided indirectly to the organ of interest, for example, by administration into the circulatory system (e.g., the tumor vasculature). Expansion and differentiation agents can be provided prior to, during or after administration of the cells, compositions, or nucleic acid compositions to increase production of the cells (e.g., T cells (e.g., CTL cells) or NK cells) in vitro or in vivo.

The presently disclosed compositions can be pharmaceutical compositions comprising the presently disclosed cells or their progenitors and a pharmaceutically acceptable carrier. Administration can be autologous or heterologous. For example, cells, or progenitors can be obtained from one subject, and administered to the same subject or a different, compatible subject. Peripheral blood derived cells or their progeny (e.g., in vivo, ex vivo or in vitro derived) can be administered via localized injection, including catheter administration, systemic injection, localized injection, intravenous injection, or parenteral administration. When administering a therapeutic composition of the presently disclosed subject matter (e.g., a pharmaceutical composition comprising a presently disclosed cell), it can be formulated in a unit dosage injectable form (solution, suspension, emulsion).

### 5. METHODS OF TREATMENT

The presently disclosed subject matter provides various methods of using the presently disclosed cells or compositions comprising thereof. The presently disclosed cells and compositions comprising thereof can be used in a therapy or medicament. For example, the presently disclosed subject matter provides methods for inducing and/or increasing an immune response in a subject in need thereof. The presently disclosed cells and compositions comprising thereof can be used for reducing tumor burden in a subject. The presently disclosed cells and compositions comprising thereof can reduce the number of tumor cells, reduce tumor size, and/or eradicate the tumor in the subject. The presently disclosed cells and compositions comprising thereof can be used for treating and/or preventing a tumor (or neoplasm) in a subject. The presently disclosed cells and compositions comprising thereof can be used for prolonging the survival of a subject suffering from a tumor. In certain embodiments, the tumor is cancer. The presently disclosed cells, compositions, and nucleic acid compositions can also be used for treating and/or preventing a pathogen infection or other infectious disease in a subject, such as an immunocompromised human subject. The presently disclosed cells, compositions, and nucleic acid compositions can also be used for treating and/or preventing an autoimmune disease in a subject. In certain embodiments, each of the above-noted method comprises administering the presently disclosed cells or a composition (e.g., a pharmaceutical composition) comprising thereof to achieve the desired effect, e.g., palliation of an existing condition or prevention of recurrence. For treatment, the amount administered is an amount effective in producing the desired effect. An effective amount can be provided in one or a series of administrations. An effective amount can be provided in a bolus or by continuous perfusion.

Non-limiting examples of tumors (or neoplasms) include blood cancers (e.g. leukemias, lymphomas, and myclomas), ovarian cancer, breast cancer, bladder cancer, brain cancer, colon cancer, intestinal cancer, liver cancer, lung cancer, pancreatic cancer, prostate cancer, skin cancer, stomach cancer, glioblastoma, throat cancer, melanoma, neuroblastoma, adenocarcinoma, glioma, soft tissue sarcoma, and various carcinomas (including prostate and small cell lung cancer). Suitable carcinomas further include any known in the field of oncology, including, but not limited to, astrocytoma, fibrosarcoma, myxosarcoma, liposarcoma, oligodendroglioma, ependymoma, medulloblastoma, primitive neural ectodermal tumor (PNET), chondrosarcoma, osteogenic sarcoma, pancreatic ductal adenocarcinoma, small and large cell lung adenocarcinomas, chordoma, angiosarcoma, endotheliosarcoma, squamous cell carcinoma, bronchoalveolar carcinoma, epithelial adenocarcinoma, and liver metastases thereof, lymphangiosarcoma, lymphangioendotheliosarcoma, hepatoma, cholangiocarcinoma, synovioma, mesothelioma, Ewing's tumor, rhabdomyosarcoma, colon carcinoma, basal cell carcinoma, sweat gland carcinoma, papillary carcinoma, sebaccous gland carcinoma, papillary adenocarcinoma, cystadenocarcinoma, medullary carcinoma, bronchogenic carcinoma, renal cell carcinoma, bile duct carcinoma, choriocarcinoma, seminoma, embryonal carcinoma, Wilms' tumor, testicular tumor, medulloblastoma, craniopharyngioma, cpendymoma, pincaloma, hemangioblastoma, acoustic neuroma, oligodendroglioma, meningioma, neuroblastoma, retinoblastoma, leukemia, multiple myeloma, Waldenstrom's macroglobulinemia, and heavy chain disease, breast tumors such as ductal and lobular adenocarcinoma, squamous and adenocarcinomas of the uterine cervix, uterine and ovarian epithelial carcinomas, prostatic adenocarcinomas, transitional squamous cell carcinoma of the bladder, B and T cell lymphomas (nodular and diffuse) plasmacytoma, acute and chronic leukemias, malignant melanoma, soft tissue sarcomas and leiomyosarcomas. In certain embodiments, the neoplasm is cancer.

In certain embodiments, the tumor and/or neoplasm is a solid tumor. Non limiting examples of solid tumor include renal cell carcinoma, non-small-cell lung cancer, lung adenocarcinoma, lung squamous cell carcinoma, lung neuroendocrine carcinoma, small-cell lung cancer, pancreatic cancer, breast cancer, astrocytoma, glioblastoma, laryngeal/pharyngeal carcinoma, EBV-associated nasopharyngeal carcinoma, and ovarian carcinoma.

In certain embodiments, the tumor and/or neoplasm is renal cell carcinoma. In certain embodiments, the tumor and/or neoplasm is ovarian cancer. In certain embodiments, the tumor and/or neoplasm is pancreatic cancer.

In certain embodiments, the tumor and/or neoplasm comprises tumor cells having a CD70 low antigen density. In certain embodiments, a cell having a CD70 low antigen density comprises a cell surface density of CD70 that is less than about 5,000 molecules per cell, less than about 4,000 molecules per cell, less than about 3,000 molecules per cell, less than about 2,000 molecules per cell, less than about 1,500 molecules per cell, less than about 1,000 molecules per cell, less than about 500 molecules per cell, less than about 200 molecules per cell, or less than about 100 molecules per cell.

In certain embodiments, the tumor and/or neoplasm comprises CD70⁺ tumor cells having low tumor cell frequency. In certain embodiments, CD70⁺ tumor cells have a frequency of less than about 50% per tumor, less than about 40% per tumor, less than about 30% per tumor, less than about 20% per tumor, less than about 15% per tumor, less than about 10% per tumor, less than about 5% per tumor, less than about 2% per tumor, or less than about 1% per tumor.

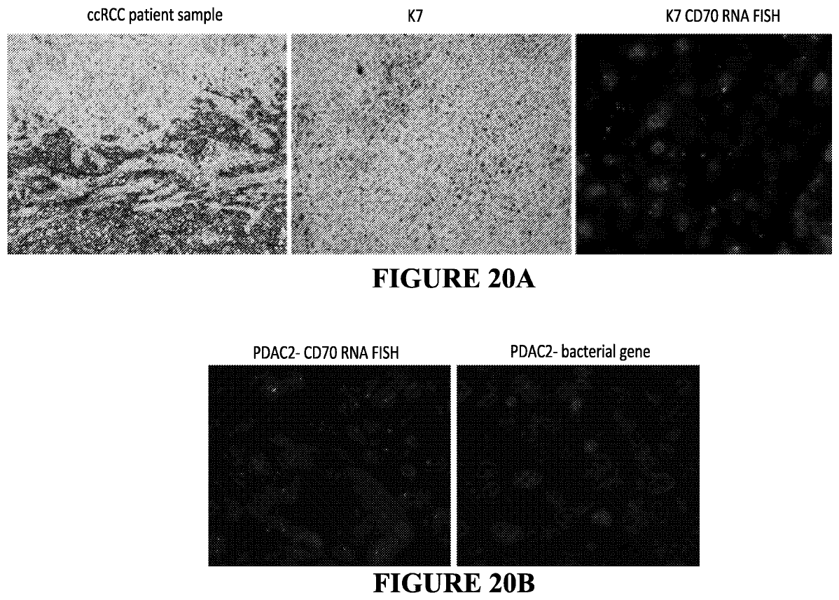

As illustrated in FIG. 20A, the low antigen density and/or low tumor cell frequency can make CD70 polypeptide levels undetectable by using certain methods known in the art including, for example and without any limitation, immunodiffusion, immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, and the like (e.g., Basic and Clinical Immunology, Sites and Terr, eds., Appleton and Lange, Norwalk, Conn, pp 217-262, 1991). The undetectable CD70 polypeptide levels generate false negative results which can lead to the lack of treatment of certain patients.

In certain embodiments, the tumor and/or neoplasm comprises a CD70 polypeptide that is not detectable by immunodiffusion, immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, or a combination thereof. In certain embodiments, the tumor and/or neoplasm comprises a CD70 polypeptide that is not detectable by immunohistochemistry (IHC).

Further, FIG. 20A shows that CD70 polynucleotide levels allow detection of low antigen density and/or low tumor cell frequency. For example, but without any limitation, the CD70 polynucleotide is detectable by RNA-seq, single cell RNA-seq, quantitative RT-PCR, single cell qPCR, Fluorescence In Situ Hybridization (FISH), RNA-FISH, MERFISH (multiplex (in situ) RNA FISH), by in situ hybridization, or a combination thereof. In certain embodiments, the tumor and/or neoplasm comprises a CD70 polynucleotide that is detectable by RNA-seq, single cell RNA-seq, quantitative RT-PCR, single cell qPCR, Fluorescence In Situ Hybridization (FISH), RNA-FISH, MERFISH (multiplex (in situ) RNA FISH), by in situ hybridization, or a combination thereof. In certain embodiments, the tumor and/or neoplasm comprises a CD70 polynucleotide that is detectable by Fluorescence In Situ Hybridization (FISH).

Additionally or alternatively, the presently disclosed subject matter provides methods for inducing and/or increasing an immune response, reducing tumor burden, treating and/or preventing a tumor (or neoplasm), and/or prolonging the survival in a subject having a tumor and/or neoplasm with undetectable CD70 polypeptide levels. In certain embodiments, the methods comprise obtaining a sample of the subject. In certain embodiments, the sample is a tumor sample.

In certain embodiments, the sample comprises a CD70 polynucleotide that is detectable by FISH. In certain embodiments, when the CD70 polynucleotide is detected by FISH in the sample, the methods comprise administering the presently disclosed cells or a composition (e.g., a pharmaceutical composition) comprising thereof to achieve the desired effect, e.g., palliation of an existing condition or prevention of recurrence.

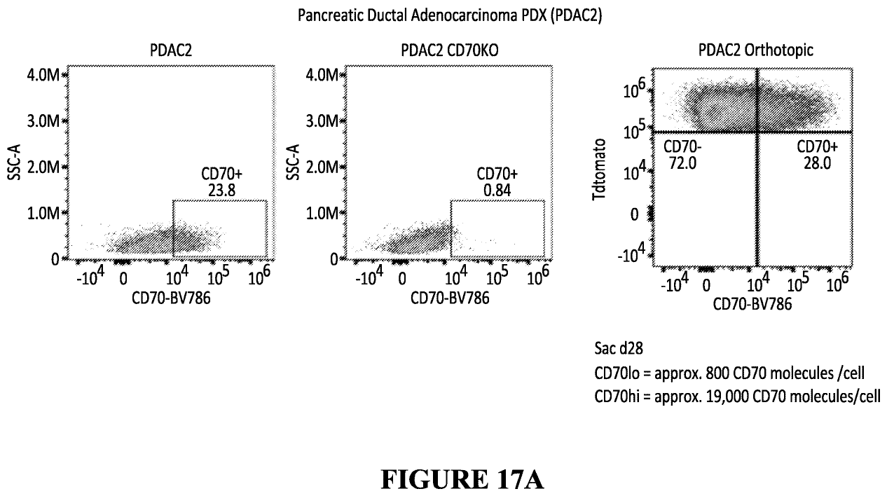

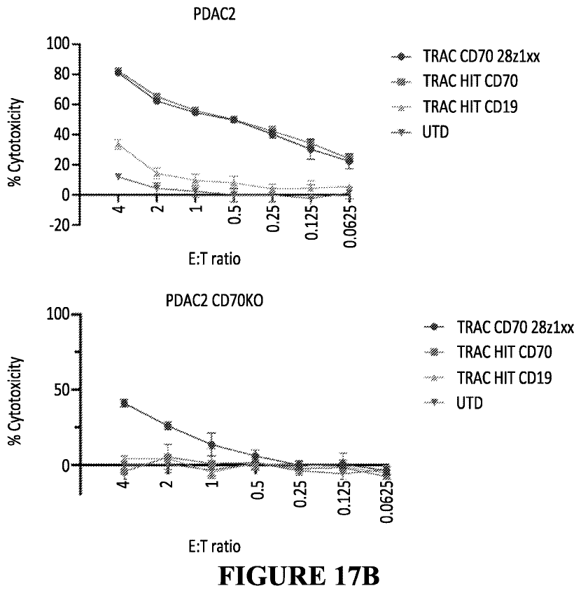

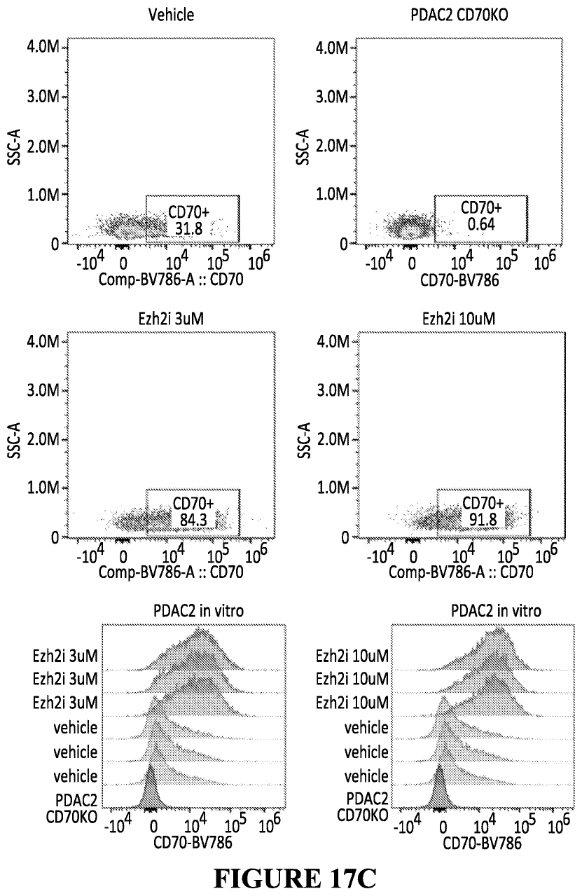

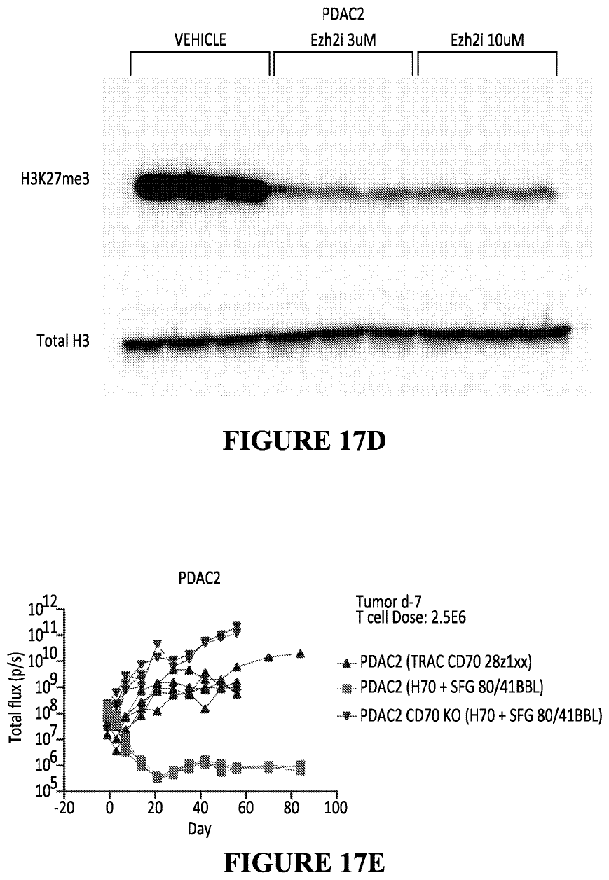

As illustrated in FIGS. 17C and 17D, Ezh2 inhibitors can increase the CD70 polypeptide levels and making these detectable. Thus, the use of Ezh2 inhibitors can identify subjects that have undetectable CD70 polypeptide levels. In certain embodiments, the sample (e.g., a tumor sample) is contacted with an Ezh2 inhibitor. Non-limiting examples of Ezh2 inhibitors include tazemetostat, 3-deazaneplanocin A (DZNep), EPZ005687, EI1, GSK126, and UNC1999. In certain embodiments, the Ezh2 inhibitor is tazemetostat. In certain embodiments, the sample is contacted with the Ezh2 inhibitor for at least about 6 hours, about 12 hours, about 18 hours, about 24 hours, about 36 hours, or about 48 hours. In certain embodiments, the sample is contacted with the Ezh2 inhibitor for up to about 1 day, up to about 2 days, up to about 3 days, up to about 4 days, up to about 5 days, up to about 6 days, up to about 7 days, up to about 8 days, up to about 9 days, up to about 10 days, up to about 12 days, up to about 12 days, up to about 13 days, or up to about 14 days. In certain embodiments, contacting the sample with the Ezh2 inhibitor increases the CD70 polypeptide levels (e.g., detectable by IHC, immunodiffusion, immunoelectrophoresis, RIA, ELISA, immunofluorescent assays, Western blotting, binder-ligand assays, immunohistochemical techniques, agglutination, complement assays, HPLC, TLC, hyperdiffusion chromatography, or a combination thereof). In certain embodiments, when the Ezh2 inhibitor increases the CD70 polypeptide levels in the sample, the methods comprise administering the presently disclosed cells or a composition (e.g., a pharmaceutical composition) comprising thereof to achieve the desired effect, e.g., palliation of an existing condition or prevention of recurrence.

In certain embodiments, the presently disclosed methods comprise a) obtaining a sample having undetectable CD70 polypeptide levels from a subject; b) detecting a CD70 polynucleotide by FISH; and c) administering the presently disclosed cells or a composition (e.g., a pharmaceutical composition) comprising thereof if the CD70 polynucleotide is detected (e.g., detected by FISH).

In certain embodiments, the presently disclosed methods comprise a) obtaining a sample having undetectable CD70 polypeptide levels from a subject; b) contacting the sample with a Ezh2 inhibitor; and c) administering the presently disclosed cells or a composition (e.g., a pharmaceutical composition) comprising thereof if the CD70 polypeptide levels are increased.

In certain embodiments, the presently disclosed methods comprise a) obtaining a sample having undetectable CD70 polypeptide levels from a subject; b) contacting the sample with a Ezh2 inhibitor; and c) administering the presently disclosed cells or a composition (e.g., a pharmaceutical composition) comprising thereof if the CD70 polypeptide is detected (e.g., detected by IHC).

The presently disclosed subject matter provides methods for treating and/or preventing a viral infection in a subject. The method can comprise administering an effective amount of the presently disclosed cells, a presently disclosed composition, or a presently disclosed nucleic acid composition to a subject having a viral infection. Non-limiting examples of viral infections include those caused by cytomegalovirus (CMV), Epstein-Barr virus (EBV), hepatitis A, B, C, D, E, F or G, human immunodeficiency virus (HIV), adenovirus, BK polyomavirus, coronavirus, coxsackievirus, poliovirus, herpes simplex type 1, herpes simplex type 2, human cytomegalovirus, human herpesvirus type 8, varicella-zoster virus, influenza virus, measles virus, mumps virus, parainfluenza virus, respiratory syncytial virus, papillomavirus, rabies virus, and Rubella virus. Other viral targets include Paramyxoviridae (e.g., pneumovirus, morbillivirus, metapneumovirus, respirovirus or rubulavirus), Adenoviridae (e.g., adenovirus), Arenaviridae (e.g., arenavirus such as lymphocytic choriomeningitis virus), Arteriviridae (e.g., porcine respiratory and reproductive syndrome virus or equine arteritis virus), Bunyaviridae (e.g., phlebovirus or hantavirus), Caliciviridae (e.g., Norwalk virus), Coronaviridae (e.g., coronavirus or torovirus), Filoviridae (e.g., Ebola-like viruses), Flaviviridae (e.g., hepacivirus or flavivirus), Herpesviridae (e.g., simplexvirus, varicellovirus, cytomegalovirus, roscolovirus, or lymphocryptovirus), Orthomyxoviridae (e.g., influenza virus or thogotovirus), Parvoviridae (e.g., parvovirus), Picomaviridae (e.g., enterovirus or hepatovirus), Poxviridae (e.g., orthopoxvirus, avipoxvirus, or leporipoxvirus), Retroviridae (e.g., lentivirus or spumavirus), Reoviridae (e.g., rotavirus), Rhabdoviridae (e.g., lyssavirus, novirhabdovirus, or vesiculovirus), and Togaviridae (e.g., alphavirus or rubivirus). In certain embodiments, the viral infections include human respiratory coronavirus, influenza viruses A-C, hepatitis viruses A to G, and herpes simplex viruses 1-9. In certain embodiments, the subject has an immunodeficiency.

The presently disclosed subject matter provides methods for treating and/or preventing a bacterial infection in a subject. The method can comprise administering an effective amount of the presently disclosed cells, a presently disclosed composition, or a presently disclosed nucleic acid composition to a subject having a bacterial infection. Bacterial infections include, but are not limited to, Mycobacteria, Rickettsia, Mycoplasma, Neisseria meningitides, Neisseria gonorrheoeae, Legionella, Vibrio cholerae, Streptococci, Staphylococcus aureus, Staphylococcus epidermidis, Pseudomonas aeruginosa, Corynobacteria diphtheriae, Clostridium spp., enterotoxigenic Eschericia coli, Bacillus anthracis, Rickettsia, Bartonella henselae, Bartonella quintana, Coxiella burnetii, chlamydia, Mycobacterium leprae, Salmonella, shigella, Yersinia enterocolitica, Yersinia pseudotuberculosis; Legionella pneumophila; Mycobacterium tuberculosis; Listeria monocytogenes; Mycoplasma spp., Pseudomonas fluorescens, Vibrio cholerae, Haemophilus influenzae, Bacillus anthracis, Treponema pallidum, Leptospira, Borrelia, Corynebacterium diphtheriae, Francisella, Brucella melitensis, Campylobacter jejuni, Enterobacter, Proteus mirabilis, Proteus, and Klebsiella pneumoniae.

The presently disclosed subject matter provides methods for treating and/or preventing an autoimmune disease in a subject. The method can comprise administering an effective amount of the presently disclosed cells, a presently disclosed composition, or a presently disclosed nucleic acid composition to a subject having an autoimmune disease.

The presently disclosed subject matter provides methods for treating and/or preventing an infectious disease in a subject. The method can comprise administering an effective amount of the presently disclosed cells, a presently disclosed composition, or a presently disclosed nucleic acid composition to a subject having an infectious disease.

Non-limiting examples of autoimmune diseases and inflammatory diseases or conditions thereof include arthritis, e.g., rheumatoid arthritis (RA), Type I diabetes, systemic lupus erythematosus (SLE), inflammatory bowel disease, ulcerative colitis, psoriasis, psoriatic arthritis, scleroderma, autoimmune thyroid disease, Grave's disease, Crohn's disease, multiple sclerosis, systemic sclerosis, asthma, organ transplant rejection, a disease or condition associated with transplant, Takayasu arteritis, giant-cell arteritis, Kawasaki disease, polyarteritis nodosa, Behcet's syndrome, Wegener's granulomatosis, ANCA-vasculitides, Churg-Strauss syndrome, microscopic polyangiitis, vasculitis of connective tissue t diseases, Hennoch-Schonlein purpura, cryoglobulinemic vasculitis, cutaneous leukocytoclastic angiitis, Sarcoidosis, Cogan's syndrome, Wiskott-Aldrich Syndrome, primary angiitis of the CNS, thromboangiitis obliterans, parancoplastic arteritis, myelodysplastic syndrome, crythema clevatum diutinum, amyloidosis, autoimmune myositis, Guillain-Barre Syndrome, histiocytosis, atopic dermatitis, pulmonary fibrosis, glomerulonephritis, Whipple's disease, Still's disease, Sjogren's syndromc, osteomyelofibrosis, chronic inflammatory demyelinating polyneuropathy, Kimura's disease, systemic sclerosis, chronic periaortitis, chronic prostatitis, idiopathic pulmonary fibrosis, chronic granulomatous disease, idiopathic, bleomycin-induced lung inflammation, cytarabine-induced lung inflammation, autoimmune thrombocytopenia, autoimmune neutropenia, autoimmune hemolytic anemia, autoimmune lymphocytopenia, chronic autoimmune thyroiditis, autoimmune hepatitis, Hashimoto's thyroiditis, atopic thyroiditis, Graves disease, autoimmune polyglandular syndrome, autoimmune Addison syndrome, and/or myasthenia gravis. In accordance with the presently disclosed subject matter, the above-described various methods can comprise administering to the subject a checkpoint immune blockade agent.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

Further modification can be introduced to the presently disclosed cells to avert or minimize the risks of immunological complications (known as “malignant T-cell transformation”), e.g., graft versus-host disease (GvHD), or when healthy tissues express the same target antigens as the tumor cells, leading to outcomes similar to GvHD. A potential solution to this problem is engineering a suicide gene into the presently disclosed cells. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T-cell elimination by administering anti-EGFR monoclonal antibody (e.g., cetuximab). EGFRt can be covalently joined to the upstream of the antigen-recognizing receptor. The suicide gene can be included within the vector comprising nucleic acids encoding a presently disclosed antigen-recognizing receptor. In this way, administration of a prodrug designed to activate the suicide gene (e.g., a prodrug (e.g., AP1903 that can activate iCasp-9) during malignant T-cell transformation (e.g., GVHD) triggers apoptosis in the suicide gene-activated cells expressing the presently disclosed antigen-recognizing receptor. The incorporation of a suicide gene into a presently disclosed antigen-recognizing receptor gives an added level of safety with the ability to eliminate the majority of receptor-expressing cells within a very short time period. A presently disclosed cell incorporated with a suicide gene can be pre-emptively eliminated at a given timepoint post the cell infusion, or eradicated at the earliest signs of toxicity.

### 6. KITS

The presently disclosed subject matter provides kits for inducing and/or enhancing an immune response and/or treating and/or preventing a neoplasm or a pathogen infection (e.g., an autoimmune disease or an infectious disease) in a subject. In certain embodiments, the kit comprises an effective amount of presently disclosed cells, a presently disclosed composition, or a presently disclosed nucleic acid composition. In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In certain non-limiting embodiments, the kit includes an isolated nucleic acid molecule encoding an antigen-recognizing receptor (e.g., a TCR-like fusion molecule) directed toward an antigen of interest in expressible form, which may optionally be comprised in the same or different vectors.

If desired, the cells, composition, or nucleic acid composition are provided together with instructions for administering the cells, composition, or nucleic acid composition to a subject having or at risk of developing a tumor (e.g., a cancer) or a pathogen infection (e.g., an infectious disease), or immune disorder (e.g., an autoimmune disease). The instructions generally include information about the use of the cell, composition or nucleic acid composition for the treatment and/or prevention of a neoplasm, or a pathogen infection (e.g., an infectious disease), or an immune disorder (e.g., an autoimmune disease). In certain embodiments, the instructions include at least one of the following: description of the therapeutic agent; dosage schedule and administration for treatment or prevention of a neoplasm, pathogen infection (e.g., an infectious disease), or immune disorder (e.g., an autoimmune disease) or symptoms thereof; precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container.

### 7. EXEMPLARY EMBODIMENTS

Embodiment 1. A method of reducing tumor burden in a subject having a renal cell carcinoma, a pancreatic cancer, or an ovarian cancer, the method comprising administering to the subject an effective amount of a cell comprising a TCR-like fusion molecule that targets CD70.

Embodiment 2. The method of embodiment 2, wherein the method reduces the number of tumor cells, reduces tumor size, and/or eradicates the tumor in the subject.

Embodiment 3. A method of reducing tumor burden in a subject, the method comprising administering to the subject an effective amount of a cell comprising a TCR-like fusion molecule that targets CD70, wherein the tumor is a renal cell carcinoma, a pancreatic cancer, or an ovarian cancer.

Embodiment 4. The method of embodiment 3, wherein the method reduces the number of tumor cells, reduces tumor size, and/or eradicates the tumor in the subject.

Embodiment 5. A method of preventing and/or treating a tumor in a subject having a renal cell carcinoma neoplasm, a renal cell carcinoma, a pancreatic cancer, or an ovarian cancer, the method comprising administering to the subject an effective amount of a cell comprising a TCR-like fusion molecule that targets CD70.

Embodiment 6. A method of preventing and/or treating a tumor in a subject, the method comprising administering to the subject an effective amount of a cell comprising a TCR-like fusion molecule that targets CD70, wherein the tumor is a renal cell carcinoma neoplasm, a renal cell carcinoma, a pancreatic cancer, or an ovarian cancer.

Embodiment 7. A method of preventing and/or treating a tumor in a subject in need thereof, the method comprising a) obtaining a tumor sample that has undetectable CD70 polypeptide levels from the subject; b) detecting a CD70 polynucleotide by FISH; and c) administering to the subject an effective amount of a cell comprising a TCR-like fusion molecule that targets CD70 if the CD70 polynucleotide is detected.

Embodiment 8. A method of preventing and/or treating a tumor in a subject in need thereof, the method comprising a) obtaining a tumor sample that has undetectable CD70 polypeptide levels from the subject; b) contacting the sample with a Ezh2 inhibitor; and c) administering to the subject an effective amount of a cell comprising a TCR-like fusion molecule that targets CD70 if the CD70 polypeptide is detected in the sample.

Embodiment 9. The method of any one of embodiments 1-8, wherein the TCR-like fusion molecule comprises i) a first antigen-binding chain comprising an antigen-binding fragment of a heavy chain variable region (VH) of an antibody; and ii) a second antigen-binding chain comprising an antigen-binding fragment of a light chain variable region (VL) of the antibody; wherein the first and second antigen-binding chains a) each comprise the TRAC polypeptide or the TRBC polypeptide, and b) bind to the second antigen, wherein the TCR-like fusion molecule binds to the second antigen in an HLA-independent manner.

Embodiment 10. The method of embodiment 9, wherein at least one of the TRAC polypeptide and the TRBC polypeptide is endogenous.

Embodiment 11. The method of embodiment 9 or 10, wherein the first and the second antigen-binding chains bind to the second antigen with a dissociation constant (KD) of about 1×10⁻⁸ M or less.

Embodiment 12. The method of any one of embodiments 9-11, wherein the first and the second antigen-binding chains bind to the second antigen with a dissociation constant (KD) of about 5×10⁻⁹ M or less.

Embodiment 13. The method of any one of embodiments 9-12, wherein the first antigen-binding chain comprises an antigen-binding fragment of a VH of an antibody and a TRBC polypeptide, and the second antigen-binding chain comprises an antigen-binding fragment of a VL of the antibody and a TRAC polypeptide.

Embodiment 14. The method of any one of embodiments 9-13, wherein the first antigen-binding chain comprises an antigen-binding fragment of a VH of an antibody and a TRAC polypeptide, and the second antigen-binding chain comprises an antigen-binding fragment of a VL of the antibody and a TRBC polypeptide.

Embodiment 15. The method of any one of embodiments 9-14, wherein i) the first antigen-binding chain comprises a CDR1 comprising the amino acid sequence set forth in SEQ ID NO: 34, a CDR2 comprising the amino acid sequence set forth in SEQ ID NO: 35, and a CDR3 comprising the amino acid sequence set forth in SEQ ID NO: 36, and the second antigen-binding chain comprises a CDR 1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 39; or ii) the first antigen-binding chain comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 39, and the second antigen-binding chain comprises a CDR1 comprising the amino acid sequence set forth in SEQ ID NO: 34, a CDR2 comprising the amino acid sequence set forth in SEQ ID NO: 35, and a CDR3 comprising the amino acid sequence set forth in SEQ ID NO: 36.

Embodiment 16. The method of any one of embodiments 9-15, wherein i) the first antigen-binding chain comprises a CDR1, a CDR2, and a CDR3 of the heavy chain variable region set forth in SEQ ID NO: 40, and the second antigen-binding chain comprises a CDR1, a CDR2, and a CDR3 of the light chain variable region set forth in SEQ ID NO: 42; or ii) the first antigen-binding chain comprises a CDR1, a CDR2, and a CDR3 of the light chain variable region set forth in SEQ ID NO: 42, and the second antigen-binding chain comprises a CDR1, a CDR2, and a CDR3 of the heavy chain variable region set forth in SEQ ID NO: 40.

Embodiment 17. The method of any one of embodiments 9-15, wherein i) the first antigen-binding chain comprises the heavy chain variable region set forth in SEQ ID NO: 40, and the second antigen-binding chain comprises a CDR1, a CDR2, and a CDR3 of the light chain variable region set forth in SEQ ID NO: 42; or ii) the first antigen-binding chain light chain variable region set forth in SEQ ID NO: 42; and the second antigen-binding chain comprises the heavy chain variable region set forth in SEQ ID NO: 40.

Embodiment 18. The method of any one of embodiments 9-17, wherein the first and second antigen binding chains are capable of associating with a CD3ζ polypeptide.

Embodiment 19. The method of embodiment 18, wherein the first and second antigen binding chains, upon binding to the second antigen, are capable of activating the CD3ζ polypeptide.

Embodiment 20. The method of embodiment 19, wherein the activation of the CD3ζ polypeptide is capable of activating the cell.

Embodiment 21. The method of any one of embodiments 9-20, wherein the cell further comprises a gene disruption of a TRAC locus.

Embodiment 22. The method of any one of embodiments 9-20, wherein the cell further comprises a gene disruption of a CD70 locus.

Embodiment 23. The method of any one of embodiments 9-20, wherein the cell further comprises a gene disruption of a TRAC locus and a CD70.

Embodiment 24. The method of any one of embodiments 1-23, wherein the tumor comprises tumor cells having a CD70 low antigen density.

Embodiment 25. The method of any one of embodiments 1-24, wherein the tumor CD70+ tumor cells having low tumor cell frequency.

Embodiment 26. The method of any one of embodiments 1-25, wherein the tumor comprises a CD70 polypeptide that is not detectable by immunodiffusion, immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, or a combination thereof. In certain embodiments, the tumor and/or neoplasm comprises a CD70 polypeptide that is not detectable by immunohistochemistry (IHC).

Embodiment 27. The method of any one of embodiments 1-26, wherein the tumor comprises a CD70 polypeptide by immunohistochemistry (IHC).

Embodiment 28. The method of any one of embodiments 1-27, wherein the cell is a cell of the lymphoid lineage or a cell of the myeloid lineage.

Embodiment 29. The method of embodiment 28, wherein the cell of the lymphoid lineage is selected from the group consisting of a T cell, a B cell, a Natural Killer (NK) cell, and a dendritic cell.

Embodiment 30. The method of any one of embodiments 1-29, wherein the cell is a T cell.

Embodiment 31. The method of embodiment 30, wherein the T cell is derived from an induced pluripotent stem cell.

Embodiment 32. The method of embodiment 30 or 31, wherein the T cell is a CD8⁺ T cell.

Embodiment 33. The method of embodiment 32, wherein the CD8⁺ T cell is CD4 independent.

Embodiment 34. The method of any one of embodiments 30-33, wherein the T cell is selected from the group consisting of a cytotoxic T lymphocyte (CTL), a γδ T cell, a tumor-infiltrating lymphocyte (TIL), a regulatory T cell, and a Natural Killer T (NKT) cell.

Embodiment 35. The method of any one of embodiments 1-34, wherein the cell further comprises a chimeric antigen receptor (CAR) that targets a second antigen.

Embodiment 36. The method of embodiment 35, wherein the CAR comprises an extracellular antigen-binding domain that binds to the first antigen, and an intracellular signaling domain that is capable of delivering an activation signal to the cell.

Embodiment 37. The method of embodiment 36, wherein the intracellular signaling domain of the CAR comprises a CD32 polypeptide.

Embodiment 38. The method of embodiment 37, wherein the CD3ζ polypeptide is a native CD3ζ polypeptide or a modified CD3ζ polypeptide.

Embodiment 39. The method of embodiment 38, wherein the modified CD3ζ polypeptide comprises a native ITAM1, an ITAM2 variant consisting of two loss-of-function mutations, and an ITAM3 variant consisting of two loss-of-function mutations.

Embodiment 40. The method of any one of embodiments 36-39, wherein the intracellular signaling domain of the CAR further comprises at least one costimulatory signaling region.

Embodiment 41. The method of embodiment 40, wherein the at least one costimulatory signaling region comprises at least an intracellular domain of a co-stimulatory molecule or a portion thereof.

Embodiment 42. The method of embodiment 41, wherein the costimulatory molecule is selected from the group consisting of CD28, 4-1BB, OX40, CD27, CD40, CD154, CD97, CD11a/CD18, ICOS, DAP-10, CD2, CD150, CD226, and NKG2D.

Embodiment 43. The method of any one of embodiments 36-42, wherein the CAR comprises a transmembrane domain.

Embodiment 44. The method of any one of embodiments 1-43, wherein the cell further comprises a chimeric co-stimulating receptor (CCR).

Embodiment 45. The method of embodiment 44, wherein the CCR comprises an extracellular antigen-binding domain that binds to the third antigen and an intracellular domain that is capable of delivering a costimulatory signal to the cell but does not alone deliver an activation signal to the cell.

Embodiment 46. The method of embodiment 45, wherein the intracellular domain of the CCR comprises at least an intracellular domain of a co-stimulatory molecule or a portion thereof.

Embodiment 47. The method of embodiment 46, wherein the costimulatory molecule is selected from the group consisting of CD28, 4-1BB, OX40, CD27, CD40, CD154, CD97, CD11a/CD18, ICOS, DAP-10, CD2, CD150, CD226, and NKG2D

Embodiment 48. The method of any one of embodiments 35-47, wherein the second antigen is a tumor antigen or a pathogen antigen.

Embodiment 49. The method of embodiment 48, wherein the tumor antigen is selected from the group consisting of CD19, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell (e.g. a cell surface antigen), ANO9, AQP2, ASIC3, ASPRV1, ATP6VOA4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD30, CD300LF, CD312, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-α, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, LICAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PR1), PSD2, PTPRJ, RDH16, receptor tyrosine-protein kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TASIR3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, and ZDHHC11.

Embodiment 50. The method of any one of embodiments 1-49, wherein the cell further comprises at least one exogenous costimulatory ligand.

Embodiment 51. The method of embodiment 50, wherein the at least one exogenous co-stimulatory ligand is selected from the group consisting of a tumor necrosis factor (TNF) family member, an immunoglobulin (Ig) superfamily member, and combinations thereof.

Embodiment 52. The method of embodiment 51, wherein the TNF family member is selected from the group consisting of 4-1BBL, OX40L, CD70, FasL, GITRL, TNF-related apoptosis-inducing ligand (TRAIL), CD30L, LIGHT (TNFSF14), CD40L.

Embodiment 53. The method of embodiment 51 or 52, wherein the Ig superfamily member is selected from the group consisting of CD80, CD86, ICOSLG, and combinations thereof.

Embodiment 54. The method of any one of embodiments 50-53, wherein the at least one exogenous costimulatory ligand comprises CD80.

Embodiment 55. The method of any one of embodiments 50-53, wherein the at least one exogenous a costimulatory ligand comprises 4-1BBL.

Embodiment 56. The method of any one of embodiments 50-53, wherein the cell comprises two exogenous costimulatory ligands.

Embodiment 57. The method of embodiment 56, wherein the at least two exogenous costimulatory ligands comprise CD80 and 4-1BBL.

Embodiment 58. The method of any one of embodiments 1-57, wherein the cell further comprises a fusion polypeptide comprising: a) an extracellular domain and a transmembrane domain of a co-stimulatory ligand, and b) an intracellular domain of a first co-stimulatory molecule.

Embodiment 59. The method of embodiment 58, wherein the co-stimulatory ligand is selected from the group consisting of a tumor necrosis factor (TNF) family member, an immunoglobulin (Ig) superfamily member, and combinations thereof.

Embodiment 60. The method of embodiment 59, wherein the TNF family member is selected from the group consisting of 4-1BBL, OX40L, CD70, GITRL, CD40L, and combinations thereof.

Embodiment 61. The method of embodiment 59 or 60, wherein the Ig superfamily member is selected from the group consisting of CD80, CD86, ICOSLG, and combinations thereof.

Embodiment 62. The method of any one of embodiments 58-61, wherein the co-stimulatory ligand is CD80.

Embodiment 63. The method of any one of embodiments 58-62, wherein the first co-stimulatory molecule is selected from the group consisting of CD28, 4-1BB, OX40, ICOS, DAP-10, CD27, CD40, NKG2D, CD2, and combinations thereof.

Embodiment 64. The method of embodiment 63, wherein the first co-stimulatory molecule is 4-1BB.

Embodiment 65. The method of any one of embodiments 58-64, wherein the co-stimulatory ligand is CD80 and the first co-stimulatory molecule is 4-1BB.

Embodiment 66. The method of any one of embodiments 58-65, wherein the fusion polypeptide further comprises an intracellular domain of a second co-stimulatory molecule.

Embodiment 67. The method of embodiment 66, wherein the second co-stimulatory molecule is selected from the group consisting of CD28, 4-1BB, OX40, ICOS, DAP-10, CD27, CD40, NKG2D, CD2, and combinations thereof.

Embodiment 68. The method of embodiment 66 or 67, wherein the second co-stimulatory molecule is CD28.

Embodiment 69. The method of any one of embodiments 61-68, wherein the co-stimulatory ligand is CD80, the first co-stimulatory molecule is 4-1BB, and the second co-stimulatory molecule is CD28.

Embodiment 70. The method of any one of embodiments 1-69, wherein the cell is autologous.

Embodiment 71. The method of any one of embodiments 1-69, wherein the cell is allogeneic.

### EXAMPLES

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides disclosed herein, and, as such, may be considered in making and practicing the presently disclosed subject matter. Particularly useful techniques for particular embodiments will be discussed in the sections that follow.

The following examples are put forth so as to provide those of ordinary skill in the art with a complete disclosure and description of how to make and use the presently disclosed cells and compositions, and are not intended to limit the scope of what the inventors regard as their invention.

### Example 1

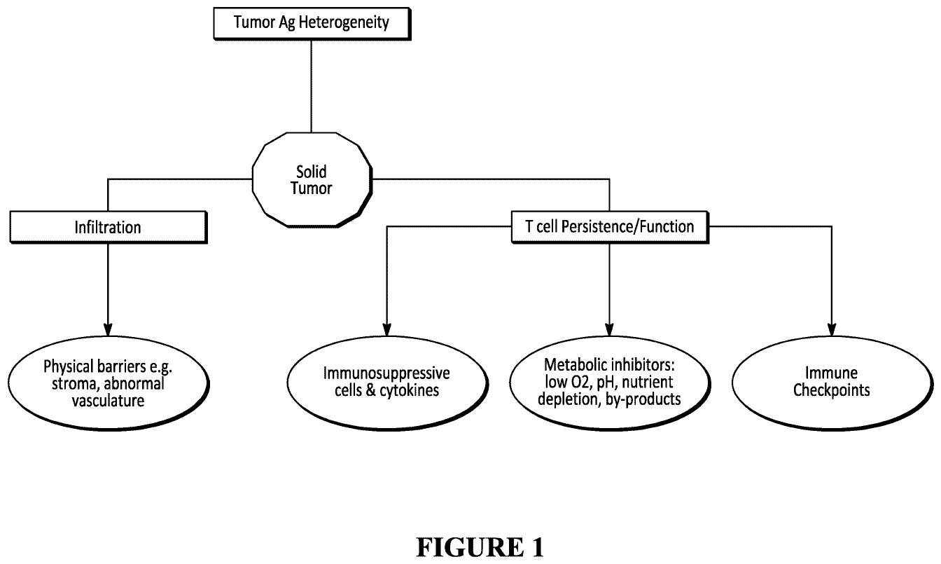

CAR-T cells to date have not been effective at eradicating solid tumors. Early phase clinical trials using a CAIX-CAR-T for renal cell carcinoma (RCC) have failed and a current clinical trial with a CD70 CAR-T for RCC has showed limited treatment response. Other clinical trials for prostate cancer, breast cancer and lung cancer (to name a few solid tumors) have failed with respect to seeing good tumor response. The major reason CAR-T cells have failed is because of tumor antigen heterogencity. See FIG. 1. It is challenging to find a tumor target that is expressed on every tumor cell, and that is not also expressed on normal tissues. In RCC, CD70 is expressed on the tumor. So far, approaches including either a CD70 CAR or a CAIX CAR, or combinations of the same with limited efficacy.

In order to study renal cell carcinoma (RCC), the presently disclosed subject matter developed two patient-derived xenograft models (K5 and K7) from clear cell RCC patients.

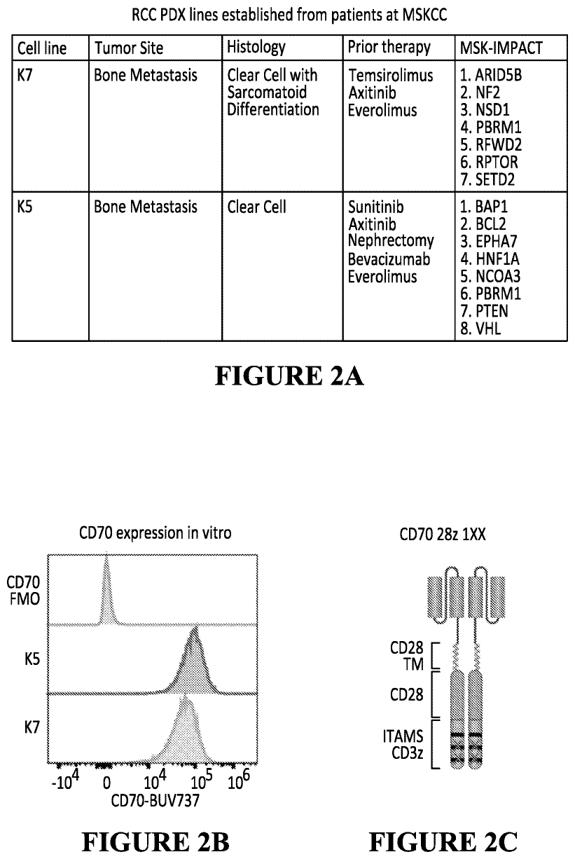

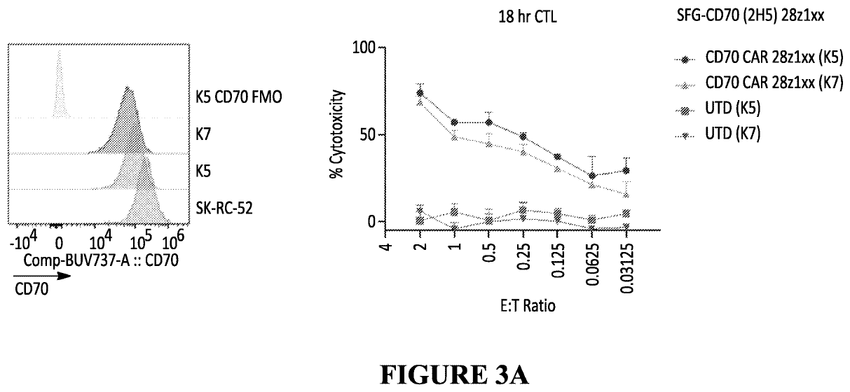

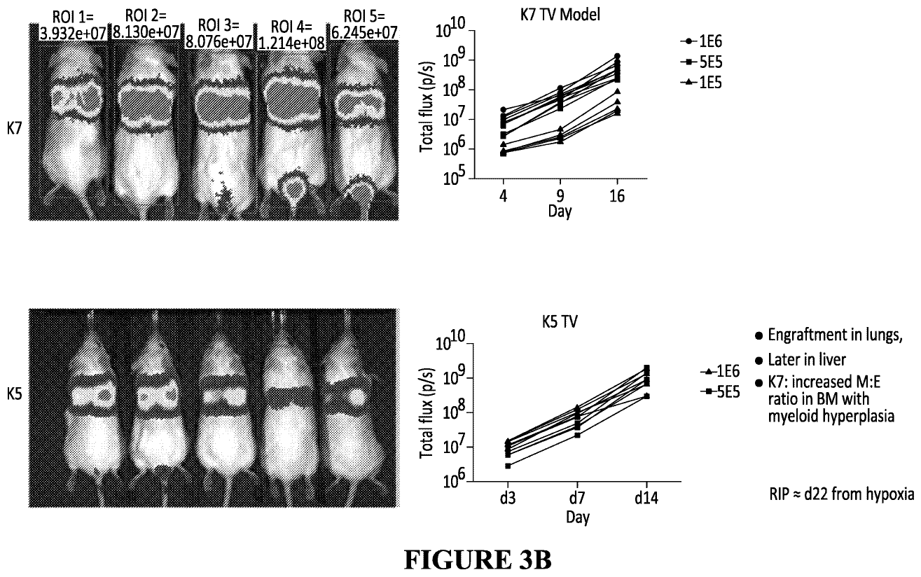

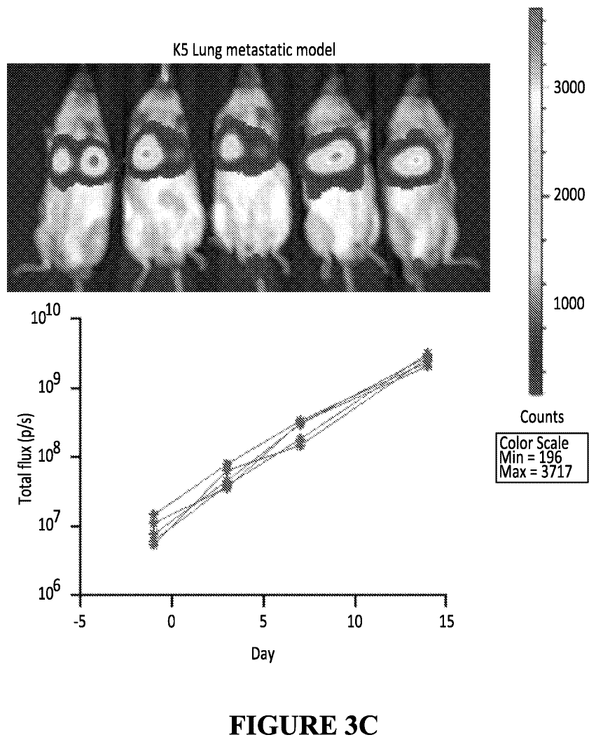

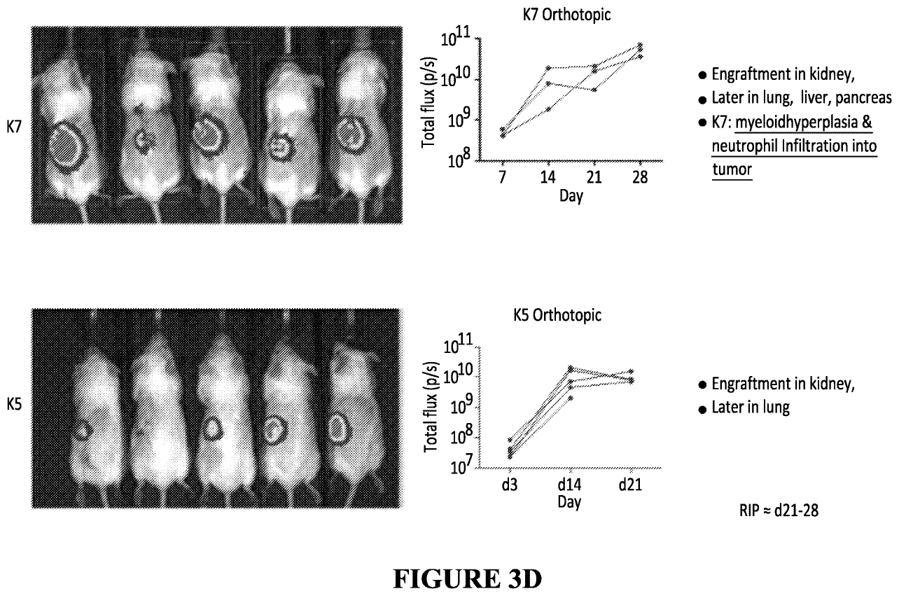

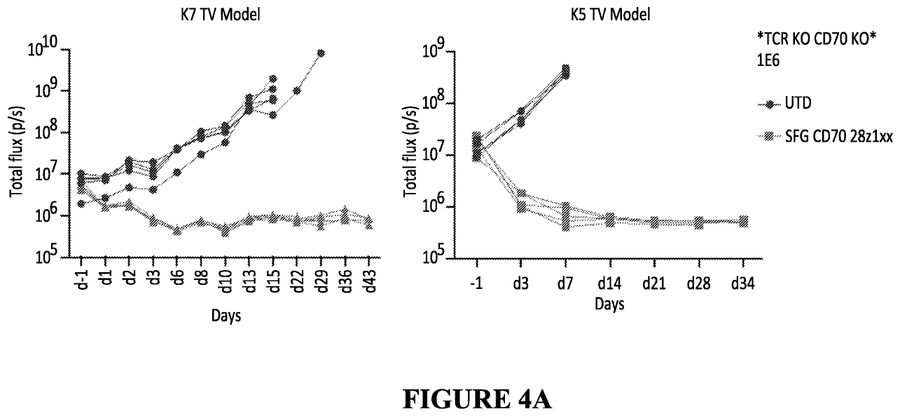

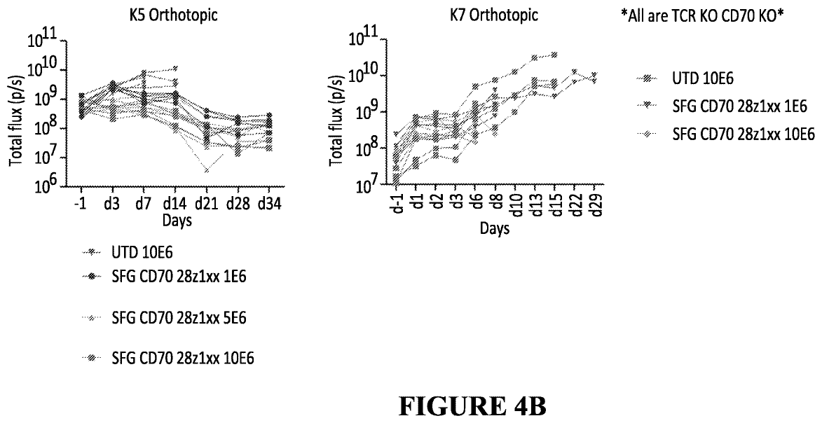

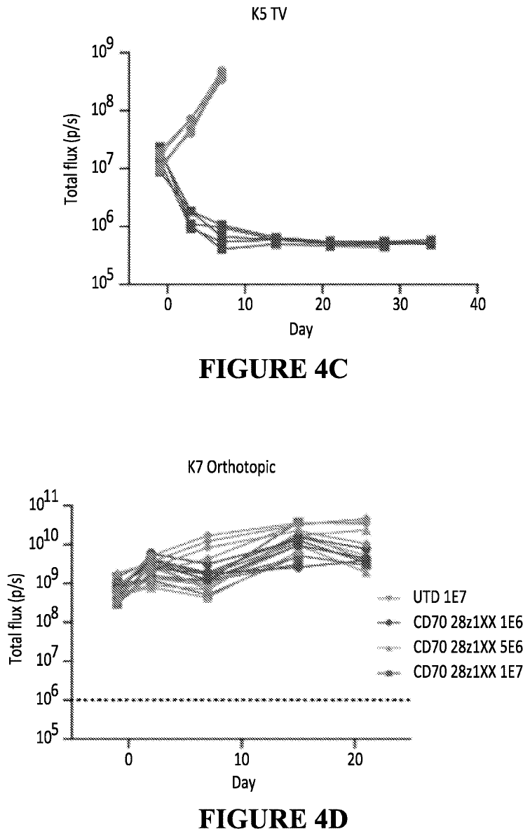

As shown in FIG. 2B, CD70 expression is highly maintained in in vitro setting. Further, T cells expressing an anti-CD70 CAR can activate cytotoxic effects in in vitro models. See FIG. 3A. Next, it was demonstrated that anti-CD70 CAR T cells were capable of inducing cytotoxicity in RCC models established by tail-vein administration but not by orthotopic kidney administration. See FIGS. 4A-4D.

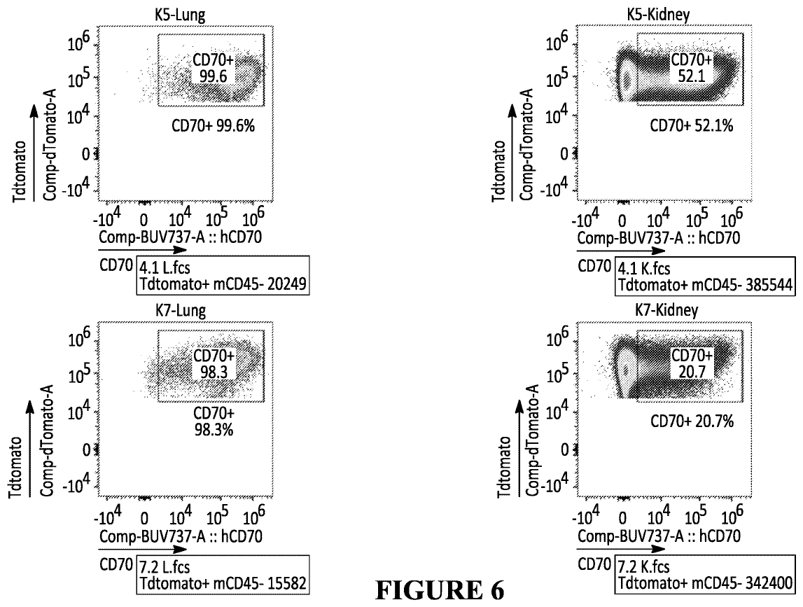

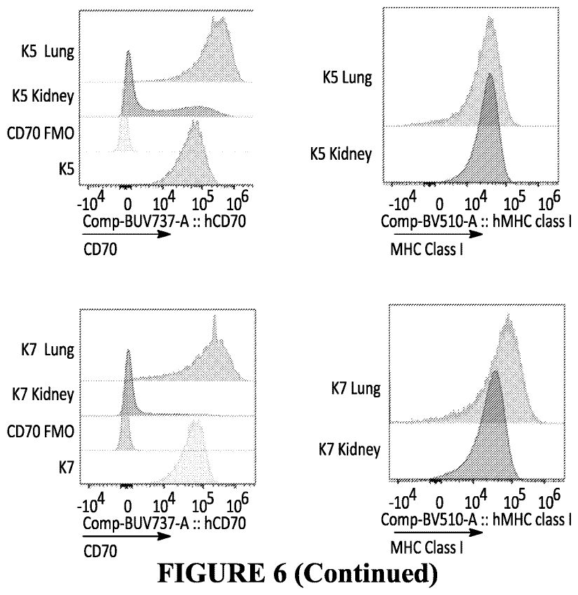

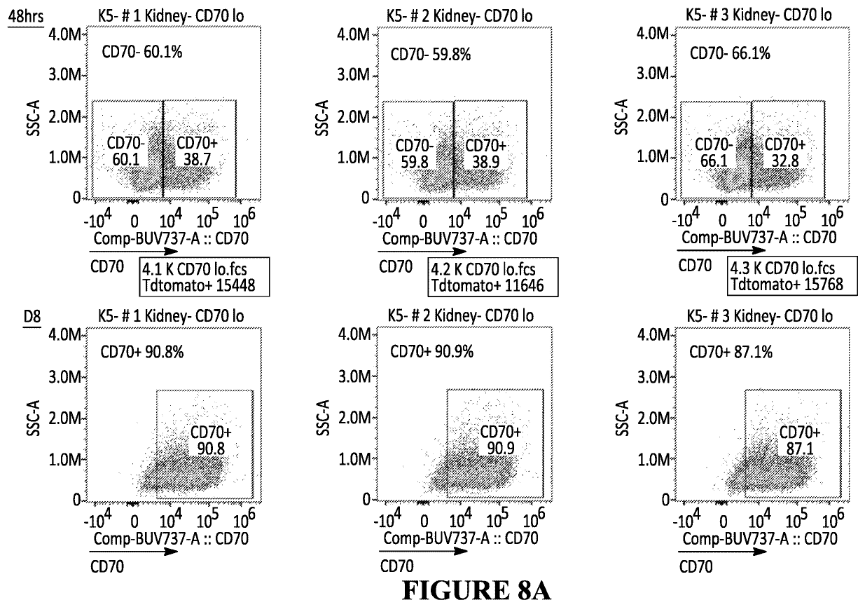

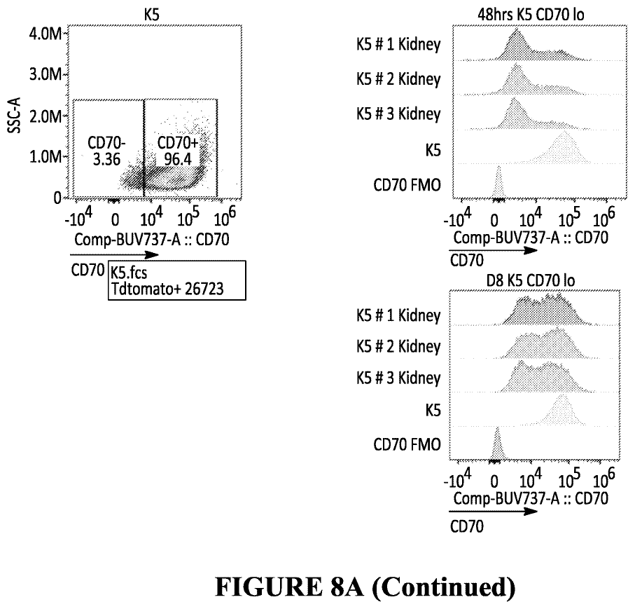

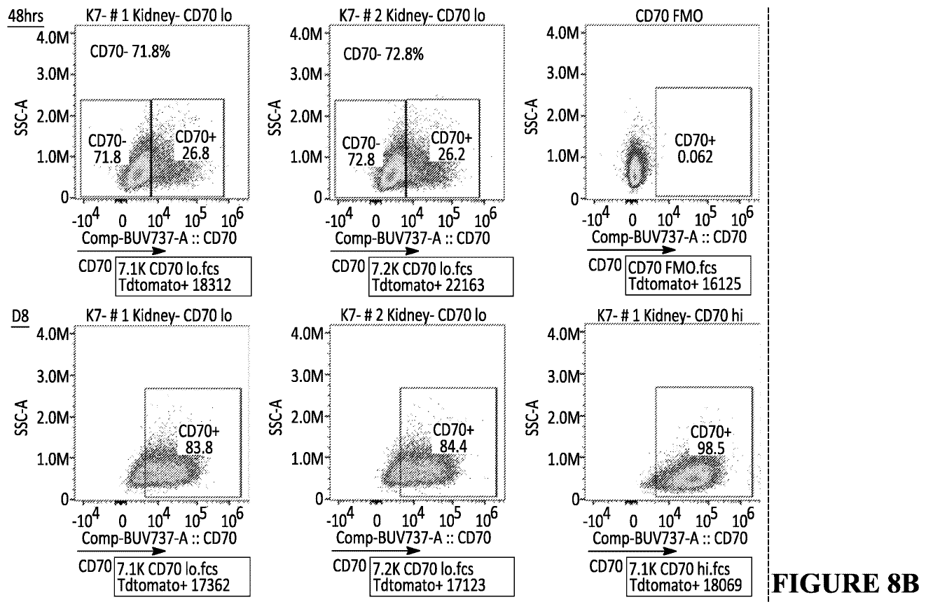

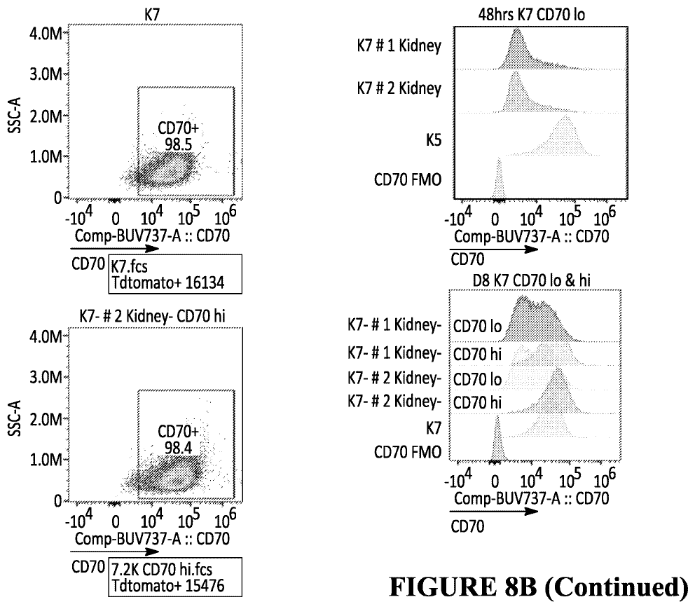

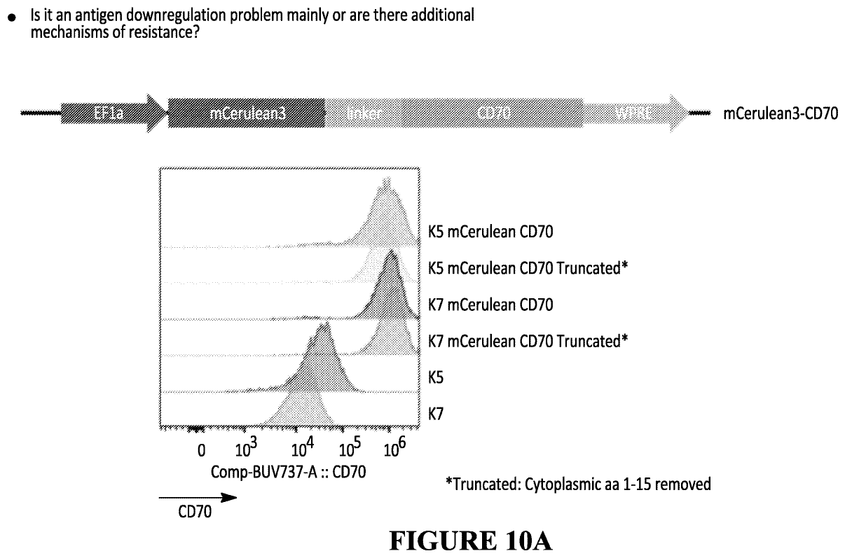

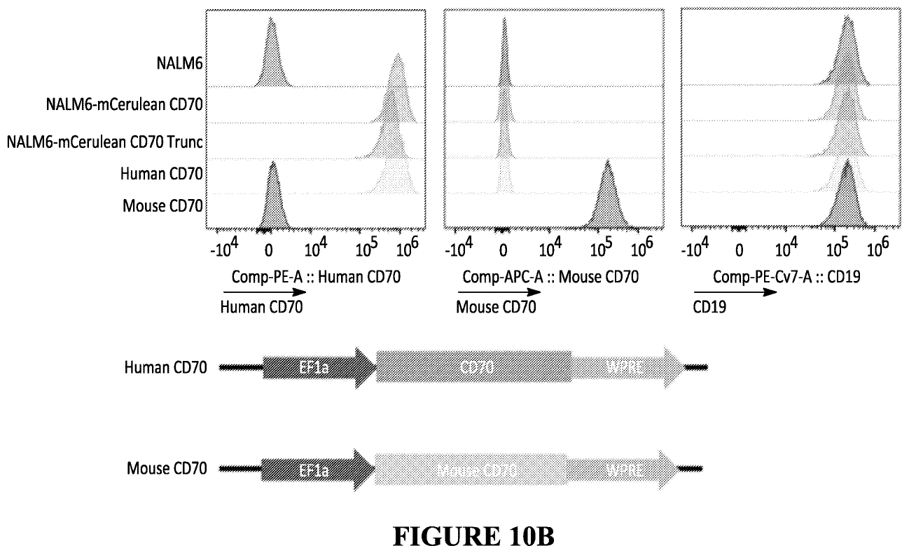

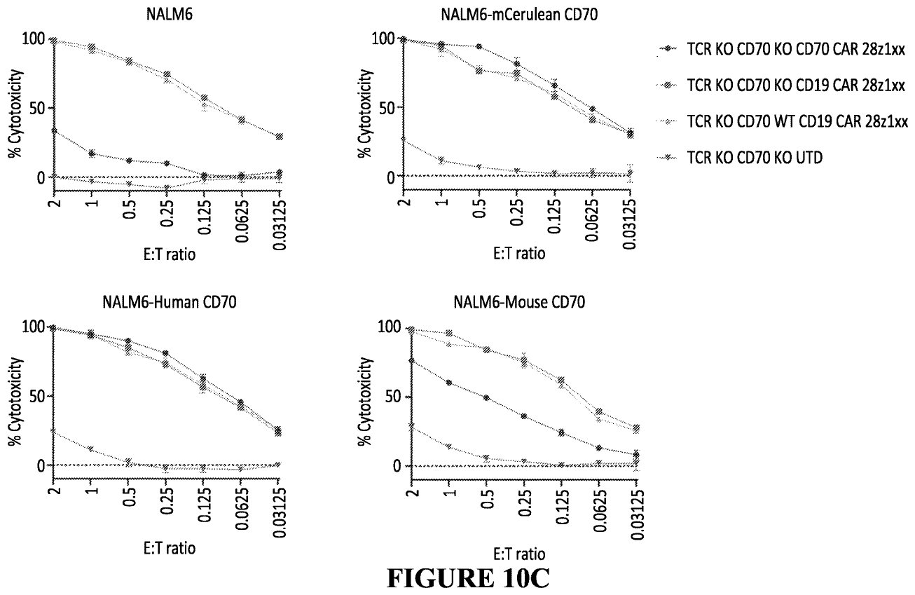

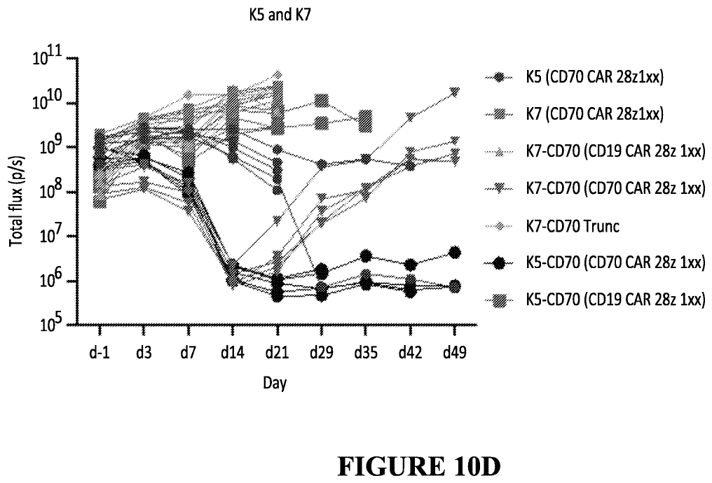

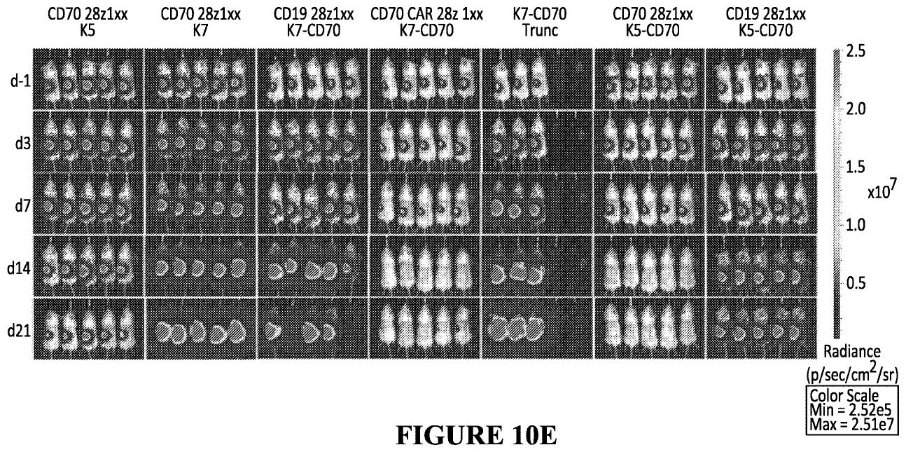

Next, it was determined whether antigen downregulation was a possible mechanism of the observed resistance. As shown in FIG. 6, CD70 was almost 100% positive in the lung but only a portion of the kidney tumor was CD70 positive. Next, it was determined that in vitro culturing of untreated K5 and K7 cells having reduced expression of CD70 restored the expression level of the antigen. See FIGS. 8A and 8B. Thus, it was explored whether expression of a CD70 fusion protein would improve the tumor clearance in in vivo models. As seen in FIGS. 10A-10E, overexpression of CD70 was sufficient to result in tumor clearance in in vivo models. Overall, these data demonstrate that i) CD70 was downregulated in the kidney but not the lung in vivo in untreated mice, ii) CD70 downregulation in vivo was reversed by culturing the “CD70 low antigen” cells in vitro, and iii) lentiviral overexpression of CD70 resulted in kidney tumor clearance by SFG CD70 28z1xx in K5 and K7 but rapid relapse in K7.

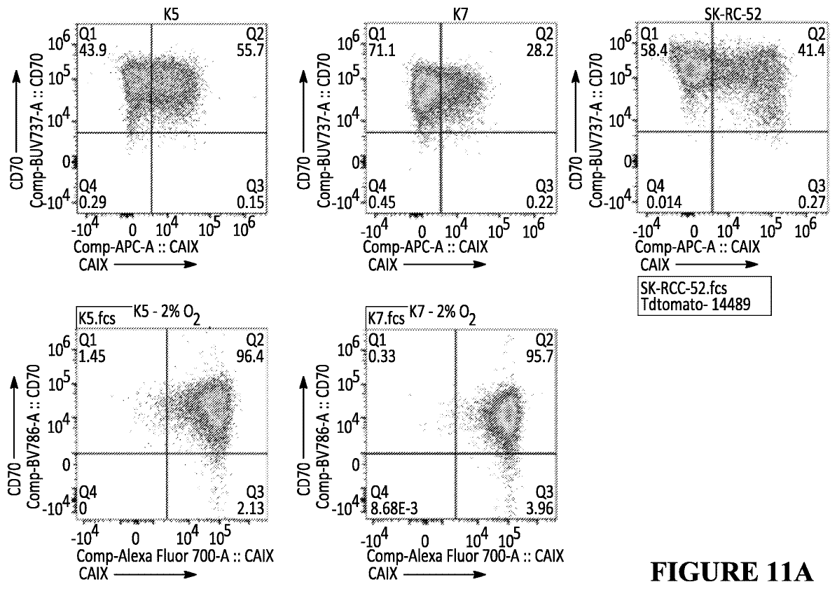

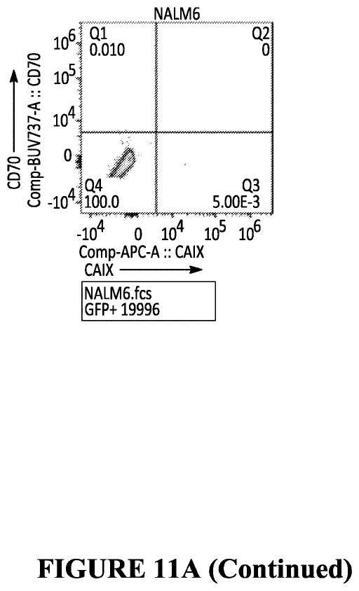

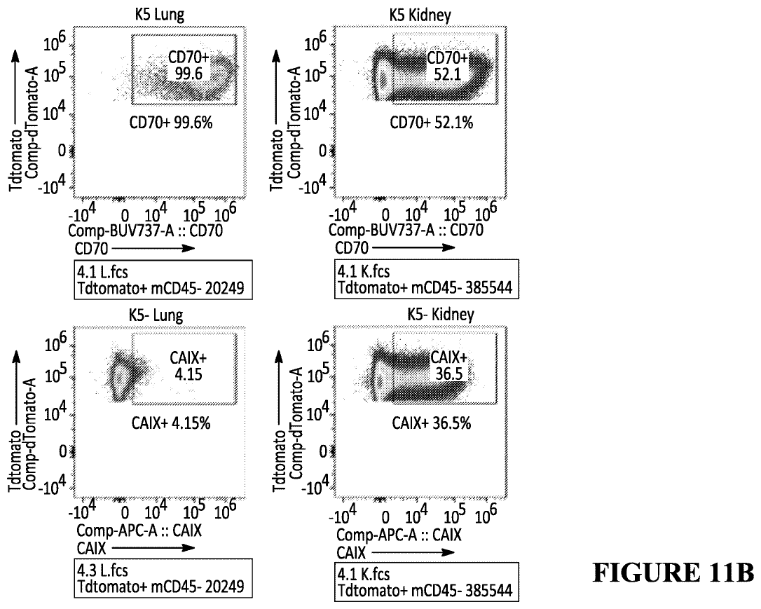

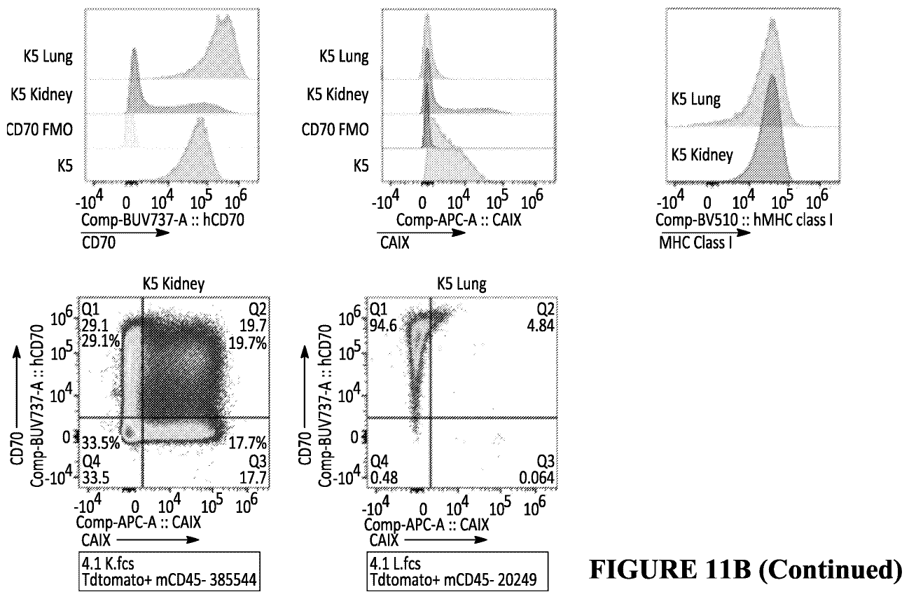

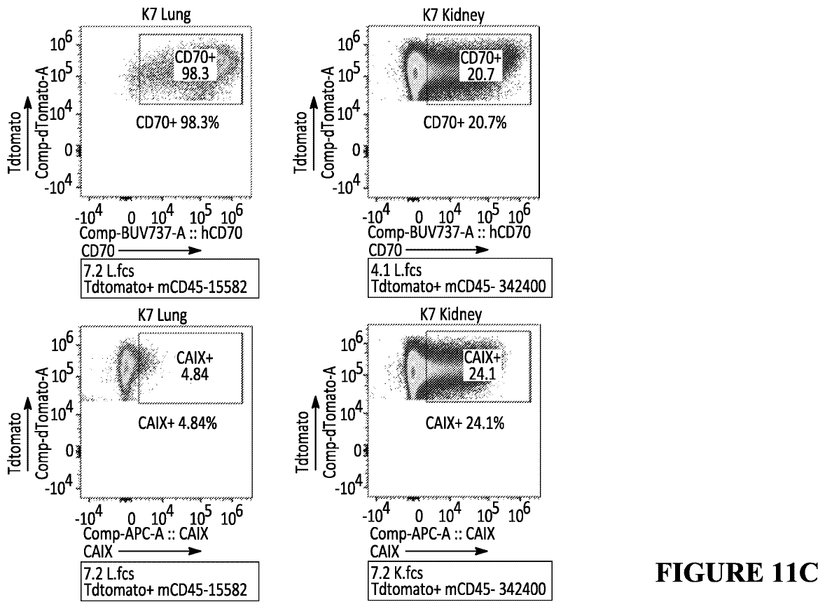

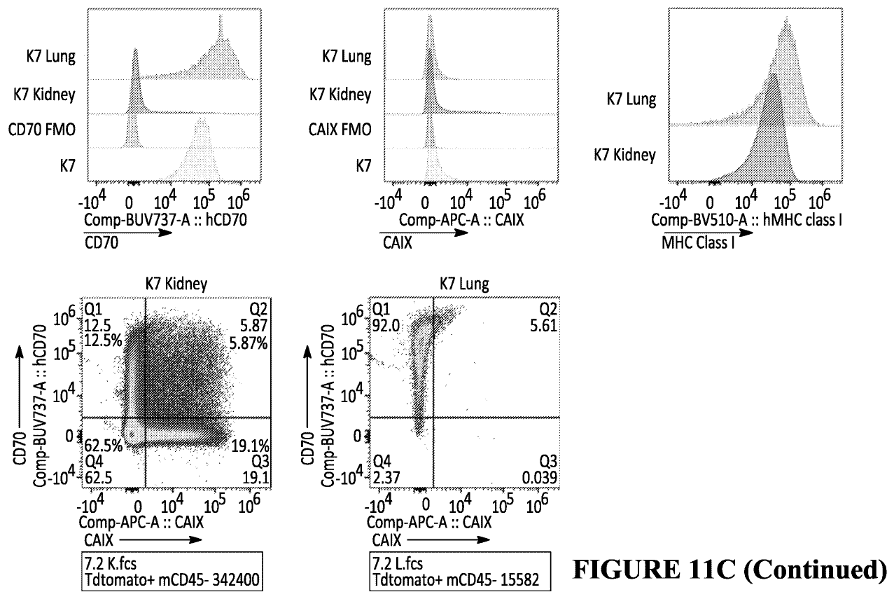

Since carbonic anhydrase IX (CAIX) is expressed in clear cell renal cell carcinoma and in sarcomatoid renal cell carcinoma, it was determined whether this antigen was regulated and heterogeneously expressed as CD70. FIGS. 11A-11C show that CAIX was expressed heterogeneously in K5 and K7 RCC cells, and that it is upregulated by hypoxia (2% O2). Transcriptomic and proteomic analysis of sorted CD70/CAIX double negative, double positive and single positive untreated kidney tumor revealed that the CD70/CAIX double negative population has very low CD70 expression detected. Thus, CAIX and CD70 are regulated target antigens in RCC.

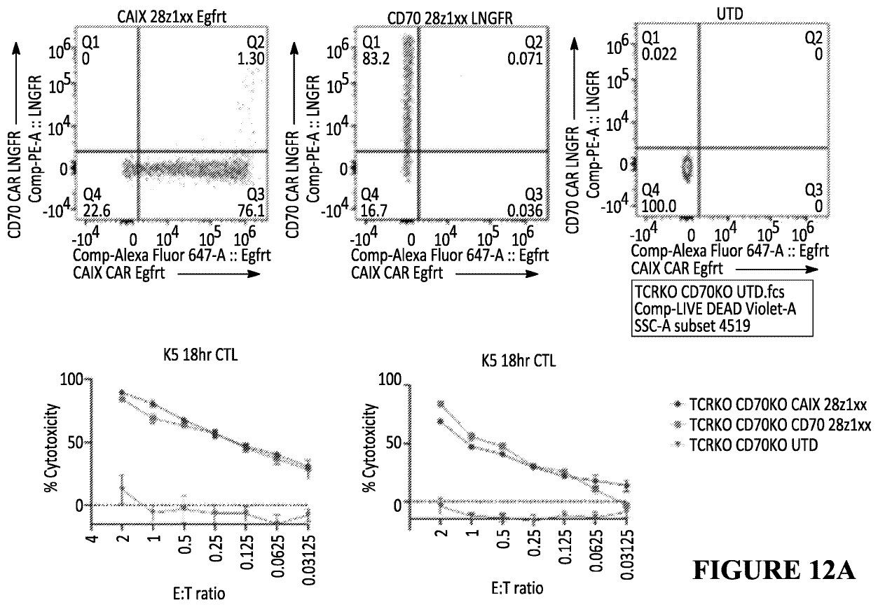

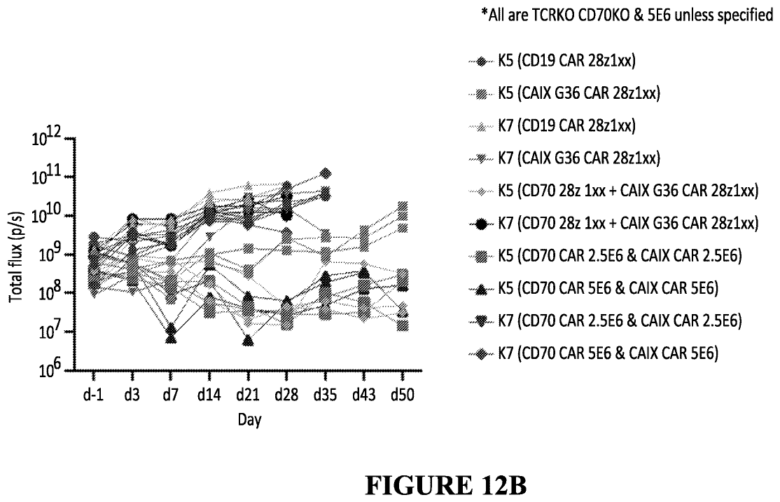

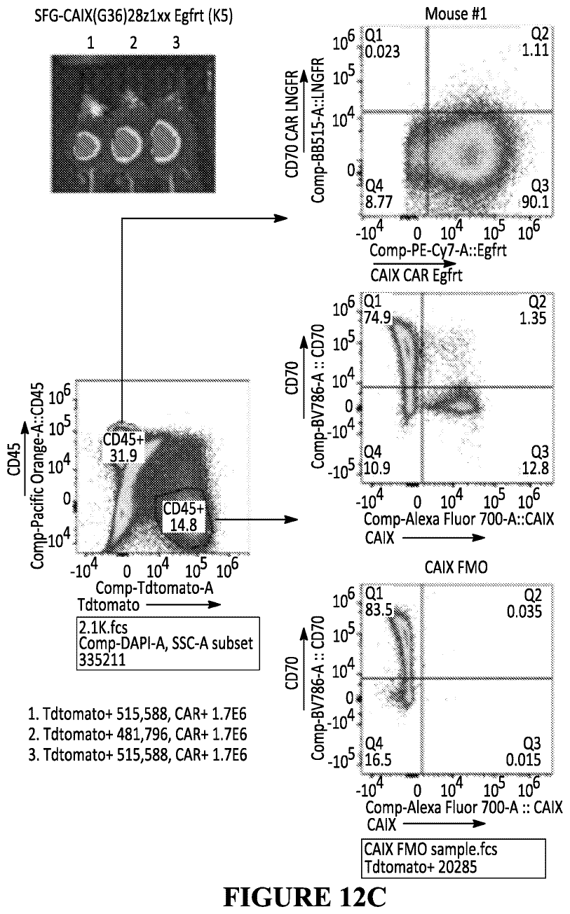

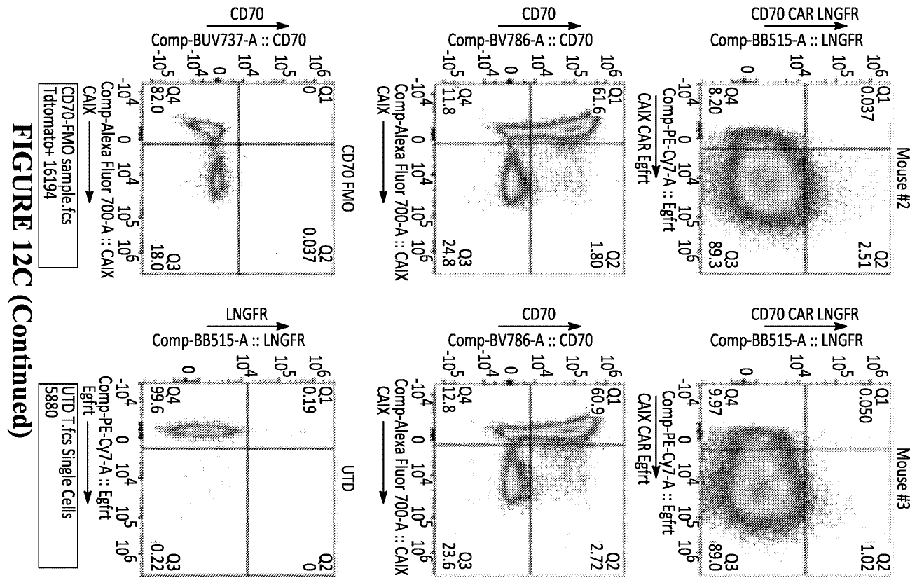

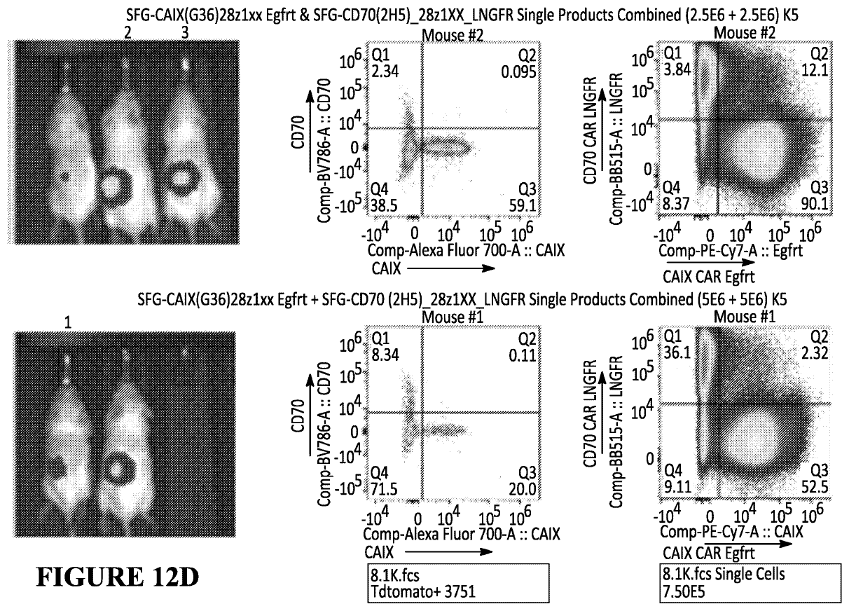

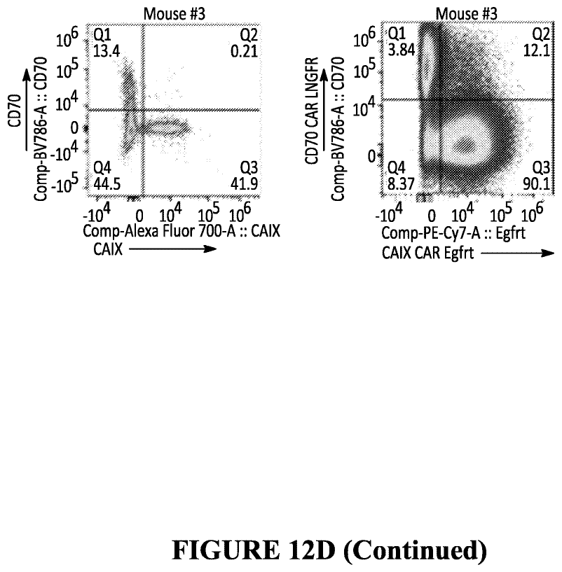

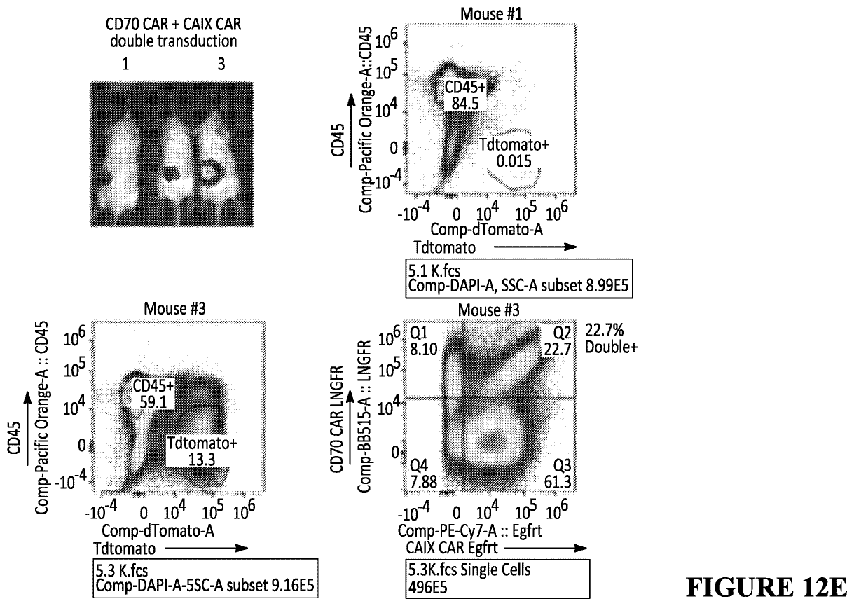

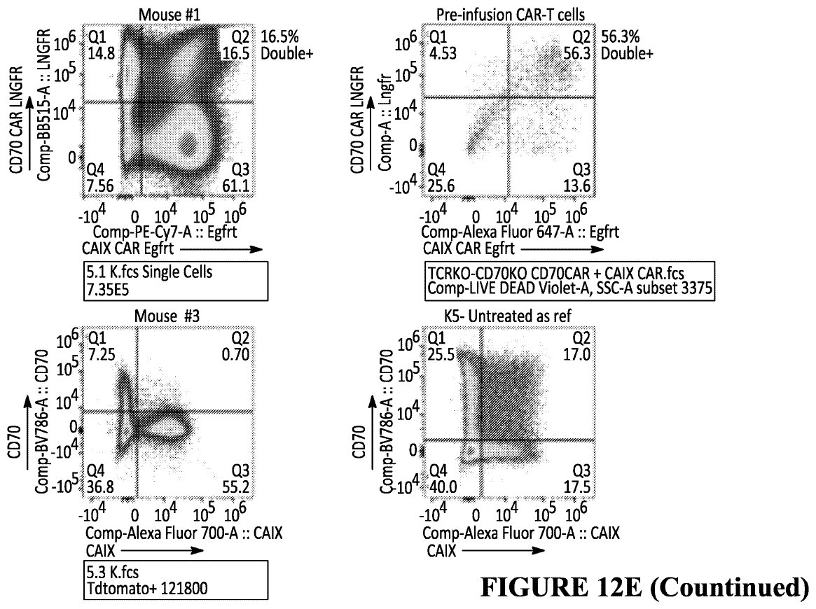

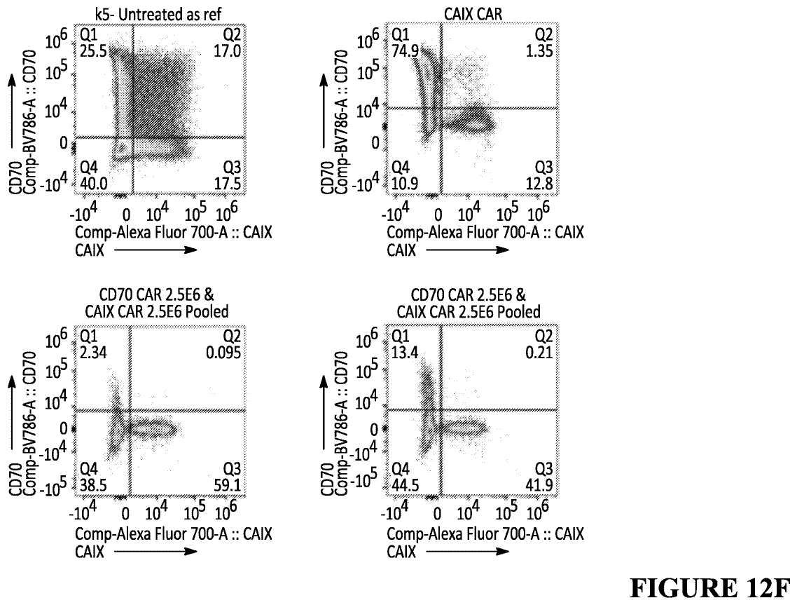

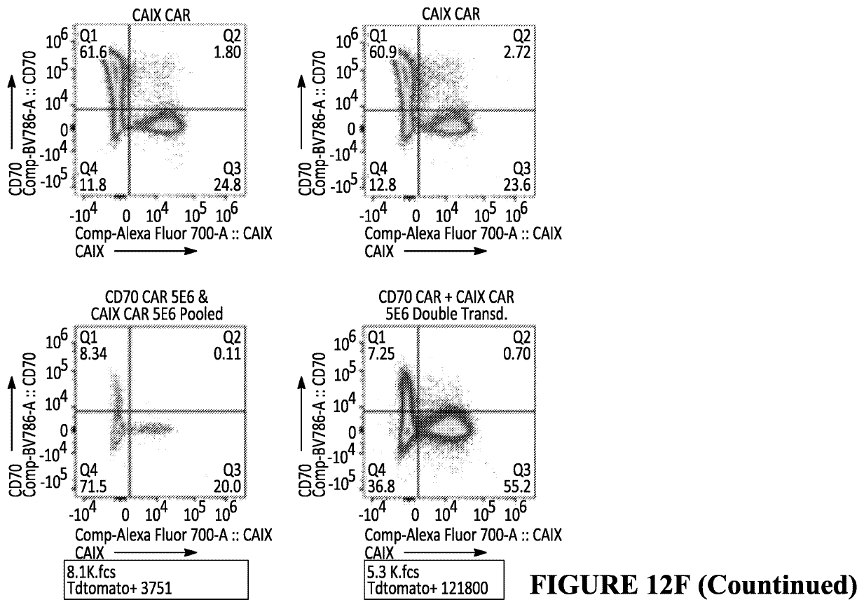

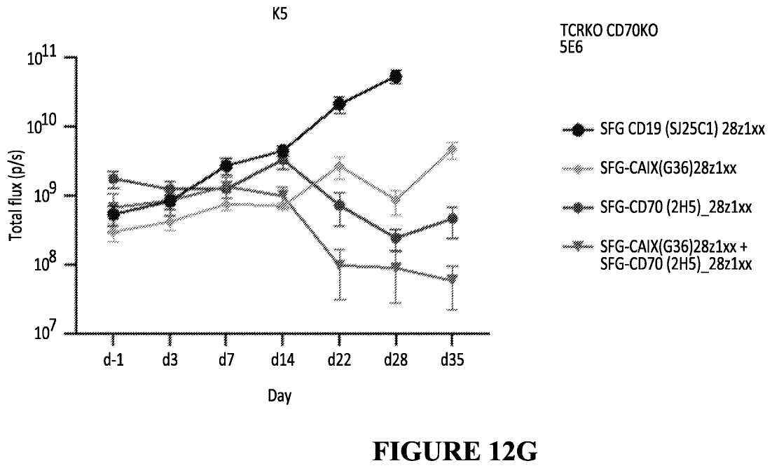

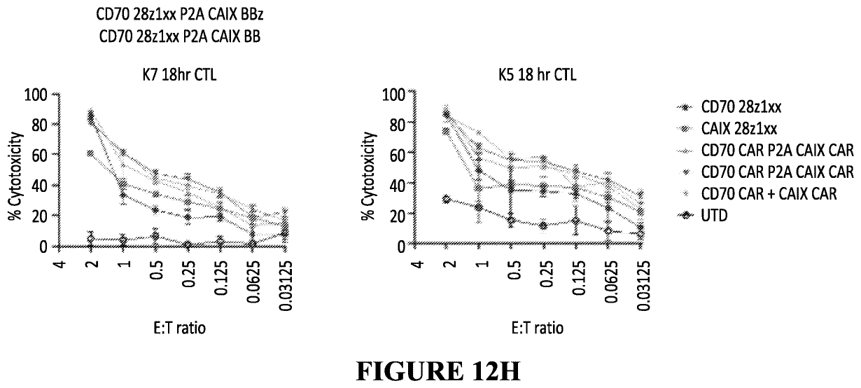

Next, a dual targeting approach using an SFG-CD70 28z1xx CAR and a SFG CAIX 28z1xx CAR (pooling two CAR-T cells or a double transduction approach) was used. Anti-CAIX CAR T cells as well as dual targeting approach including anti-CD70 CAR T cell were capable of in vitro killing. See FIGS. 12A and 12B. However, in vivo studies demonstrated that CD70 and CAIX dual targeting did not result in tumor clearance because of residual low density antigen populations. See FIGS. 12C-12I. In conclusion, CD70/CAIX dual targeting improved tumor control in K5 cell but not K7 cells. Further, it was observed a significant amount of cells having low density antigen and that there is a modest improvement in tumor control but a “CD70/CAIX double negative” population remains.

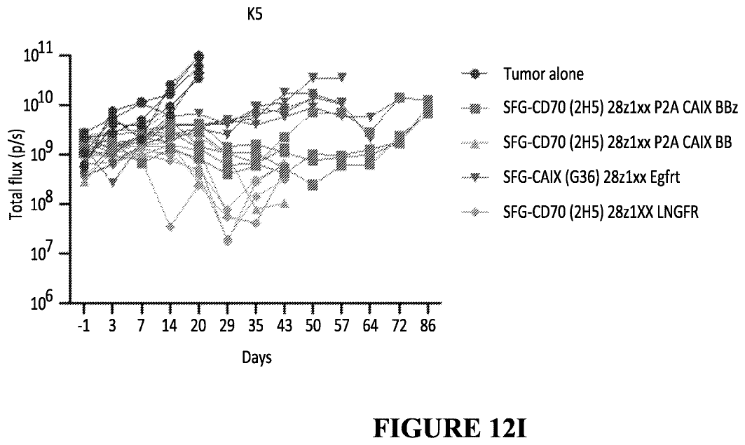

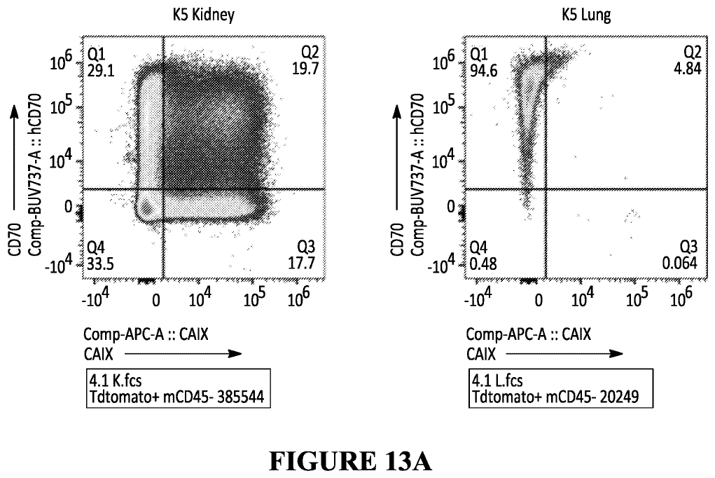

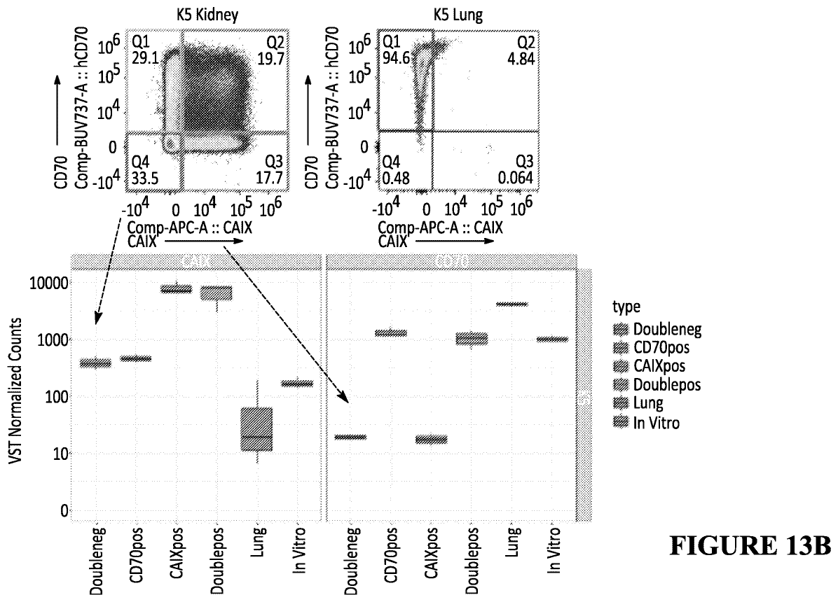

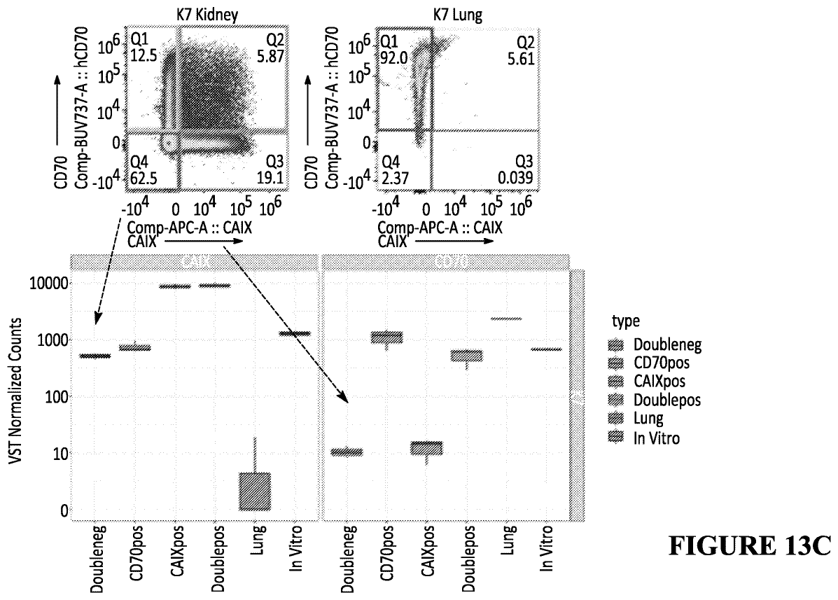

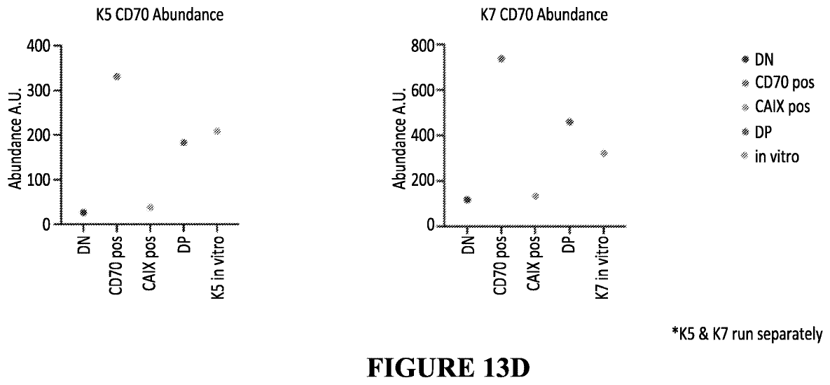

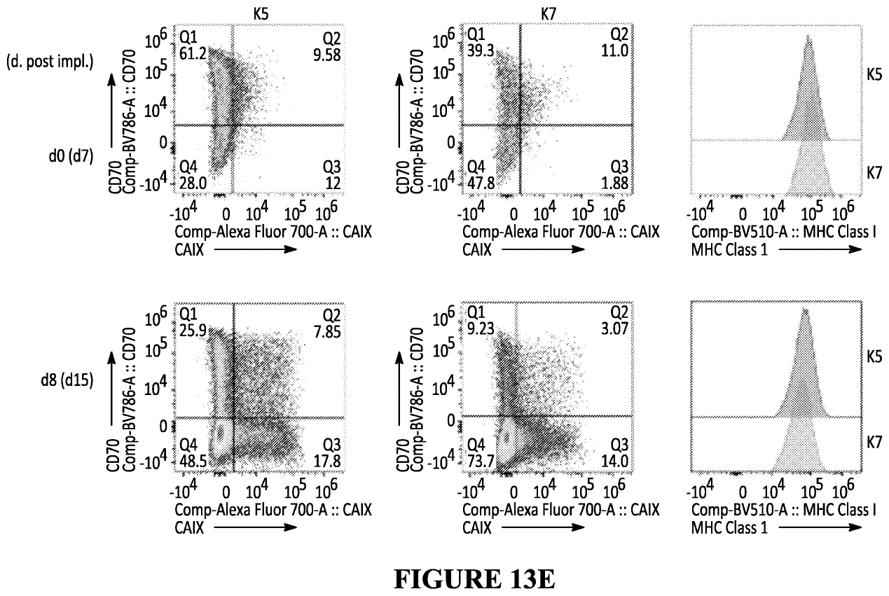

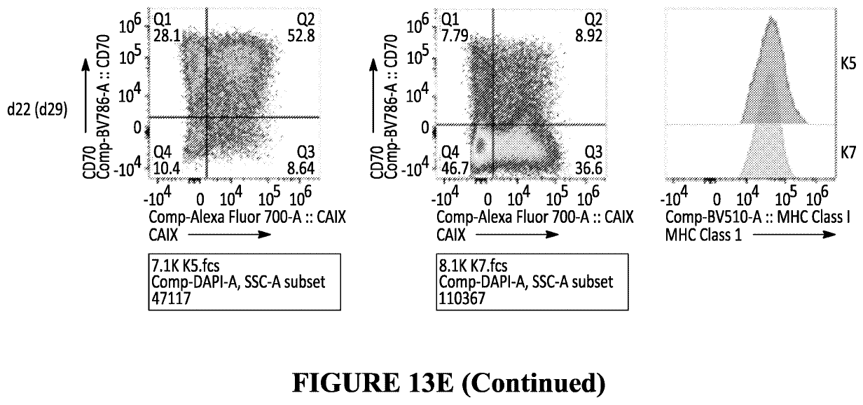

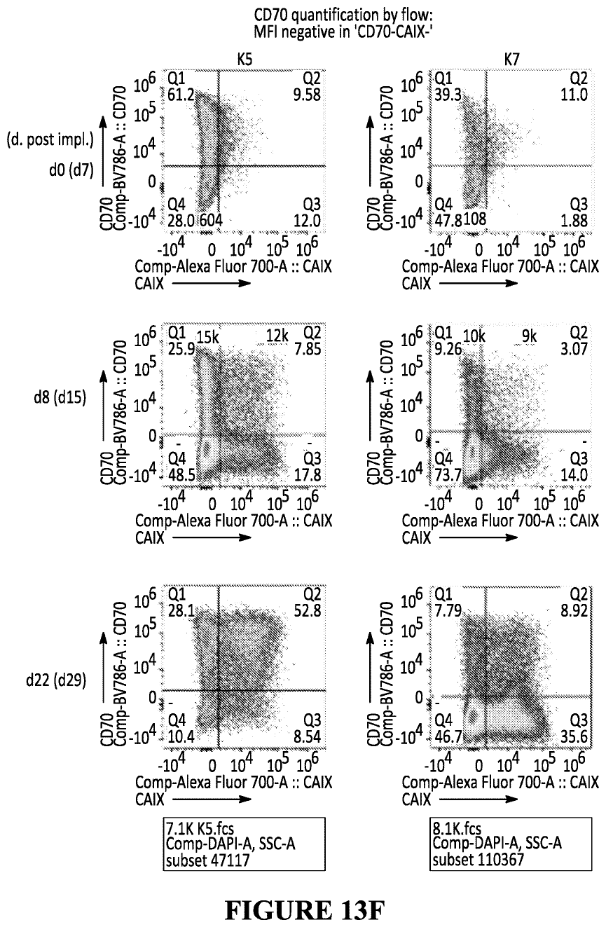

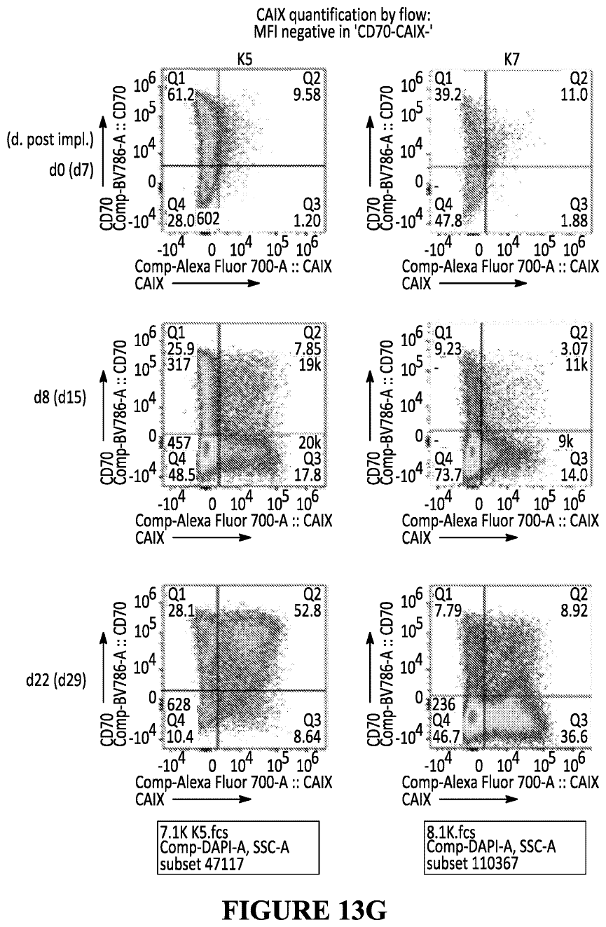

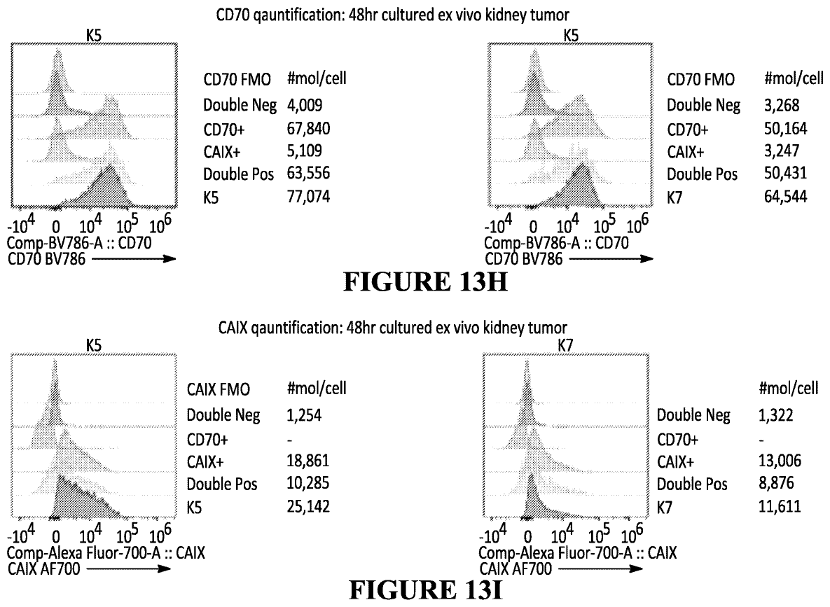

The presently disclosed subject matter discovered that RCC includes tumor cells including “low density antigen” population of cells having CD70 low density (between 500 to 2,000 molecules per cell) that can be targeted by HIT CD70. RNA sequencing showed this “CD70 lo” population of tumor cells which could be a more aggressive fraction of the tumor with cancer stem cell pathways enriched. In addition, in vivo dynamic regulation of CD70 expression with the low population being able to differentiate into the high population. See FIG. 13E.

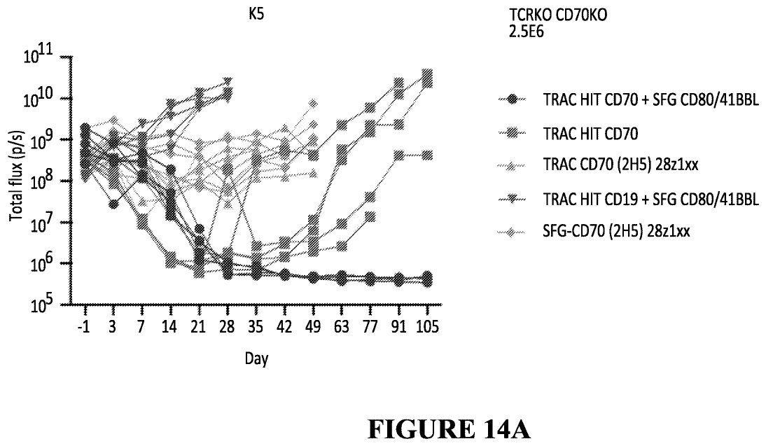

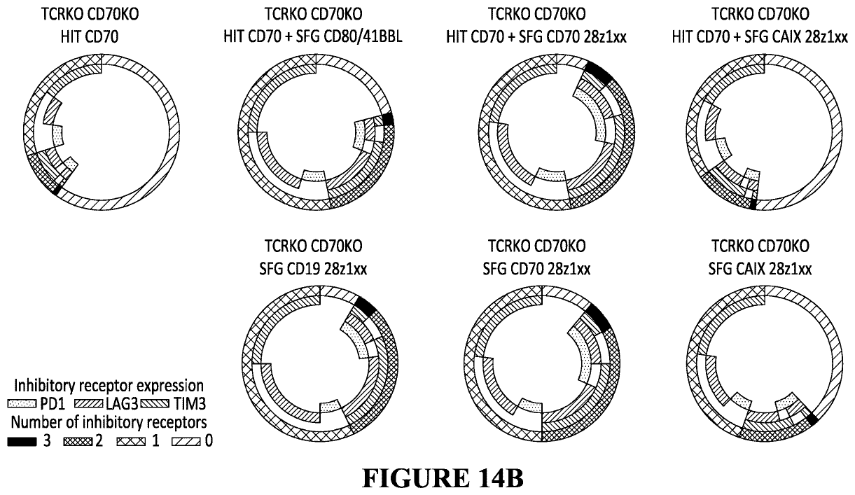

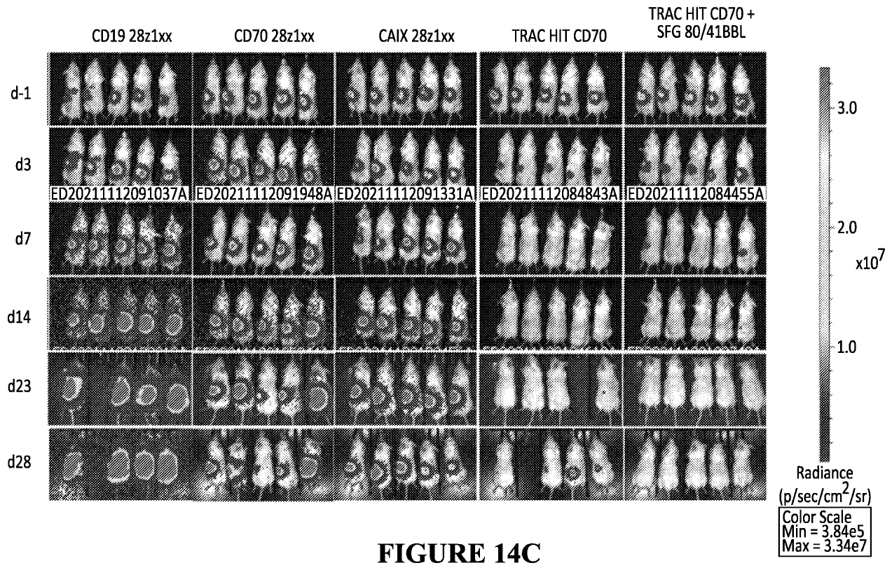

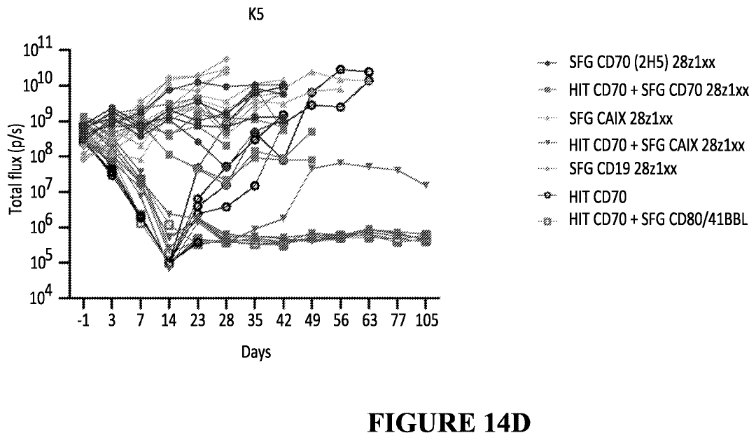

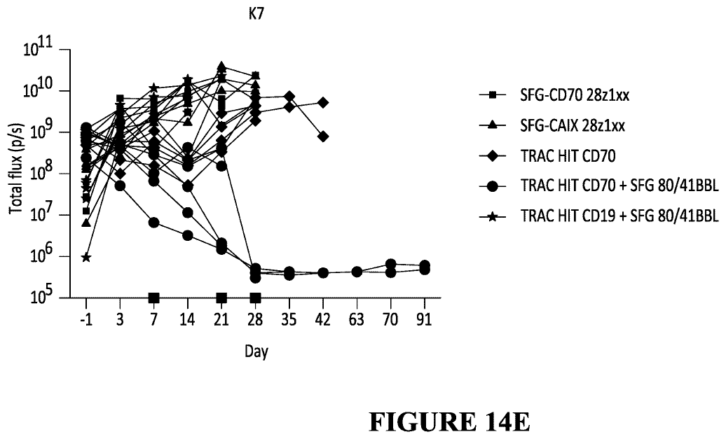

Thus, it was hypothesized that a 70-HIT could completely eradicate the tumor because it is targeting the more aggressive tumor population/cancer stem cell like population. Indeed, as shown in FIGS. 14A-14E, 70-HIT cells co-expressing a co-stimulatory ligand (e.g., including a CD80 polypeptide and a 4-1BBL, disclosed in Section 2.2) were able to address a broad spectrum of expression (e.g., low density antigen). This approach successfully overcome antigen heterogeneity observed in RCC.

The presently disclosed 70-HIT can be used to treat other solid tumors that express CD70 and that have heterogeneous CD70 expression (e.g., glioblastoma, ovarian cancer, and pancreatic cancer).

Here, the presently disclosed subject matter shows that HIT is a superior strategy to CAR to target heterogenous targets in solid tumors. Other tumor targets like PMSA, mesothelin, CEA may now be able to be targeted with the knowledge that the expression is no longer positive vs negative but a spectrum of expression.

In the context of RCC and other solid tumors, HIT+SFG 80/41BBL was effective against K5 and K7 tumors. The presently disclosed 70-HIT co-expressing a co-stimulatory ligand (e.g., including a CD80 polypeptide and a 4-1BBL) represents a safe and effective therapy for RCC and for other solid tumors.

### Example 2

CD70 is a cancer antigen that is expressed on cell surface membrane of clear cell RCC (ccRCC) tumor and not normal kidney (Jilaveanu et al., Hum. Path. 2012). Importantly, its expression is retained in metastatic tissue. However, CD70 expression is heterogeneous. IHC ccRCC tumor microarrays identified 22% of ccRCC cases to have ≥50% tumor cells positive for CD70 expression (Ye et al. J. Clinical Onc. 2022). Thus, the presently disclosed subject matter determined whether CD70 represents an immunotherapeutic target in RCC.

In order to study renal cell carcinoma (RCC), two patient-derived xenograft models (K5 and K7) from clear cell RCC patients were developed. Details of the K5 and K7 cell lines are described in the table below and in FIG. 2A, while expression levels of CD70 are depicted in FIG. 2B.

| | Tumor | | Prior | MSK- |
|---|---|---|---|---|
| Cell line | Site | Histology | therapy | IMPACT |
| K7 | Bone | Clear Cell with | Temsirolimus | 1. ARID5B |
| | Metastasis | Sarcomatoid | Axitinib | 2. NF2 |
| | | Differentiation | Everolimus | 3. NSD1 |
| | | | | 4. PBRM1 |
| | | | | 5. RFWD2 |
| | | | | 6. RPTOR |
| | | | | 7. SETD2 |
| K5 | Bone | Clear Cell | Sunitinib | 1. BAP1 |
| | Metastasis | | Axitinib | 2. BCL2 |
| | | | Nephrectomy | 3. EPHA7 |
| | | | Bevacizumab | 4. HNF1A |
| | | | Everolimus | 5. NCOA3 |
| | | | | 6. PBRM1 |
| | | | | 7. PTEN |
| | | | | 8. VHL |

Injection of these two cell lines in the tail vein led to engraftment in the lungs and later in the liver (FIGS. 3B and 3C).

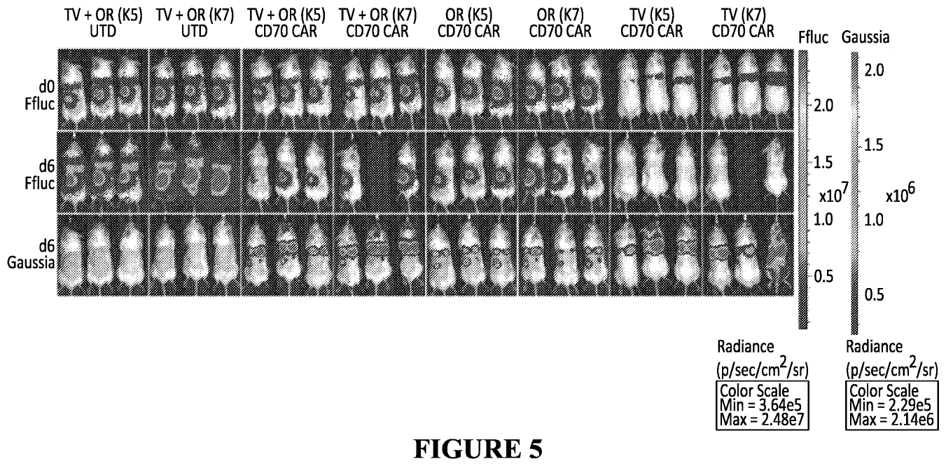

Next, it was determined the ability of T cells expressing an anti-CD70 CAR to kill in vitro K5 and K7 cells. Briefly, T cells were engineered to overexpress a CAR targeting CD70 and including the 1XX domain (see FIG. 2C and Section 2.1 for details). As seen in FIGS. 3B, anti-CD70 CAR T cells were able to induce killing of K5 and K7 cells in vitro. However, while in vivo killing effects of the anti-CD70 CAR T cells were observed in tumors established in lungs, no effect was observed in primary site orthotopic RCC models (FIGS. 4A-4D). FIG. 5 shows that this effect was not due to different ability to reach the tumor site. Thus, differential CD70 expression leads to differential killing between tumor sited.

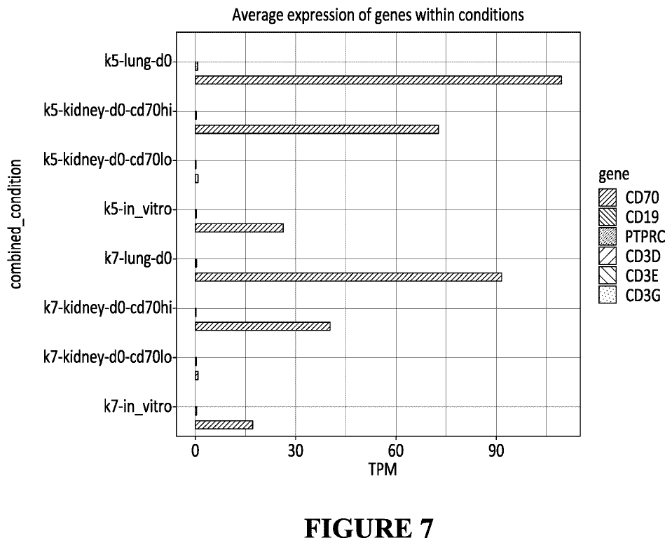

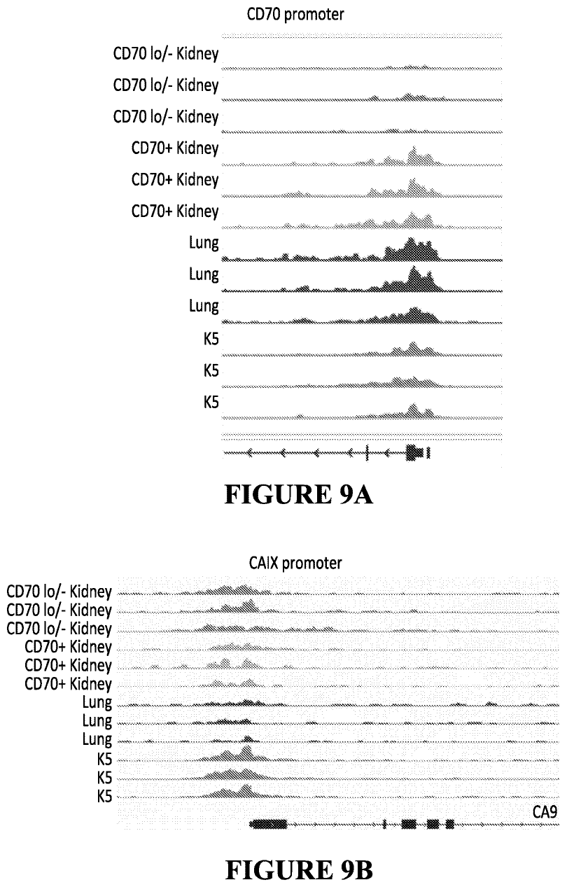

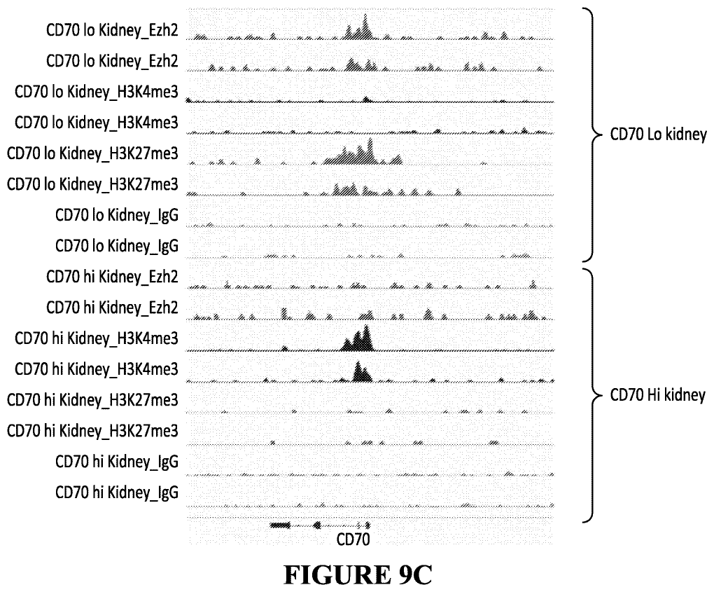

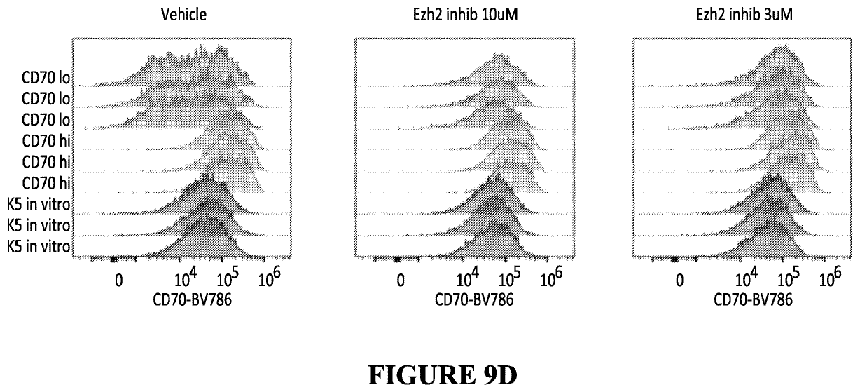

Next, the presently disclosed subject matter determined the mechanisms behind the observed resistance. As seen in FIGS. 4A-4D, tumors established at orthotopic sites were not innately resistant to CAR treatments. Thus, it was hypothesized that CD70 could have had a role in the observed resistance. FACS analysis showed a differential expression of CD70 between untreated lung and kidney tumors established by the K5 and K7 cell lines (FIG. 6). Since these tumors are enriched in cells expressing low levels of CD70 (CD70 lo⁻) (FIG. 7), these cells were cultured in vitro to study their expression profile. Notably, CD70 lo⁻ cells restored the expression of CD70 over time (FIGS. 8A and 8B). Epigenetic analysis showed that CD70 locus but not CAIX promoter locus are epigenetically regulated (FIGS. 9A and 9B) and that the CD70 promoter is accessible by Ezh2 protein (FIG. 9C). Thus, it was determined if the inhibition of Ezh2 could regulate the CD70 expression. As seen in FIG. 9D, treatment with tazemetostat restored the expression level of CD70 in CD70 lo⁻ cells. Thus, the presently disclosed subject matter shows that CD70 expression is epigenetically regulated at different organ sites (e.g., primary vs metastatic) within the same mouse by Ezh2-mediated H3K27me3 inhibition and that in vitro inhibition of Ezh2 leads to CD70 restoration in CD70 lo kidney tumor.

To further confirm the observed data, K5 and K7 cells were engineered to overexpress CD70 and were engrafted to establish tumors. As seen in FIGS. 10D and 10E, overexpression of CD70 in orthotopic models resulted in tumor clearance. Overall, these data show that i) differential CD70 expression leads to differential killing between sites; ii) CD70 expression is epigenetically regulated at different organ sites (primary vs metastatic) within the same mouse by Ezh2-mediated H3K27me3 inhibition; iii) in vitro inhibition of Ezh2 leads to CD70 restoration in CD70 lo kidney tumor; iv) CD70 CAR T cells cannot clear orthotopic site because CD70 is downregulated in vivo below the detection threshold of a CAR T cell; and v) exogenous CD70 overexpression enables CAR killing. Carbonic Anhydrase 9 (CAIX) is a tumor antigen expressed in several solid tumors including, for example, ccRCC, GBM, ovarian, and colorectal cancer (Campos, N.S.P.d et al. Cancers 2022). CAIX is induced by hypoxia and its expression on normal tissues is observed in intrahepatic biliary ducts, gastric mucosa, and duodenum. A first generation anti-CAIX CAR T cell therapy for metastatic ccRCC showed no clinical response and toxicity (Lamers et al. 2016). In view of these features, the role of CAIX and CD70 was further studied.

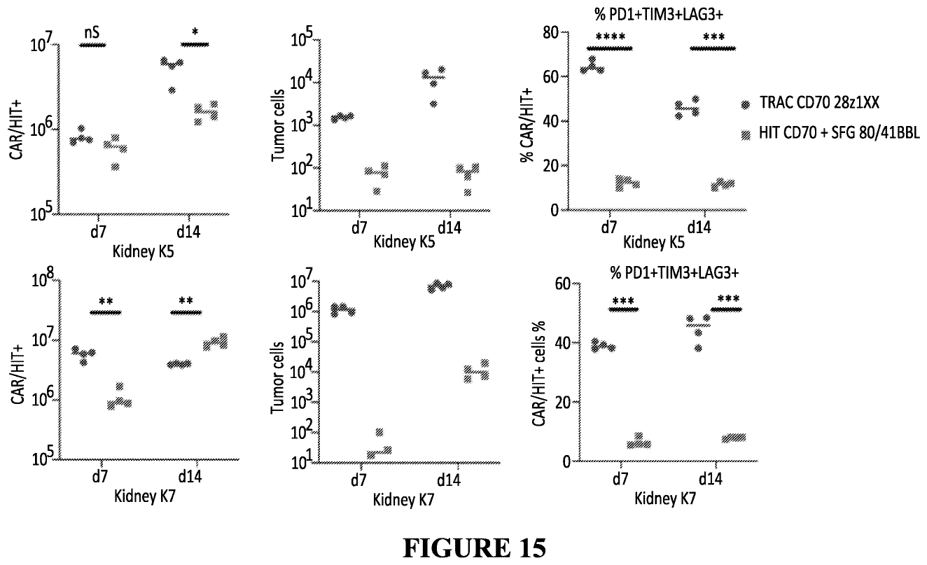

In light of these findings, the inventors of the presently disclosed subject matter wanted to determine whether alternative antigen-recognizing receptors and the inclusion of fusion polypeptides that can provide a co-stimulation signal to the cell could revert the resistance of K5- and K7-derived kidney orthotopic tumors. T cells were engineered to express CAR targeting CD70 and including a 1XX domain or a HIT receptor targeting CD70 using the same antigen-binding fragments of the CAR; in addition, certain T cells were further engineered to include a CD80 polypeptide and a 4-1BBL as disclosed in Section 2.2. As seen in FIGS. 14A-14E and 15, T cell expressing the HIT receptor targeting CD70 and the fusion protein (e.g., one having SEQ ID NO: 76) were able to induce complete response in K5- and K7-derived kidney orthotopic tumors. This effect was due to the exhaustion of CAR T cells as demonstrated by the higher expression profile of PD1, TIM3, and LAG3 observed in CAR T cells as compared to the HIT cells (FIG. 15).

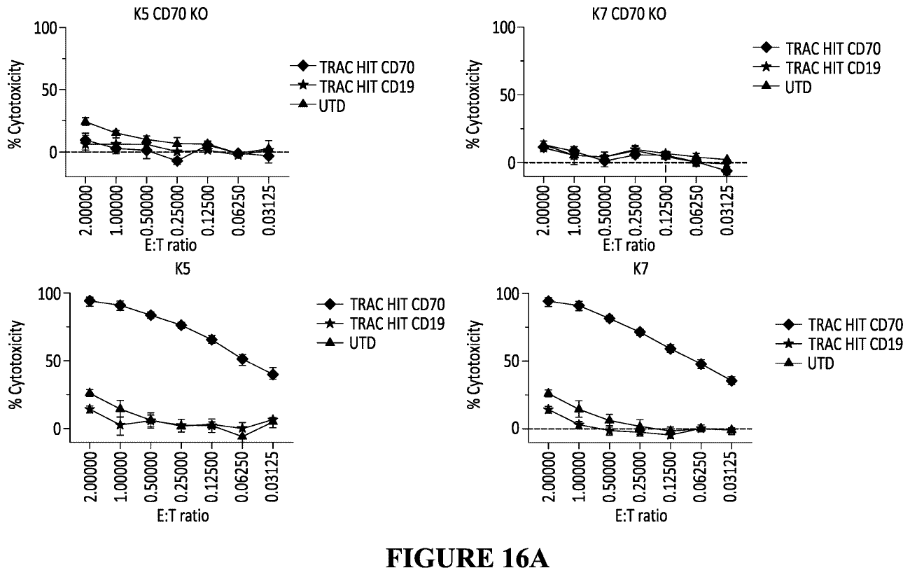

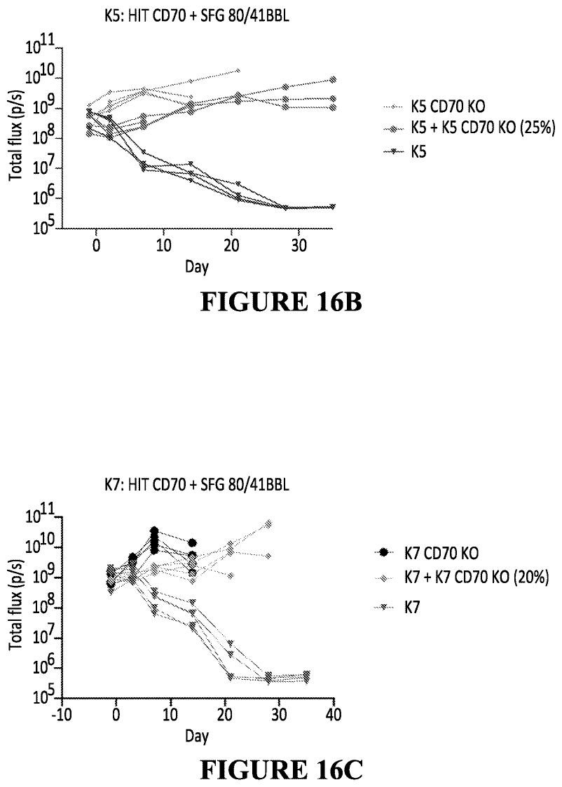

Further, the presently disclosed subject matter determined that HIT CD70 T cell efficacy was not due to bystander killing of CD70 negative tumor (FIGS. 16A-16C) since knocking out CD70 on K5 or K7 PDX lines rendered tumors resistant to HIT CD70 killing. Overall, the presently disclosed data show that i) CD70 expression was not binary but rather a spectrum of expression ranging from high to very low; ii) a highly sensitive CD70 HIT T cell could effectively target this very low CD70 population; iii) CAR T cells were up to 40-50% positive for three markers of exhaustion at day 7 and day 14 compared to less than 15% of HIT T cells; and iv) HIT CD70 T cell efficacy not due to bystander killing of CD70 negative tumor.

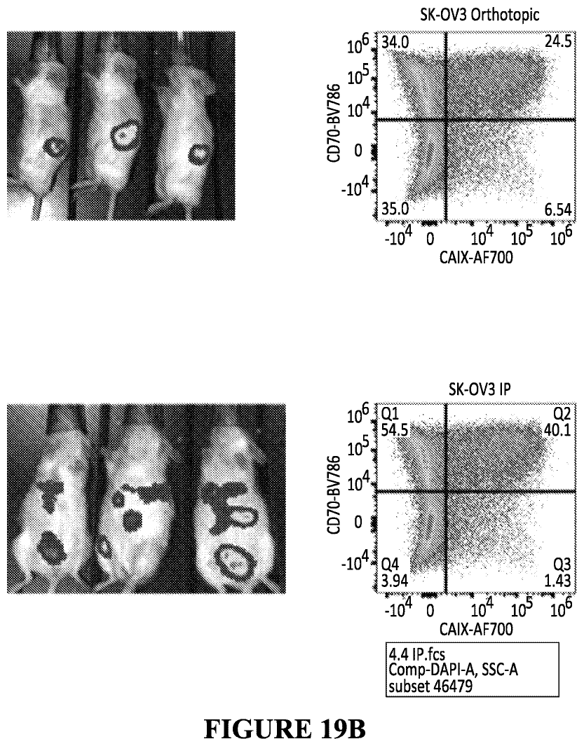

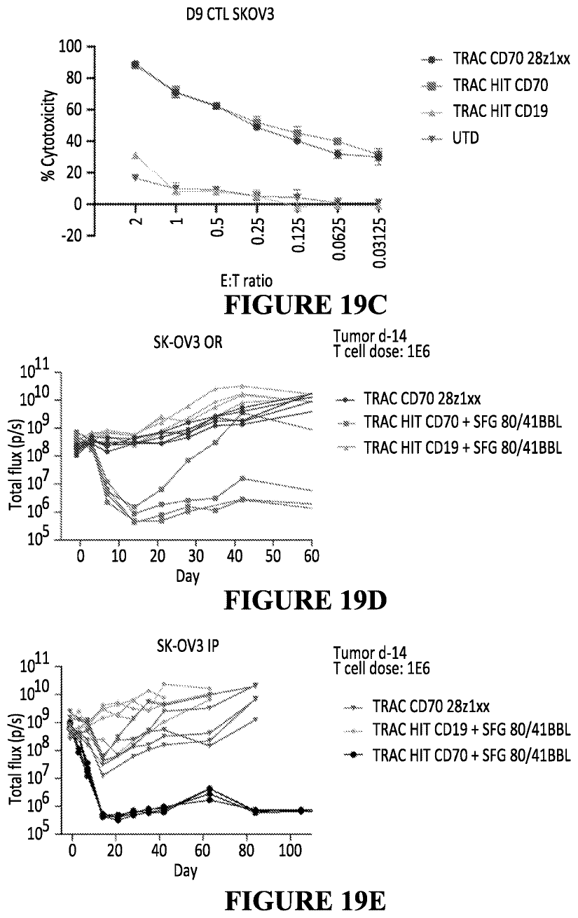

Recently, adoptive cell therapies comprising an anti-CD70 CAR have been developed with response observed only in tumors expressing high levels of CD70 (Srour et al., Cancer Research 83.8_Supplement (2023): CT011-CT011). Therefore, the presently disclosed subject matter studies whether CD70 lo⁻ cells were observed in other tumors. SK-OV3 is a cell line capable of developing ovarian orthotopic and intra-peritoneal cancer that expressed CD70 (FIGS. 19A and 19B). Importantly, administration of cells expressing the HIT receptor targeting CD70 and the fusion protein (e.g., one having SEQ ID NO: 76) were able to induce complete and durable response and to overcome tumor (FIGS. 19C-19E).

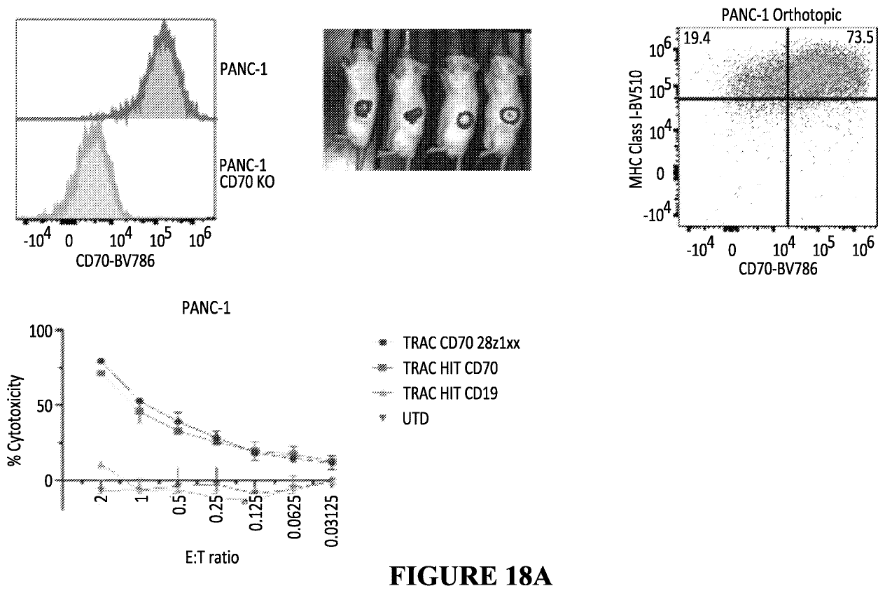

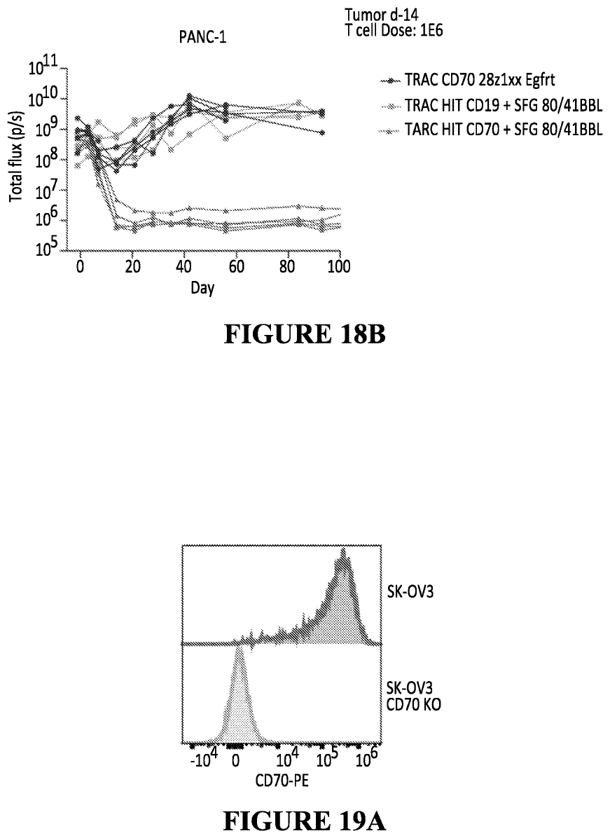

Next, it was determined whether cells expressing the HIT receptor targeting CD70 and the fusion protein (e.g., one having SEQ ID NO: 76) could revert the effect of resistant pancreatic cancers. PANC-1 cell lines were analyzed for their CD70 expression profile and for the ability to establish an orthotopic cancer (FIG. 18A). Notably, cells expressing the HIT receptor targeting CD70 and the fusion protein (e.g., one having SEQ ID NO: 76) were able to induce complete response and to overcome tumor challenging in pancreatic cancer, which was achieved because of the presence of CD70 lo⁻ cells (FIG. 18B). Further, a pancreatic ductal adenocarcinoma PDX (PDAC2) was analyzed for CD70 expression. Notably, in vitro this aggressive pancreatic PDX was heterogenous for CD70 with only approximately 23% positive CD70 expression in vitro and 30% CD70 positive in vivo at orthotopic site in pancreas (FIGS. 17A-17B). Interestingly, cells expressing the HIT receptor targeting CD70 and the fusion protein (e.g., one having SEQ ID NO: 76) were able to induce complete response in the pancreatic PDAC2 orthotopic model, and this effect was specific for CD70 as knocking out CD70 on PDAC2 tumor made tumor resistant to HIT CD70 T cell killing (FIG. 17E). It was confirmed that the CD70 locus was epigenetically regulated in PDAC2 (an alternative pancreatic cancer cell type; see FIGS. 17C and 17D). These data confirm the heterogeneity of CD70 in pancreatic, ovarian, and kidney cancer as well as its epigenetic regulation.

Finally, it was determined whether conventional diagnostic methods (e.g., IHC) could identify these tumors that have CD70 lo⁻ cells. As seen in FIGS. 20A, tumors including CD70 lo⁻ cells resulted negatives to IHC detection highlighting the limitations of conventional diagnostic methods.

In conclusion, the presently disclosed subject matter shows that i) HIT outperforms CAR in RCC, pancreatic and ovarian cancers, which have heterogeneous CD70 expression in vivo; ii) the mechanism of CD70 downregulation by Ezh2 inhibition could be conserved across CD70 heterogeneous tumors; iii) in a CD70 heterogeneous PDAC2 tumor, Ezh2 inhibition in vitro can restore CD70 expression indicating that a partially CD70 positive tumor may belie tumor with very low CD70 expression, which can be unmasked by Ezh2 inhibition; iv) conventional IHC does not capture very low CD70 expression and an RNA FISH approach could be applied as a clinical diagnostic tool; v) it could be necessary to re-stratify patients who might now benefit from treatment who would otherwise have been discounted based on partial/negative CD70 expression; and vi) identification that CD70 negative tumors could have very low CD70 expression opens up the path forward for either a single targeting approach or HIT CD70 as the base in a dual targeting approach.

### EMBODIMENTS OF THE PRESENTLY DISCLOSED SUBJECT MATTER

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method of reducing tumor burden in a subject having a renal cell carcinoma, a pancreatic cancer, or an ovarian cancer, the method comprising administering to the subject an effective amount of a cell comprising a TCR-like fusion molecule that targets CD70.

2. The method of claim 2, wherein the method reduces the number of tumor cells, reduces tumor size, and/or eradicates the tumor in the subject.

3. A method of reducing tumor burden in a subject, the method comprising administering to the subject an effective amount of a cell comprising a TCR-like fusion molecule that targets CD70, wherein the tumor is a renal cell carcinoma, a pancreatic cancer, or an ovarian cancer.

4. The method of claim 3, wherein the method reduces the number of tumor cells, reduces tumor size, and/or eradicates the tumor in the subject.

5. A method of preventing and/or treating a tumor in a subject having a renal cell carcinoma neoplasm, a renal cell carcinoma, a pancreatic cancer, or an ovarian cancer, the method comprising administering to the subject an effective amount of a cell comprising a TCR-like fusion molecule that targets CD70.

6. A method of preventing and/or treating a tumor in a subject, the method comprising administering to the subject an effective amount of a cell comprising a TCR-like fusion molecule that targets CD70, wherein the tumor is a renal cell carcinoma neoplasm, a renal cell carcinoma, a pancreatic cancer, or an ovarian cancer.

7. A method of preventing and/or treating a tumor in a subject in need thereof, the method comprising
a) obtaining a tumor sample that has undetectable CD70 polypeptide levels from the subject;
b) detecting a CD70 polynucleotide by FISH; and
c) administering to the subject an effective amount of a cell comprising a TCR-like fusion molecule that targets CD70 if the CD70 polynucleotide is detected.

8. A method of preventing and/or treating a tumor in a subject in need thereof, the method comprising
a) obtaining a tumor sample that has undetectable CD70 polypeptide levels from the subject;
b) contacting the sample with a Ezh2 inhibitor; and
c) administering to the subject an effective amount of a cell comprising a TCR-like fusion molecule that targets CD70 if the CD70 polypeptide is detected in the sample.

9. The method of any one of claims 1-8, wherein the TCR-like fusion molecule comprises i) a first antigen-binding chain comprising an antigen-binding fragment of a heavy chain variable region (VH) of an antibody; and ii) a second antigen-binding chain comprising an antigen-binding fragment of a light chain variable region (VL) of the antibody; wherein the first and second antigen-binding chains a) each comprise the TRAC polypeptide or the TRBC polypeptide, and b) bind to the second antigen, wherein the TCR-like fusion molecule binds to the second antigen in an HLA-independent manner.

10. The method of claim 9, wherein at least one of the TRAC polypeptide and the TRBC polypeptide is endogenous.

11. The method of claim 9, wherein the first and the second antigen-binding chains bind to the second antigen with a dissociation constant (KD) of about 1×10⁻⁸ M or less.

12. The method of claim 9, wherein the first and the second antigen-binding chains bind to the second antigen with a dissociation constant (KD) of about 5×10⁻⁹ M or less.

13. The method of claim 9, wherein the first antigen-binding chain comprises an antigen-binding fragment of a VH of an antibody and a TRBC polypeptide, and the second antigen-binding chain comprises an antigen-binding fragment of a VL of the antibody and a TRAC polypeptide.

14. The method of claim 9, wherein the first antigen-binding chain comprises an antigen-binding fragment of a VH of an antibody and a TRAC polypeptide, and the second antigen-binding chain comprises an antigen-binding fragment of a VL of the antibody and a TRBC polypeptide.

15. The method of claim 9, wherein
i) the first antigen-binding chain comprises a CDR1 comprising the amino acid sequence set forth in SEQ ID NO: 34, a CDR2 comprising the amino acid sequence set forth in SEQ ID NO: 35, and a CDR3 comprising the amino acid sequence set forth in SEQ ID NO: 36, and the second antigen-binding chain comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 39; or
ii) the first antigen-binding chain comprises a CDR1 comprising the amino acid sequence set forth in SEQ ID NO: 37, a CDR2 comprising the amino acid sequence set forth in SEQ ID NO: 38, and a CDR3 comprising the amino acid sequence set forth in SEQ ID NO: 39, and the second antigen-binding chain comprises a CDR1 comprising the amino acid sequence set forth in SEQ ID NO: 34, a CDR2 comprising the amino acid sequence set forth in SEQ ID NO: 35, and a CDR3 comprising the amino acid sequence set forth in SEQ ID NO: 36.

16. The method of claim 9, wherein
i) the first antigen-binding chain comprises a CDR1, a CDR2, and a CDR3 of the heavy chain variable region set forth in SEQ ID NO: 40, and the second antigen-binding chain comprises a CDR1, a CDR2, and a CDR3 of the light chain variable region set forth in SEQ ID NO: 42; or
ii) the first antigen-binding chain comprises a CDR1, a CDR2, and a CDR3 of the light chain variable region set forth in SEQ ID NO: 42, and the second antigen-binding chain comprises a CDR1, a CDR2, and a CDR3 of the heavy chain variable region set forth in SEQ ID NO: 40.

17. The method of claim 9, wherein
i) the first antigen-binding chain comprises the heavy chain variable region set forth in SEQ ID NO: 40, and the second antigen-binding chain comprises a CDR1, a CDR2, and a CDR3 of the light chain variable region set forth in SEQ ID NO: 42; or
ii) the first antigen-binding chain light chain variable region set forth in SEQ ID NO: 42; and the second antigen-binding chain comprises the heavy chain variable region set forth in SEQ ID NO: 40.

18. The method of claim 9, wherein the first and second antigen binding chains are capable of associating with a CD3ζ polypeptide.

19. The method of claim 18, wherein the first and second antigen binding chains, upon binding to the second antigen, are capable of activating the CD3ζ polypeptide.

20. The method of claim 19, wherein the activation of the CD3ζ polypeptide is capable of activating the cell.

21. The method of claim 9, wherein the cell further comprises a gene disruption of a TRAC locus.

22. The method of claim 9, wherein the cell further comprises a gene disruption of a CD70 locus.

23. The method of claim 9, wherein the cell further comprises a gene disruption of a TRAC locus and a CD70.

24. The method of claim 9, wherein the tumor comprises tumor cells having a CD70 low antigen density.

25. The method of claim 9, wherein the tumor comprises a low frequency of CD70⁺ tumor cells.

26. The method of claim 9, wherein the tumor comprises a CD70 polypeptide that is not detectable by immunodiffusion, immunoelectrophoresis, radioimmunoassay (RIA), enzyme-linked immunosorbent assays (ELISAs), immunofluorescent assays, Western blotting, binder-ligand assays, immunohistochemical techniques, agglutination, complement assays, high performance liquid chromatography (HPLC), thin layer chromatography (TLC), hyperdiffusion chromatography, or a combination thereof.

27. The method of claim 26, wherein the tumor comprises a CD70 polypeptide that is not detectable by immunohistochemistry (IHC).

28. The method of any one of claims 1-27, wherein the cell is a cell of the lymphoid lineage or a cell of the myeloid lineage.

29. The method of claim 28, wherein the cell of the lymphoid lineage is selected from the group consisting of a T cell, a B cell, a Natural Killer (NK) cell, and a dendritic cell.

30. The method of any one of claims 1-29, wherein the cell is a T cell.

31. The method of claim 30, wherein the T cell is derived from an induced pluripotent stem cell.

32. The method of claim 30 or 31, wherein the T cell is a CD8⁺ T cell.

33. The method of claim 32, wherein the CD8⁺ T cell is CD4 independent.

34. The method of any one of claims 30-33, wherein the T cell is selected from the group consisting of a cytotoxic T lymphocyte (CTL), a γδ T cell, a tumor-infiltrating lymphocyte (TIL), a regulatory T cell, and a Natural Killer T (NKT) cell.

35. The method of any one of claims 1-34, wherein the cell further comprises a chimeric antigen receptor (CAR) that targets a second antigen.

36. The method of claim 35, wherein the CAR comprises an extracellular antigen-binding domain that binds to the first antigen, and an intracellular signaling domain that is capable of delivering an activation signal to the cell.

37. The method of claim 36, wherein the intracellular signaling domain of the CAR comprises a CD3ζ polypeptide.

38. The method of claim 37, wherein the CD3ζ polypeptide is a native CD3ζ polypeptide or a modified CD3ζ polypeptide.

39. The method of claim 38, wherein the modified CD3ζ polypeptide comprises a native ITAM1, an ITAM2 variant consisting of two loss-of-function mutations, and an ITAM3 variant consisting of two loss-of-function mutations.

40. The method of any one of claims 36-39, wherein the intracellular signaling domain of the CAR further comprises at least one costimulatory signaling region.

41. The method of claim 40, wherein the at least one costimulatory signaling region comprises at least an intracellular domain of a co-stimulatory molecule or a portion thereof.

42. The method of claim 41, wherein the costimulatory molecule is selected from the group consisting of CD28, 4-1BB, OX40, CD27, CD40, CD154, CD97, CD11a/CD18, ICOS, DAP-10, CD2, CD150, CD226, and NKG2D.

43. The method of any one of claims 36-42, wherein the CAR comprises a transmembrane domain.

44. The method of any one of claims 1-43, wherein the cell further comprises a chimeric co-stimulating receptor (CCR).

45. The method of claim 44, wherein the CCR comprises an extracellular antigen-binding domain that binds to the third antigen and an intracellular domain that is capable of delivering a costimulatory signal to the cell but does not alone deliver an activation signal to the cell.

46. The method of claim 45, wherein the intracellular domain of the CCR comprises at least an intracellular domain of a co-stimulatory molecule or a portion thereof.

47. The method of claim 46, wherein the costimulatory molecule is selected from the group consisting of CD28, 4-1BB, OX40, CD27, CD40, CD154, CD97, CD11a/CD18, ICOS, DAP-10, CD2, CD150, CD226, and NKG2D

48. The method of any one of claims 35-47, wherein the second antigen is a tumor antigen or a pathogen antigen.

49. The method of claim 48, wherein the tumor antigen is selected from the group consisting of CD19, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell (e.g. a cell surface antigen), ANO9, AQP2, ASIC3, ASPRV1, ATP6VOA4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD30, CD300LF, CD312, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-α, FOLR2, FRMD5, GABRB2, GAS2, GD2, GD3, GDPD3, GNA14, GNAZ, GPR153, GPR56, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PR1), PSD2, PTPRJ, RDH16, receptor tyrosine-protein kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, and ZDHHC11.

50. The method of any one of claims 1-49, wherein the cell further comprises at least one exogenous costimulatory ligand.

51. The method of claim 50, wherein the at least one exogenous co-stimulatory ligand is selected from the group consisting of a tumor necrosis factor (TNF) family member, an immunoglobulin (Ig) superfamily member, and combinations thereof.

52. The method of claim 51, wherein the TNF family member is selected from the group consisting of 4-1BBL, OX40L, CD70, FasL, GITRL, TNF-related apoptosis-inducing ligand (TRAIL), CD30L, LIGHT (TNFSF14), CD40L.

53. The method of claim 51 or 52, wherein the Ig superfamily member is selected from the group consisting of CD80, CD86, ICOSLG, and combinations thereof.

54. The method of any one of claims 50-53, wherein the at least one exogenous costimulatory ligand comprises CD80.

55. The method of any one of claims 50-53, wherein the at least one exogenous a costimulatory ligand comprises 4-1BBL.

56. The method of any one of claims 50-53, wherein the cell comprises two exogenous costimulatory ligands.

57. The method of claim 56, wherein the at least two exogenous costimulatory ligands comprise CD80 and 4-1BBL.

58. The method of any one of claims 1-57, wherein the cell further comprises a fusion polypeptide comprising: a) an extracellular domain and a transmembrane domain of a co-stimulatory ligand, and b) an intracellular domain of a first co-stimulatory molecule.

59. The method of claim 58, wherein the co-stimulatory ligand is selected from the group consisting of a tumor necrosis factor (TNF) family member, an immunoglobulin (Ig) superfamily member, and combinations thereof.

60. The method of claim 59, wherein the TNF family member is selected from the group consisting of 4-1BBL, OX40L, CD70, GITRL, CD40L, and combinations thereof.

61. The method of claim 59 or 60, wherein the Ig superfamily member is selected from the group consisting of CD80, CD86, ICOSLG, and combinations thereof.

62. The method of any one of claims 58-61, wherein the co-stimulatory ligand is CD80.

63. The method of any one of claims 58-62, wherein the first co-stimulatory molecule is selected from the group consisting of CD28, 4-1BB, OX40, ICOS, DAP-10, CD27, CD40, NKG2D, CD2, and combinations thereof.

64. The method of claim 63, wherein the first co-stimulatory molecule is 4-1BB.

65. The method of any one of claims 58-64, wherein the co-stimulatory ligand is CD80 and the first co-stimulatory molecule is 4-1BB.

66. The method of any one of claims 58-65, wherein the fusion polypeptide further comprises an intracellular domain of a second co-stimulatory molecule.

67. The method of claim 66, wherein the second co-stimulatory molecule is selected from the group consisting of CD28, 4-1BB, OX40, ICOS, DAP-10, CD27, CD40, NKG2D, CD2, and combinations thereof.

68. The method of claim 66 or 67, wherein the second co-stimulatory molecule is CD28.

69. The method of any one of claims 61-67, wherein the co-stimulatory ligand is CD80, the first co-stimulatory molecule is 4-1BB, and the second co-stimulatory molecule is CD28.

70. The method of any one of claims 1-69, wherein the cell is autologous.

71. The method of any one of claims 1-69, wherein the cell is allogeneic.
